(19) United States
(12) Patent Application Publication
(10) Pub. No.: US 2024/0254515 A1
(43) Pub. Date: Aug. 1, 2024

(54) FLEXIBLE EXPRESSION VECTOR SYSTEMS AND APPLICATION OF SAME TO VACCINES AND IMMUNOTHERAPEUTICS

(71) Applicants: The University Of British Columbia, Vancouver, British Columbia (CA); The James Hutton Institute, Dundee (GB)

(72) Inventors: Wilfred A. JEFFERIES, Surrey, British Columbia (CA); Paolo RIBECA, Edinburgh, Scotland (GB)

(73) Assignees: The University Of British Columbia, Vancouver, British Columbia (CA); The James Hutton Institute, Dundee (GB)

(21) Appl. No.: 18/564,149

(22) PCT Filed: May 26, 2022
(86) PCT No.: PCT/CA2022/050841

§ 371 Date: Nov. 27, 2023

Related U.S. Application Data
(60) Provisional application No. 63/193,177, filed on May 26, 2021.

(51) Int. Cl. C12N 15/86; A61K 39/00; A61K 39/20; A61K 39/215; A61K 48/00; A61P 31/14; A61P 37/04

(52) U.S. Cl. CPC C12N 15/86; A61K 39/20; A61K 39/215; A61K 48/005; A61P 31/14; A61P 37/04; A61K 2039/53; A61K 2039/55555; A61K 2039/575; C12N 2770/20043; C12N 2770/20044; C12N 2770/36143; C12N 2770/36144; C12N 2770/36244

(57) ABSTRACT

The present invention relates to an expression vector that encodes all or a portion of replicon proteins from a positive stranded virus, wherein expression of the replicon proteins is under the control of CMV and T7 promoters, and wherein expression of a payload is under the control of a sub-genomic promoter. Also provided are methods of using the vector in therapeutics and vaccines.

### FIELD OF THE INVENTION

This invention generally pertains to flexible vector systems to express peptides and nucleic acids, and their application to vaccines and immunotherapeutics.

### BACKGROUND OF THE INVENTION

An outbreak of pneumonia like disease termed COVID-19 caused by a novel coronavirus, SARS-COV-2, has spread across the world and become a global pandemic. The COVID-19 pandemic illustrates how essential it is for public health bodies to foster a fast response capability based on technically innovative vaccines. First generation vaccines targeting SARS-CoV-2 have been developed by BioNTech/Pfizer, Moderna, Oxford/Astra Zeneca and others. These first-generation vaccines all target spike protein: the Oxford/Astra Zeneca vaccine uses an adenoviral vector; the vaccines by Moderna and Pfizer are RNA based; the vaccine by Imperial College London relies upon self-amplifying RNA.

These first-generation SARS-COV-2 vaccines, and their fast development cycle (a few months from design to test), were instrumental to lower the burden of the COVID-19 pandemic on healthcare systems and keep mortality figures lower than in the pre-vaccine phase. However, many of these first-generation vaccines have significant weaknesses—mainly the fact they do not induce sterilising immunity (i.e., vaccinated persons are still able to catch and spread the disease) and that emergent COVID-19 strains can escape vaccine immunity.

The Self-amplifying mRNA (SAM) vaccine platform is composed of a non-viral, engineered replicon that drive high levels of expression of encoding antigens. Very low doses are required (mgs) as tens of thousands of copies are made by transfected cells. They may be delivered via intramuscular (i.m.), in the same manner as earlier RNA or DNA vaccines, and can be encapsulated within an adenovirus or another vector to further boost performance. Such vaccines are not only capable of inducing humoral and cellular immunity, but also avoiding the induction of anti-vector immunity, while lacking the risk of genome integration into the host genome. In addition to this, the expression of the antigen caused by the inoculation of mRNA is transient, and thus there are no concerns of T cell exhaustion due to continuous exposure of the antigen. Furthermore, nucleic acid-based vaccine manufacturing is safe and time-saving, and bypasses the need to grow highly pathogenic organisms at a large scale, resulting in a lower risk of contamination with live infectious reagents and accidental release of dangerous pathogens.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An objective of the present invention is to provide flexible expression vector systems and their application to vaccines and immunotherapeutics.

In accordance with an aspect of the invention, there is provided an expression vector that encodes all or a portion of replicon proteins from a positive stranded RNA virus, optionally the vector is a self-amplifying plasmid DNA vector or self-amplifying plasmid RNA vector. In certain embodiments, the expression of the replicon proteins is under the control of CMV and T7 promoters, and wherein expression of a payload is under the control of a sub-genomic promoter. In certain embodiments, the virus is SARS-COV-2, Venezuelan Equine Encephalitis virus (VEEV) or Rubella virus (RUBV). In certain embodiments, the vector encodes replicon proteins from SARS-COV-2 and has the structure set forth in any one of Tables 1 to 4. In certain embodiments, the vector encodes replicon proteins from VEEV and has the structure set forth in Table 5. In certain embodiments, the vector encodes replicon proteins from RUBV and has the structure set forth in Table 6. In certain embodiments, the vector encodes one or more payloads. In certain embodiments, one or more payloads contain a ribosome binding site or other translation initiation sequence, such as a Kozak motif. In certain embodiments, each payload is a collection of peptides. Optionally, the peptides are separated by cleavage motifs for one or more proteases, expresses either by the virus or the host cell. The payload can possibly start with suitable ribosome binding site sequences and possibly contain, for instance at the 5′ and/or 3′ ends, sequences enhancing transcription and/or translation and/or controlling post-translational modifications, for instance localisation in cellular compartments. Optionally the payload has the structure of set forth in any one of Tables 7-10. In certain embodiments, one or more payloads contain sequences enhancing or controlling transcription or translation. In certain embodiments, one or more payloads contain sequences controlling post-translational processing such as localisation in cellular compartments. In certain embodiments, the peptides are separated by protease cleavage motifs and hence subsequently cleaved by either viral or host cell proteases.

In accordance with an aspect of the invention, there is provided a vector having the sequence as set forth in any one of SEQ ID NOs 1 to 12:

In accordance with an aspect of the invention, there is provided a pharmaceutical composition comprising the vector of the present invention and a pharmaceutically acceptable carrier, optionally the vector is formulated in a lipid nanoparticle.

In accordance with an aspect of the invention, there is provided a method of delivering a payload of interest to a cell, the method comprising contacting the cell with the vector of the invention which expresses the payload.

In accordance with an aspect of the invention, there is provided a method of treating, protecting against, and/or preventing disease associated with an infectious agent in a subject, said method comprising administering the vector of the invention, wherein said vector expresses a therapeutic polypeptide or RNA effective against said infectious agent.

In accordance with an aspect of the invention, there is provided a method of stimulating an antigen-specific immune response, said method comprising administering said method comprising administering the vector of the invention, wherein said vector expresses one or more immunogens or epitopes from said infectious agent, optionally the infectious agent is a positive stranded virus and said vector expresses replicon proteins from the same positive stranded virus.

In accordance with another aspect of the invention, there is provided a dual mammalian prokaryotic promoter. In specific embodiments, there is provided a dual promoter CMV and T7. In accordance with another aspect of the invention, there is provided an expression vector system comprises a dual mammalian prokaryotic promoter.

### BRIEF DESCRIPTION OF THE DRAWINGS

These and other features of the invention will become more apparent in the following detailed description in which reference is made to the appended drawings.

FIG. 1 provides a map of a self-amplifying plasmid DNA vector with dual promoter (CMV and T7) and encoding the replicon proteins from the SARS-COV-2 genome of an embodiment of the invention. CMV promoter and T7 promoter will drive synthesis of in vivo or in vitro transcribed mRNA respectively encoding all the replicon proteins necessary for self-amplification of mRNAs. Subsequently, the sub-genomic promoter drives expression of the downstream exemplary payload; GFP by the RNA dependent RNA polymerase from the SARS-COV-2 replicon proteins.

FIG. 2 provides the map of a vector of an embodiment of the present invention based on a partial SARS-COV-2 replicon. In this embodiment, the vector comprises the CMV and T7 promoters and the EGFP gene as an exemplary payload.

FIG. 3 provides the map of a vector of an embodiment of the present invention based on SARS-COV-2 and encodes replicon proteins NSP1 to NSP16. In this embodiment, the vector comprises a multi-cloning site for inserting a sequence encoding the payload.

FIG. 4 provides the map of a vector of an embodiment of the present invention based on SARS-COV-2 and encodes replicon proteins NSP1 to NSP16. In this embodiment, the vector comprises the CMV and T7 promoters and a multi-cloning site for inserting a sequence encoding the payload.

FIG. 5 provides the map of a vector of an embodiment of the present invention based on SARS-COV-2 and encodes replicon proteins NSP1 to NSP16. In this embodiment, the vector comprises the CMV and T7 promoters and the sequence encoding the exemplary payload EGFP.

FIG. 6 provides the map of a vector of an embodiment of the present invention based on a full VEEV replicon. In this embodiment, the vector comprises the CMV and T7 promoters and the EGFP gene as an exemplary payload.

FIG. 7 provides the map of a vector of an embodiment of the present invention based on the VEEV replicon.

FIG. 8 provides the map of a vector of an embodiment of the present invention based on the VEEV replicon and encodes the replicon proteins (NSP1 to NSP4) from the VEE genome. In this embodiment, the vector comprises the CMV and T7 promoters and the EGFP gene as an exemplary payload.

FIG. 9 provides the map of a vector of an embodiment of the present invention based on the VEEV replicon and encodes the replicon proteins (NSP1 to NSP4) from the VEE genome

FIG. 10 provides the map of the self-amplifying (SA) plasmid DNA vector with dual promoter (CMV and T7) and encodes the replicon proteins (NSP1 to NSP4) from the VEE genome. CMV promoter and T7 promoter will drive synthesis of in vivo or in vitro transcribed mRNA respectively encoding all the replicon proteins necessary for self-amplification of mRNAs. Subsequently, the sub-genomic promoter drives expression of the downstream gene; GFP by the RNA dependent RNA polymerase from the VEE replicon proteins.

FIG. 11 provides the map of a vector of an embodiment of the present invention based on SARS-COV-2. In this embodiment, the vector comprises the CBA and T7 promoters and the sequence encoding the exemplary payload EGFP.

FIG. 12 provides the map of a vector of an embodiment of the present invention based on VEE (CBA+T7-Vee-GFP). In this embodiment, the vector comprises the CBA and T7 promoters and the sequence encoding the exemplary payload EGFP.

FIG. 13 provides the map of a vector of an embodiment of the present invention based on SARS-COV-2 (CBA+T7-FullCovid.OUTPUT). In this embodiment, the vector comprises the CBA and T7 promoters and the sequence encoding the exemplary payload EGFP.

FIG. 14 provides time course images after transfection (HEK293+CMV+T7_VEE_EGFP). EGFP positive cells increases in number even until 85 hr—Proves SAM for EGFP and eliminates the need of in vitro transcription by T7 Pol.

FIG. 15 provides molecular biological evidence for SAM by RT-PCR on the mRNA from transfected HEK293 to identify negative strand mRNA for EGFP. TR: mRNA from transfected HEK293 with CMV+T7-Vee_EGFP. IVT: In Vitro transcribed mRNA from CMV+T7-Vee-EGFP; −RT: Without Reverse Transcription; +RT: Reverse transcribed with EGFP FWD primer (5′-CATGAAGCAGCACGACTTCT-3′) and REV primers (5′-CTGCTTGTCGGCCATGATATAG-3′) for TR and IVT samples respectively.

FIG. 16 provides a western blot on HEK293 Cells transfected with Delta variant spike vaccines to validate the protein expression. 1. Cell lysate of HEK 293 cells transfected with the vector having Spike (S1+S2 ECD); 2. Cell lysate of HEK 293 Cells with the vector having Spike (S1+S2 ECD) fused with HLA signal sequence, transmembrane domain and cytoplasmic domain. 3. Cell lysate from HEK 293 cells with the vector having Spike (S1+S2 ECD) fused with Cd74 cytoplasmic domain and HLA transmembrane domain; 4 Protein size marker; 5. Cell lysate from HEK 293 cells transfected with the vector having EGFP gene in the same vector backbone (Negative Control).

FIG. 17 provides a vaccine protocol used for a self-amplifying (sa)DNA vaccine targeting SARS-COV-2 of an embodiment of the invention.

FIG. 18 illustrates the anti-spike ELISA protocol.

FIG. 19 illustrates IgG responses comparing a SaRNA vaccine and a self-amplifying (sa)DNA vaccine targeting SARS-COV-2 of an embodiment of the invention measured on Delta spike plates at day 28 post vaccination.

FIG. 20 illustrates IgA and IgM responses of a self-amplifying (sa)DNA vaccine targeting SARS-COV-2 of an embodiment of the invention (eGFP is the negative control).

FIG. 21 illustrates IgG responses to self-amplifying (sa)RNA vaccines targeting SARS-COV-2 of an embodiment of the invention measured on Delta spike plates.

FIG. 22 illustrates construct characterization using flow.

### DETAILED DESCRIPTION OF THE INVENTION

The present invention provides expression vectors, optionally self-amplifying vectors and the uses of such vectors. The vectors may be utilized in vitro and/or in vivo. In certain embodiments, the vectors are for use in therapeutics, including but not limited to the use of the vectors in vaccines and immunotherapeutics.

Positive stranded viruses, including viruses belonging to the orders Nidovirales, Martellivirales and Hepelivirales are characterized by the presence of (1) a replicon (i.e., a set of genes able to replicate the original RNA genome) which is first expressed as a polyprotein and then cleaved into mature peptides by one or more viral proteases; and (2) a set of (possibly nested) subgenomic RNAs, which encode for a number of structural proteins The number of viral proteases, mature peptides and sub-genomic RNAs varies depending on the virus considered. However, the particular nature and replication strategy of the viruses considered, with the presence of a replicon/payload structure, viral proteases and sub-genomic RNAs, allows for the creation of a derived vector with a doubly configurable mechanism which is particularly well suited to the delivery of peptide-based vaccines.

Accordingly, in some embodiments, the present invention provides expression vectors based on positive stranded viruses, including but not limited to viruses belonging to the orders Nidovirales, Martellivirales and Hepelivirales and uses thereof. In particular, in certain embodiments, the present invention provides a vector, including but not limited to a self-amplifying plasmid DNA vector, that encodes all or a portion of replicon proteins from a positive virus of interest and includes a multi-cloning site to allow insertion of a sequence of a payload of interest.

In some embodiments of the invention, the vector is a plasmid DNA vector encoding the replicon from a positive stranded virus where the expression of the replicon proteins is driven by a eukaryotic promoter.

As used herein, the term promoter includes promoters and promoters plus enhancer elements.

In some embodiments of the invention, the vector is a plasmid DNA vector encoding the replicon from a positive stranded virus where the expression of the replicon proteins is driven by a mammalian promoter.

In some embodiments of the invention, the vector is a plasmid DNA vector encoding the replicon from a positive stranded virus where the expression of the replicon proteins is driven by a eukaryotic promoter and a prokaryotic promoter or a dual eukaryotic prokaryotic promoter. In some embodiments the promoter is a fused dual mammalian prokaryotic promoter.

Accordingly, there is provided a dual mammalian prokaryotic promoter, optionally a fused dual mammalian prokaryotic promoter. In specific embodiments, there is provided a dual promoter CMV and T7. A worker skilled in the art would readily appreciate that such dual promoters may be used in a variety of expression vector systems, including but not limited to expression systems like pox viruses, adenoviruses, lenti, plasmid, transposon etc. Accordingly, in certain embodiments, there is provided a dual promoter for use in expression systems.

In some embodiments of the invention, the vector is a plasmid DNA vector encoding the replicon from a positive stranded virus where the expression of the replicon proteins is driven by a mammalian promoter and a prokaryotic promoter or a dual mammal prokaryotic promoter. In some embodiments the promoter is a fused dual mammalian prokaryotic promoter.

The eukaryotic promoter may be constitutive, inducible or tissue specific. Exemplary eukaryotic promoters include but are not limited to CMV, EF1a, SV40, PGK1 (human or mouse), Ubc, human beta actin, CAG, TRE, UAS, Ac5, Polyhedrin, CaMKlla, GAL1, 10, TEF1, GDS, ADH1, CaMV35S, Ubi, H1 and U6.

Exemplary mammalian promoters include but are not limited to CMV, EF1a, SV40, PGK1, Ubc, human beta actin, CAG, H1 and U6. Exemplary prokaryotic promoters include but are not limited to T7, T7lac, Sp6, araBAD, trp, lac, Ptac and pL.

In certain embodiments, the mammalian promoter is tissue specific. Exemplary tissue specific promoters include but are not limited to B29 promoter, CD14 promoter, CD43 promoter, CD45 promoter, CD68 promoter, Desmin promoter, promoter, Elastase-1 promoter, Endoglin promoter, Fibronectin promoter, Flt-1 promoter, GFAP promoter, GPllb promoter, ICAM-2 promoter, mIFN-β promoter, Mb promoter, Nphsl promoter, OG-2 promoter, SP-B promoter, SYN1 promoter, WASP promoter, SV40/bAlb promoter, SV40/hAlb promoter, SV40/CD43 promoter, SV40/CD45 promoter and NSE/RU5′ promoter.

In specific embodiments, the vector is a DNA plasmid driven by a CMV promoter with or without a T7 promoter. In such embodiments, once the plasmid enters the cell, the plasmid DNA will drive expression of the positive stranded RNA replicon that will in turn drive replication of the negative strand RNA that will begin the self-amplifying mRNA cycle.

In more specific embodiments, the vector is a self-amplifying plasmid DNA vector with dual promoter (CMV and T7) encoding all or a portion of the replicon proteins from the SARS-COV-2 genome. In this embodiment, the CMV promoter and T7 promoter will drive synthesis of in vivo or in vitro transcribed mRNA respectively encoding all the replicon proteins necessary for self-amplification of mRNAs. Subsequently, one or more sub-genomic promoters drive expression of downstream payloads by the RNA dependent RNA polymerase from the SARS-COV-2 replicon proteins.

In other more specific embodiments, the vector is a self-amplifying plasmid DNA vector with dual promoter (CMV and T7) and encoding all or a portion of the replicon proteins from the VEE genome. In this embodiment, the CMV promoter and T7 promoter will drive synthesis of in vivo or in vitro transcribed mRNA respectively encoding all the replicon proteins necessary for self-amplification of mRNAs. Subsequently, one or more sub-genomic promoters drive expression of downstream payloads by the RNA dependent RNA polymerase from the VEE replicon proteins.

In certain embodiments, the self-amplifying plasmid DNA vector comprises the Chicken Beta Actin (CBA) and T7 promoter.

### Order Nidovirales

In certain embodiments of the invention, the vector is derived from viruses belonging to the family Arteriviridae, including but not limited to viruses belonging to the genus Arterivirus. In certain embodiments, the vector is derived from viruses belonging to the family Coronaviridae.

In specific embodiments the vector is derived from viruses belonging to the subfamily Coronavirinae. In more specific embodiments, the vector is derived from viruses belonging to the genuses Alphacoronavirus, Betacoronavirus, Gammacoronavirus, and Deltacoronavirus. In certain embodiments, the vector is derived from viruses belonging to subfamily Torovirinae. In more specific embodiments, the vector is derived from viruses belonging to the genus Torovirus). Other related viruses infecting humans or other organisms targeted by the delivery system may be considered in other embodiments.

In some embodiments, shorter forms of replicons, derived from the original nidoviral replicon by deleting one or more viral genes, are used. In specific embodiments, some shortened replicons have a size similar to, or shorter than, that of alphaviral vectors.

In particular embodiments of the invention, the vector is derived from SARS-COV-2 (the causative agent of COVID-19). The complete genome of SARS-COV-2 is known in the art and is published under GenBank Accession NC_045512 (Nature 579 (7798), 265-269 (2020)). The sequence of variants of SARS-COV-2 are also known in the art.

In certain embodiments, a vaccine vector based on the SARS-COV-2 replicon or portion thereof induces better immunity against SARS-COV-2 than what would be achieved by using a different viral vector.

In certain embodiments of invention, the vector is made of the full viral replicon (i.e., the 5′ leader sequence, followed by the viral replicase gene), followed by the payload, followed by the viral 3′ terminal segment.

In certain embodiments of the invention, the full replicon is the SARS-COV-2 replicon, as per (using the notation employed in GenBank accession NC_045512.2) the following Table 1:

TABLE 1
Structure of a vector based on the full SARS-CoV-2 replicon

| Gene / mature peptide | NC_045512.2 genomic range |
|---|---|
| 5′ UTR | 1 . . . 265 |
| ORF1ab, without stop codon | 266 . . . 21552 |
| Payload | None (user specified) |
| Linker starting with TRS (see next section | 29531 . . . 29557 |
| “Payload formulation”) - begins with stop | |
| codon | |
| ORF10 | 29558 . . . 29674 |
| 3′ UTR | 29675 . . . 29903 |

In other embodiments, the replicon consists of the above without the ORF10 gene (i.e., without viral nucleotides 29558 . . . 29674). In such embodiment the structure of the vector is as follows:

TABLE 2
Structure of a vector based on the SARS-
CoV-2 replicon lacking the ORF10 gene

| Gene / mature peptide | NC_045512.2 genomic range |
|---|---|
| 5′ UTR | 1 . . . 265 |
| ORF1ab, without stop codon | 266 . . . 21552 |
| Payload | None (user specified) |
| Linker starting with TRS (see next section | 29531 . . . 29557 |
| “Payload formulation”) - begins with stop | |
| codon | |
| 3' UTR | 29675 . . . 29903 |

In other embodiments of the invention, the replicon is a shortened SARS-COV-2 replicon whereby the viral genes from nsp2 to nsp4 have been deleted. The sequence of this embodiment in terms of genomic ranges of NC_045512.2 is detailed in the following Table 8a:

TABLE 3
Structure of a vector based on a partial SARS-COV-2 replicon

| Gene/mature peptide | NC_045512.2 genomic range |
|---|---|
| 5′ UTR | 1..265 |
| Leader protein | 266..805 |
| GCTGTTTTGCAG | 10043..10054 |
| (encodes the first 4 AAs of the MRS, see | |
| next section “Payload formulation”) | |
| 3C-like proteinase/Mpro | 10055..10972 |
| nsp6 | 10973..11842 |
| nsp7 | 11843..12091 |
| nsp8 | 12092..12685 |
| nsp9 | 12686..13024 |
| nsp10 | 13025..13441 |
| RNA-dependent RNA polymerase | join (13442..13468, 13468..16236) |
| Helicase | 16237..18039 |
| Exonuclease | 18040..19620 |
| endoRNAse | 19621..20658 |
| 2′-O-ribose-methyltransferase, | 20659..21552 |
| without stop codon | |
| Payload | None (user specified) |
| Linker starting with TRS (see next section | 29531..29557 |
| “Payload formulation”)-begins with stop | |
| codon | |
| ORF10 | 29558..29674 |
| 3′ UTR | 29675..29903 |

In other embodiments, the replicon consists of the above without the ORF10 gene (i.e., without viral nucleotides 29558 . . . 29674). In such embodiments, the structure of the vector is as follows:

TABLE 4
Structure of a vector based on a partial SARS-CoV-2 replicon

| Gene/mature peptide | NC_045512.2 genomic range |
|---|---|
| 5′ UTR | 1..265 |
| Leader protein | 266..805 |
| GCTGTTTTGCAG | 10043..10054 |
| (encodes the first 4 AAs of the MRS, see | |
| next section “Payload formulation”) | |
| 3C-like proteinase/Mpro | 10055..10972 |
| nsp6 | 10973..11842 |
| nsp7 | 11843..12091 |
| nsp8 | 12092..12685 |
| nsp9 | 12686..13024 |
| nsp10 | 13025..13441 |
| RNA-dependent RNA polymerase | join (13442..13468, 13468..16236) |
| Helicase | 16237..18039 |
| Exonuclease | 18040..19620 |
| endoRNAse | 19621..20658 |
| 2′-O-ribose-methyltransferase, | 20659..21552 |
| without stop codon | |
| Payload | None (user specified) |
| Linker starting with TRS (see next section | 29531..29557 |
| “Payload formulation”)-begins with stop | |
| codon | |
| 3′ UTR | 29675..29903 |

Non-limiting exemplary vectors based on SARS-COV-2 are shown in Figures

### Order Martellivirales

In some embodiments of the invention, the vector is derived from viruses belonging to the family Togaviridae, including but not limited to viruses belonging to the genus Alphavirus. In certain embodiments, the virus can be any virus belonging to any of the seven major alphavirus complexes, namely: the Barmah Forest virus complex; the Eastern equine encephalitis complex; the Middelburg virus complex; the Ndumu virus complex; the Semliki Forest virus complex; the Venezuelan equine encephalitis complex; the Western equine encephalitis complex (and/or any other similar virus that should be discovered or classified as belonging to the order Martellivirales in the future). Other related viruses infecting humans or the organism targeted by the delivery system may be considered in other embodiments. In some embodiments, shorter forms of replicons, derived from the original viral replicon by deleting one or more viral genes is used.

In particular embodiments of the invention, the vector is derived from VEEV (the causative agent of Venezuelan Equine Encephalitis). The complete genome of VEEV is known in the art and is published under GenBank Accession NC_001449.

In certain embodiments, a vaccine vector based on the VEEV replicon or portion thereof induces better immunity against VEE than what would be achieved by using a different viral vector.

In certain embodiments of the invention, the vector is made of the full viral replicon (i.e. the 5′ leader sequence, followed by the viral replicase gene), followed by the payload, followed by the viral 3′ terminal segment.

In certain embodiments of the invention, the full replicon is the VEEV replicon, as per (using the notation employed in GenBank accession NC_001449.1) the following Table:

TABLE 5
Structure of a vector based on the full VEEV replicon

| Gene / mature peptide | NC_001449.1 genomic range |
|---|---|
| 5' UTR | 1 . . . 44 |
| NS gene, without TRS (see next section | 45 . . . 7512 |
| “Payload formulation”) | |
| Payload | None |
| 3′ UTR, polyA (includes stop codon) | 11327 . . . 11444 |

Non-limiting exemplary vectors based on VEEV are shown in Figures

### Order Hepelivirales

In certain embodiments of the invention, the vector is derived from viruses belonging to the family Matonaviridae, including but not limited to viruses belonging to the genus Rubivirus. Other related viruses infecting humans or the organism targeted by the delivery system may be considered in other embodiments. In some embodiments, shorter forms of replicons, derived from the original viral replicon by deleting one or more viral genes may be used.

In particular embodiments of the invention, the vector is derived from RUBV (the causative agent of rubella). The complete genome of RUBV is known in the art and is published under GenBank Accession NC_001545.

In certain embodiments, a vaccine vector based on the RUBV replicon induces better immunity against rubella than what would be achieved by using a different viral vector.

In certain embodiments of the invention, the vector is made of the full viral replicon (i.e. the 5′ leader sequence, followed by the viral replicase gene), followed by the payload, followed by the viral 3′ terminal segment. This sequence is only indicative and does not represent the only possibility to embody the idea described in this invention.

In another embodiment, the replicon is obtained by taking the 5′-most part of the virus, up to the viral transcription-regulating sequence for the first sub-genomic mRNA. No 3′ terminal segment is added, in order to increase viral replication in certain situations.

In one embodiment of this invention, the full replicon is the RUBV replicon, as per (using the notation employed in GenBank accession NC_001545.2) the following Table 10:

TABLE 6
Structure of a vector based on the full RUBV replicon

| Gene / mature peptide | NC_001545.2 genomic range |
|---|---|
| 5′ UTR | 1 . . . 40 |
| NS gene, with stop codon | 41 . . . 6390 |
| Non-coding linker between replicon and | 6391 . . . 6407 |
| structural payload genes, without TRS (see | |
| next section “Payload formulation”) | |
| Payload | None (user specified) |
| 3′ UTR, polyA | 9701 . . . 9762 |

### Payload Formulation

The vectors of the present invention may be utilized to express a variety of payloads, including one or more nucleic acids, one or more peptides and one or more polypeptides.

In certain embodiments, the payload is RNA, including but not limited to siRNA and shRNA. In certain embodiments, the payload is one or more polypeptides. The polypeptide(s) may be any polypeptide. Exemplary polypeptides including but not limited to immunogens; epitopes; antibodies, SFv; immunomodulatory molecules including but not limited to cytokines; growth factors; fusion proteins; CRISPR CAS9 or other recombinase system and any other therapeutic proteins.

In certain embodiments, the payload comprises one or more immunogens and/or epitopes alone or in combination with one or more other polypeptides. The one or more immunogens and/or epitopes can be from one or more pathogens or one or more cancer immunogens and/or epitopes.

In certain embodiments, at least one payload is a recombinant protein, siRNA, IncRNA, microRNA or an aptamer. Exemplary proteins include but are not limited to an antibody, Bispecific T Cells Engager (BiTE), nanobody, chemokine, cytokine, growth factor or angiogenesis inhibitors.

In certain embodiments, the payload is a suicide protein. In certain embodiments, the payload is thymidine kinase. In such embodiments, ganciclovir is administered to kill cells expressing thymidine kinase.

A vaccine vector based on a particular viral replicon or portion thereof may induce better immunity against the particular viral pathogen than what would be achieved by using a different viral vector. Accordingly, in certain embodiments, a vector based on a particular viral replicon or portion thereof is utilized to express immunogens and/or epitopes from the same viral pathogen. For example, a viral vector derived from SARS-COV-2 replicon or portion thereof is utilized to express SARS-COV-2 immunogens and/or epitopes; a vector derived from VEEV is utilized to express VEEV immunogens and/or epitopes; a vector derived from RUBV is utilized to express RUBV epitopes; and so on). In other embodiments, the vectors may be utilized to express unrelated immunogens and/or epitopes.

In certain embodiments, the vector is derived from the SARS-COV-2 replicon or portion thereof and expresses one or more immunogens/epitopes from one or more SARS-COV-2 proteins.

Exemplary immunogens/epitopes include immunogens/epitopes from one or more of SARS-CoV2 Spike, N, M, NSP1, NSP2, Proteinase 3CL-Pro, NSP7, NSP8, NSP9, NSP10, helicase, exonuclease, endonuclease, methyltransferase, ORF6, N protein, ORF10, papain-like protease, NSP4, RNA dependent RNA polymerase, ORF7a, ORF8, fragments and variants thereof. In certain embodiments, the one or more SARs-COV-2 proteins comprise Spike protein.

In certain embodiments, the vector is derived from the VEEV replicon or portion thereof and expresses one or more immunogens/epitopes from one or more VEEV proteins.

In certain embodiments, the vector is derived from the RUBV replicon or portion thereof and expresses one or more immunogens/epitopes from one or more RUBV proteins.

In certain embodiments, the payload comprises a collection of peptides. An exemplary method of formulating a payload made of a collection of peptides is as follows: The peptides can be split into subset of peptides, named Subset1, Subset2, etc. In one embodiment of this invention, the total lengths of the peptides in each subset are chosen so as to make the overall lengths of the subsets as close as possible. In other embodiments, the lengths are chosen according to the measured abundances of each subgenomic RNAs produced by the vector of choice, in order to make the number of expressed peptides as balanced as possible.

In one embodiment of the invention, a generic virus belonging to any of the orders Nidovirales, Martellivirlaes, or Hepelivirales is utilized as the source for the vector, as described above. the viral Transcription-Regulation Sequence (TRS) that comes before each viral sub-genomic mRNA, and the amino-acid recognition/cleavage sequence for the main viral protease (Protease Recognition Sequence, PRS) is determined or known in the art. Both sequences depend on the virus of choice; given the sequence of the viral genome, a worker skilled in the art could readily determine the sequences. In some embodiments of the invention, the PRS corresponds to a cleavage sequence for any host-specific endogenous protease. A worker skilled in the could readily determine such sequences.

In certain embodiments, the payload is formulated as per the following Table (Peptide(1,1) denotes the first peptide of the first subset, Peptide(2,1) the second peptide of the first subset, and so on; the last peptide of subset i will be Peptide(nᵢ,i); backtranslate( ) is a function translating a peptide sequence back to DNA, and possibly performing other operations such as codon optimization and removal of spurious signals):

TABLE 7
Structure of a payload for vectors of certain embodiments of the invention

| Element | Note |
|---|---|
| TRS | Beginning of the first artificial subgenomic |
| | mRNA |
| backtranslate(Peptide(1, 1)) | Backtranslation of the first peptide belonging |
| | to subset 1 |
| Backtranslate(PRS) | Backtranslation of the PRS |
| backtranslate(Peptide(1, 2)) | Backtranslation of the second peptide |
| | belonging to subset 1 |
| Backtranslate(PRS) | Backtranslation of the PRS |
| . . . | (more backtranslated peptides separated by |
| | backtranslated PRS) |
| backtranslate(Peptide(1, n₁)) | Backtranslation of the last peptide belonging to |
| | subset 1 |
| TRS | Beginning of the second artificial subgenomic |
| | mRNA |
| backtranslate(Peptide(2, 1)) | Backtranslation of the first peptide belonging |
| | to subset 2 |
| Backtranslate(PRS) | Backtranslation of the PRS |
| backtranslate(Peptide(2, 2)) | Backtranslation of the second peptide |
| | belonging to subset 2 |
| Backtranslate(PRS) | Backtranslation of the PRS |
| . . . | (more backtranslated peptides separated by |
| | backtranslated PRS) |
| backtranslate(Peptide(2, n₂)) | Backtranslation of the last peptide belonging to |
| | subset 2 |
| . . . | (more artificial subgenomic mRNAs) |
| TRS | Beginning of the last artificial subgenomic |
| | mRNA |
| backtranslate(Peptide(m, 1)) | Backtranslation of the first peptide belonging |
| | to the last subset, subset m |
| Backtranslate(PRS) | Backtranslation of the PRS |
| backtranslate(Peptide(m, 2)) | Backtranslation of the second peptide |
| | belonging to the last subset, subset m |
| Backtranslate(PRS) | Backtranslation of the PRS |
| . . . | (more backtranslated peptides separated by |
| | backtranslated PRS) |
| backtranslate(Peptide(m, nₘ)) | Backtranslation of the last peptide belonging to |
| | the last subset, subset m. |

In certain embodiments of the invention, the number of subgenomic mRNAs is close to that of the subgenomic mRNAs present in the virus the vector is derived from.

In another embodiment, the vector is derived from the SARS-COV-2 genome. In such an embodiment, the TRS comprises ACGAAC, and the PRS comprises the motif [AVTP][TKRV]LQ[AS], where letters in square brackets indicate alternative amino acids and the letters are listed in order of decreasing frequency—in specific embodiments the PRS comprises ATLQA. The payload is then formulated in terms of the following Table:

TABLE 8
Structure of a payload for a vector based on SARS-CoV-2 of certain
embodiments of the invention

| Element | Note |
|---|---|
| TAAACGAAC | TRS-Beginning of the first artificial |
| | subgenomic mRNA (includes a leading stop |
| | codon) |
| backtranslate(Peptide(1,1)) | Backtranslation of the first peptide belonging |
| | to subset 1 |
| GCAACCTTACAAGCT | Backtranslation of the PRS, ATLQA |
| backtranslate(Peptide(1,2)) | Backtranslation of the second peptide |
| | belonging to subset 1 |
| GCAACCTTACAAGCT | Backtranslation of the PRS, ATLQA |
| ... | (more backtranslated peptides separated by |
| | backtranslated PRS) |
| backtranslate(Peptide(1,n₁)) | Backtranslation of the last peptide belonging to |
| | subset 1 |
| TAAACGAAC | TRS-Beginning of the second artificial |
| | subgenomic mRNA (includes a leading stop |
| | codon) |
| backtranslate(Peptide(2,1)) | Backtranslation of the first peptide belonging |
| | to subset 2 |
| GCAACCTTACAAGCT | Backtranslation of the PRS, ATLQA |
| backtranslate(Peptide(2,2)) | Backtranslation of the second peptide |
| | belonging to subset 2 |
| GCAACCTTACAAGCT | Backtranslation of the PRS, ATLQA |
| ... | (more backtranslated peptides separated by |
| | backtranslated PRS) |
| backtranslate(Peptide(2,n₂)) | Backtranslation of the last peptide belonging to |
| | subset 2 |
| ... | (more artificial subgenomic mRNAs) |
| TAAACGAAC | TRS-Beginning of the last artificial |
| | subgenomic mRNA (includes a leading stop |
| | codon) |
| backtranslate(Peptide(m, 1)) | Backtranslation of the first peptide belonging |
| | to the last subset, subset m |
| GCAACCTTACAAGCT | Backtranslation of the PRS, ATLQA |
| backtranslate(Peptide(m,2)) | Backtranslation of the second peptide |
| | belonging to the last subset, subset m |
| GCAACCTTACAAGCT | Backtranslation of the PRS, ATLQA |
| ... | (more backtranslated peptides separated by |
| | backtranslated PRS) |
| backtranslate(Peptide(m,nₘ)) | Backtranslation of the last peptide belonging to |
| | the last subset, subset m. |

In another embodiment, the vector is derived from the VEEV genome. In such embodiments, the TRS comprises CTCTCTACGGCTAACCTGAATGGA, and the PRS comprises the motif QEAGAG. The payload is then formulated in terms of the following Table:

TABLE 9
Structure of a payload for a vector based on VEEV of certain embodiments of
the present invention

| Element | Note |
|---|---|
| CTCTCTACGGCTAACCTGAATGGA | TRS-Beginning of the first artificial |
| | subgenomic mRNA (does not include a |
| | leading stop codon) |
| backtranslate(Peptide(1,1)) | Backtranslation of the first peptide belonging |
| | to subset 1 |
| CAAGAGGCTGGGGCCGGC | Backtranslation of the PRS, QEAGAG |
| backtranslate(Peptide(1,2)) | Backtranslation of the second peptide |
| | belonging to subset 1 |
| CAAGAGGCTGGGGCCGGC | Backtranslation of the PRS, QEAGAG |
| ... | (more backtranslated peptides separated by |
| | backtranslated PRS) |
| backtranslate(Peptide(1,n₁)) | Backtranslation of the last peptide belonging to |
| | subset 1 |
| TAACTCTCTACGGCTAACCTGAATGGA | TRS-Beginning of the second artificial |
| | subgenomic mRNA (includes a leading stop |
| | codon) |
| backtranslate(Peptide(2,1)) | Backtranslation of the first peptide belonging |
| | to subset 2 |
| CAAGAGGCTGGGGCCGGC | Backtranslation of the PRS, QEAGAG |
| backtranslate(Peptide(2,2)) | Backtranslation of the second peptide |
| | belonging to subset 2 |
| CAAGAGGCTGGGGCCGGC | Backtranslation of the PRS, QEAGAG |
| ... | (more backtranslated peptides separated by |
| | backtranslated PRS) |
| backtranslate(Peptide(2,n₂)) | Backtranslation of the last peptide belonging to |
| | subset 2 |
| | (more artificial subgenomic mRNAs) |
| TAACTCTCTACGGCTAACCTGAATGGA | TRS-Beginning of the last artificial |
| | subgenomic mRNA (includes a leading stop |
| | codon) |
| backtranslate(Peptide(m, 1)) | Backtranslation of the first peptide belonging |
| | to the last subset, subset m |
| CAAGAGGCTGGGGCCGGC | Backtranslation of the PRS, QEAGAG |
| backtranslate(Peptide(m,2)) | Backtranslation of the second peptide |
| | belonging to the last subset, subset m |
| CAAGAGGCTGGGGCCGGC | Backtranslation of the PRS, QEAGAG |
| ... | (more backtranslated peptides separated by |
| | backtranslated PRS) |
| backtranslate(Peptide(m,nₘ)) | Backtranslation of the last peptide belonging to |
| | the last subset, subset m. |

In yet another embodiment, the vector is derived from the RUBV genome. In such a case, the TRS comprises GCCTTTAATCTTACCTACTCTAACCAGGTCATCACCCAC, and the PRS comprises to the amino acid sequence LALAA, which is compatible with [L][AVS][LS][AG][AQ], the recognition motif for the endogenous eukaryotic signal peptidase I, SPase I. The payload is then formulated in terms of the following Table:

TABLE 10
Structure of a payload for a vector based on RUBV of certain embodiments of
the present invention

| Element | Note |
|---|---|
| GCCTTTAATCTTACCTACTCTAACCAGGTCATCACCCAC | TRS-Beginning of the first |
| | artificial subgenomic mRNA |
| | (does not include a leading |
| | stop codon) |
| backtranslate(Peptide(1,1)) | Backtranslation of the first |
| | peptide belonging to subset |
| | 1 |
| CTGGCCCTGGCCGCC | Backtranslation of the PRS, |
| | LALAA |
| backtranslate(Peptide(1,2)) | Backtranslation of the |
| | second peptide belonging to |
| | subset 1 |
| CTGGCCCTGGCCGCC | Backtranslation of the PRS, |
| | LALAA |
| ... | (more backtranslated |
| | peptides separated by |
| | backtranslated PRS) |
| backtranslate(Peptide(1,n₁)) | Backtranslation of the last |
| | peptide belonging to subset |
| | 1 |
| TAAGCCTTTAATCTTACCTACTCTAACCAGGTCATCACCCAC | TRS-Beginning of the |
| | second artificial subgenomic |
| | mRNA (includes a leading |
| | stop codon) |
| backtranslate(Peptide(2,1)) | Backtranslation of the first |
| | peptide belonging to subset |
| | 2 |
| CTGGCCCTGGCCGCC | Backtranslation of the PRS, |
| | LALAA |
| backtranslate(Peptide(2,2)) | Backtranslation of the |
| | second peptide belonging to |
| | subset 2 |
| CTGGCCCTGGCCGCC | Backtranslation of the PRS, |
| | LALAA |
| ... | (more backtranslated |
| | peptides separated by |
| | backtranslated PRS) |
| backtranslate(Peptide(2,n₂)) | Backtranslation of the last |
| | peptide belonging to subset |
| | 2 |
| ... | (more artificial subgenomic |
| | mRNAs) |
| TAAGCCTTTAATCTTACCTACTCTAACCAGGTCATCACCCAC | TRS-Beginning of the last |
| | artificial subgenomic mRNA |
| | (includes a leading stop |
| | codon) |
| backtranslate(Peptide(m, 1)) | Backtranslation of the first |
| | peptide belonging to the last |
| | subset, subset m |
| CAAGAGGCTGGGGCCGGC | Backtranslation of the PRS, |
| | QEAGAG |
| backtranslate(Peptide(m,2)) | Backtranslation of the |
| | second peptide belonging to |
| | the last subset, subset m |
| CAAGAGGCTGGGGCCGGC | Backtranslation of the PRS, |
| | QEAGAG |
| ... | (more backtranslated |
| | peptides separated by |
| | backtranslated PRS) |
| backtranslate(Peptide(m,nₘ)) | Backtranslation of the last |
| | peptide belonging to the last |
| | subset, subset m. |

In other embodiments, payloads for vectors derived from other viruses can be constructed following the same rules, provided that suitable choices are made for the TRS and the PRS sequence—how to do it will be straightforward to many people skilled in the field.

### Pharmaceutical Compositions and Vaccine Formulations

The present invention further comprises pharmaceutical compositions and vaccine formulations. The pharmaceutical compositions and vaccines formulations may also comprise pharmaceutically acceptable carriers, excipients and/or adjuvants. Adjuvants and carriers suitable for administering genetic vaccines and immunogens are known in the art. Conventional carriers and adjuvants are for example reviewed in Kiyono et al. 1996.

A vaccine adjuvant is a component that potentiates the immune responses to an antigen and/or modulates it towards the desired immune responses. A vaccine may include one or more adjuvants. Exemplary adjuvants include mineral salts including but not limited to aluminium salts (such as amorphous aluminum hydroxyphosphate sulfate (AAHS), aluminum hydroxide, aluminum phosphate, potassium aluminum sulfate (Alum)) and calcium phosphate gels; Oil emulsions and surfactant based formulations, including but not limited to MF59, QS21 (purified saponin), AS02 [SBAS2] (oil-in-water emulsion+MPL+QS-21), Montanide ISA-51 and ISA-720 (immunoprec water-in-oil emulsion); Particulate adjuvants, including but not limited to virosomes (unilamellar liposomal vehicles incorporating influenza haemagglutinin), AS04 ([SBAS4] Al salt with MPL), ISCOMS (structured complex of saponins and lipids), polylactide co-glycolide (PLG). And; microbial derivatives (natural and synthetic), including but not limited to monophosphoryl lipid A (MPL), Detox (MPL+M. phlei cell wall skeleton), AGP [RC-529] (synthetic acylated monosaccharide), DC_Chol (lipoidal immunostimulators able to self mmunopr into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, including but not limited to hGM-CSF or hIL-12 (cytokines that can be administered either as protein or plasmid encoded), Immudaptin (C3d tandem array) and inert vehicles, such as gold particles.

The pharmaceutical compositions and vaccine formulations may also comprise a stabilizer. Suitable stabilizers are known in the art and include but are not limited to amino acids, antioxidants, cyclodextrins, proteins, sugars/sugar alcohols, and surfactants. See for example Morefield, AAPS J 2011 June; 13(2): 191-200; https://www.ncbi.nlm.nih.gov/pmc/articles/PMC3085699/).

The vectors can be incorporated into liposomes, microspheres or other polymer matrices. Liposomes can consist of phospholipids or other lipids, and can be nontoxic, physiologically acceptable and metabolizable carriers that are relatively simple to make and administer.

Previously, it has been found that a SARS-COV-2 SAM lipid nanoparticle (LNP) vaccine induced high neutralizing antibody titers in mice (Mckay et al., Nat Commun 11, 3523 (2020). https://doi.org/10.1038/s41467-020-17409-9). Briefly, the LNP (described in US patent U.S. Pat. No. 10,221,127) contains an ionizable cationic lipid phosphatidylcholine/cholesterol/PEG-lipid. The SAM RNA were encapsulated in LNP using a self-assembly process in which an aqueous solution of SAM RNA at pH=4.0 is rapidly mixed with an ethanolic lipid mixture. LNP.

Accordingly, in certain embodiments, the pharmaceutical compositions and vaccines formulations comprise lipid nanoparticle delivery formulations of vector. Optionally, the lipid is cationic. Appropriate cationic lipids are known in the art. Non-limiting examples include phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA). Also see for example, U.S. Pat. No. 10,221,127 (incorporated by reference) and Reichmuth A M et al. (Therapeutic Delivery. 2016; 7(5):319-334. DOI: 10.4155/tde-2016-0006). In specific embodiments, the LNPs comprise an ionizable cationic lipid (phosphatidylcholine:cholesterol/PEG-lipid (50:10:38.5:1.5 mol/mol). In certain embodiments, the vector to total lipid ratio in the LNP is approximately 0.05 (wt/wt). In certain embodiments, the LNPs have a diameter of ˜80 nm.

In certain embodiments, charge-altering releasable transporters (CARTs) are used to deliver the vectors. In certain embodiments, the vector is formulated as a VLP.

### Methods of Use

The present invention further provides a method of delivering a payload of interest to a cell, the method comprising contacting the cell (either in vitro or in vivo) with a vector of the present invention which expresses the payload. The cell may be a prokaryotic or eukaryotic cell. In certain embodiments, expression of the payload prevents, delays and/or treats disease.

The vector may be administered to a variety of subjects. Including but not limited to prokaryotes and eukaryotes. In certain embodiments, the vector the subject is a human or other animals, including but not limited to other mammals, such as non-human primates, cats, dogs, equines (including but not limited to horses, donkeys and zebras), camels, sheep, goats, and bovines (including but not limited to cows).

In certain embodiments, the vectors of the present invention are used as a vaccine. Accordingly, also provided herein is a method of treating, protecting against, and/or preventing disease associated with the infectious agent in a subject in need thereof by administering the vaccine to the subject. For example, a worker skilled in the art would readily appreciate that a SARS-COV-2 vaccine may be used treating, protecting against, and/or preventing disease associated with SARS-COV-2 (i.e. COVID 19). Administration of the vaccine to the subject can induce or elicit a specific immune response against the vaccine target in the subject.

The induced immune response can be used to treat, prevent, and/or protect against disease related to the vaccine target. For example, a SARS-COV-2 vaccine to the subject can induce or elicit a specific immune response against the SARS-COV-2 virus in the subject. The induced immune response provides the subject administered the vaccine with protection against the vaccine target, such as a SARS-COV-2 vaccine provides resistance to SARS-COV-2.

The induced immune response can include an induced humoral immune response and/or an induced cellular immune response. The induced humoral immune response can include IgG antibodies and/or neutralizing antibodies that are reactive to the antigen. The induced cellular immune response can include a CD8+ T cell response. The number of vaccine doses for effective treatment can be 1, 2, 3, 4, 5, 6, 7, 8, 9, or 10.

The vector can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The vector can be administered prophylactically or therapeutically.

The vector can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). The vector can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vector can be delivered via a variety of routes. Typical delivery routes include parenteral administration, e.g., intradermal, intramuscular or subcutaneous delivery. Other routes include oral administration, intranasal, and intravaginal routes. The vector can be delivered to the interstitial spaces of tissues of an individual (Felgner et al., U.S. Pat. Nos. 5,580,859 and 5,703,055. The vector can also be administered to muscle, or can be administered via intradermal or subcutaneous injections, or transdermally, such as by iontophoresis. Epidermal administration of the vector can also be employed. Epidermal administration can involve mechanically or chemically irritating the outermost layer of epidermis.

The vector can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vector can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vector can be administered via electroporation, such as by a method described in U.S. Pat. No. 7,664,545. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359. The electroporation may be carried out via a minimally invasive device.

The vector may be used in imaging. For example, the vector may express a fluorescent protein.

### EXAMPLES

### Example: Vectors Based on SARS-COV-2 Replicon or Partial Replicon

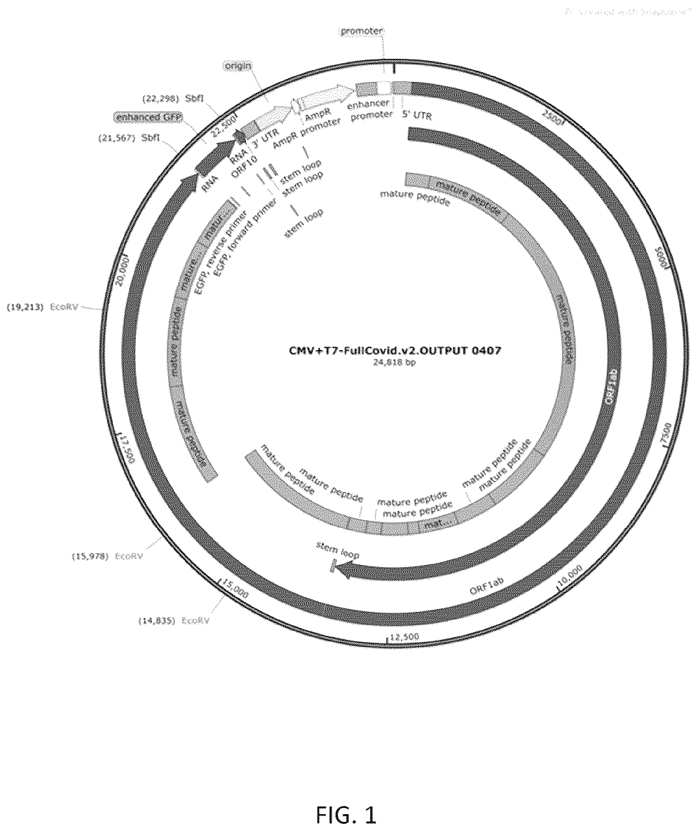

FIG. 1 provides the map of a vector of an embodiment of the present invention based on a SARS-COV-2 replicon, with the EGFP gene as exemplary payload. The vector consists of the ORF1ab gene. The payload consists of the EGFP gene. In addition to the vector and the payload, the construct contains an origin of replication, a bacterial promoter, and an AmpR gene acting as a selection marker, useful when the construct is used as a plasmid; and a human CMV enhancer/promoter, useful when the construct is used as a DNA/RNA vector in humans. The features present in the construct are listed in the following Table:

TABLE
Sequence of a vector based on the full SARS-CoV-2 replicon (in GenBank format)

| | |
|---|---|
| LOCUS | Exported 24818 bp ds-DNA circular SYN 07-APR-2021 |
| DEFINITION | construct. |
| ACCESSION | . |
| VERSION | . |
| KEYWORDS | . |
| SOURCE | synthetic DNA construct |
| ORGANISM | synthetic DNA construct |
| REFERENCE | 1 (bases 1 to 24818) |
| AUTHORS | . |
| TITLE | Direct Submission |
| JOURNAL | Exported Thursday, May 20, 2021 from SnapGene Viewer 5.2.4 |
| | https://www.snapgene.com |
| FEATURES | Location/Qualifiers |
| source | 1..24818 |
| | /organism = ″synthetic DNA construct″ |
| 5′UTR | 1..265 |
| gene | 266..21552 |
| | /gene = ″ORF1ab″ |
| | /locus_tag = ″GU280_gp01″ |
| | /label = ORF1ab |
| CDS | /db_xref = ″GenelD:43740578″ |
| | 266..21552 |
| | /codon_start = 1 |
| | /gene = ″ORF1ab″ |
| | /locus_tag = ″GU280_gp01″ |
| | /product = ″ORF1ab polyprotein″ |
| | /label = ORF1ab |
| | /note = ″/ribosomal_slippage, note = pp1ab; translated by −1 |
| | ribosomal frameshift″ |
| | /db_xref = ″GenelD:43740578″ |
| | /protein_id = ″YP_009724389.1″ |

/translation = ″MESLVPGFNEKTHVQLSLPVLQVRDVLVRGFGDSVEEVLSEARQH
LKDGTCGLVEVEKGVLPQLEQPYVFIKRSDARTAPHGHVMVELVAELEGIQYGRSGETL
GVLVPHVGEIPVAYRKVLLRKNGNKGAGGHSYGADLKSFDLGDELGTDPYEDFQENWNT
KHSSGVTRELMRELNGGAYTRYVDNNFCGPDGYPLECIKDLLARAGKASCTLSEQLDFI
DTKRGVYCCREHEHEIAWYTERSEKSYELQTPFEIKLAKKFDTFNGECPNFVFPLNSII
KTIQPRVEKKKLDGFMGRIRSVYPVASPNECNQMCLSTLMKCDHCGETSWQTGDFVKAT
CEFCGTENLTKEGATTCGYLPQNAVVKIYCPACHNSEVGPEHSLAEYHNESGLKTILRK
GGRTIAFGGCVFSYVGCHNKCAYWVPRASANIGCNHTGVVGEGSEGLNDNLLEILQKEK
VNINIVGDFKLNEEIAIILASFSASTSAFVETVKGLDYKAFKQIVESCGNFKVTKGKAK
KGAWNIGEQKSILSPLYAFASEAARVVRSIFSRTLETAQNSVRVLQKAAITILDGISQY
SLRLIDAMMFTSDLATNNLVVMAYITGGVVQLTSQWLTNIFGTVYEKLKPVLDWLEEKF
KEGVEFLRDGWEIVKFISTCACEIVGGQIVTCAKEIKESVQTFFKLVNKFLALCADSII
IGGAKLKALNLGETFVTHSKGLYRKCVKSREETGLLMPLKAPKEIIFLEGETLPTEVLT
EEVVLKTGDLQPLEQPTSEAVEAPLVGTPVCINGLMLLEIKDTEKYCALAPNMMVTNNT
FTLKGGAPTKVTFGDDTVIEVQGYKSVNITFELDERIDKVLNEKCSAYTVELGTEVNEF
ACVVADAVIKTLQPVSELLTPLGIDLDEWSMATYYLFDESGEFKLASHMYCSFYPPDED
EEEGDCEEEEFEPSTQYEYGTEDDYQGKPLEFGATSAALQPEEEQEEDWLDDDSQQTVG
QQDGSEDNQTTTIQTIVEVQPQLEMELTPVVQTIEVNSFSGYLKLTDNVYIKNADIVEE
AKKVKPTVVVNAANVYLKHGGGVAGALNKATNNAMQVESDDYIATNGPLKVGGSCVLSG
HNLAKHCLHVVGPNVNKGEDIQLLKSAYENFNQHEVLLAPLLSAGIFGADPIHSLRVCV
DTVRTNVYLAVFDKNLYDKLVSSFLEMKSEKQVEQKIAEIPKEEVKPFITESKPSVEQR
KQDDKKIKACVEEVTTTLEETKFLTENLLLYIDINGNLHPDSATLVSDIDITFLKKDAP
YIVGDVVQEGVLTAVVIPTKKAGGTTEMLAKALRKVPTDNYITTYPGQGLNGYTVEEAK
TVLKKCKSAFYILPSIISNEKQEILGTVSWNLREMLAHAEETRKLMPVCVETKAIVSTI
QRKYKGIKIQEGVVDYGARFYFYTSKTTVASLINTLNDLNETLVTMPLGYVTHGLNLEE
AARYMRSLKVPATVSVSSPDAVTAYNGYLTSSSKTPEEHFIETISLAGSYKDWSYSGQS
TQLGIEFLKRGDKSVYYTSNPTTFHLDGEVITFDNLKTLLSLREVRTIKVFTTVDNINL
HTQVVDMSMTYGQQFGPTYLDGADVTKIKPHNSHEGKTFYVLPNDDTLRVEAFEYYHTT
DPSFLGRYMSALNHTKKWKYPQVNGLTSIKWADNNCYLATALLTLQQIELKFNPPALQD
AYYRARAGEAANFCALILAYCNKTVGELGDVRETMSYLFQHANLDSCKRVLNVVCKTCG
QQQTTLKGVEAVMYMGTLSYEQFKKGVQIPCTCGKQATKYLVQQESPFVMMSAPPAQYE
LKHGTFTCASEYTGNYQCGHYKHITSKETLYCIDGALLTKSSEYKGPITDVFYKENSYT
TTIKPVTYKLDGVVCTEIDPKLDNYYKKDNSYFTEQPIDLVPNQPYPNASFDNFKFVCD
NIKFADDLNQLTGYKKPASRELKVTFFPDLNGDVVAIDYKHYTPSFKKGAKLLHKPIVW
HVNNATNKATYKPNTWCIRCLWSTKPVETSNSFDVLKSEDAQGMDNLACEDLKPVSEEV
VENPTIQKDVLECNVKTTEVVGDIILKPANNSLKITEEVGHTDLMAAYVDNSSLTIKKP
NELSRVLGLKTLATHGLAAVNSVPWDTIANYAKPFLNKVVSTTTNIVTRCLNRVCTNYM
PYFFTLLLQLCTFTRSTNSRIKASMPTTIAKNTVKSVGKFCLEASFNYLKSPNFSKLIN
IIIWFLLLSVCLGSLIYSTAALGVLMSNLGMPSYCTGYREGYLNSTNVTIATYCTGSIP
CSVCLSGLDSLDTYPSLETIQITISSFKWDLTAFGLVAEWFLAYILFTRFFYVLGLAAI
MQLFFSYFAVHFISNSWLMWLIINLVQMAPISAMVRMYIFFASFYYVWKSYVHVVDGCN
SSTCMMCYKRNRATRVECTTIVNGVRRSFYVYANGGKGFCKLHNWNCVNCDTFCAGSTF
ISDEVARDLSLQFKRPINPTDQSSYIVDSVTVKNGSIHLYFDKAGQKTYERHSLSHFVN
LDNLRANNTKGSLPINVIVFDGKSKCEESSAKSASVYYSQLMCQPILLLDQALVSDVGD
SAEVAVKMFDAYVNTFSSTFNVPMEKLKTLVATAEAELAKNVSLDNVLSTFISAARQGF
VDSDVETKDVVECLKLSHQSDIEVTGDSCNNYMLTYNKVENMTPRDLGACIDCSARHIN
AQVAKSHNIALIWNVKDFMSLSEQLRKQIRSAAKKNNLPFKLTCATTRQVVNVVTTKIA
LKGGKIVNNWLKQLIKVTLVFLFVAAIFYLITPVHVMSKHTDFSSEIIGYKAIDGGVTR
DIASTDTCFANKHADFDTWFSQRGGSYTNDKACPLIAAVITREVGFVVPGLPGTILRTT
NGDFLHFLPRVFSAVGNICYTPSKLIEYTDFATSACVLAAECTIFKDASGKPVPYCYDT
NVLEGSVAYESLRPDTRYVLMDGSIIQFPNTYLEGSVRVVTTFDSEYCRHGTCERSEAG
VCVSTSGRWVLNNDYYRSLPGVFCGVDAVNLLTNMFTPLIQPIGALDISASIVAGGIVA
IVVTCLAYYFMRFRRAFGEYSHVVAFNTLLFLMSFTVLCLTPVYSFLPGVYSVIYLYLT
FYLTNDVSFLAHIQWMVMFTPLVPFWITIAYIICISTKHFYWFFSNYLKRRVVFNGVSF
STFEEAALCTFLLNKEMYLKLRSDVLLPLTQYNRYLALYNKYKYFSGAMDTTSYREAAC
CHLAKALNDFSNSGSDVLYQPPQTSITSAVLQSGFRKMAFPSGKVEGCMVQVTCGTTTL
NGLWLDDVVYCPRHVICTSEDMLNPNYEDLLIRKSNHNFLVQAGNVQLRVIGHSMQNCV
LKLKVDTANPKTPKYKFVRIQPGQTFSVLACYNGSPSGVYQCAMRPNFTIKGSFLNGSC
GSVGFNIDYDCVSFCYMHHMELPTGVHAGTDLEGNFYGPFVDRQTAQAAGTDTTITVNV
LAWLYAAVINGDRWFLNRFTTTLNDFNLVAMKYNYEPLTQDHVDILGPLSAQTGIAVLD
MCASLKELLQNGMNGRTILGSALLEDEFTPFDVVRQCSGVTFQSAVKRTIKGTHHWLLL
TILTSLLVLVQSTQWSLFFFLYENAFLPFAMGIIAMSAFAMMFVKHKHAFLCLFLLPSL
ATVAYFNMVYMPASWVMRIMTWLDMVDTSLSGFKLKDCVMYASAVVLLILMTARTVYDD
GARRVWTLMNVLTLVYKVYYGNALDQAISMWALIISVTSNYSGVVTTVMFLARGIVFMC
VEYCPIFFITGNTLQCIMLVYCFLGYFCTCYFGLFCLLNRYFRLTLGVYDYLVSTQEFR
YMNSQGLLPPKNSIDAFKLNIKLLGVGGKPCIKVATVQSKMSDVKCTSVVLLSVLQQLR
VESSSKLWAQCVQLHNDILLAKDTTEAFEKMVSLLSVLLSMQGAVDINKLCEEMLDNRA
TLQAIASEFSSLPSYAAFATAQEAYEQAVANGDSEVVLKKLKKSLNVAKSEFDRDAAMQ
RKLEKMADQAMTQMYKQARSEDKRAKVTSAMQTMLFTMLRKLDNDALNNIINNARDGCV
PLNIIPLTTAAKLMVVIPDYNTYKNTCDGTTFTYASALWEIQQVVDADSKIVQLSEISM
DNSPNLAWPLIVTALRANSAVKLQNNELSPVALRQMSCAAGTTQTACTDDNALAYYNTT
KGGRFVLALLSDLQDLKWARFPKSDGTGTIYTELEPPCRFVTDTPKGPKVKYLYFIKGL
NNLNRGMVLGSLAATVRLQAGNATEVPANSTVLSFCAFAVDAAKAYKDYLASGGQPITN
CVKMLCTHTGTGQAITVTPEANMDQESFGGASCCLYCRCHIDHPNPKGFCDLKGKYVQI
PTTCANDPVGFTLKNTVCTVCGMWKGYGCSCDQLREPMLQSADAQSFLNRVCGVSAARL
TPCGTGTSTDVVYRAFDIYNDKVAGFAKFLKTNCCRFQEKDEDDNLIDSYFVVKRHTFS
NYQHEETIYNLLKDCPAVAKHDFFKFRIDGDMVPHISRQRLTKYTMADLVYALRHFDEG
NCDTLKEILVTYNCCDDDYFNKKDWYDFVENPDILRVYANLGERVRQALLKTVQFCDAM
RNAGIVGVLTLDNQDLNGNWYDFGDFIQTTPGSGVPVVDSYYSLLMPILTLTRALTAES
HVDTDLTKPYIKWDLLKYDFTEERLKLFDRYFKYWDQTYHPNCVNCLDDRCILHCANFN
VLFSTVFPPTSFGPLVRKIFVDGVPFVVSTGYHFRELGVVHNQDVNLHSSRLSFKELLV
YAADPAMHAASGNLLLDKRTTCFSVAALTNNVAFQTVKPGNFNKDFYDFAVSKGFFKEG
SSVELKHFFFAQDGNAAISDYDYYRYNLPTMCDIRQLLFVVEVVDKYFDCYDGGCINAN
QVIVNNLDKSAGFPFNKWGKARLYYDSMSYEDQDALFAYTKRNVIPTITQMNLKYAISA
KNRARTVAGVSICSTMTNRQFHQKLLKSIAATRGATVVIGTSKFYGGWHNMLKTVYSDV
ENPHLMGWDYPKCDRAMPNMLRIMASLVLARKHTTCCSLSHRFYRLANECAQVLSEMVM
CGGSLYVKPGGTSSGDATTAYANSVFNICQAVTANVNALLSTDGNKIADKYVRNLQHRL
YECLYRNRDVDTDFVNEFYAYLRKHFSMMILSDDAVVCFNSTYASQGLVASIKNFKSVL
YYQNNVFMSEAKCWTETDLTKGPHEFCSQHTMLVKQGDDYVYLPYPDPSRILGAGCFVD
DIVKTDGTLMIERFVSLAIDAYPLTKHPNQEYADVFHLYLQYIRKLHDELTGHMLDMYS
VMLTNDNTSRYWEPEFYEAMYTPHTVLQAVGACVLCNSQTSLRCGACIRRPFLCCKCCY
DHVISTSHKLVLSVNPYVCNAPGCDVTDVTQLYLGGMSYYCKSHKPPISFPLCANGQVF
GLYKNTCVGSDNVTDFNAIATCDWTNAGDYILANTCTERLKLFAAETLKATEETFKLSY
GIATVREVLSDRELHLSWEVGKPRPPLNRNYVFTGYRVTKNSKVQIGEYTFEKGDYGDA
VVYRGTTTYKLNVGDYFVLTSHTVMPLSAPTLVPQEHYVRITGLYPTLNISDEFSSNVA
NYQKVGMQKYSTLQGPPGTGKSHFAIGLALYYPSARIVYTACSHAAVDALCEKALKYLP
IDKCSRIIPARARVECFDKFKVNSTLEQYVFCTVNALPETTADIVVFDEISMATNYDLS
VVNARLRAKHYVYIGDPAQLPAPRTLLTKGTLEPEYFNSVCRLMKTIGPDMFLGTCRRC
PAEIVDTVSALVYDNKLKAHKDKSAQCFKMFYKGVITHDVSSAINRPQIGVVREFLTRN
PAWRKAVFISPYNSQNAVASKILGLPTQTVDSSQGSEYDYVIFTQTTETAHSCNVNRFN
VAITRAKVGILCIMSDRDLYDKLQFTSLEIPRRNVATLQAENVTGLFKDCSKVITGLHP
TQAPTHLSVDTKFKTEGLCVDIPGIPKDMTYRRLISMMGFKMNYQVNGYPNMFITREEA
IRHVRAWIGFDVEGCHATREAVGTNLPLQLGFSTGVNLVAVPTGYVDTPNNTDFSRVSA
KPPPGDQFKHLIPLMYKGLPWNVVRIKIVQMLSDTLKNLSDRVVFVLWAHGFELTSMKY
FVKIGPERTCCLCDRRATCFSTASDTYACWHHSIGFDYVYNPFMIDVQQWGFTGNLQSN
HDLYCQVHGNAHVASCDAIMTRCLAVHECFVKRVDWTIEYPIIGDELKINAACRKVQHM
VVKAALLADKFPVLHDIGNPKAIKCVPQADVEWKFYDAQPCSDKAYKIEELFYSYATHS
DKFTDGVCLFWNCNVDRYPANSIVCRFDTRVLSNLNLPGCDGGSLYVNKHAFHTPAFDK
SAFVNLKQLPFFYYSDSPCESHGKQVVSDIDYVPLKSATCITRONLGGAVCRHHANEYR
LYLDAYNMMISAGFSLWVYKQFDTYNLWNTFTRLQSLENVAFNVVNKGHFDGQQGEVPV
SIINNTVYTKVDGVDVELFENKTTLPVNVAFELWAKRNIKPVPEVKILNNLGVDIAANT
VIWDYKRDAPAHISTIGVCSMTDIAKKPTETICAPLTVFFDGRVDGQVDLFRNARNGVL
ITEGSVKGLQPSVGPKQASLNGVTLIGEAVKTQFNYYKKVDGVVQQLPETYFTQSRNLQ
EFKPRSQMEIDFLELAMDEFIERYKLEGYAFEHIVYGDFSHSQLGGLHLLIGLAKRFKE
SPFELEDFIPMDSTVKNYFITDAQTGSSKCVCSVIDLLLDDFVEIIKSQDLSVVSKVVK
VTIDYTEISFMLWCKDGHVETFYPKLQSSQAWQPGVAMPNLYKMQRMLLEKCDLQNYGD
SATLPKGIMMNVAKYTQLCQYLNTLTLAVPYNMRVIHFGAGSDKGVAPGTAVLRQWLPT
GTLLVDSDLNDFVSDADSTLIGDCATVHTANKWDLIISDMYDPKTKNVTKENDSKEGFF
TYICGFIQQKLALGGSVAIKITEHSWNADLYKLMGHFAWWTAFVTNVNASSSEAFLIGC
NYLGKPREQIDGYVMHANYIFWRNTNPIQLSSYSLFDMSKFPLKLRGTAVMSLKEGQIN
DMILSLLSKGRLIIRENNRVVISSDVLVNN″

| | |
|---|---|
| CDS | 266..13483 |
| | /codon_start = 1 |
| | /gene = ″ORF1ab″ |
| | /locus_tag = ″GU280_gp01″ |
| | /product = ″ORF1a polyprotein″ |
| | /label = ORF1ab |
| | /note = ″pp1a″ |
| | /db_xref = ″GeneID:43740578″ |
| | /protein_id = ″YP_009725295.1″ |

/translation = ″MESLVPGFNEKTHVQLSLPVLQVRDVLVRGFGDSVEEVLSEARQH
LKDGTCGLVEVEKGVLPQLEQPYVFIKRSDARTAPHGHVMVELVAELEGIQYGRSGETL
GVLVPHVGEIPVAYRKVLLRKNGNKGAGGHSYGADLKSFDLGDELGTDPYEDFQENWNT
KHSSGVTRELMRELNGGAYTRYVDNNFCGPDGYPLECIKDLLARAGKASCTLSEQLDFI
DTKRGVYCCREHEHEIAWYTERSEKSYELQTPFEIKLAKKFDTFNGECPNFVFPLNSII
KTIQPRVEKKKLDGFMGRIRSVYPVASPNECNQMCLSTLMKCDHCGETSWQTGDFVKAT
CEFCGTENLTKEGATTCGYLPQNAVVKIYCPACHNSEVGPEHSLAEYHNESGLKTILRK
GGRTIAFGGCVFSYVGCHNKCAYWVPRASANIGCNHTGVVGEGSEGLNDNLLEILQKEK
VNINIVGDFKLNEEIAIILASFSASTSAFVETVKGLDYKAFKQIVESCGNFKVTKGKAK
KGAWNIGEQKSILSPLYAFASEAARVVRSIFSRTLETAQNSVRVLQKAAITILDGISQY
SLRLIDAMMFTSDLATNNLVVMAYITGGVVQLTSQWLTNIFGTVYEKLKPVLDWLEEKF
KEGVEFLRDGWEIVKFISTCACEIVGGQIVTCAKEIKESVQTFFKLVNKFLALCADSII
IGGAKLKALNLGETFVTHSKGLYRKCVKSREETGLLMPLKAPKEIIFLEGETLPTEVLT
EEVVLKTGDLQPLEQPTSEAVEAPLVGTPVCINGLMLLEIKDTEKYCALAPNMMVTNNT
FTLKGGAPTKVTFGDDTVIEVQGYKSVNITFELDERIDKVLNEKCSAYTVELGTEVNEF
ACVVADAVIKTLQPVSELLTPLGIDLDEWSMATYYLFDESGEFKLASHMYCSFYPPDED
EEEGDCEEEEFEPSTQYEYGTEDDYQGKPLEFGATSAALQPEEEQEEDWLDDDSQQTVG
QQDGSEDNQTTTIQTIVEVQPQLEMELTPVVQTIEVNSFSGYLKLTDNVYIKNADIVEE
AKKVKPTVVVNAANVYLKHGGGVAGALNKATNNAMQVESDDYIATNGPLKVGGSCVLSG
HNLAKHCLHVVGPNVNKGEDIQLLKSAYENFNQHEVLLAPLLSAGIFGADPIHSLRVCV
DTVRTNVYLAVFDKNLYDKLVSSFLEMKSEKQVEQKIAEIPKEEVKPFITESKPSVEQR
KQDDKKIKACVEEVTTTLEETKFLTENLLLYIDINGNLHPDSATLVSDIDITFLKKDAP
YIVGDVVQEGVLTAVVIPTKKAGGTTEMLAKALRKVPTDNYITTYPGQGLNGYTVEEAK
TVLKKCKSAFYILPSIISNEKQEILGTVSWNLREMLAHAEETRKLMPVCVETKAIVSTI
QRKYKGIKIQEGVVDYGARFYFYTSKTTVASLINTLNDLNETLVTMPLGYVTHGLNLEE
AARYMRSLKVPATVSVSSPDAVTAYNGYLTSSSKTPEEHFIETISLAGSYKDWSYSGQS
TQLGIEFLKRGDKSVYYTSNPTTFHLDGEVITFDNLKTLLSLREVRTIKVFTTVDNINL
HTQVVDMSMTYGQQFGPTYLDGADVTKIKPHNSHEGKTFYVLPNDDTLRVEAFEYYHTT
DPSFLGRYMSALNHTKKWKYPQVNGLTSIKWADNNCYLATALLTLQQIELKFNPPALQD
AYYRARAGEAANFCALILAYCNKTVGELGDVRETMSYLFQHANLDSCKRVLNVVCKTCG
QQQTTLKGVEAVMYMGTLSYEQFKKGVQIPCTCGKQATKYLVQQESPFVMMSAPPAQYE
LKHGTFTCASEYTGNYQCGHYKHITSKETLYCIDGALLTKSSEYKGPITDVFYKENSYT
TTIKPVTYKLDGVVCTEIDPKLDNYYKKDNSYFTEQPIDLVPNQPYPNASFDNFKFVCD
NIKFADDLNQLTGYKKPASRELKVTFFPDLNGDVVAIDYKHYTPSFKKGAKLLHKPIVW
HVNNATNKATYKPNTWCIRCLWSTKPVETSNSFDVLKSEDAQGMDNLACEDLKPVSEEV
VENPTIQKDVLECNVKTTEVVGDIILKPANNSLKITEEVGHTDLMAAYVDNSSLTIKKP
NELSRVLGLKTLATHGLAAVNSVPWDTIANYAKPFLNKVVSTTTNIVTRCLNRVCTNYM
PYFFTLLLQLCTFTRSTNSRIKASMPTTIAKNTVKSVGKFCLEASFNYLKSPNFSKLIN
IIIWFLLLSVCLGSLIYSTAALGVLMSNLGMPSYCTGYREGYLNSTNVTIATYCTGSIP
CSVCLSGLDSLDTYPSLETIQITISSFKWDLTAFGLVAEWFLAYILFTRFFYVLGLAAI
MQLFFSYFAVHFISNSWLMWLIINLVQMAPISAMVRMYIFFASFYYVWKSYVHVVDGCN
SSTCMMCYKRNRATRVECTTIVNGVRRSFYVYANGGKGFCKLHNWNCVNCDTFCAGSTF
ISDEVARDLSLQFKRPINPTDQSSYIVDSVTVKNGSIHLYFDKAGQKTYERHSLSHFVN
LDNLRANNTKGSLPINVIVFDGKSKCEESSAKSASVYYSQLMCQPILLLDQALVSDVGD
SAEVAVKMFDAYVNTFSSTFNVPMEKLKTLVATAEAELAKNVSLDNVLSTFISAARQGF
VDSDVETKDVVECLKLSHQSDIEVTGDSCNNYMLTYNKVENMTPRDLGACIDCSARHIN
AQVAKSHNIALIWNVKDFMSLSEQLRKQIRSAAKKNNLPFKLTCATTRQVVNVVTTKIA
LKGGKIVNNWLKQLIKVTLVFLFVAAIFYLITPVHVMSKHTDFSSEIIGYKAIDGGVTR
DIASTDTCFANKHADFDTWFSQRGGSYTNDKACPLIAAVITREVGFVVPGLPGTILRTT
NGDFLHFLPRVFSAVGNICYTPSKLIEYTDFATSACVLAAECTIFKDASGKPVPYCYDT
NVLEGSVAYESLRPDTRYVLMDGSIIQFPNTYLEGSVRVVTTFDSEYCRHGTCERSEAG
VCVSTSGRWVLNNDYYRSLPGVFCGVDAVNLLTNMFTPLIQPIGALDISASIVAGGIVA
IVVTCLAYYFMRFRRAFGEYSHVVAFNTLLFLMSFTVLCLTPVYSFLPGVYSVIYLYLT
FYLTNDVSFLAHIQWMVMFTPLVPFWITIAYIICISTKHFYWFFSNYLKRRVVFNGVSF
STFEEAALCTFLLNKEMYLKLRSDVLLPLTQYNRYLALYNKYKYFSGAMDTTSYREAAC
CHLAKALNDFSNSGSDVLYQPPQTSITSAVLQSGFRKMAFPSGKVEGCMVQVTCGTTTL
NGLWLDDVVYCPRHVICTSEDMLNPNYEDLLIRKSNHNFLVQAGNVQLRVIGHSMQNCV
LKLKVDTANPKTPKYKFVRIQPGQTFSVLACYNGSPSGVYQCAMRPNFTIKGSFLNGSC
GSVGFNIDYDCVSFCYMHHMELPTGVHAGTDLEGNFYGPFVDRQTAQAAGTDTTITVNV
LAWLYAAVINGDRWFLNRFTTTLNDFNLVAMKYNYEPLTQDHVDILGPLSAQTGIAVLD
MCASLKELLQNGMNGRTILGSALLEDEFTPFDVVRQCSGVTFQSAVKRTIKGTHHWLLL
TILTSLLVLVQSTQWSLFFFLYENAFLPFAMGIIAMSAFAMMFVKHKHAFLCLFLLPSL
ATVAYFNMVYMPASWVMRIMTWLDMVDTSLSGFKLKDCVMYASAVVLLILMTARTVYDD
GARRVWTLMNVLTLVYKVYYGNALDQAISMWALIISVTSNYSGVVTTVMFLARGIVFMC
VEYCPIFFITGNTLQCIMLVYCFLGYFCTCYFGLFCLLNRYFRLTLGVYDYLVSTQEFR
YMNSQGLLPPKNSIDAFKLNIKLLGVGGKPCIKVATVQSKMSDVKCTSVVLLSVLQQLR
VESSSKLWAQCVQLHNDILLAKDTTEAFEKMVSLLSVLLSMQGAVDINKLCEEMLDNRA
TLQAIASEFSSLPSYAAFATAQEAYEQAVANGDSEVVLKKLKKSLNVAKSEFDRDAAMQ
RKLEKMADQAMTQMYKQARSEDKRAKVTSAMQTMLFTMLRKLDNDALNNIINNARDGCV
PLNIIPLTTAAKLMVVIPDYNTYKNTCDGTTFTYASALWEIQQVVDADSKIVQLSEISM
DNSPNLAWPLIVTALRANSAVKLQNNELSPVALRQMSCAAGTTQTACTDDNALAYYNTT
KGGRFVLALLSDLQDLKWARFPKSDGTGTIYTELEPPCRFVTDTPKGPKVKYLYFIKGL
NNLNRGMVLGSLAATVRLQAGNATEVPANSTVLSFCAFAVDAAKAYKDYLASGGQPITN
CVKMLCTHTGTGQAITVTPEANMDQESFGGASCCLYCRCHIDHPNPKGFCDLKGKYVQI
PTTCANDPVGFTLKNTVCTVCGMWKGYGCSCDQLREPMLQSADAQSFLNGFAV″

| | |
|---|---|
| mat_peptide | 266..805 |
| | /gene = ″ORF1ab″ |
| | /locus_tag = ″GU280_gp01″ |
| | /product = ″leader protein″ |
| | /note = ″nsp1; produced by both pp1a and pp1ab″ |
| | /note = ″/protein_id = YP_009725297.1″ |
| mat_peptide | 806..2719 |
| | /gene = ″ORF1ab″ |
| | /locus_tag = ″GU280_gp01″ |
| | /product = ″nsp2″ |
| | /note = ″produced by both pp1a and pp1ab″ |
| | /note = ″/protein_id = YP_009725298.1″ |
| mat_peptide | 2720..8554 |
| | /gene = ″ORF1ab″ |
| | /locus_tag = ″GU280_gp01″ |
| | /product = ″nsp3″ |
| | /note = ″former nsp1; conserved domains are: N-terminalacidic |
| | (Ac), predicted phosphoesterase, papain-like proteinase, |
| | Y-domain, transmembrane domain 1 (TM1), adenosine |
| | diphosphate-ribose 1″-phosphatase (ADRP); produced by both |
| | pp1a and pp1ab″ |
| | /note = ″/protein_id = YP_009725299.1″ |
| mat_peptide | 8555..10054 |
| | /gene = ″ORF1ab″ |
| | /locus_tag = ″GU280_gp01″ |
| | /product = ″nsp4″ |
| | /note = ″nsp4B_TM; contains transmembrane domain 2 |
| | (TM2); produced by both pp1a and pp1ab″ |
| | /note = ″/protein_id = YP_009725300.1″ |
| mat_peptide | 10055..10972 |
| | /gene = ″ORF1ab″ |
| | /locus_tag = ″GU280_gp01″ |
| | /product = ″3C-like proteinase″ |
| | /note = ″nsp5A_3CLpro and nsp5B_3CLpro; main |
| | proteinase(Mpro); mediates cleavages downstream of nsp4. 3D |
| | structure of the SARSr-CoV homolog has been determined |
| | (Yang et al., 2003); produced by both pp1a and pp1ab″ |
| | /note = ″/protein_id = YP_009725301.1″ |
| mat_peptide | 10973..11842 |
| | /gene = ″ORF1ab″ |
| | /locus_tag = ″GU280_gp01″ |
| | /product = ″nsp6″ |
| | /note = ″nsp6_TM; putative transmembrane domain; produced |
| | byboth pp1a and pp1ab″ |
| | /note = ″/protein_id = YP_009725302.1″ |
| mat_peptide | 11843..12091 |
| | /gene = ″ORF1ab″ |
| | /locus_tag = ″GU280_gp01″ |
| | /product = ″nsp7″ |
| | /note = ″produced by both pp1a and pp1ab″ |
| | /note = ″/protein_id = YP_009725303.1″ |
| mat_peptide | 12092..12685 |
| | /gene = ″ORF1ab″ |
| | /locus_tag = ″GU280_gp01″ |
| | /product = ″nsp8″ |
| | /note = ″produced by both pp1a and pp1ab″ |
| | /note = ″/protein_id = YP_009725304.1″ |
| mat_peptide | 12686..13024 |
| | /gene = ″ORF1ab″ |
| | /locus_tag = ″GU280_gp01″ |
| | /product = ″nsp9″ |
| | /note = ″ssRNA-binding protein; produced by both pp1a |
| | andpp1ab″ |
| | /note = ″/protein_id = YP_009725305.1″ |
| mat_peptide | 13025..13441 |
| | /gene = ″ORF1ab″ |
| | /locus_tag = ″GU280_gp01″ |
| | /product = ″nsp10″ |
| | /note = ″nsp10_CysHis; formerly known as growth-factor-like |
| | protein (GFL); produced by both pp1a and pp1ab″ |
| | /note = ″/protein_id = YP_009725306.1″ |
| mat_peptide | 13442 .. 16236 |
| | /gene = ″ORF1ab″ |
| | /locus_tag = ″GU280_gp01″ |
| | /product = ″RNA-dependent RNA polymerase″ |
| | /note = ″nsp12; NiRAN and RdRp; produced by pp1ab only″ |
| | /note = ″/protein_id = YP_009725307.1″ |
| mat_peptide | 13442..13480 |
| | /gene = ″ORF1ab″ |
| | /locus_tag = ″GU280_gp01″ |
| | /product = ″nsp11″ |
| | /note = ″produced by pp1a only″ |
| | /note = ″/protein_id = YP_009725312.1″ |
| stem_loop | 13476..13503 |
| | /gene = ″ORF1ab″ |
| | /locus_tag = ″GU280_gp01″ |
| | /function = ″Coronavirus frameshifting stimulation |
| | elementstem-loop 1″ |
| | /inference = ″COORDINATES:same species:Rfam-release-14.1″ |
| stem_loop | 13488..13542 |
| | /gene = ″ORF1ab″ |
| | /locus_tag = ″GU280_gp01″ |
| | /function = ″Coronavirus frameshifting stimulation |
| | elementstem-loop 2″ |
| | /inference = ″COORDINATES:same species:profile″ |
| mat_peptide | 16237..18039 |
| | /gene = ″ORF1ab″ |
| | /locus_tag = ″GU280_gp01″ |
| | /product = ″helicase″ |
| | /note = ″nsp13_ZBD, nsp13_TB, and nsp_HEL1core; zinc-binding |
| | domain (ZD), NTPase/helicase domain (HEL), RNA |
| | 5′-triphosphatase; produced by pp1ab only″ |
| | /note = ″/protein_id = YP_009725308.1″ |
| mat_peptide | 18040..19620 |
| | /gene = ″ORF1ab″ |
| | /locus_tag = ″GU280_gp01″ |
| | /product = ″3′-to-5′ exonuclease″ |
| | /note = ″nsp14A2_ExoN and nsp14B_NMT; produced by pp1abonly″ |
| | /note = ″/protein_id = YP_009725309.1″ |
| mat_peptide | 19621 .. 20658 |
| | /gene = ″ORF1ab″ |
| | /locus_tag = ″GU280_gp01″ |
| | /product = ″endoRNAse″ |
| | /note = ″nsp15-A1 and nsp15B-NendoU; produced by pp1ab only″ |
| | /note = ″/protein_id = YP_009725310.1″ |
| mat_peptide | 20659..21552 |
| | /gene = ″ORF1ab″ |
| | /locus_tag = ″GU280_gp01″ |
| | /product = ″2′-O-ribose methyltransferase″ |
| | /note = ″nsp16_OMT; 2′-o-MT; produced by pp1ab only″ |
| | /note = ″/protein_id = YP_009725311.1″ |
| misc_feature | 21553..21561 |
| | /label = nonstandard type: RNA |
| | /note = ″Transcription regulation sequence-begins withstop |
| | codon″ |
| regulatory | 21570..21579 |
| | /regulatory_class = ″other″ |
| | /note = ″vertebrate consensus sequence for strong |
| | initiationof translation (Kozak, 1987)-likely unnecessary |
| | if used with Covid vector″ |
| CDS | 21576 .. 22292 |
| | /codon_start = 1 |
| | /product = ″enhanced GFP″ |
| | /label = enhanced GFP |
| | /note = ″mammalian codon-optimized, without stop codon″ |

/translation = ″MVSKGEELFTGVVPILVELDGDVNGHKFSVSGEGEGDATYGKLTL
KFICTTGKLPVPWPTLVTTLTYGVQCFSRYPDHMKQHDFFKSAMPEGYVQERTIFFKDD
GNYKTRAEVKFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPNEKRDHMVLL
EFVTAAGITLGMDELYK″

| | |
|---|---|
| primer_bind | complement(21621..21642) |
| | /label = EGFP, reverse primer |
| | /note = ″EGFP, reverse primer″ |
| primer_bind | complement(21882..21901) |
| | /label = For distinguishing EGFP variants, reverse primer |
| | /note = ″For distinguishing EGFP variants, reverse primer″ |
| primer_bind | 22229..22250 |
| | /label = EGFP, forward primer |
| | /note = ″EGFP, forward primer″ |
| misc_feature | 22302..22328 |
| | /label = nonstandard type: RNA |
| | /note = ″linker starting with TRS (begins with stop codon)″ |
| gene | 22329..22445 |
| | /gene = ″ORF10″ |
| | /locus_tag = ″GU280_gp11″ |
| | /label = ORF10 |
| | /db_xref = ″GenelD:43740576″ |
| CDS | 22329..22445 |
| | /codon_start = 1 |
| | /gene = ″ORF10″ |
| | /locus_tag = ″GU280_gp11″ |
| | /product = ″ORF10 protein″ |
| | /label = ORF10 |
| | /db_xref = ″GeneID:43740576″ |
| | /protein_id = ″YP_009725255.1″ |
| | /translation = ″MGYINVFAFPFTIYSLLLCRMNSRNYIAQVDVVNFNLT″ |
| stem_loop | 22380 .. 22415 |
| | /gene = ″ORF10″ |
| | /locus_tag = ″GU280_gp11″ |
| | /function = ″Coronavirus 3′ UTR pseudoknot stem-loop 1″ |
| | /inference = ″COORDINATES:same species: Rfam-release-14.1″ |
| stem_loop | 22400..22428 |
| | /gene = ″ORF10″ |
| | /locus_tag = ″GU280_gp11″ |
| | /function = ″Coronavirus 3′ UTR pseudoknot stem-loop 2″ |
| | /inference = ″COORDINATES:same species: Rfam-release-14.1″ |
| 3′UTR | 22446..22674 |
| stem_loop | 22499..22539 |
| | /function = ″Coronavirus 3′ stem-loop II-like motif (s2m)″ |
| | /note = ″basepair exception: alignment to the Rfam |
| | modelimplies coordinates 29740:29758 form a noncanonical |
| | C:T basepair, but the homologous positions form a highly |
| | conserved C:G basepair in other viruses, including SARS |
| | (NC_004718.3)″ |
| | /inference = ″COORDINATES:same species:Rfam-release-14.1″ |
| rep_origin | 22683..23271 |
| | /direction = RIGHT |
| | /note = ″high-copy-number ColE1/pMB1/pBR322/pUC origin of |
| | replication″ |
| primer_bind | 23172..23191 |
| | /label = pBR322 origin, forward primer |
| | /note = ″pBR322 origin, forward primer″ |
| promoter | 23272..23376 |
| | /gene = ″bla″ |
| | /label = AmpR promoter |
| CDS | 23377..24237 |
| | /codon_start = 1 |
| | /gene = ″bla″ |
| | /product = ″beta-lactamase″ |
| | /label = AmpR |
| | /note = ″confers resistance to ampicillin, carbenicillin, and |
| | related antibiotics″ |

/translation = ″MSIQHFRVALIPFFAAFCLPVFAHPETLVKVKDAEDQLGARVGYI
ELDLNSGKILESFRPEERFPMMSTFKVLLCGAVLSRVDAGQEQLGRRIHYSQNDLVEYS
PVTEKHLTDGMTVRELCSAAITMSDNTAANLLLTTIGGPKELTAFLHNMGDHVTRLDRW
EPELNEAIPNDERDTTMPVAMATTLRKLLTGELLTLASRQQLIDWMEADKVAGPLLRSA
LPAGWFIADKSGAGERGSRGIIAALGPDGKPSRIVVIYTTGSQATMDERNRQIAEIGAS
LIKHW″

| | |
|---|---|
| enhancer | 24266..24569 |
| | /note = ″human cytomegalovirus immediate early enhancer″ |
| promoter | 24570..24773 |
| promoter | /note = ″human cytomegalovirus (CMV) immediate earlypromoter″ |
| | 24800..24818 |
| | /note = ″T7 promoter″ |

The DNA sequence of the construct is listed in the following Table:

Sequence of a vector based on the full SARS-COV-2 replicon

| | Nucleic Acid Sequence (Description of what is encoded. Sequence | |
|---|---|---|
| | represented as DNA for corresponding RNA sequence T is replaced with | |
| Sequence | U). The number at the beginning of each line represents the coordinate (1- | |
| ID number | based) of the first nucleotide in that line | |
| 1 | CMV + T7_FullCOVID | |
| | 1 | attaaaggtt tataccttcc caggtaacaa accaaccaac tttcgatctc ttgtagatct |
| | 61 | gttctctaaa cgaactttaa aatctgtgtg gctgtcactc ggctgcatgc ttagtgcact |
| | 121 | cacgcagtat aattaataac taattactgt cgttgacagg acacgagtaa ctcgtctatc |
| | 181 | ttctgcaggc tgcttacggt ttcgtccgtg ttgcagccga tcatcagcac atctaggttt |
| | 241 | cgtccgggtg tgaccgaaag gtaagatgga gagccttgtc cctggtttca acgagaaaac |
| | 301 | acacgtccaa ctcagtttgc ctgttttaca ggttcgcgac gtgctcgtac gtggctttgg |
| | 361 | agactccgtg gaggaggtct tatcagaggc acgtcaacat cttaaagatg gcacttgtgg |
| | 421 | cttagtagaa gttgaaaaag gcgttttgcc tcaacttgaa cagccctatg tgttcatcaa |
| | 481 | acgttcggat gctcgaactg cacctcatgg tcatgttatg gttgagctgg tagcagaact |
| | 541 | cgaaggcatt cagtacggtc gtagtggtga gacacttggt gtccttgtcc ctcatgtggg |
| | 601 | cgaaatacca gtggcttacc gcaaggttct tcttcgtaag aacggtaata aaggagctgg |
| | 661 | tggccatagt tacggcgccg atctaaagtc atttgactta ggcgacgagc ttggcactga |
| | 721 | tccttatgaa gattttcaag aaaactggaa cactaaacat agcagtggtg ttacccgtga |
| | 781 | actcatgcgt gagcttaacg gaggggcata cactcgctat gtcgataaca acttctgtgg |
| | 841 | ccctgatggc taccctcttg agtgcattaa agaccttcta gcacgtgctg gtaaagcttc |
| | 901 | atgcactttg tccgaacaac tggactttat tgacactaag aggggtgtat actgctgccg |
| | 961 | tgaacatgag catgaaattg cttggtacac ggaacgttct gaaaagagct atgaattgca |
| | 1021 | gacacctttt gaaattaaat tggcaaagaa atttgacacc ttcaatgggg aatgtccaaa |
| | 1081 | ttttgtattt cccttaaatt ccataatcaa gactattcaa ccaagggttg aaaagaaaaa |
| | 1141 | gcttgatggc tttatgggta gaattcgatc tgtctatcca gttgcgtcac caaatgaatg |
| | 1201 | caaccaaatg tgcctttcaa ctctcatgaa gtgtgatcat tgtggtgaaa cttcatggca |
| | 1261 | gacgggcgat tttgttaaag ccacttgcga attttgtggc actgagaatt tgactaaaga |
| | 1321 | aggtgccact acttgtggtt acttacccca aaatgctgtt gttaaaattt attgtccagc |
| | 1381 | atgtcacaat tcagaagtag gacctgagca tagtcttgcc gaataccata atgaatctgg |
| | 1441 | cttgaaaacc attcttcgta agggtggtcg cactattgcc tttggaggct gtgtgttctc |
| | 1501 | ttatgttggt tgccataaca agtgtgccta ttgggttcca cgtgctagcg ctaacatagg |
| | 1561 | ttgtaaccat acaggtgttg ttggagaagg ttccgaaggt cttaatgaca accttcttga |
| | 1621 | aatactccaa aaagagaaag tcaacatcaa tattgttggt gactttaaac ttaatgaaga |
| | 1681 | gatcgccatt attttggcat ctttttctgc ttccacaagt gcttttgtgg aaactgtgaa |
| | 1741 | aggtttggat tataaagcat tcaaacaaat tgttgaatcc tgtggtaatt ttaaagttac |
| | 1801 | aaaaggaaaa gctaaaaaag gtgcctggaa tattggtgaa cagaaatcaa tactgagtcc |
| | 1861 | tctttatgca tttgcatcag aggctgctcg tgttgtacga tcaattttct cccgcactct |
| | 1921 | tgaaactgct caaaattctg tgcgtgtttt acagaaggcc gctataacaa tactagatgg |
| | 1981 | aatttcacag tattcactga gactcattga tgctatgatg ttcacatctg atttggctac |
| | 2041 | taacaatcta gttgtaatgg cctacattac aggtggtgtt gttcagttga cttcgcagtg |
| | 2101 | gctaactaac atctttggca ctgtttatga aaaactcaaa cccgtccttg attggcttga |
| | 2161 | agagaagttt aaggaaggtg tagagtttct tagagacggt tgggaaattg ttaaatttat |
| | 2221 | ctcaacctgt gcttgtgaaa ttgtcggtgg acaaattgtc acctgtgcaa aggaaattaa |
| | 2281 | ggagagtgtt cagacattct ttaagcttgt aaataaattt ttggctttgt gtgctgactc |
| | 2341 | tatcattatt ggtggagcta aacttaaagc cttgaattta ggtgaaacat ttgtcacgca |
| | 2401 | ctcaaaggga ttgtacagaa agtgtgttaa atccagagaa gaaactggcc tactcatgcc |
| | 2461 | tctaaaagcc ccaaaagaaa ttatcttctt agagggagaa acacttccca cagaagtgtt |
| | 2521 | aacagaggaa gttgtcttga aaactggtga tttacaacca ttagaacaac ctactagtga |
| | 2581 | agctgttgaa gctccattgg ttggtacacc agtttgtatt aacgggctta tgttgctcga |
| | 2641 | aatcaaagac acagaaaagt actgtgccct tgcacctaat atgatggtaa caaacaatac |
| | 2701 | cttcacactc aaaggcggtg caccaacaaa ggttactttt ggtgatgaca ctgtgataga |
| | 2761 | agtgcaaggt tacaagagtg tgaatatcac ttttgaactt gatgaaagga ttgataaagt |
| | 2821 | acttaatgag aagtgctctg cctatacagt tgaactcggt acagaagtaa atgagttcgc |
| | 2881 | ctgtgttgtg gcagatgctg tcataaaaac tttgcaacca gtatctgaat tacttacacc |
| | 2941 | actgggcatt gatttagatg agtggagtat ggctacatac tacttatttg atgagtctgg |
| | 3001 | tgagtttaaa ttggcttcac atatgtattg ttctttctac cctccagatg aggatgaaga |
| | 3061 | agaaggtgat tgtgaagaag aagagtttga gccatcaact caatatgagt atggtactga |
| | 3121 | agatgattac caaggtaaac ctttggaatt tggtgccact tctgctgctc ttcaacctga |
| | 3181 | agaagagcaa gaagaagatt ggttagatga tgatagtcaa caaactgttg gtcaacaaga |
| | 3241 | cggcagtgag gacaatcaga caactactat tcaaacaatt gttgaggttc aacctcaatt |
| | 3301 | agagatggaa cttacaccag ttgttcagac tattgaagtg aatagtttta gtggttattt |
| | 3361 | aaaacttact gacaatgtat acattaaaaa tgcagacatt gtggaagaag ctaaaaaggt |
| | 3421 | aaaaccaaca gtggttgtta atgcagccaa tgtttacctt aaacatggag gaggtgttgc |
| | 3481 | aggagcctta aataaggcta ctaacaatgc catgcaagtt gaatctgatg attacatagc |
| | 3541 | tactaatgga ccacttaaag tgggtggtag ttgtgtttta agcggacaca atcttgctaa |
| | 3601 | acactgtctt catgttgtcg gcccaaatgt taacaaaggt gaagacattc aacttcttaa |
| | 3661 | gagtgcttat gaaaatttta atcagcacga agttctactt gcaccattat tatcagctgg |
| | 3721 | tatttttggt gctgacccta tacattcttt aagagtttgt gtagatactg ttcgcacaaa |
| | 3781 | tgtctactta gctgtctttg ataaaaatct ctatgacaaa cttgtttcaa gctttttgga |
| | 3841 | aatgaagagt gaaaagcaag ttgaacaaaa gatcgctgag attcctaaag aggaagttaa |
| | 3901 | gccatttata actgaaagta aaccttcagt tgaacagaga aaacaagatg ataagaaaat |
| | 3961 | caaagcttgt gttgaagaag ttacaacaac tctggaagaa actaagttcc tcacagaaaa |
| | 4021 | cttgttactt tatattgaca ttaatggcaa tcttcatcca gattctgcca ctcttgttag |
| | 4081 | tgacattgac atcactttct taaagaaaga tgctccatat atagtgggtg atgttgttca |
| | 4141 | agagggtgtt ttaactgctg tggttatacc tactaaaaag gctggtggca ctactgaaat |
| | 4201 | gctagcgaaa gctttgagaa aagtgccaac agacaattat ataaccactt acccgggtca |
| | 4261 | gggtttaaat ggttacactg tagaggaggc aaagacagtg cttaaaaagt gtaaaagtgc |
| | 4321 | cttttacatt ctaccatcta ttatctctaa tgagaagcaa gaaattcttg gaactgtttc |
| | 4381 | ttggaatttg cgagaaatgc ttgcacatgc agaagaaaca cgcaaattaa tgcctgtctg |
| | 4441 | tgtggaaact aaagccatag tttcaactat acagcgtaaa tataagggta ttaaaataca |
| | 4501 | agagggtgtg gttgattatg gtgctagatt ttacttttac accagtaaaa caactgtagc |
| | 4561 | gtcacttatc aacacactta acgatctaaa tgaaactctt gttacaatgc cacttggcta |
| | 4621 | tgtaacacat ggcttaaatt tggaagaagc tgctcggtat atgagatctc tcaaagtgcc |
| | 4681 | agctacagtt tctgtttctt cacctgatgc tgttacagcg tataatggtt atcttacttc |
| | 4741 | ttcttctaaa acacctgaag aacattttat tgaaaccatc tcacttgctg gttcctataa |
| | 4801 | agattggtcc tattctggac aatctacaca actaggtata gaatttctta agagaggtga |
| | 4861 | taaaagtgta tattacacta gtaatcctac cacattccac ctagatggtg aagttatcac |
| | 4921 | ctttgacaat cttaagacac ttctttcttt gagagaagtg aggactatta aggtgtttac |
| | 4981 | aacagtagac aacattaacc tccacacgca agttgtggac atgtcaatga catatggaca |
| | 5041 | acagtttggt ccaacttatt tggatggagc tgatgttact aaaataaaac ctcataattc |
| | 5101 | acatgaaggt aaaacatttt atgttttacc taatgatgac actctacgtg ttgaggcttt |
| | 5161 | tgagtactac cacacaactg atcctagttt tctgggtagg tacatgtcag cattaaatca |
| | 5221 | cactaaaaag tggaaatacc cacaagttaa tggtttaact tctattaaat gggcagataa |
| | 5281 | caactgttat cttgccactg cattgttaac actccaacaa atagagttga agtttaatcc |
| | 5341 | acctgctcta caagatgctt attacagagc aagggctggt gaagctgcta acttttgtgc |
| | 5401 | acttatctta gcctactgta ataagacagt aggtgagtta ggtgatgtta gagaaacaat |
| | 5461 | gagttacttg tttcaacatg ccaatttaga ttcttgcaaa agagtcttga acgtggtgtg |
| | 5521 | taaaacttgt ggacaacagc agacaaccct taagggtgta gaagctgtta tgtacatggg |
| | 5581 | cacactttct tatgaacaat ttaagaaagg tgttcagata ccttgtacgt gtggtaaaca |
| | 5641 | agctacaaaa tatctagtac aacaggagtc accttttgtt atgatgtcag caccacctgc |
| | 5701 | tcagtatgaa cttaagcatg gtacatttac ttgtgctagt gagtacactg gtaattacca |
| | 5761 | gtgtggtcac tataaacata taacttctaa agaaactttg tattgcatag acggtgcttt |
| | 5821 | acttacaaag tcctcagaat acaaaggtcc tattacggat gttttctaca aagaaaacag |
| | 5881 | ttacacaaca accataaaac cagttactta taaattggat ggtgttgttt gtacagaaat |
| | 5941 | tgaccctaag ttggacaatt attataagaa agacaattct tatttcacag agcaaccaat |
| | 6001 | tgatcttgta ccaaaccaac catatccaaa cgcaagcttc gataatttta agtttgtatg |
| | 6061 | tgataatatc aaatttgctg atgatttaaa ccagttaact ggttataaga aacctgcttc |
| | 6121 | aagagagctt aaagttacat ttttccctga cttaaatggt gatgtggtgg ctattgatta |
| | 6181 | taaacactac acaccctctt ttaagaaagg agctaaattg ttacataaac ctattgtttg |
| | 6241 | gcatgttaac aatgcaacta ataaagccac gtataaacca aatacctggt gtatacgttg |
| | 6301 | tctttggagc acaaaaccag ttgaaacatc aaattcgttt gatgtactga agtcagagga |
| | 6361 | cgcgcaggga atggataatc ttgcctgcga agatctaaaa ccagtctctg aagaagtagt |
| | 6421 | ggaaaatcct accatacaga aagacgttct tgagtgtaat gtgaaaacta ccgaagttgt |
| | 6481 | aggagacatt atacttaaac cagcaaataa tagtttaaaa attacagaag aggttggcca |
| | 6541 | cacagatcta atggctgctt atgtagacaa ttctagtctt actattaaga aacctaatga |
| | 6601 | attatctaga gtattaggtt tgaaaaccct tgctactcat ggtttagctg ctgttaatag |
| | 6661 | tgtcccttgg gatactatag ctaattatgc taagcctttt cttaacaaag ttgttagtac |
| | 6721 | aactactaac atagttacac ggtgtttaaa ccgtgtttgt actaattata tgccttattt |
| | 6781 | ctttacttta ttgctacaat tgtgtacttt tactagaagt acaaattcta gaattaaagc |
| | 6841 | atctatgccg actactatag caaagaatac tgttaagagt gtcggtaaat tttgtctaga |
| | 6901 | ggcttcattt aattatttga agtcacctaa tttttctaaa ctgataaata ttataatttg |
| | 6961 | gtttttacta ttaagtgttt gcctaggttc tttaatctac tcaaccgctg ctttaggtgt |
| | 7021 | tttaatgtct aatttaggca tgccttctta ctgtactggt tacagagaag gctatttgaa |
| | 7081 | ctctactaat gtcactattg caacctactg tactggttct ataccttgta gtgtttgtct |
| | 7141 | tagtggttta gattctttag acacctatcc ttctttagaa actatacaaa ttaccatttc |
| | 7201 | atcttttaaa tgggatttaa ctgcttttgg cttagttgca gagtggtttt tggcatatat |
| | 7261 | tcttttcact aggtttttct atgtacttgg attggctgca atcatgcaat tgtttttcag |
| | 7321 | ctattttgca gtacatttta ttagtaattc ttggcttatg tggttaataa ttaatcttgt |
| | 7381 | acaaatggcc ccgatttcag ctatggttag aatgtacatc ttctttgcat cattttatta |
| | 7441 | tgtatggaaa agttatgtgc atgttgtaga cggttgtaat tcatcaactt gtatgatgtg |
| | 7501 | ttacaaacgt aatagagcaa caagagtcga atgtacaact attgttaatg gtgttagaag |
| | 7561 | gtccttttat gtctatgcta atggaggtaa aggcttttgc aaactacaca attggaattg |
| | 7621 | tgttaattgt gatacattct gtgctggtag tacatttatt agtgatgaag ttgcgagaga |
| | 7681 | cttgtcacta cagtttaaaa gaccaataaa tcctactgac cagtcttctt acatcgttga |
| | 7741 | tagtgttaca gtgaagaatg gttccatcca tctttacttt gataaagctg gtcaaaagac |
| | 7801 | ttatgaaaga cattctctct ctcattttgt taacttagac aacctgagag ctaataacac |
| | 7861 | taaaggttca ttgcctatta atgttatagt ttttgatggt aaatcaaaat gtgaagaatc |
| | 7921 | atctgcaaaa tcagcgtctg tttactacag tcagcttatg tgtcaaccta tactgttact |
| | 7981 | agatcaggca ttagtgtctg atgttggtga tagtgcggaa gttgcagtta aaatgtttga |
| | 8041 | tgcttacgtt aatacgtttt catcaacttt taacgtacca atggaaaaac tcaaaacact |
| | 8101 | agttgcaact gcagaagctg aacttgcaaa gaatgtgtcc ttagacaatg tcttatctac |
| | 8161 | ttttatttca gcagctcggc aagggtttgt tgattcagat gtagaaacta aagatgttgt |
| | 8221 | tgaatgtctt aaattgtcac atcaatctga catagaagtt actggcgata gttgtaataa |
| | 8281 | ctatatgctc acctataaca aagttgaaaa catgacaccc cgtgaccttg gtgcttgtat |
| | 8341 | tgactgtagt gcgcgtcata ttaatgcgca ggtagcaaaa agtcacaaca ttgctttgat |
| | 8401 | atggaacgtt aaagatttca tgtcattgtc tgaacaacta cgaaaacaaa tacgtagtgc |
| | 8461 | tgctaaaaag aataacttac cttttaagtt gacatgtgca actactagac aagttgttaa |
| | 8521 | tgttgtaaca acaaagatag cacttaaggg tggtaaaatt gttaataatt ggttgaagca |
| | 8581 | gttaattaaa gttacacttg tgttcctttt tgttgctgct attttctatt taataacacc |
| | 8641 | tgttcatgtc atgtctaaac atactgactt ttcaagtgaa atcataggat acaaggctat |
| | 8701 | tgatggtggt gtcactcgtg acatagcatc tacagatact tgttttgcta acaaacatgc |
| | 8761 | tgattttgac acatggttta gccagcgtgg tggtagttat actaatgaca aagcttgccc |
| | 8821 | attgattgct gcagtcataa caagagaagt gggttttgtc gtgcctggtt tgcctggcac |
| | 8881 | gatattacgc acaactaatg gtgacttttt gcatttctta cctagagttt ttagtgcagt |
| | 8941 | tggtaacatc tgttacacac catcaaaact tatagagtac actgactttg caacatcagc |
| | 9001 | ttgtgttttg gctgctgaat gtacaatttt taaagatgct tctggtaagc cagtaccata |
| | 9061 | ttgttatgat accaatgtac tagaaggttc tgttgcttat gaaagtttac gccctgacac |
| | 9121 | acgttatgtg ctcatggatg gctctattat tcaatttcct aacacctacc ttgaaggttc |
| | 9181 | tgttagagtg gtaacaactt ttgattctga gtactgtagg cacggcactt gtgaaagatc |
| | 9241 | agaagctggt gtttgtgtat ctactagtgg tagatgggta cttaacaatg attattacag |
| | 9301 | atctttacca ggagttttct gtggtgtaga tgctgtaaat ttacttacta atatgtttac |
| | 9361 | accactaatt caacctattg gtgctttgga catatcagca tctatagtag ctggtggtat |
| | 9421 | tgtagctatc gtagtaacat gccttgccta ctattttatg aggtttagaa gagcttttgg |
| | 9481 | tgaatacagt catgtagttg cctttaatac tttactattc cttatgtcat tcactgtact |
| | 9541 | ctgtttaaca ccagtttact cattcttacc tggtgtttat tctgttattt acttgtactt |
| | 9601 | gacattttat cttactaatg atgtttcttt tttagcacat attcagtgga tggttatgtt |
| | 9661 | cacaccttta gtacctttct ggataacaat tgcttatatc atttgtattt ccacaaagca |
| | 9721 | tttctattgg ttctttagta attacctaaa gagacgtgta gtctttaatg gtgtttcctt |
| | 9781 | tagtactttt gaagaagctg cgctgtgcac ctttttgtta aataaagaaa tgtatctaaa |
| | 9841 | gttgcgtagt gatgtgctat tacctcttac gcaatataat agatacttag ctctttataa |
| | 9901 | taagtacaag tattttagtg gagcaatgga tacaactagc tacagagaag ctgcttgttg |
| | 9961 | tcatctcgca aaggctctca atgacttcag taactcaggt tctgatgttc tttaccaacc |
| | 10021 | accacaaacc tctatcacct cagctgtttt gcagagtggt tttagaaaaa tggcattccc |
| | 10081 | atctggtaaa gttgagggtt gtatggtaca agtaacttgt ggtacaacta cacttaacgg |
| | 10141 | tctttggctt gatgacgtag tttactgtcc aagacatgtg atctgcacct ctgaagacat |
| | 10201 | gcttaaccct aattatgaag atttactcat tcgtaagtct aatcataatt tcttggtaca |
| | 10261 | ggctggtaat gttcaactca gggttattgg acattctatg caaaattgtg tacttaagct |
| | 10321 | taaggttgat acagccaatc ctaagacacc taagtataag tttgttcgca ttcaaccagg |
| | 10381 | acagactttt tcagtgttag cttgttacaa tggttcacca tctggtgttt accaatgtgc |
| | 10441 | tatgaggccc aatttcacta ttaagggttc attccttaat ggttcatgtg gtagtgttgg |
| | 10501 | ttttaacata gattatgact gtgtctcttt ttgttacatg caccatatgg aattaccaac |
| | 10561 | tggagttcat gctggcacag acttagaagg taacttttat ggaccttttg ttgacaggca |
| | 10621 | aacagcacaa gcagctggta cggacacaac tattacagtt aatgttttag cttggttgta |
| | 10681 | cgctgctgtt ataaatggag acaggtggtt tctcaatcga tttaccacaa ctcttaatga |
| | 10741 | ctttaacctt gtggctatga agtacaatta tgaacctcta acacaagacc atgttgacat |
| | 10801 | actaggacct ctttctgctc aaactggaat tgccgtttta gatatgtgtg cttcattaaa |
| | 10861 | agaattactg caaaatggta tgaatggacg taccatattg ggtagtgctt tattagaaga |
| | 10921 | tgaatttaca ccttttgatg ttgttagaca atgctcaggt gttactttcc aaagtgcagt |
| | 10981 | gaaaagaaca atcaagggta cacaccactg gttgttactc acaattttga cttcactttt |
| | 11041 | agttttagtc cagagtactc aatggtcttt gttctttttt ttgtatgaaa atgccttttt |
| | 11101 | accttttgct atgggtatta ttgctatgtc tgcttttgca atgatgtttg tcaaacataa |
| | 11161 | gcatgcattt ctctgtttgt ttttgttacc ttctcttgcc actgtagctt attttaatat |
| | 11221 | ggtctatatg cctgctagtt gggtgatgcg tattatgaca tggttggata tggttgatac |
| | 11281 | tagtttgtct ggttttaagc taaaagactg tgttatgtat gcatcagctg tagtgttact |
| | 11341 | aatccttatg acagcaagaa ctgtgtatga tgatggtgct aggagagtgt ggacacttat |
| | 11401 | gaatgtcttg acactcgttt ataaagttta ttatggtaat gctttagatc aagccatttc |
| | 11461 | catgtgggct cttataatct ctgttacttc taactactca ggtgtagtta caactgtcat |
| | 11521 | gtttttggcc agaggtattg tttttatgtg tgttgagtat tgccctattt tcttcataac |
| | 11581 | tggtaataca cttcagtgta taatgctagt ttattgtttc ttaggctatt tttgtacttg |
| | 11641 | ttactttggc ctcttttgtt tactcaaccg ctactttaga ctgactcttg gtgtttatga |
| | 11701 | ttacttagtt tctacacagg agtttagata tatgaattca cagggactac tcccacccaa |
| | 11761 | gaatagcata gatgccttca aactcaacat taaattgttg ggtgttggtg gcaaaccttg |
| | 11821 | tatcaaagta gccactgtac agtctaaaat gtcagatgta aagtgcacat cagtagtctt |
| | 11881 | actctcagtt ttgcaacaac tcagagtaga atcatcatct aaattgtggg ctcaatgtgt |
| | 11941 | ccagttacac aatgacattc tcttagctaa agatactact gaagcctttg aaaaaatggt |
| | 12001 | ttcactactt tctgttttgc tttccatgca gggtgctgta gacataaaca agctttgtga |
| | 12061 | agaaatgctg gacaacaggg caaccttaca agctatagcc tcagagttta gttcccttcc |
| | 12121 | atcatatgca gcttttgcta ctgctcaaga agcttatgag caggctgttg ctaatggtga |
| | 12181 | ttctgaagtt gttcttaaaa agttgaagaa gtctttgaat gtggctaaat ctgaatttga |
| | 12241 | ccgtgatgca gccatgcaac gtaagttgga aaagatggct gatcaagcta tgacccaaat |
| | 12301 | gtataaacag gctagatctg aggacaagag ggcaaaagtt actagtgcta tgcagacaat |
| | 12361 | gcttttcact atgcttagaa agttggataa tgatgcactc aacaacatta tcaacaatgc |
| | 12421 | aagagatggt tgtgttccct tgaacataat acctcttaca acagcagcca aactaatggt |
| | 12481 | tgtcatacca gactataaca catataaaaa tacgtgtgat ggtacaacat ttacttatgc |
| | 12541 | atcagcattg tgggaaatcc aacaggttgt agatgcagat agtaaaattg ttcaacttag |
| | 12601 | tgaaattagt atggacaatt cacctaattt agcatggcct cttattgtaa cagctttaag |
| | 12661 | ggccaattct gctgtcaaat tacagaataa tgagcttagt cctgttgcac tacgacagat |
| | 12721 | gtcttgtgct gccggtacta cacaaactgc ttgcactgat gacaatgcgt tagcttacta |
| | 12781 | caacacaaca aagggaggta ggtttgtact tgcactgtta tccgatttac aggatttgaa |
| | 12841 | atgggctaga ttccctaaga gtgatggaac tggtactatc tatacagaac tggaaccacc |
| | 12901 | ttgtaggttt gttacagaca cacctaaagg tcctaaagtg aagtatttat actttattaa |
| | 12961 | aggattaaac aacctaaata gaggtatggt acttggtagt ttagctgcca cagtacgtct |
| | 13021 | acaagctggt aatgcaacag aagtgcctgc caattcaact gtattatctt tctgtgcttt |
| | 13081 | tgctgtagat gctgctaaag cttacaaaga ttatctagct agtgggggac aaccaatcac |
| | 13141 | taattgtgtt aagatgttgt gtacacacac tggtactggt caggcaataa cagttacacc |
| | 13201 | ggaagccaat atggatcaag aatcctttgg tggtgcatcg tgttgtctgt actgccgttg |
| | 13261 | ccacatagat catccaaatc ctaaaggatt ttgtgactta aaaggtaagt atgtacaaat |
| | 13321 | acctacaact tgtgctaatg accctgtggg ttttacactt aaaaacacag tctgtaccgt |
| | 13381 | ctgcggtatg tggaaaggtt atggctgtag ttgtgatcaa ctccgcgaac ccatgcttca |
| | 13441 | gtcagctgat gcacaatcgt ttttaaacgg gtttgcggtg taagtgcagc ccgtcttaca |
| | 13501 | ccgtgcggca caggcactag tactgatgtc gtatacaggg cttttgacat ctacaatgat |
| | 13561 | aaagtagctg gttttgctaa attcctaaaa actaattgtt gtcgcttcca agaaaaggac |
| | 13621 | gaagatgaca atttaattga ttcttacttt gtagttaaga gacacacttt ctctaactac |
| | 13681 | caacatgaag aaacaattta taatttactt aaggattgtc cagctgttgc taaacatgac |
| | 13741 | ttctttaagt ttagaataga cggtgacatg gtaccacata tatcacgtca acgtcttact |
| | 13801 | aaatacacaa tggcagacct cgtctatgct ttaaggcatt ttgatgaagg taattgtgac |
| | 13861 | acattaaaag aaatacttgt cacatacaat tgttgtgatg atgattattt caataaaaag |
| | 13921 | gactggtatg attttgtaga aaacccagat atattacgcg tatacgccaa cttaggtgaa |
| | 13981 | cgtgtacgcc aagctttgtt aaaaacagta caattctgtg atgccatgcg aaatgctggt |
| | 14041 | attgttggtg tactgacatt agataatcaa gatctcaatg gtaactggta tgatttcggt |
| | 14101 | gatttcatac aaaccacgcc aggtagtgga gttcctgttg tagattctta ttattcattg |
| | 14161 | ttaatgccta tattaacctt gaccagggct ttaactgcag agtcacatgt tgacactgac |
| | 14221 | ttaacaaagc cttacattaa gtgggatttg ttaaaatatg acttcacgga agagaggtta |
| | 14281 | aaactctttg accgttattt taaatattgg gatcagacat accacccaaa ttgtgttaac |
| | 14341 | tgtttggatg acagatgcat tctgcattgt gcaaacttta atgttttatt ctctacagtg |
| | 14401 | ttcccaccta caagttttgg accactagtg agaaaaatat ttgttgatgg tgttccattt |
| | 14461 | gtagtttcaa ctggatacca cttcagagag ctaggtgttg tacataatca ggatgtaaac |
| | 14521 | ttacatagct ctagacttag ttttaaggaa ttacttgtgt atgctgctga ccctgctatg |
| | 14581 | cacgctgctt ctggtaatct attactagat aaacgcacta cgtgcttttc agtagctgca |
| | 14641 | cttactaaca atgttgcttt tcaaactgtc aaacccggta attttaacaa agacttctat |
| | 14701 | gactttgctg tgtctaaggg tttctttaag gaaggaagtt ctgttgaatt aaaacacttc |
| | 14761 | ttctttgctc aggatggtaa tgctgctatc agcgattatg actactatcg ttataatcta |
| | 14821 | ccaacaatgt gtgatatcag acaactacta tttgtagttg aagttgttga taagtacttt |
| | 14881 | gattgttacg atggtggctg tattaatgct aaccaagtca tcgtcaacaa cctagacaaa |
| | 14941 | tcagctggtt ttccatttaa taaatggggt aaggctagac tttattatga ttcaatgagt |
| | 15001 | tatgaggatc aagatgcact tttcgcatat acaaaacgta atgtcatccc tactataact |
| | 15061 | caaatgaatc ttaagtatgc cattagtgca aagaatagag ctcgcaccgt agctggtgtc |
| | 15121 | tctatctgta gtactatgac caatagacag tttcatcaaa aattattgaa atcaatagcc |
| | 15181 | gccactagag gagctactgt agtaattgga acaagcaaat tctatggtgg ttggcacaac |
| | 15241 | atgttaaaaa ctgtttatag tgatgtagaa aaccctcacc ttatgggttg ggattatcct |
| | 15301 | aaatgtgata gagccatgcc taacatgctt agaattatgg cctcacttgt tcttgctcgc |
| | 15361 | aaacatacaa cgtgttgtag cttgtcacac cgtttctata gattagctaa tgagtgtgct |
| | 15421 | caagtattga gtgaaatggt catgtgtgg ggttcactat atgttaaacc aggtggaacc |
| | 15481 | tcatcaggag atgccacaac tgcttatgct aatagtgttt ttaacatttg tcaagctgtc |
| | 15541 | acggccaatg ttaatgcact tttatctact gatggtaaca aaattgccga taagtatgtc |
| | 15601 | cgcaatttac aacacagact ttatgagtgt ctctatagaa atagagatgt tgacacagac |
| | 15661 | tttgtgaatg agttttacgc atatttgcgt aaacatttct caatgatgat actctctgac |
| | 15721 | gatgctgttg tgtgtttcaa tagcacttat gcatctcaag gtctagtggc tagcataaag |
| | 15781 | aactttaagt cagttcttta ttatcaaaac aatgttttta tgtctgaagc aaaatgttgg |
| | 15841 | actgagactg accttactaa aggacctcat gaattttgct ctcaacatac aatgctagtt |
| | 15901 | aaacagggtg atgattatgt gtaccttcct tacccagatc catcaagaat cctaggggcc |
| | 15961 | ggctgttttg tagatgatat cgtaaaaaca gatggtacac ttatgattga acggttcgtg |
| | 16021 | tctttagcta tagatgctta cccacttact aaacatccta atcaggagta tgctgatgtc |
| | 16081 | tttcatttgt acttacaata cataagaaag ctacatgatg agttaacagg acacatgtta |
| | 16141 | gacatgtatt ctgttatgct tactaatgat aacacttcaa ggtattggga acctgagttt |
| | 16201 | tatgaggcta tgtacacacc gcatacagtc ttacaggctg ttggggcttg tgttctttgc |
| | 16261 | aattcacaga cttcattaag atgtggtgct tgcatacgta gaccattctt atgttgtaaa |
| | 16321 | tgctgttacg accatgtcat atcaacatca cataaattag tcttgtctgt taatccgtat |
| | 16381 | gtttgcaatg ctccaggttg tgatgtcaca gatgtgactc aactttactt aggaggtatg |
| | 16441 | agctattatt gtaaatcaca taaaccaccc attagttttc cattgtgtgc taatggacaa |
| | 16501 | gtttttggtt tatataaaaa tacatgtgtt ggtagcgata atgttactga ctttaatgca |
| | 16561 | attgcaacat gtgactggac aaatgctggt gattacattt tagctaacac ctgtactgaa |
| | 16621 | agactcaagc tttttgcagc agaaacgctc aaagctactg aggagacatt taaactgtct |
| | 16681 | tatggtattg ctactgtacg tgaagtgctg tctgacagag aattacatct ttcatgggaa |
| | 16741 | gttggtaaac ctagaccacc acttaaccga aattatgtct ttactggtta tcgtgtaact |
| | 16801 | aaaaacagta aagtacaaat aggagagtac acctttgaaa aaggtgacta tggtgatgct |
| | 16861 | gttgtttacc gaggtacaac aacttacaaa ttaaatgttg gtgattattt tgtgctgaca |
| | 16921 | tcacatacag taatgccatt aagtgcacct acactagtgc cacaagagca ctatgttaga |
| | 16981 | attactggct tatacccaac actcaatatc tcagatgagt tttctagcaa tgttgcaaat |
| | 17041 | tatcaaaagg ttggtatgca aaagtattct acactccagg gaccacctgg tactggtaag |
| | 17101 | agtcattttg ctattggcct agctctctac tacccttctg ctcgcatagt gtatacagct |
| | 17161 | tgctctcatg ccgctgttga tgcactatgt gagaaggcat taaaatattt gcctatagat |
| | 17221 | aaatgtagta gaattatacc tgcacgtgct cgtgtagagt gttttgataa attcaaagtg |
| | 17281 | aattcaacat tagaacagta tgtcttttgt actgtaaatg cattgcctga gacgacagca |
| | 17341 | gatatagttg tctttgatga aatttcaatg gccacaaatt atgatttgag tgttgtcaat |
| | 17401 | gccagattac gtgctaagca ctatgtgtac attggcgacc ctgctcaatt acctgcacca |
| | 17461 | cgcacattgc taactaaggg cacactagaa ccagaatatt tcaattcagt gtgtagactt |
| | 17521 | atgaaaacta taggtccaga catgttcctc ggaacttgtc ggcgttgtcc tgctgaaatt |
| | 17581 | gttgacactg tgagtgcttt ggtttatgat aataagctta aagcacataa agacaaatca |
| | 17641 | gctcaatgct ttaaaatgtt ttataagggt gttatcacgc atgatgtttc atctgcaatt |
| | 17701 | aacaggccac aaataggcgt ggtaagagaa ttccttacac gtaaccctgc ttggagaaaa |
| | 17761 | gctgtcttta tttcacctta taattcacag aatgctgtag cctcaaagat tttgggacta |
| | 17821 | ccaactcaaa ctgttgattc atcacagggc tcagaatatg actatgtcat attcactcaa |
| | 17881 | accactgaaa cagctcactc ttgtaatgta aacagattta atgttgctat taccagagca |
| | 17941 | aaagtaggca tactttgcat aatgtctgat agagaccttt atgacaagtt gcaatttaca |
| | 18001 | agtcttgaaa ttccacgtag gaatgtggca actttacaag ctgaaaatgt aacaggactc |
| | 18061 | tttaaagatt gtagtaaggt aatcactggg ttacatccta cacaggcacc tacacacctc |
| | 18121 | agtgttgaca ctaaattcaa aactgaaggt ttatgtgttg acatacctgg catacctaag |
| | 18181 | gacatgacct atagaagact catctctatg atgggtttta aaatgaatta tcaagttaat |
| | 18241 | ggttacccta acatgtttat cacccgcgaa gaagctataa gacatgtacg tgcatggatt |
| | 18301 | ggcttcgatg tcgaggggtg tcatgctact agagaagctg ttggtaccaa tttaccttta |
| | 18361 | cagctaggtt tttctacagg tgttaaccta gttgctgtac ctacaggtta tgttgataca |
| | 18421 | cctaataata cagatttttc cagagttagt gctaaaccac cgcctggaga tcaatttaaa |
| | 18481 | cacctcatac cacttatgta caaaggactt ccttggaatg tagtgcgtat aaagattgta |
| | 18541 | caaatgttaa gtgacacact taaaaatctc tctgacagag tcgtatttgt cttatgggca |
| | 18601 | catggctttg agttgacatc tatgaagtat tttgtgaaaa taggacctga gcgcacctgt |
| | 18661 | tgtctatgtg atagacgtgc cacatgcttt tccactgctt cagacactta tgcctgttgg |
| | 18721 | catcattcta ttggatttga ttacgtctat aatccgttta tgattgatgt tcaacaatgg |
| | 18781 | ggttttacag gtaacctaca aagcaaccat gatctgtatt gtcaagtcca tggtaatgca |
| | 18841 | catgtagcta gttgtgatgc aatcatgact aggtgtctag ctgtccacga gtgctttgtt |
| | 18901 | aagcgtgttg actggactat tgaatatcct ataattggtg atgaactgaa gattaatgcg |
| | 18961 | gcttgtagaa aggttcaaca catggttgtt aaagctgcat tattagcaga caaattccca |
| | 19021 | gttcttcacg acattggtaa ccctaaagct attaagtgtg tacctcaagc tgatgtagaa |
| | 19081 | tggaagttct atgatgcaca gccttgtagt gacaaagctt ataaaataga agaattattc |
| | 19141 | tattcttatg ccacacattc tgacaaattc acagatggtg tatgcctatt ttggaattgc |
| | 19201 | aatgtcgata gatatcctgc taattccatt gtttgtagat ttgacactag agtgctatct |
| | 19261 | aaccttaact tgcctggttg tgatggtggc agtttgtatg taaataaaca tgcattccac |
| | 19321 | acaccagctt ttgataaaag tgcttttgtt aatttaaaac aattaccatt tttctattac |
| | 19381 | tctgacagtc catgtgagtc tcatggaaaa caagtagtgt cagatataga ttatgtacca |
| | 19441 | ctaaagtctg ctacgtgtat aacacgttgc aatttaggtg gtgctgtctg tagacatcat |
| | 19501 | gctaatgagt acagattgta tctcgatgct tataacatga tgatctcagc tggctttagc |
| | 19561 | ttgtgggttt acaaacaatt tgatacttat aacctctgga acacttttac aagacttcag |
| | 19621 | agtttagaaa atgtggcttt taatgttgta aataagggac actttgatgg acaacagggt |
| | 19681 | gaagtaccag tttctatcat taataacact gtttacacaa aagttgatgg tgttgatgta |
| | 19741 | gaattgtttg aaaataaaac aacattacct gttaatgtag catttgagct ttgggctaag |
| | 19801 | cgcaacatta aaccagtacc agaggtgaaa atactcaata atttgggtgt ggacattgct |
| | 19861 | gctaatactg tgatctggga ctacaaaaga gatgctccag cacatatatc tactattggt |
| | 19921 | gtttgttcta tgactgacat agccaagaaa ccaactgaaa cgatttgtgc accactcact |
| | 19981 | gtcttttttg atggtagagt tgatggtcaa gtagacttat ttagaaatgc ccgtaatggt |
| | 20041 | gttcttatta cagaaggtag tgttaaaggt ttacaaccat ctgtaggtcc caaacaagct |
| | 20101 | agtcttaatg gagtcacatt aattggagaa gccgtaaaaa cacagttcaa ttattataag |
| | 20161 | aaagttgatg gtgttgtcca acaattacct gaaacttact ttactcagag tagaaattta |
| | 20221 | caagaattta aacccaggag tcaaatggaa attgatttct tagaattagc tatggatgaa |
| | 20281 | ttcattgaac ggtataaatt agaaggctat gccttcgaac atatcgttta tggagatttt |
| | 20341 | agtcatagtc agttaggtgg tttacatcta ctgattggac tagctaaacg ttttaaggaa |
| | 20401 | tcaccttttg aattagaaga ttttattcct atggacagta cagttaaaaa ctatttcata |
| | 20461 | acagatgcgc aaacaggttc atctaagtgt gtgtgttctg ttattgattt attacttgat |
| | 20521 | gattttgttg aaataataaa atcccaagat ttatctgtag tttctaaggt tgtcaaagtg |
| | 20581 | actattgact atacagaaat ttcatttatg ctttggtgta aagatggcca tgtagaaaca |
| | 20641 | ttttacccaa aattacaatc tagtcaagcg tggcaaccgg gtgttgctat gcctaatctt |
| | 20701 | tacaaaatgc aaagaatgct attagaaaag tgtgaccttc aaaattatgg tgatagtgca |
| | 20761 | acattaccta aaggcataat gatgaatgtc gcaaaatata ctcaactgtg tcaatattta |
| | 20821 | aacacattaa cattagctgt accctataat atgagagtta tacattttgg tgctggttct |
| | 20881 | gataaaggag ttgcaccagg tacagctgtt ttaagacagt ggttgcctac gggtacgctg |
| | 20941 | cttgtcgatt cagatcttaa tgactttgtc tctgatgcag attcaacttt gattggtgat |
| | 21001 | tgtgcaactg tacatacagc taataaatgg gatctcatta ttagtgatat gtacgaccct |
| | 21061 | aagactaaaa atgttacaaa agaaaatgac tctaaagagg gttttttcac ttacatttgt |
| | 21121 | gggtttatac aacaaaagct agctcttgga ggttccgtgg ctataaagat aacagaacat |
| | 21181 | tcttggaatg ctgatcttta taagctcatg ggacacttcg catggtggac agcctttgtt |
| | 21241 | actaatgtga atgcgtcatc atctgaagca tttttaattg gatgtaatta tcttggcaaa |
| | 21301 | ccacgcgaac aaatagatgg ttatgtcatg catgcaaatt acatattttg gaggaataca |
| | 21361 | aatccaattc agttgtcttc ctattcttta tttgacatga gtaaatttcc ccttaaatta |
| | 21421 | aggggtactg ctgttatgtc tttaaaagaa ggtcaaatca atgatatgat tttatctctt |
| | 21481 | cttagtaaag gtagacttat aattagagaa aacaacagag ttgttatttc tagtgatgtt |
| | 21541 | cttgttaaca actaaacgaa ccctgcaggg ccaccatggt gagcaagggc gaggagctgt |
| | 21601 | tcaccggggt ggtgcccatc ctggtcgagc tggacggcga cgtaaacggc cacaagttca |
| | 21661 | gcgtgtccgg cgagggcgag ggcgatgcca cctacggcaa gctgaccctg aagttcatct |
| | 21721 | gcaccaccgg caagctgccc gtgccctggc ccaccctcgt gaccaccctg acctacggcg |
| | 21781 | tgcagtgctt cagccgctac cccgaccaca tgaagcagca cgacttcttc aagtccgcca |
| | 21841 | tgcccgaagg ctacgtccag gagcgcacca tcttcttcaa ggacgacggc aactacaaga |
| | 21901 | cccgcgccga ggtgaagttc gagggcgaca ccctggtgaa ccgcatcgag ctgaagggca |
| | 21961 | tcgacttcaa ggaggacggc aacatcctgg ggcacaagct ggagtacaac tacaacagcc |
| | 22021 | acaacgtcta tatcatggcc gacaagcaga agaacggcat caaggtgaac ttcaagatcc |
| | 22081 | gccacaacat cgaggacggc agcgtgcagc tcgccgacca ctaccagcag aacaccccca |
| | 22141 | tcggcgacgg ccccgtgctg ctgcccgaca accactacct gagcacccag tccgccctga |
| | 22201 | gcaaagaccc caacgagaag cgcgatcaca tggtcctgct ggagttcgtg accgccgccg |
| | 22261 | ggatcactct cggcatggac gagctgtaca agcctgcagg ttaaactcat gcagaccaca |
| | 22321 | caaggcagat gggctatata aacgttttcg cttttccgtt tacgatatat agtctactct |
| | 22381 | tgtgcagaat gaattctcgt aactacatag cacaagtaga tgtagttaac tttaatctca |
| | 22441 | catagcaatc tttaatcagt gtgtaacatt agggaggact tgaaagagcc accacatttt |
| | 22501 | caccgaggcc acgcggagta cgatcgagtg tacagtgaac aatgctaggg agagctgcct |
| | 22561 | atatggaaga gccctaatgt gtaaaattaa ttttagtagt gctatcccca tgtgatttta |
| | 22621 | atagcttctt aggagaatga caaaaaaaaa aaaaaaaaaa aaaaaaaaaa aaaagcggcc |
| | 22681 | gcttgagatc ctttttttct gcgcgtaatc tgctgcttgc aaacaaaaaa accaccgcta |
| | 22741 | ccagcggtgg tttgtttgcc ggatcaagag ctaccaactc tttttccgaa ggtaactggc |
| | 22801 | ttcagcagag cgcagatacc aaatactgtc cttctagtgt agccgtagtt aggccaccac |
| | 22861 | ttcaagaact ctgtagcacc gcctacatac ctcgctctgc taatcctgtt accagtggct |
| | 22921 | gctgccagtg gcgataagtc gtgtcttacc gggttggact caagacgata gttaccggat |
| | 22981 | aaggcgcagc ggtcgggctg aacggggggt tcgtgcacac agcccagctt ggagcgaacg |
| | 23041 | acctacaccg aactgagata cctacagcgt gagctatgag aaagcgccac gcttcccgaa |
| | 23101 | gggagaaagg cggacaggta tccggtaagc ggcagggtcg gaacaggaga gcgcacgagg |
| | 23161 | gagcttccag ggggaaacgc ctggtatctt tatagtcctg tcgggtttcg ccacctctga |
| | 23221 | cttgagcgtc gatttttgtg atgctcgtca ggggggcgga gcctatggaa acgcggaacc |
| | 23281 | cctatttgtt tatttttcta aatacattca aatatgtatc cgctcatgag acaataaccc |
| | 23341 | tgataaatgc ttcaataata ttgaaaaagg aagagtatga gtattcaaca tttccgtgtc |
| | 23401 | gcccttattc ccttttttgc ggcattttgc cttcctgttt ttgctcaccc agaaacgctg |
| | 23461 | gtgaaagtaa aagatgctga agatcagttg ggtgcacgag tgggttacat cgaactggat |
| | 23521 | ctcaacagcg gtaagatcct tgagagtttt cgccccgaag aacgttttcc aatgatgagc |
| | 23581 | acttttaaag ttctgctatg tggcgcggta ttatcccgtg ttgacgccgg gcaagagcaa |
| | 23641 | ctcggtcgcc gcatacacta ttctcagaat gacttggttg agtactcacc agtcacagaa |
| | 23701 | aagcatctta cggatggcat gacagtaaga gaattatgca gtgctgccat aaccatgagt |
| | 23761 | gataacactg cggccaactt acttctgaca acgatcggag gaccgaagga gctaaccgct |
| | 23821 | tttttgcaca acatggggga tcatgtaact cgccttgatc gttgggaacc ggagctgaat |
| | 23881 | gaagccatac caaacgacga gcgtgacacc acgatgcctg tagcaatggc aacaacgttg |
| | 23941 | cgcaaactat taactggcga actacttact ctagcttccc ggcaacaatt aatagactgg |
| | 24001 | atggaggcgg ataaagttgc aggaccactt ctgcgctcgg cccttccggc tggctggttt |
| | 24061 | attgctgata aatctggagc cggtgagcgt gggtctcgcg gtatcattgc agcactgggg |
| | 24121 | ccagatggta agccctcccg tatcgtagtt atctacacga cggggagtca ggcaactatg |
| | 24181 | gatgaacgaa atagacagat cgctgagata ggtgcctcac tgattaagca ttggtaagcg |
| | 24241 | atcgcaacgc cagcaacgcg agctccgtta cataacttac ggtaaatggc ccgcctggct |
| | 24301 | gaccgcccaa cgacccccgc ccattgacgt caataatgac gtatgttccc atagtaacgc |
| | 24361 | caatagggac tttccattga cgtcaatggg tggagtattt acggtaaact gcccacttgg |
| | 24421 | cagtacatca agtgtatcat atgccaagta cgccccctat tgacgtcaat gacggtaaat |
| | 24481 | ggcccgcctg gcattatgcc cagtacatga ccttatggga ctttcctact tggcagtaca |
| | 24541 | tctacgtatt agtcatcgct attaccatgg tgatgcggtt ttggcagtac atcaatgggc |
| | 24601 | gtggatagcg gtttgactca cggggatttc caagtctcca ccccattgac gtcaatggga |
| | 24661 | gtttgttttg gcaccaaaat caacgggact ttccaaaatg tcgtaacaac tccgccccat |
| | 24721 | tgacgcaaat gggcggtagg cgtgtacggt gggaggtcta tataagcaga gctcgtttag |
| | 24781 | tgaaccgtca gaattttgta atacgactca ctataggg |

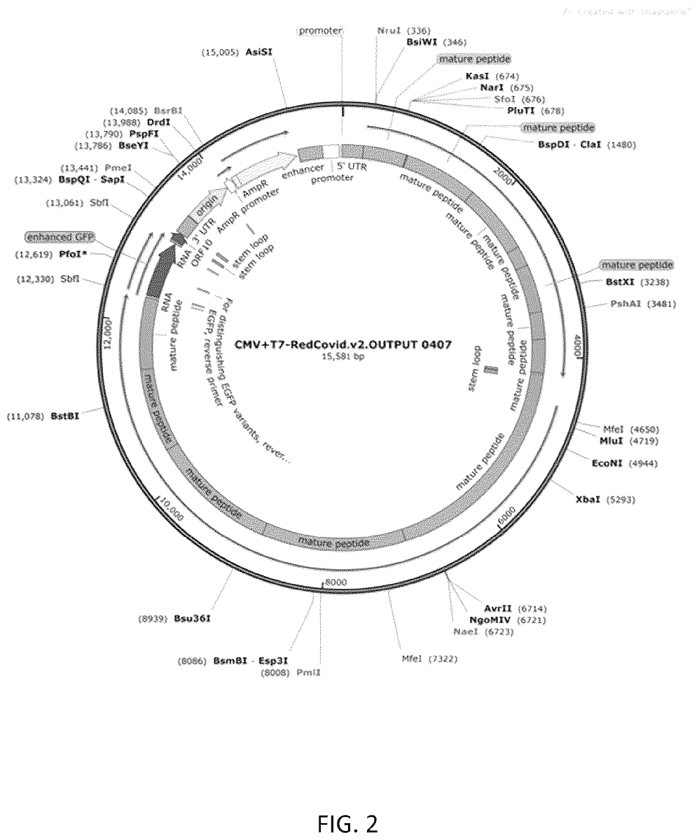

FIG. 2 illustrates a vector based on a partial SARS-COV-2 replicon, with the EGFP gene as payload. The vector consists of the ORF1ab gene from which genes nsp2, nsp3, and nsp4 have been removed. The exemplary payload consists of the EGFP gene. In addition to the vector and the payload, the construct contains an origin of replication, a bacterial promoter, and an AmpR gene acting as a selection marker, useful when the construct is used as a plasmid; and a human CMV enhancer/promoter, useful when the construct is used as a DNA/RNA vector in humans.

The features present in the construct are listed in the following table:

Sequence of a vector based on a partial SARS-CoV-2 replicon (in GenBank format)

| | |
|---|---|
| LOCUS | Exported 15581 bp ds-DNA circular SYN 07-APR-2021 |
| DEFINITIN | construct. |
| ACCESSION | . |
| VERSION | . |
| KEYWORDS | . |
| SOURCE | synthetic DNA construct |
| ORGANISM | synthetic DNA construct |
| REFERENCE | 1 (bases 1 to 15581) |
| AUTHORS | . |
| TITLE | Direct Submission |
| JOURNAL | Exported Thursday, May 20, 2021 from SnapGene Viewer 5.2.4 |
| | https://www.snapgene.com |
| FEATURES | Location/Qualifiers |
| source | 1..15581 |
| | /organism = ″synthetic DNA construct″ |
| source | 13056..13063 |
| 5′UTR | 1..265 |
| mat_peptide | 266..805 |
| | /gene = ″ORF1ab″ |
| | /locus_tag = ″GU280_gp01″ |
| | /product = ″leader protein″ |
| | /note = ″nsp1; produced by both pp1a and pp1ab″ |
| | /note = ″/protein_id = YP_009725297.1″ |
| mat_peptide | 806..817 |
| | /note = ″linker-encodes the first 4 AAs of the MRS (Mpro |
| | cleavage site)″ |
| mat_peptide | 818..1735 |
| | /gene = ″ORF1ab″ |
| | /locus_tag = ″GU280_gp01″ |
| | /product = ″3C-like proteinase″ |
| | /note = ″nsp5A_3CLpro and nsp5B_3CLpro; main |
| | proteinase(Mpro); mediates cleavages downstream of nsp4. 3D |
| | structure of the SARSr-CoV homolog has been determined |
| | (Yang et al., 2003); produced by both pp1a and pp1ab″ |
| | /note = ″/protein_id = YP_009725301.1″ |
| mat_peptide | 1736..2605 |
| | /gene = ″ORF1ab″ |
| | /locus_tag = ″GU280_gp01″ |
| | /product = ″nsp6″ |
| | /note = ″nsp6_TM; putative transmembrane domain; produced |
| | byboth pp1a and pp1ab″ |
| | /note = ″/protein_id = YP_009725302.1″ |
| mat_peptide | 2606..2854 |
| | /gene = ″ORF1ab″ |
| | /locus_tag = ″GU280_gp01″ |
| | /product = ″nsp7″ |
| | /note = ″produced by both pp1a and pp1ab″ |
| | /note = ″/protein_id = YP_009725303.1″ |
| mat_peptide | 2855..3448 |
| | /gene = ″ORF1ab″ |
| | /locus_tag = ″GU280_gp01″ |
| | /product = ″nsp8′ |
| | /note = ″produced by both pp1a and pp1ab″ |
| | /note = ″/protein_id = YP_009725304.1″ |
| mat_peptide | 3449..3787 |
| | /gene = ″ORF1ab″ |
| | /locus_tag = ″GU280_gp01″ |
| | /product = ″nsp9″ |
| | /note = ″ssRNA-binding protein; produced by both pp1a |
| | andpp1ab″ |
| | /note = ″/protein_id = YP_009725305.1″ |
| mat_peptide | 3788..4204 |
| | /gene = ″ORF1ab″ |
| | /locus_tag = ″GU280_gp01″ |
| | /product = ″nsp10″ |
| | /note = ″nsp10_CysHis; formerly known as growth-factor-like |
| | protein (GFL); produced by both pp1a and pp1ab″ |
| | /note = ″/protein_id = YP_009725306.1″ |
| | 4205..6999 |
| mat_peptide | /gene = ″ORF1ab″ |
| | /locus_tag = ″GU280_gp01″ |
| | /product = ″RNA-dependent RNA polymerase″ |
| | /note = ″nsp12; NiRAN and RdRp; produced by pp1ab only″ |
| | /note = ″/protein_id = YP_009725307.1″ |
| mat_peptide | 4205..4243 |
| | /gene = ″ORF1ab″ |
| | /locus_tag = ″GU280_gp01″ |
| | /product = ″nsp11″ |
| | /note = ″produced by pp1a only″ |
| | /note = ″/protein_id = YP_009725312.1″ |
| stem_loop | 4239..4266 |
| | /gene = ″ORF1ab″ |
| | /locus_tag = ″GU280_gp01″ |
| | /function = ″Coronavirus frameshifting stimulation |
| | elementstem-loop 1″ |
| | /inference = ″COORDINATES:same species:Rfam-release-14.1″ |
| stem_loop | 4251..4305 |
| | /gene = ″ORF1ab″ |
| | /locus_tag = ″GU280_gp01″ |
| | /function = ″Coronavirus frameshifting stimulation |
| | elementstem-loop 2″ |
| | /inference = ″COORDINATES:same species:profile″ |
| mat_peptide | 7000..8802 |
| | /gene = ″ORF1ab″ |
| | /locus_tag = ″GU280_gp01″ |
| | /product = ″helicase″ |
| | /note = ″nsp13_ZBD, nsp13_TB, and nsp_HEL1core; zinc-binding |
| | domain (ZD), NTPase/helicase domain (HEL), RNA |
| | 5′-triphosphatase; produced by pp1ab only″ |
| | /note = ″/protein_id = YP_009725308.1″ |
| mat_peptide | 8803..10383 |
| | /gene = ″ORF1ab″ |
| | /locus_tag = ″GU280_gp01″ |
| | /product = ″3′-to-5′ exonuclease″ |
| | /note = ″nsp14A2_ExoN and nsp14B_NMT; produced by pp1abonly″ |
| | /note = ″/protein_id = YP_009725309.1″ |
| mat_peptide | 10384..11421 |
| | /gene = ″ORF1ab″ |
| | /locus_tag = ″GU280_gp01″ |
| | /product = ″endoRNAse″ |
| | /note = ″nsp15-A1 and nsp15B-NendoU; produced by pp1ab only″ |
| | /note = ″/protein_id = YP_009725310.1″ |
| mat_peptide | 11422..12315 |
| | /gene = ″ORF1ab″ |
| | /locus_tag = ″GU280_gp01″ |
| | /product = ″2′-O-ribose methyltransferase″ |
| | /note = ″nsp16_OMT; 2′-o-MT; produced by pp1ab only″ |
| | /note = ″/protein_id = YP_009725311.1″ |
| misc_feature | 12316..12324 |
| | /label = nonstandard type: RNA |
| | /note = ″Transcription regulation sequence-begins withstop |
| | codon″ |
| regulatory | 12333..12342 |
| | /regulatory_class = ″other″ |
| | /note = ″vertebrate consensus sequence for strong |
| | initiationof translation (Kozak, 1987)-likely unnecessary |
| | if used with Covid vector″ |
| CDS | 12339..13055 |
| | /codon_start = 1 |
| | /product = ″enhanced GFP″ |
| | /label = enhanced GFP |
| | /note = ″mammalian codon-optimized, without stop codon″ |

/translation = ″MVSKGEELFTGVVPILVELDGDVNGHKFSVSGEGEGDATYGKLTL
KFICTTGKLPVPWPTLVTTLTYGVQCFSRYPDHMKQHDFFKSAMPEGYVQERTIFFKDD
GNYKTRAEVKFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPNEKRDHMVLL
EFVTAAGITLGMDELYK″

| | |
|---|---|
| primer_bind | complement (12384..12405) |
| | /label = EGFP, reverse primer |
| | /note = ″EGFP, reverse primer″ |
| primer_bind | complement (12645..12664) |
| | /label = For distinguishing EGFP variants, reverse primer |
| | /note = ″For distinguishing EGFP variants, reverse primer″ |
| primer_bind | 12992..13013 |
| | /label = EGFP, forward primer |
| | /note = ″EGFP, forward primer″ |
| misc_feature | 13065..13091 |
| | /label = nonstandard type: RNA |
| | /note = ″linker starting with TRS (begins with stop codon)″ |
| gene | 13092..13208 |
| | /gene = ″ORF10″ |
| | /locus_tag = ″GU280_gp11″ |
| | /label = ORF10 |
| | /db_xref = ″GenelD:43740576″ |
| CDS | 13092..13208 |
| | /codon_start = 1 |
| | /gene = ″ORF10″ |
| | /locus_tag = ″GU280_gp11″ |
| | /product = ″ORF10 protein″ |
| | /label = ORF10 |
| | /db_xref = ″GeneID:43740576″ |
| | /protein_id = ″YP_009725255.1″ |
| | /translation = ″MGYINVFAFPFTIYSLLLCRMNSRNYIAQVDVVNFNLT″ |
| stem_loop | 13143..13178 |
| | /gene = ″ORF10″ |
| | /locus_tag = ″GU280_gp11″ |
| | /function = ″Coronavirus 3′ UTR pseudoknot stem-loop 1″ |
| | /inference = ″COORDINATES:same species:Rfam-release-14.1″ |
| stem_loop | 13163 .. 13191 |
| | /gene = ″ORF10″ |
| | /locus_tag = ″GU280_gp11″ |
| | /function = ″Coronavirus 3′ UTR pseudoknot stem-loop 2″ |
| | /inference = ″COORDINATES:same species: Rfam-release-14.1″ |
| 3′UTR | 13209..13437 |
| stem_loop | 13262..13302 |
| | /function = ″Coronavirus 3′ stem-loop II-like motif (s2m)″ |
| | /note = ″basepair exception: alignment to the Rfam |
| | modelimplies coordinates 29740:29758 form a noncanonical |
| | C:T basepair, but the homologous positions form a highly |
| | conserved C:G basepair in other viruses, including SARS |
| | (NC_004718.3)″ |
| | /inference = ″COORDINATES:same species: Rfam-release-14.1″ |
| rep_origin | 13446..14034 |
| | /direction = RIGHT |
| | /note = ″high-copy-number ColE1/pMB1/pBR322/pUC origin of |
| | replication″ |
| primer_bind | 13935..13954 |
| | /label = pBR322 origin, forward primer |
| | /note = ″pBR322 origin, forward primer″ |
| promoter | 14035..14139 |
| | /gene = ″bla″ |
| | /label = AmpR promoter |
| CDS | 14140..15000 |
| | /codon_start = 1 |
| | /gene = ″bla″ |
| | /product = ″beta-lactamase″ |
| | /label = AmpR |
| | /note = ″confers resistance to ampicillin, carbenicillin, and |
| | related antibiotics″ |

/translation = ″MSIQHFRVALIPFFAAFCLPVFAHPETLVKVKDAEDQLGARVGYI
ELDLNSGKILESFRPEERFPMMSTFKVLLCGAVLSRVDAGQEQLGRRIHYSQNDLVEYS
PVTEKHLTDGMTVRELCSAAITMSDNTAANLLLTTIGGPKELTAFLHNMGDHVTRLDRW
EPELNEAIPNDERDTTMPVAMATTLRKLLTGELLTLASRQQLIDWMEADKVAGPLLRSA
LPAGWFIADKSGAGERGSRGIIAALGPDGKPSRIVVIYTTGSQATMDERNRQIAEIGAS
LIKHW″

| | |
|---|---|
| enhancer | 15029..15332 |
| | /note = ″human cytomegalovirus immediate early enhancer″ |
| promoter | 15333..15536 |
| | /note = ″human cytomegalovirus (CMV) immediate earlypromoter″ |
| promoter | 15563..15581 |
| | /note = ″T7 promoter″ |

The DNA sequence of the construct is listed in the following Table 4:

TABLE 4
Sequence of a vector based on the full SARS-CoV-2 replicon

| | Nucleic Acid Sequence (Description of what is encoded. Sequence |
|---|---|
| | represented as DNA for corresponding RNA sequence T is replaced |
| Sequence | with U). The number at the beginning of each line represents the |
| ID number | coordinate (1-based) of the first nucleotide in that line |
| 2 | CMV+T7_RedCOVID |
| | 1 attaaaggtt tataccttcc caggtaacaa accaaccaac tttcgatctc ttgtagatct |
| | 61 gttctctaaa cgaactttaa aatctgtgtg gctgtcactc ggctgcatgc ttagtgcact |
| | 121 cacgcagtat aattaataac taattactgt cgttgacagg acacgagtaa ctcgtctatc |
| | 181 ttctgcaggc tgcttacggt ttcgtccgtg ttgcagccga tcatcagcac atctaggttt |
| | 241 cgtccgggtg tgaccgaaag gtaagatgga gagccttgtc cctggtttca acgagaaaac |
| | 301 acacgtccaa ctcagtttgc ctgttttaca ggttcgcgac gtgctcgtac gtggctttgg |
| | 361 agactccgtg gaggaggtct tatcagaggc acgtcaacat cttaaagatg gcacttgtgg |
| | 421 cttagtagaa gttgaaaaag gcgttttgcc tcaacttgaa cagccctatg tgttcatcaa |
| | 481 acgttcggat gctcgaactg cacctcatgg tcatgttatg gttgagctgg tagcagaact |
| | 541 cgaaggcatt cagtacggtc gtagtggtga gacacttggt gtccttgtcc ctcatgtggg |
| | 601 cgaaatacca gtggcttacc gcaaggttct tcttcgtaag aacggtaata aaggagctgg |
| | 661 tggccatagt tacggcgccg atctaaagtc atttgactta ggcgacgagc ttggcactga |
| | 721 tccttatgaa gattttcaag aaaactggaa cactaaacat agcagtggtg ttacccgtga |
| | 781 actcatgcgt gagcttaacg gaggggctgt tttgcagagt ggttttagaa aaatggcatt |
| | 841 cccatctggt aaagttgagg gttgtatggt acaagtaact tgtggtacaa ctacacttaa |
| | 901 cggtctttgg cttgatgacg tagtttactg tccaagacat gtgatctgca cctctgaaga |
| | 961 catgcttaac cctaattatg aagatttact cattcgtaag tctaatcata atttcttggt |
| | 1021 acaggctggt aatgttcaac tcagggttat tggacattct atgcaaaatt gtgtacttaa |
| | 1081 gcttaaggtt gatacagcca atcctaagac acctaagtat aagtttgttc gcattcaacc |
| | 1141 aggacagact ttttcagtgt tagcttgtta caatggttca ccatctggtg tttaccaatg |
| | 1201 tgctatgagg cccaatttca ctattaaggg ttcattcctt aatggttcat gtggtagtgt |
| | 1261 tggttttaac atagattatg actgtgtctc tttttgttac atgcaccata tggaattacc |
| | 1321 aactggagtt catgctggca cagacttaga aggtaacttt tatggacctt ttgttgacag |
| | 1381 gcaaacagca caagcagctg gtacggacac aactattaca gttaatgttt tagcttggtt |
| | 1441 gtacgctgct gttataaatg gagacaggtg gtttctcaat cgatttacca caactcttaa |
| | 1501 tgactttaac cttgtggcta tgaagtacaa ttatgaacct ctaacacaag accatgttga |
| | 1561 catactagga cctctttctg ctcaaactgg aattgccgtt ttagatatgt gtgcttcatt |
| | 1621 aaaagaatta ctgcaaaatg gtatgaatgg acgtaccata ttgggtagtg ctttattaga |
| | 1681 agatgaattt acaccttttg atgttgttag acaatgctca ggtgttactt tccaaagtgc |
| | 1741 agtgaaaaga acaatcaagg gtacacacca ctggttgtta ctcacaattt tgacttcact |
| | 1801 tttagtttta gtccagagta ctcaatggtc tttgttcttt tttttgtatg aaaatgcctt |
| | 1861 tttacctttt gctatgggta ttattgctat gtctgctttt gcaatgatgt ttgtcaaaca |
| | 1921 taagcatgca tttctctgtt tgtttttgtt accttctctt gccactgtag cttattttaa |
| | 1981 tatggtctat atgcctgcta gttgggtgat gcgtattatg acatggttgg atatggttga |
| | 2041 tactagtttg tctggtttta agctaaaaga ctgtgttatg tatgcatcag ctgtagtgtt |
| | 2101 actaatcctt atgacagcaa gaactgtgta tgatgatggt gctaggagag tgtggacact |
| | 2161 tatgaatgtc ttgacactcg tttataaagt ttattatggt aatgctttag atcaagccat |
| | 2221 ttccatgtgg gctcttataa tctctgttac ttctaactac tcaggtgtag ttacaactgt |
| | 2281 catgtttttg gccagaggta ttgtttttat gtgtgttgag tattgcccta ttttcttcat |
| | 2341 aactggtaat acacttcagt gtataatgct agtttattgt ttcttaggct atttttgtac |
| | 2401 ttgttacttt ggcctctttt gtttactcaa ccgctacttt agactgactc ttggtgttta |
| | 2461 tgattactta gtttctacac aggagtttag atatatgaat tcacagggac tactcccacc |
| | 2521 caagaatagc atagatgcct tcaaactcaa cattaaattg ttgggtgttg gtggcaaacc |
| | 2581 ttgtatcaaa gtagccactg tacagtctaa aatgtcagat gtaaagtgca catcagtagt |
| | 2641 cttactctca gttttgcaac aactcagagt agaatcatca tctaaattgt gggctcaatg |
| | 2701 tgtccagtta cacaatgaca ttctcttagc taaagatact actgaagcct ttgaaaaaat |
| | 2761 ggtttcacta ctttctgttt tgctttccat gcagggtgct gtagacataa acaagctttg |
| | 2821 tgaagaaatg ctggacaaca gggcaacctt acaagctata gcctcagagt ttagttccct |
| | 2881 tccatcatat gcagcttttg ctactgctca agaagcttat gagcaggctg ttgctaatgg |
| | 2941 tgattctgaa gttgttctta aaaagttgaa gaagtctttg aatgtggcta aatctgaatt |
| | 3001 tgaccgtgat gcagccatgc aacgtaagtt ggaaaagatg gctgatcaag ctatgaccca |
| | 3061 aatgtataaa caggctagat ctgaggacaa gagggcaaaa gttactagtg ctatgcagac |
| | 3121 aatgcttttc actatgctta gaaagttgga taatgatgca ctcaacaaca ttatcaacaa |
| | 3181 tgcaagagat ggttgtgttc ccttgaacat aatacctctt acaacagcag ccaaactaat |
| | 3241 ggttgtcata ccagactata acacatataa aaatacgtgt gatggtacaa catttactta |
| | 3301 tgcatcagca ttgtgggaaa tccaacaggt tgtagatgca gatagtaaaa ttgttcaact |
| | 3361 tagtgaaatt agtatggaca attcacctaa tttagcatgg cctcttattg taacagcttt |
| | 3421 aagggccaat tctgctgtca aattacagaa taatgagctt agtcctgttg cactacgaca |
| | 3481 gatgtcttgt gctgccggta ctacacaaac tgcttgcact gatgacaatg cgttagctta |
| | 3541 ctacaacaca acaaagggag gtaggtttgt acttgcactg ttatccgatt tacaggattt |
| | 3601 gaaatgggct agattcccta agagtgatgg aactggtact atctatacag aactggaacc |
| | 3661 accttgtagg tttgttacag acacacctaa aggtcctaaa gtgaagtatt tatactttat |
| | 3721 taaaggatta aacaacctaa atagaggtat ggtacttggt agtttagctg ccacagtacg |
| | 3781 tctacaagct ggtaatgcaa cagaagtgcc tgccaattca actgtattat ctttctgtgc |
| | 3841 ttttgctgta gatgctgcta aagcttacaa agattatcta gctagtgggg gacaaccaat |
| | 3901 cactaattgt gttaagatgt tgtgtacaca cactggtact ggtcaggcaa taacagttac |
| | 3961 accggaagcc aatatggatc aagaatcctt tggtggtgca tcgtgttgtc tgtactgccg |
| | 4021 ttgccacata gatcatccaa atcctaaagg attttgtgac ttaaaaggta agtatgtaca |
| | 4081 aatacctaca acttgtgcta atgaccctgt gggttttaca cttaaaaaca cagtctgtac |
| | 4141 cgtctgcggt atgtggaaag gttatggctg tagttgtgat caactccgcg aacccatgct |
| | 4201 tcagtcagct gatgcacaat cgtttttaaa cgggtttgcg gtgtaagtgc agcccgtctt |
| | 4261 acaccgtgcg gcacaggcac tagtactgat gtcgtataca gggcttttga catctacaat |
| | 4321 gataaagtag ctggttttgc taaattccta aaaactaatt gttgtcgctt ccaagaaaag |
| | 4381 gacgaagatg acaatttaat tgattcttac tttgtagtta agagacacac tttctctaac |
| | 4441 taccaacatg aagaaacaat ttataattta cttaaggatt gtccagctgt tgctaaacat |
| | 4501 gacttcttta agtttagaat agacggtgac atggtaccac atatatcacg tcaacgtctt |
| | 4561 actaaataca caatggcaga cctcgtctat gctttaaggc attttgatga aggtaattgt |
| | 4621 gacacattaa aagaaatact tgtcacatac aattgttgtg atgatgatta tttcaataaa |
| | 4681 aaggactggt atgattttgt agaaaaccca gatatattac gcgtatacgc caacttaggt |
| | 4741 gaacgtgtac gccaagcttt gttaaaaaca gtacaattct gtgatgccat gcgaaatgct |
| | 4801 ggtattgttg gtgtactgac attagataat caagatctca atggtaactg gtatgatttc |
| | 4861 ggtgatttca tacaaaccac gccaggtagt ggagttcctg ttgtagattc ttattattca |
| | 4921 ttgttaatgc ctatattaac cttgaccagg gctttaactg cagagtcaca tgttgacact |
| | 4981 gacttaacaa agccttacat taagtgggat ttgttaaaat atgacttcac ggaagagagg |
| | 5041 ttaaaactct ttgaccgtta ttttaaatat tgggatcaga cataccaccc aaattgtgtt |
| | 5101 aactgtttgg atgacagatg cattctgcat tgtgcaaact ttaatgtttt attctctaca |
| | 5161 gtgttcccac ctacaagttt tggaccacta gtgagaaaaa tatttgttga tggtgttcca |
| | 5221 tttgtagttt caactggata ccacttcaga gagctaggtg ttgtacataa tcaggatgta |
| | 5281 aacttacata gctctagact tagttttaag gaattacttg tgtatgctgc tgaccctgct |
| | 5341 atgcacgctg cttctggtaa tctattacta gataaacgca ctacgtgctt ttcagtagct |
| | 5401 gcacttacta acaatgttgc ttttcaaact gtcaaacccg gtaattttaa caaagacttc |
| | 5461 tatgactttg ctgtgtctaa gggtttcttt aaggaaggaa gttctgttga attaaaacac |
| | 5521 ttcttctttg ctcaggatgg taatgctgct atcagcgatt atgactacta tcgttataat |
| | 5581 ctaccaacaa tgtgtgatat cagacaacta ctatttgtag ttgaagttgt tgataagtac |
| | 5641 tttgattgtt acgatggtgg ctgtattaat gctaaccaag tcatcgtcaa caacctagac |
| | 5701 aaatcagctg gttttccatt taataaatgg ggtaaggcta gactttatta tgattcaatg |
| | 5761 agttatgagg atcaagatgc acttttcgca tatacaaaac gtaatgtcat ccctactata |
| | 5821 actcaaatga atcttaagta tgccattagt gcaaagaata gagctcgcac cgtagctggt |
| | 5881 gtctctatct gtagtactat gaccaataga cagtttcatc aaaaattatt gaaatcaata |
| | 5941 gccgccacta gaggagctac tgtagtaatt ggaacaagca aattctatgg tggttggcac |
| | 6001 aacatgttaa aaactgttta tagtgatgta gaaaaccctc accttatggg ttgggattat |
| | 6061 cctaaatgtg atagagccat gcctaacatg cttagaatta tggcctcact tgttcttgct |
| | 6121 cgcaaacata caacgtgttg tagcttgtca caccgtttct atagattagc taatgagtgt |
| | 6181 gctcaagtat tgagtgaaat ggtcatgtgt ggcggttcac tatatgttaa accaggtgga |
| | 6241 acctcatcag gagatgccac aactgcttat gctaatagtg tttttaacat ttgtcaagct |
| | 6301 gtcacggcca atgttaatgc acttttatct actgatggta acaaaattgc cgataagtat |
| | 6361 gtccgcaatt tacaacacag actttatgag tgtctctata gaaatagaga tgttgacaca |
| | 6421 gactttgtga atgagtttta cgcatatttg cgtaaacatt tctcaatgat gatactctct |
| | 6481 gacgatgctg ttgtgtgttt caatagcact tatgcatctc aaggtctagt ggctagcata |
| | 6541 aagaacttta agtcagttct ttattatcaa aacaatgttt ttatgtctga agcaaaatgt |
| | 6601 tggactgaga ctgaccttac taaaggacct catgaatttt gctctcaaca tacaatgcta |
| | 6661 gttaaacagg gtgatgatta tgtgtacctt ccttacccag atccatcaag aatcctaggg |
| | 6721 gccggctgtt ttgtagatga tatcgtaaaa acagatggta cacttatgat tgaacggttc |
| | 6781 gtgtctttag ctatagatgc ttacccactt actaaacatc ctaatcagga gtatgctgat |
| | 6841 gtctttcatt tgtacttaca atacataaga aagctacatg atgagttaac aggacacatg |
| | 6901 ttagacatgt attctgttat gcttactaat gataacactt caaggtattg ggaacctgag |
| | 6961 ttttatgagg ctatgtacac accgcataca gtcttacagg ctgttggggc ttgtgttctt |
| | 7021 tgcaattcac agacttcatt aagatgtggt gcttgcatac gtagaccatt cttatgttgt |
| | 7081 aaatgctgtt acgaccatgt catatcaaca tcacataaat tagtcttgtc tgttaatccg |
| | 7141 tatgtttgca atgctccagg ttgtgatgtc acagatgtga ctcaacttta cttaggaggt |
| | 7201 atgagctatt attgtaaatc acataaacca cccattagtt ttccattgtg tgctaatgga |
| | 7261 caagtttttg gtttatataa aaatacatgt gttggtagcg ataatgttac tgactttaat |
| | 7321 gcaattgcaa catgtgactg gacaaatgct ggtgattaca ttttagctaa cacctgtact |
| | 7381 gaaagactca agctttttgc agcagaaacg ctcaaagcta ctgaggagac atttaaactg |
| | 7441 tcttatggta ttgctactgt acgtgaagtg ctgtctgaca gagaattaca tctttcatgg |
| | 7501 gaagttggta aacctagacc accacttaac cgaaattatg tctttactgg ttatcgtgta |
| | 7561 actaaaaaca gtaaagtaca aataggagag tacacctttg aaaaaggtga ctatggtgat |
| | 7621 gctgttgttt accgaggtac aacaacttac aaattaaatg ttggtgatta ttttgtgctg |
| | 7681 acatcacata cagtaatgcc attaagtgca cctacactag tgccacaaga gcactatgtt |
| | 7741 agaattactg gcttataccc aacactcaat atctcagatg agttttctag caatgttgca |
| | 7801 aattatcaaa aggttggtat gcaaaagtat tctacactcc agggaccacc tggtactggt |
| | 7861 aagagtcatt ttgctattgg cctagctctc tactaccctt ctgctcgcat agtgtataca |
| | 7921 gcttgctctc atgccgctgt tgatgcacta tgtgagaagg cattaaaata tttgcctata |
| | 7981 gataaatgta gtagaattat acctgcacgt gctcgtgtag agtgttttga taaattcaaa |
| | 8041 gtgaattcaa cattagaaca gtatgtcttt tgtactgtaa atgcattgcc tgagacgaca |
| | 8101 gcagatatag ttgtctttga tgaaatttca atggccacaa attatgattt gagtgttgtc |
| | 8161 aatgccagat tacgtgctaa gcactatgtg tacattggcg accctgctca attacctgca |
| | 8221 ccacgcacat tgctaactaa gggcacacta gaaccagaat atttcaattc agtgtgtaga |
| | 8281 cttatgaaaa ctataggtcc agacatgttc ctcggaactt gtcggogttg tcctgctgaa |
| | 8341 attgttgaca ctgtgagtgc tttggtttat gataataagc ttaaagcaca taaagacaaa |
| | 8401 tcagctcaat gctttaaaat gttttataag ggtgttatca cgcatgatgt ttcatctgca |
| | 8461 attaacaggc cacaaatagg cgtggtaaga gaattcctta cacgtaaccc tgcttggaga |
| | 8521 aaagctgtct ttatttcacc ttataattca cagaatgctg tagcctcaaa gattttggga |
| | 8581 ctaccaactc aaactgttga ttcatcacag ggctcagaat atgactatgt catattcact |
| | 8641 caaaccactg aaacagctca ctcttgtaat gtaaacagat ttaatgttgc tattaccaga |
| | 8701 gcaaaagtag gcatactttg cataatgtct gatagagacc tttatgacaa gttgcaattt |
| | 8761 acaagtcttg aaattccacg taggaatgtg gcaactttac aagctgaaaa tgtaacagga |
| | 8821 ctctttaaag attgtagtaa ggtaatcact gggttacatc ctacacaggc acctacacac |
| | 8881 ctcagtgttg acactaaatt caaaactgaa ggtttatgtg ttgacatacc tggcatacct |
| | 8941 aaggacatga cctatagaag actcatctct atgatgggtt ttaaaatgaa ttatcaagtt |
| | 9001 aatggttacc ctaacatgtt tatcacccgc gaagaagcta taagacatgt acgtgcatgg |
| | 9061 attggcttcg atgtcgaggg gtgtcatgct actagagaag ctgttggtac caatttacct |
| | 9121 ttacagctag gtttttctac aggtgttaac ctagttgctg tacctacagg ttatgttgat |
| | 9181 acacctaata atacagattt ttccagagtt agtgctaaac caccgcctgg agatcaattt |
| | 9241 aaacacctca taccacttat gtacaaagga cttccttgga atgtagtgcg tataaagatt |
| | 9301 gtacaaatgt taagtgacac acttaaaaat ctctctgaca gagtcgtatt tgtcttatgg |
| | 9361 gcacatggct ttgagttgac atctatgaag tattttgtga aaataggacc tgagcgcacc |
| | 9421 tgttgtctat gtgatagacg tgccacatgc ttttccactg cttcagacac ttatgcctgt |
| | 9481 tggcatcatt ctattggatt tgattacgtc tataatccgt ttatgattga tgttcaacaa |
| | 9541 tggggtttta caggtaacct acaaagcaac catgatctgt attgtcaagt ccatggtaat |
| | 9601 gcacatgtag ctagttgtga tgcaatcatg actaggtgtc tagctgtcca cgagtgcttt |
| | 9661 gttaagcgtg ttgactggac tattgaatat cctataattg gtgatgaact gaagattaat |
| | 9721 gcggcttgta gaaaggttca acacatggtt gttaaagctg cattattagc agacaaattc |
| | 9781 ccagttcttc acgacattgg taaccctaaa gctattaagt gtgtacctca agctgatgta |
| | 9841 gaatggaagt tctatgatgc acagccttgt agtgacaaag cttataaaat agaagaatta |
| | 9901 ttctattctt atgccacaca ttctgacaaa ttcacagatg gtgtatgcct attttggaat |
| | 9961 tgcaatgtcg atagatatcc tgctaattcc attgtttgta gatttgacac tagagtgcta |
| | 10021 tctaacctta acttgcctgg ttgtgatggt ggcagtttgt atgtaaataa acatgcattc |
| | 10081 cacacaccag cttttgataa aagtgctttt gttaatttaa aacaattacc atttttctat |
| | 10141 tactctgaca gtccatgtga gtctcatgga aaacaagtag tgtcagatat agattatgta |
| | 10201 ccactaaagt ctgctacgtg tataacacgt tgcaatttag gtggtgctgt ctgtagacat |
| | 10261 catgctaatg agtacagatt gtatctcgat gcttataaca tgatgatctc agctggcttt |
| | 10321 agcttgtggg tttacaaaca atttgatact tataacctct ggaacacttt tacaagactt |
| | 10381 cagagtttag aaaatgtggc ttttaatgtt gtaaataagg gacactttga tggacaacag |
| | 10441 ggtgaagtac cagtttctat cattaataac actgtttaca caaaagttga tggtgttgat |
| | 10501 gtagaattgt ttgaaaataa aacaacatta cctgttaatg tagcatttga gctttgggct |
| | 10561 aagcgcaaca ttaaaccagt accagaggtg aaaatactca ataatttggg tgtggacatt |
| | 10621 gctgctaata ctgtgatctg ggactacaaa agagatgctc cagcacatat atctactatt |
| | 10681 ggtgtttgtt ctatgactga catagccaag aaaccaactg aaacgatttg tgcaccactc |
| | 10741 actgtctttt ttgatggtag agttgatggt caagtagact tatttagaaa tgcccgtaat |
| | 10801 ggtgttctta ttacagaagg tagtgttaaa ggtttacaac catctgtagg tcccaaacaa |
| | 10861 gctagtctta atggagtcac attaattgga gaagccgtaa aaacacagtt caattattat |
| | 10921 aagaaagttg atggtgttgt ccaacaatta cctgaaactt actttactca gagtagaaat |
| | 10981 ttacaagaat ttaaacccag gagtcaaatg gaaattgatt tcttagaatt agctatggat |
| | 11041 gaattcattg aacggtataa attagaaggc tatgccttcg aacatatcgt ttatggagat |
| | 11101 tttagtcata gtcagttagg tggtttacat ctactgattg gactagctaa acgttttaag |
| | 11161 gaatcacctt ttgaattaga agattttatt cctatggaca gtacagttaa aaactatttc |
| | 11221 ataacagatg cgcaaacagg ttcatctaag tgtgtgtgtt ctgttattga tttattactt |
| | 11281 gatgattttg ttgaaataat aaaatcccaa gatttatctg tagtttctaa ggttgtcaaa |
| | 11341 gtgactattg actatacaga aatttcattt atgctttggt gtaaagatgg ccatgtagaa |
| | 11401 acattttacc caaaattaca atctagtcaa gcgtggcaac cgggtgttgc tatgcctaat |
| | 11461 ctttacaaaa tgcaaagaat gctattagaa aagtgtgacc ttcaaaatta tggtgatagt |
| | 11521 gcaacattac ctaaaggcat aatgatgaat gtcgcaaaat atactcaact gtgtcaatat |
| | 11581 ttaaacacat taacattagc tgtaccctat aatatgagag ttatacattt tggtgctggt |
| | 11641 tctgataaag gagttgcacc aggtacagct gttttaagac agtggttgcc tacgggtacg |
| | 11701 ctgcttgtcg attcagatct taatgacttt gtctctgatg cagattcaac tttgattggt |
| | 11761 gattgtgcaa ctgtacatac agctaataaa tgggatctca ttattagtga tatgtacgac |
| | 11821 cctaagacta aaaatgttac aaaagaaaat gactctaaag agggtttttt cacttacatt |
| | 11881 tgtgggttta tacaacaaaa gctagctctt ggaggttccg tggctataaa gataacagaa |
| | 11941 cattcttgga atgctgatct ttataagctc atgggacact tcgcatggtg gacagccttt |
| | 12001 gttactaatg tgaatgcgtc atcatctgaa gcatttttaa ttggatgtaa ttatcttggc |
| | 12061 aaaccacgcg aacaaataga tggttatgtc atgcatgcaa attacatatt ttggaggaat |
| | 12121 acaaatccaa ttcagttgtc ttcctattct ttatttgaca tgagtaaatt tccccttaaa |
| | 12181 ttaaggggta ctgctgttat gtctttaaaa gaaggtcaaa tcaatgatat gattttatct |
| | 12241 cttcttagta aaggtagact tataattaga gaaaacaaca gagttgttat ttctagtgat |
| | 12301 gttcttgtta acaactaaac gaaccctgca gggccaccat ggtgagcaag ggcgaggagc |
| | 12361 tgttcaccgg ggtggtgccc atcctggtcg agctggacgg cgacgtaaac ggccacaagt |
| | 12421 tcagcgtgtc cggcgagggc gagggcgatg ccacctacgg caagctgacc ctgaagttca |
| | 12481 tctgcaccac cggcaagctg cccgtgccct ggcccaccct cgtgaccacc ctgacctacg |
| | 12541 gcgtgcagtg cttcagccgc taccccgacc acatgaagca gcacgacttc ttcaagtccg |
| | 12601 ccatgcccga aggctacgtc caggagcgca ccatcttctt caaggacgac ggcaactaca |
| | 12661 agacccgcgc cgaggtgaag ttcgagggcg acaccctggt gaaccgcatc gagctgaagg |
| | 12721 gcatcgactt caaggaggac ggcaacatcc tggggcacaa gctggagtac aactacaaca |
| | 12781 gccacaacgt ctatatcatg gccgacaagc agaagaacgg catcaaggtg aacttcaaga |
| | 12841 tccgccacaa catcgaggac ggcagcgtgc agctcgccga ccactaccag cagaacaccc |
| | 12901 ccatcggcga cggccccgtg ctgctgcccg acaaccacta cctgagcacc cagtccgccc |
| | 12961 tgagcaaaga ccccaacgag aagcgcgatc acatggtcct gctggagttc gtgaccgccg |
| | 13021 ccgggatcac tctcggcatg gacgagctgt acaagcctgc aggttaaact catgcagacc |
| | 13081 acacaaggca gatgggctat ataaacgttt tcgcttttcc gtttacgata tatagtctac |
| | 13141 tcttgtgcag aatgaattct cgtaactaca tagcacaagt agatgtagtt aactttaatc |
| | 13201 tcacatagca atctttaatc agtgtgtaac attagggagg acttgaaaga gccaccacat |
| | 13261 tttcaccgag gccacgcgga gtacgatcga gtgtacagtg aacaatgcta gggagagctg |
| | 13321 cctatatgga agagccctaa tgtgtaaaat taattttagt agtgctatcc ccatgtgatt |
| | 13381 ttaatagctt cttaggagaa tgacaaaaaa aaaaaaaaaa aaaaaaaaaa aaaaaaagtt |
| | 13441 taaacttgag atcctttttt tctgcgcgta atctgctgct tgcaaacaaa aaaaccaccg |
| | 13501 ctaccagcgg tggtttgttt gccggatcaa gagctaccaa ctctttttcc gaaggtaact |
| | 13561 ggcttcagca gagcgcagat accaaatact gtccttctag tgtagccgta gttaggccac |
| | 13621 cacttcaaga actctgtagc accgcctaca tacctcgctc tgctaatcct gttaccagtg |
| | 13681 gctgctgcca gtggcgataa gtcgtgtctt accgggttgg actcaagacg atagttaccg |
| | 13741 gataaggcgc agcggtcggg ctgaacgggg ggttcgtgca cacagcccag cttggagcga |
| | 13801 acgacctaca ccgaactgag atacctacag cgtgagctat gagaaagcgc cacgcttccc |
| | 13861 gaagggagaa aggcggacag gtatccggta agcggcaggg toggaacagg agagcgcacg |
| | 13921 agggagcttc cagggggaaa cgcctggtat ctttatagtc ctgtcgggtt tcgccacctc |
| | 13981 tgacttgagc gtcgattttt gtgatgctcg tcaggggggc ggagcctatg gaaacgcgga |
| | 14041 acccctattt gtttattttt ctaaatacat tcaaatatgt atccgctcat gagacaataa |
| | 14101 ccctgataaa tgcttcaata atattgaaaa aggaagagta tgagtattca acatttccgt |
| | 14161 gtcgccctta ttcccttttt tgcggcattt tgccttcctg tttttgctca cccagaaacg |
| | 14221 ctggtgaaag taaaagatgc tgaagatcag ttgggtgcac gagtgggtta catcgaactg |
| | 14281 gatctcaaca gcggtaagat ccttgagagt tttcgccccg aagaacgttt tccaatgatg |
| | 14341 agcactttta aagttctgct atgtggcgcg gtattatccc gtgttgacgc cgggcaagag |
| | 14401 caactcggtc gccgcataca ctattctcag aatgacttgg ttgagtactc accagtcaca |
| | 14461 gaaaagcatc ttacggatgg catgacagta agagaattat gcagtgctgc cataaccatg |
| | 14521 agtgataaca ctgcggccaa cttacttctg acaacgatcg gaggaccgaa ggagctaacc |
| | 14581 gcttttttgc acaacatggg ggatcatgta actcgccttg atcgttggga accggagctg |
| | 14641 aatgaagcca taccaaacga cgagcgtgac accacgatgc ctgtagcaat ggcaacaacg |
| | 14701 ttgcgcaaac tattaactgg cgaactactt actctagctt cccggcaaca attaatagac |
| | 14761 tggatggagg cggataaagt tgcaggacca cttctgcgct cggcccttcc ggctggctgg |
| | 14821 tttattgctg ataaatctgg agccggtgag cgtgggtctc gcggtatcat tgcagcactg |
| | 14881 gggccagatg gtaagccctc ccgtatcgta gttatctaca cgacggggag tcaggcaact |
| | 14941 atggatgaac gaaatagaca gatcgctgag ataggtgcct cactgattaa gcattggtaa |
| | 15001 gcgatcgcaa cgccagcaac gcgagctccg ttacataact tacggtaaat ggcccgcctg |
| | 15061 gctgaccgcc caacgacccc cgcccattga cgtcaataat gacgtatgtt cccatagtaa |
| | 15121 cgccaatagg gactttccat tgacgtcaat gggtggagta tttacggtaa actgcccact |
| | 15181 tggcagtaca tcaagtgtat catatgccaa gtacgccccc tattgacgtc aatgacggta |
| | 15241 aatggcccgc ctggcattat gcccagtaca tgaccttatg ggactttcct acttggcagt |
| | 15301 acatctacgt attagtcatc gctattacca tggtgatgcg gttttggcag tacatcaatg |
| | 15361 ggcgtggata gcggtttgac tcacggggat ttccaagtct ccaccccatt gacgtcaatg |
| | 15421 ggagtttgtt ttggcaccaa aatcaacggg actttccaaa atgtcgtaac aactccgccc |
| | 15481 cattgacgca aatgggggt aggcgtgtac ggtgggaggt ctatataagc agagctcgtt |
| | 15541 tagtgaaccg tcagaatttt gtaatacgac tcactatagg g |

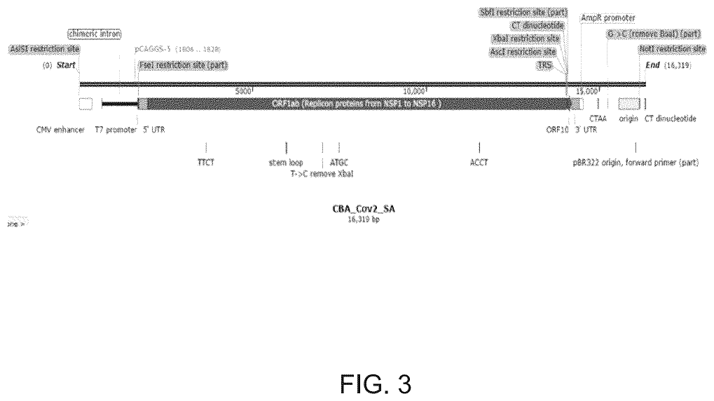

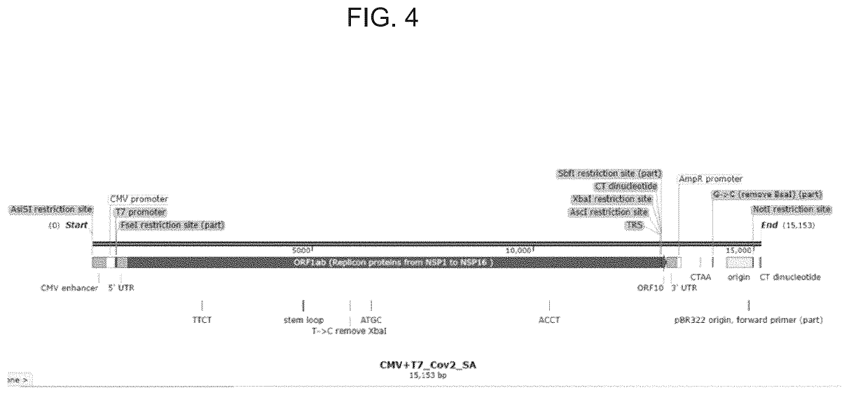

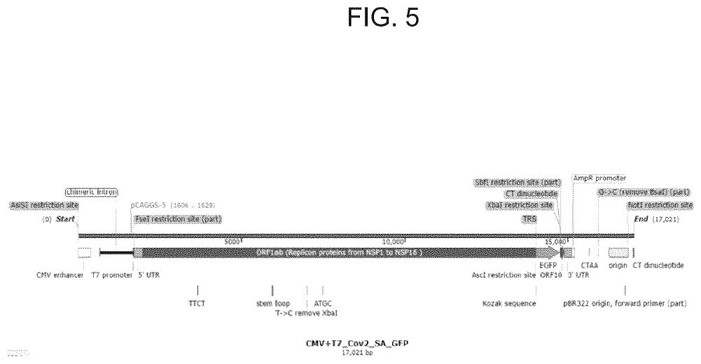

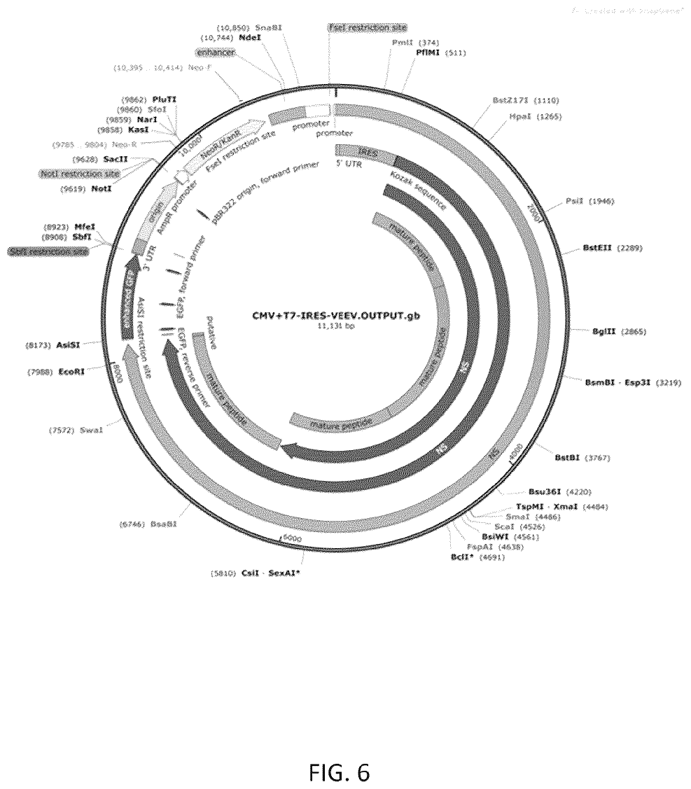

DNA sequence of CBA_CoV2_SA
(map set forth in FIG. 3).
gcgatcgcgacattgattattgactagttattaatagtaatcaat
tacggggtcattagttcatagcccatatatggagttccgcgttac
ataacttacggtaaatggcccgcctggctgaccgcccaacgaccc
ccgcccattgacgtcaataatgacgtatgttcccatagtaacgcc
aatagggactttccattgacgtcaatgggtggagtatttacggta
aactgcccacttggcagtacatcaagtgtatcatatgccaagtac
gccccctattgacgtcaatgacggtaaatggcccgcctggcatta
tgcccagtacatgaccttatgggactttcctacttggcagtacat
ctacgtattagtcatcgctattaccatggtcgaggtgagccccac
gttctgcttcactctccccatctcccccccctccccacccccaat
tttgtatttatttattttttaattattttgtgcagcgatgggggg
ggggggggggggggcgcgccagggggggggggggggggggggggg
gggggggggggggggggggggggggcggcggcagccaatcagagc
ggcgcgctccgaaagtttccttttatggcgaggcggcggcggcgg
cggccctataaaaagcgaagcgcgcggggggggagtcgctgcgcg
ctgccttcgccccgtgccccgctccgccgccgcctcgcgccgccc
gccccggctctgactgaccgcgttactcccacaggtgagcggggg
gacggcccttctcctccgggctgtaattagcgcttggtttaatga
cggcttgtttcttttctgtggctgcgtgaaagccttgaggggctc
cgggagggccctttgtgcggggggagcggctcggggggtgcgtgc
gtgtgtgtgtgcgtggggagcgccgcgtgcggctccgcgctgccc
ggcggctgtgagcgctgcgggcgcggcgcggggctttgtgcgctc
cgcagtgtgcgcgaggggagcgcggccgggggcggtgccccgcgg
tgcggggggggctgcgaggggaacaaaggctgcgtgcggggtgtg
tgcgtgggggggtgagcagggggtgtgggcgcgtcggtcgggctg
caaccccccctgcacccccctccccgagttgctgagcacggcccg
gcttcgggtgcggggctccgtacggggcgtggcgcggggctcgcc
gtgccgggcggggggggcggcaggtgggggtgccgggcggggggg
gccgcctcgggccggggggggctcggggggggggcgcggcggccc
ccggagcgccggcggctgtcgaggcgcggcgagccgcagccattg
ccttttatggtaatcgtgcgagagggcgcagggacttcctttgtc
ccaaatctgtgcggagccgaaatctgggaggcgccgccgcacccc
ctctagcgggcgcggggcgaagcggtgcggcgccggcaggaagga
aatgggcggggagggccttcgtgcgtcgccgcgccgccgtcccct
tctccctctccagcctcggggctgtccgcggggggacggctgcct
tcgggggggacggggcagggcggggttcggcttctggcgtgtgac
cggcggctctagagcctctgctaaccatgttcatgccttcttctt
tttcctacagtaatacgactcactatagggggccggccattaaag
gtttataccttcccaggtaacaaaccaaccaactttcgatctctt
gtagatctgttctctaaacgaactttaaaatctgtgtggctgtca
ctcggctgcatgcttagtgcactcacgcagtataattaataacta
attactgtcgttgacaggacacgagtaactcgtctatcttctgca
ggctgcttacggtttcgtccgtgttgcagccgatcatcagcacat
ctaggtttcgtccgggtgtgaccgaaaggtaagatggagagcctt
gtccctggtttcaacgagaaaacacacgtccaactcagtttgcct
gttttacaggttcgcgacgtgctcgtacgtggctttggagactcc
gtggaggaggtcttatcagaggcacgtcaacatcttaaagatggc
acttgtggcttagtagaagttgaaaaaggcgttttgcctcaactt
gaacagccctatgtgttcatcaaacgttcggatgctcgaactgca
cctcatggtcatgttatggttgagctggtagcagaactcgaaggc
attcagtacggtcgtagtggtgagacacttggtgtccttgtccct
catgtgggcgaaataccagtggcttaccgcaaggttcttcttcgt
aagaacggtaataaaggagctggtggccatagttacggcgccgat
ctaaagtcatttgacttaggcgacgagcttggcactgatccttat
gaagattttcaagaaaactggaacactaaacatagcagtggtgtt
acccgtgaactcatgcgtgagcttaacggaggggcaactttacaa
agtggttttagaaaaatggcattcccatctggtaaagttgagggt
tgtatggtacaagtaacttgtggtacaactacacttaacggtctt
tggcttgatgacgtagtttactgtccaagacatgtgatctgcacc
tctgaagacatgcttaaccctaattatgaagatttactcattcgt
aagtctaatcataatttcttggtacaggctggtaatgttcaactc
agggttattggacattctatgcaaaattgtgtacttaagcttaag
gttgatacagccaatcctaagacacctaagtataagtttgttcgc
attcaaccaggacagactttttcagtgttagcttgttacaatggt
tcaccatctggtgtttaccaatgtgctatgaggcccaatttcact
attaagggttcattccttaatggttcatgtggtagtgttggtttt
aacatagattatgactgtgtctctttttgttacatgcaccatatg
gaattaccaactggagttcatgctggcacagacttagaaggtaac
ttttatggaccttttgttgacaggcaaacagcacaagcagctggt
acggacacaactattacagttaatgttttagcttggttgtacgct
gctgttataaatggagacaggtggtttctcaatcgatttaccaca
actcttaatgactttaaccttgtggctatgaagtacaattatgaa
cctctaacacaagaccatgttgacatactaggacctctttctgct
caaactggaattgccgttttagatatgtgtgcttcattaaaagaa
ttactgcaaaatggtatgaatggacgtaccatattgggtagtgct
ttattagaagatgaatttacaccttttgatgttgttagacaatgc
tcaggtgttactttccaaagtgcagtgaaaagaacaatcaagggt
acacaccactggttgttactcacaattttgacttcacttttagtt
ttagtccagagtactcaatggtctttgttcttttttttgtatgaa
aatgcctttttaccttttgctatgggtattattgctatgtctgct
tttgcaatgatgtttgtcaaacataagcatgcatttctctgtttg
tttttgttaccttctcttgccactgtagcttattttaatatggtc
tatatgcctgctagttgggtgatgcgtattatgacatggttggat
atggttgatactagtttgtctggttttaagctaaaagactgtgtt
atgtatgcatcagctgtagtgttactaatccttatgacagcaaga
actgtgtatgatgatggtgctaggagagtgtggacacttatgaat
gtcttgacactcgtttataaagtttattatggtaatgctttagat
caagccatttccatgtgggctcttataatctctgttacttctaac
tactcaggtgtagttacaactgtcatgtttttggccagaggtatt
gtttttatgtgtgttgagtattgccctattttcttcataactggt
aatacacttcagtgtataatgctagtttattgtttcttaggctat
ttttgtacttgttactttggcctcttttgtttactcaaccgctac
tttagactgactcttggtgtttatgattacttagtttctacacag
gagtttagatatatgaattcacagggactactcccacccaagaat
agcatagatgccttcaaactcaacattaaattgttgggtgttggt
ggcaaaccttgtatcaaagtagccactgtacagtctaaaatgtca
gatgtaaagtgcacatcagtagtcttactctcagttttgcaacaa
ctcagagtagaatcatcatctaaattgtgggctcaatgtgtccag
ttacacaatgacattctcttagctaaagatactactgaagccttt
gaaaaaatggtttcactactttctgttttgctttccatgcagggt
gctgtagacataaacaagctttgtgaagaaatgctggacaacagg
gcaaccttacaagctatagcctcagagtttagttcccttccatca
tatgcagcttttgctactgctcaagaagcttatgagcaggctgtt
gctaatggtgattctgaagttgttcttaaaaagttgaagaagtct
ttgaatgtggctaaatctgaatttgaccgtgatgcagccatgcaa
cgtaagttggaaaagatggctgatcaagctatgacccaaatgtat
aaacaggctagatctgaggacaagagggcaaaagttactagtgct
atgcagacaatgcttttcactatgcttagaaagttggataatgat
gcactcaacaacattatcaacaatgcaagagatggttgtgttccc
ttgaacataatacctcttacaacagcagccaaactaatggttgtc
ataccagactataacacatataaaaatacgtgtgatggtacaaca
tttacttatgcatcagcattgtgggaaatccaacaggttgtagat
gcagatagtaaaattgttcaacttagtgaaattagtatggacaat
tcacctaatttagcatggcctcttattgtaacagctttaagggcc
aattctgctgtcaaattacagaataatgagcttagtcctgttgca
ctacgacagatgtcttgtgctgccggtactacacaaactgcttgc
actgatgacaatgcgttagcttactacaacacaacaaagggaggt
aggtttgtacttgcactgttatccgatttacaggatttgaaatgg
gctagattccctaagagtgatggaactggtactatctatacagaa
ctggaaccaccttgtaggtttgttacagacacacctaaaggtcct
aaagtgaagtatttatactttattaaaggattaaacaacctaaat
agaggtatggtacttggtagtttagctgccacagtacgtctacaa
gctggtaatgcaacagaagtgcctgccaattcaactgtattatct
ttctgtgcttttgctgtagatgctgctaaagcttacaaagattat
ctagctagtgggggacaaccaatcactaattgtgttaagatgttg
tgtacacacactggtactggtcaggcaataacagttacaccggaa
gccaatatggatcaagaatcctttggtggtgcatcgtgttgtctg
tactgccgttgccacatagatcatccaaatcctaaaggattttgt
gacttaaaaggtaagtatgtacaaatacctacaacttgtgctaat
gaccctgtgggttttacacttaaaaacacagtctgtaccgtctgc
ggtatgtggaaaggttatggctgtagttgtgatcaactccgcgaa
cccatgcttcagtcagctgatgcacaatcgtttttaaacgggttt
gcggtgtaagtgcagcccgtcttacaccgtgcggcacaggcacta
gtactgatgtcgtatacagggcttttgacatctacaatgataaag
tagctggttttgctaaattcctaaaaactaattgttgtcgcttcc
aagaaaaggacgaagatgacaatttaattgattcttactttgtag
ttaagagacacactttctctaactaccaacatgaagaaacaattt
ataatttacttaaggattgtccagctgttgctaaacatgacttct
ttaagtttagaatagacggtgacatggtaccacatatatcacgtc
aacgtcttactaaatacacaatggcagacctcgtctatgctttaa
ggcattttgatgaaggtaattgtgacacattaaaagaaatacttg
tcacatacaattgttgtgatgatgattatttcaataaaaaggact
ggtatgattttgtagaaaacccagatatattacgcgtatacgcca
acttaggtgaacgtgtacgccaagctttgttaaaaacagtacaat
tctgtgatgccatgcgaaatgctggtattgttggtgtactgacat
tagataatcaagatctcaatggtaactggtatgatttcggtgatt
tcatacaaaccacgccaggtagtggagttcctgttgtagattctt
attattcattgttaatgcctatattaaccttgaccagggctttaa
ctgcagagtcacatgttgacactgacttaacaaagccttacatta
agtgggatttgttaaaatatgacttcacggaagagaggttaaaac
tctttgaccgttattttaaatattgggatcagacataccacccaa
attgtgttaactgtttggatgacagatgcattctgcattgtgcaa
actttaatgttttattctctacagtgttcccacctacaagttttg
gaccactagtgagaaaaatatttgttgatggtgttccatttgtag
tttcaactggataccacttcagagagctaggtgttgtacataatc
aggatgtaaacttacatagctccagacttagttttaaggaattac
ttgtgtatgctgctgaccctgctatgcacgctgcttctggtaatc
tattactagataaacgcactacgtgcttttcagtagctgcactta
ctaacaatgttgcttttcaaactgtcaaacccggtaattttaaca
aagacttctatgactttgctgtgtctaagggtttctttaaggaag
gaagttctgttgaattaaaacacttcttctttgctcaggatggta
atgctgctatcagcgattatgactactatcgttataatctaccaa
caatgtgtgatatcagacaactactatttgtagttgaagttgttg
ataagtactttgattgttacgatggtggctgtattaatgctaacc
aagtcatcgtcaacaacctagacaaatcagctggttttccattta
ataaatggggtaaggctagactttattatgattcaatgagttatg
aggatcaagatgcacttttcgcatatacaaaacgtaatgtcatcc
ctactataactcaaatgaatcttaagtatgccattagtgcaaaga
atagagctcgcaccgtagctggtgtctctatctgtagtactatga
ccaatagacagtttcatcaaaaattattgaaatcaatagccgcca
ctagaggagctactgtagtaattggaacaagcaaattctatggtg
gttggcacaacatgttaaaaactgtttatagtgatgtagaaaacc
ctcaccttatgggttgggattatcctaaatgtgatagagccatgc
ctaacatgcttagaattatggcctcacttgttcttgctcgcaaac
atacaacgtgttgtagcttgtcacaccgtttctatagattagcta
atgagtgtgctcaagtattgagtgaaatggtcatgtgtggcggtt
cactatatgttaaaccaggtggaacctcatcaggagatgccacaa
ctgcttatgctaatagtgtttttaacatttgtcaagctgtcacgg
ccaatgttaatgcacttttatctactgatggtaacaaaattgccg
ataagtatgtccgcaatttacaacacagactttatgagtgtctct
atagaaatagagatgttgacacagactttgtgaatgagttttacg
catatttgcgtaaacatttctcaatgatgatactctctgacgatg
ctgttgtgtgtttcaatagcacttatgcatctcaaggtctagtgg
ctagcataaagaactttaagtcagttctttattatcaaaacaatg
tttttatgtctgaagcaaaatgttggactgagactgaccttacta
aaggacctcatgaattttgctctcaacatacaatgctagttaaac
agggtgatgattatgtgtaccttccttacccagatccatcaagaa
tcctaggggccggctgttttgtagatgatatcgtaaaaacagatg
gtacacttatgattgaacggttcgtgtctttagctatagatgctt
acccacttactaaacatcctaatcaggagtatgctgatgtctttc
atttgtacttacaatacataagaaagctacatgatgagttaacag
gacacatgttagacatgtattctgttatgcttactaatgataaca
cttcaaggtattgggaacctgagttttatgaggctatgtacacac
cgcatacagtcttacaggctgttggggcttgtgttctttgcaatt
cacagacttcattaagatgtggtgcttgcatacgtagaccattct
tatgttgtaaatgctgttacgaccatgtcatatcaacatcacata
aattagtcttgtctgttaatccgtatgtttgcaatgctccaggtt
gtgatgtcacagatgtgactcaactttacttaggaggtatgagct
attattgtaaatcacataaaccacccattagttttccattgtgtg
ctaatggacaagtttttggtttatataaaaatacatgtgttggta
gcgataatgttactgactttaatgcaattgcaacatgtgactgga
caaatgctggtgattacattttagctaacacctgtactgaaagac
tcaagctttttgcagcagaaacgctcaaagctactgaggagacat
ttaaactgtcttatggtattgctactgtacgtgaagtgctgtctg
acagagaattacatctttcatgggaagttggtaaacctagaccac
cacttaaccgaaattatgtctttactggttatcgtgtaactaaaa
acagtaaagtacaaataggagagtacacctttgaaaaaggtgact
atggtgatgctgttgtttaccgaggtacaacaacttacaaattaa
atgttggtgattattttgtgctgacatcacatacagtaatgccat
taagtgcacctacactagtgccacaagagcactatgttagaatta
ctggcttatacccaacactcaatatctcagatgagttttctagca
atgttgcaaattatcaaaaggttggtatgcaaaagtattctacac
tccagggaccacctggtactggtaagagtcattttgctattggcc
tagctctctactacccttctgctcgcatagtgtatacagcttgct
ctcatgccgctgttgatgcactatgtgagaaggcattaaaatatt
tgcctatagataaatgtagtagaattatacctgcacgtgctcgtg
tagagtgttttgataaattcaaagtgaattcaacattagaacagt
atgtcttttgtactgtaaatgcattgcctgagactacagcagata
tagttgtctttgatgaaatttcaatggccacaaattatgatttga
gtgttgtcaatgccagattacgtgctaagcactatgtgtacattg
gcgaccctgctcaattacctgcaccacgcacattgctaactaagg
gcacactagaaccagaatatttcaattcagtgtgtagacttatga
aaactataggtccagacatgttcctcggaacttgtcggcgttgtc
ctgctgaaattgttgacactgtgagtgctttggtttatgataata
agcttaaagcacataaagacaaatcagctcaatgctttaaaatgt
tttataagggtgttatcacgcatgatgtttcatctgcaattaaca
ggccacaaataggcgtggtaagagaattccttacacgtaaccctg
cttggagaaaagctgtctttatttcaccttataattcacagaatg
ctgtagcctcaaagattttgggactaccaactcaaactgttgatt
catcacagggctcagaatatgactatgtcatattcactcaaacca
ctgaaacagctcactcttgtaatgtaaacagatttaatgttgcta
ttaccagagcaaaagtaggcatactttgcataatgtctgatagag
acttatatgacaagttgcaatttacaagtcttgaaattccacgta
ggaatgtggcaactttacaagctgaaaatgtaacaggactcttta
aagattgtagtaaggtaatcactgggttacatcctacacaggcac
ctacacacctcagtgttgacactaaattcaaaactgaaggtttat
gtgttgacatacctggcatacctaaggacatgacctatagaagac
tcatctctatgatgggttttaaaatgaattatcaagttaatggtt
accctaacatgtttatcacccgcgaagaagctataagacatgtac
gtgcatggattggcttcgatgtcgaggggtgtcatgctactagag
aagctgttggtaccaatttacctttacagctaggtttttctacag
gtgttaacctagttgctgtacctacaggttatgttgatacaccta
ataatacagatttttccagagttagtgctaaaccaccgcctggag
atcaatttaaacacctcataccacttatgtacaaaggacttcctt
ggaatgtagtgcgtataaagattgtacaaatgttaagtgacacac
ttaaaaatctctctgacagagtcgtatttgtcttatgggcacatg
gctttgagttgacatctatgaagtattttgtgaaaataggacctg
agcgcacctgttgtctatgtgatagacgtgccacatgcttttcca
ctgcttcagacacttatgcctgttggcatcattctattggatttg
attacgtctataatccgtttatgattgatgttcaacaatggggtt
ttacaggtaacctacaaagcaaccatgatctgtattgtcaagtcc
atggtaatgcacatgtagctagttgtgatgcaatcatgactaggt
gtctagctgtccacgagtgctttgttaagcgtgttgactggacta
ttgaatatcctataattggtgatgaactgaagattaatgcggctt
gtagaaaggttcaacacatggttgttaaagctgcattattagcag
acaaattcccagttcttcacgacattggtaaccctaaagctatta
agtgtgtacctcaagctgatgtagaatggaagttctatgatgcac
agccttgtagtgacaaagcttataaaatagaagaattattctatt
cttatgccacacattctgacaaattcacagatggtgtatgcctat
tttggaattgcaatgtcgatagatatcctgctaattccattgttt
gtagatttgacactagagtgctatctaaccttaacttgcctggtt
gtgatggtggcagtttgtatgtaaataaacatgcattccacacac
cagcttttgataaaagtgcttttgttaatttaaaacaattaccat
ttttctattactctgacagtccatgtgagtctcatggaaaacaag
tagtgtcagatatagattatgtaccactaaagtctgctacgtgta
taacacgttgcaatttaggtggtgctgtctgtagacatcatgcta
atgagtacagattgtatctcgatgcttataacatgatgatctcag
ctggctttagcttgtgggtttacaaacaatttgatacttataacc
tctggaacacttttacaagacttcagagtttagaaaatgtggctt
ttaatgttgtaaataagggacactttgatggacaacagggtgaag
taccagtttctatcattaataacactgtttacacaaaagttgatg
gtgttgatgtagaattgtttgaaaataaaacaacattacctgtta
atgtagcatttgagctttgggctaagcgcaacattaaaccagtac
cagaggtgaaaatactcaataatttgggtgtggacattgctgcta
atactgtgatctgggactacaaaagagatgctccagcacatatat
ctactattggtgtttgttctatgactgacatagccaagaaaccaa
ctgaaacgatttgtgcaccactcactgtcttttttgatggtagag
ttgatggtcaagtagacttatttagaaatgcccgtaatggtgttc
ttattacagaaggtagtgttaaaggtttacaaccatctgtaggtc
ccaaacaagctagtcttaatggagtcacattaattggagaagccg
taaaaacacagttcaattattataagaaagttgatggtgttgtcc
aacaattacctgaaacttactttactcagagtagaaatttacaag
aatttaaacccaggagtcaaatggaaattgatttcttagaattag
ctatggatgaattcattgaacggtataaattagaaggctatgcct
tcgaacatatcgtttatggagattttagtcatagtcagttaggtg
gtttacatctactgattggactagctaaacgttttaaggaatcac
cttttgaattagaagattttattcctatggacagtacagttaaaa
actatttcataacagatgcgcaaacaggttcatctaagtgtgtgt
gttctgttattgatttattacttgatgattttgttgaaataataa
aatcccaagatttatctgtagtttctaaggttgtcaaagtgacta
ttgactatacagaaatttcatttatgctttggtgtaaagatggcc
atgtagaaacattttacccaaaattacaatctagtcaagcgtggc
aaccgggtgttgctatgcctaatctttacaaaatgcaaagaatgc
tattagaaaagtgtgaccttcaaaattatggtgatagtgcaacat
tacctaaaggcataatgatgaatgtcgcaaaatatactcaactgt
gtcaatatttaaacacattaacattagctgtaccctataatatga
gagttatacattttggtgctggttctgataaaggagttgcaccag
gtacagctgttttaagacagtggttgcctacgggtacgctgcttg
tcgattcagatcttaatgactttgtctctgatgcagattcaactt
tgattggtgattgtgcaactgtacatacagctaataaatgggatc
tcattattagtgatatgtacgaccctaagactaaaaatgttacaa
aagaaaatgactctaaagagggttttttcacttacatttgtgggt
ttatacaacaaaagctagctcttggaggttccgtggctataaaga
taacagaacattcttggaatgctgatctttataagctcatgggac
acttcgcatggtggacagcctttgttactaatgtgaatgcgtcat
catctgaagcatttttaattggatgtaattatcttggcaaaccac
gcgaacaaatagatggttatgtcatgcatgcaaattacatatttt
ggaggaatacaaatccaattcagttgtcttcctattctttatttg
acatgagtaaatttccccttaaattaaggggtactgctgttatgt
ctttaaaagaaggtcaaatcaatgatatgattttatctcttctta
gtaaaggtagacttataattagagaaaacaacagagttgttattt
ctagtgatgttcttgttaacaactaaACGAACggcgcgccagaat
ttatacgtctcaatgctctagactcctgcaggatgggctatataa
acgttttcgcttttccgtttacgatatatagtctactcttgtgca
gaatgaattctcgtaactacatagcacaagtagatgtagttaact
ttaatctcacatagcaatctttaatcagtgtgtaacattagggag
gacttgaaagagccaccacattttcaccgaggccacgcggagtac
gatcgagtgtacagtgaacaatgctagggagagctgcctatatgg
aagagccctaatgtgtaaaattaattttagtagtgctatccccat
gtgattttaatagcttcttaggagaatgacaaaaaaaaaaaaaaa
aaaaaaaaaaaaaaaaaacgcggaacccctatttgtttatttttc
taaatacattcaaatatgtatccgctcatgagacaataaccctga
taaatgcttcaataatattgaaaaaggaagagtatgagtattcaa
catttccgtgtcgcccttattcccttttttgcggcattttgcctt
cctgtttttgctcacccagaaacgctggtgaaagtaaaagatgct
gaagatcagttgggtgcacgagtgggttacatcgaactggatctc
aacagcggtaagatccttgagagttttcgccccgaagaacgtttt
ccaatgatgagcacttttaaagttctgctatgtggcgcggtatta
tcccgtgttgacgccgggcaagagcaactcggtcgccgcatacac
tattctcagaatgacttggttgagtactcaccagtcacagaaaag
catcttacggatggcatgacagtaagagaattatgcagtgctgcc
ataaccatgagtgataacactgcggccaacttacttctgacaacg
atcggaggaccgaaggagctaaccgcttttttgcacaacatgggg
gatcatgtaactcgccttgatcgttgggaaccggagctgaatgaa
gccataccaaacgacgagcgtgacaccacgatgcctgtagcaatg
gcaacaacgttgcgcaaactattaactggcgaactacttactcta
gcttcccggcaacaattaatagactggatggaggcggataaagtt
gcaggaccacttctgcgctcggcccttccggctggctggtttatt
gctgataaatctggagccggtgagcgtggctctcgcggtatcatt
gcagcactggggccagatggtaagccccccgtatcgtagttatct
acacgacggggagtcaggcaactatggatgaacgaaatagacaga
tcgctgagataggtgcctcactgattaagcattggtaactgtcag
accaagtttactcatatatactttagattgatttaaaacttcatt
tttaatttaaaaggatctaggtgaagatcctttttgataatctca
tgaccaaaatcccttaacgtgagttttcgttccactgagcgtcag
accccgtagaaaagatcaaaggatcttcttgagatcctttttttc
tgcgcgtaatctgctgcttgcaaacaaaaaaaccaccgctaccag
cggtggtttgtttgccggatcaagagctaccaactctttttccga
aggtaactggcttcagcagagcgcagataccaaatactgtccttc
tagtgtagccgtagttaggccaccacttcaagaactctgtagcac
cgcctacatacctcgctctgctaatcctgttaccagtggctgctg
ccagtggcgataagtcgtgtcttaccgggttggactcaagacgat
agttaccggataaggcgcagcggtcgggctgaacggggggttcgt
gcacacagcccagcttggagcgaacgacctacaccgaactgagat
acctacagcgtgagctatgagaaagcgccacgcttcccgaaggga
gaaaggcggacaggtatccggtaagcggcagggtcggaacaggag
agcgcacgagggagcttccagggggaaacgcctggtatctttata
gtcctgtcgggtttcgccacctctgacttgagcgtcgatttttgt
gatgctcgtcaggggggcggagcctatggaaagcggccgcaacgc
cagcaacgcgagctcattaaggggtactgctgttatgtctttaaa
agaaggtcaaatcaatgatatgattttatctcttcttagtaaagg
tagacttataattagagaaaacaacagagttgttatttctagtga
tgttcttgttaacaactaact
DNA sequence of CMV (map set forth in FIG. 4):
gcgatcgccgttacataacttacggtaaatggcccgcctggctga
ccgcccaacgacccccgcccattgacgtcaataatgacgtatgtt
cccatagtaacgccaatagggactttccattgacgtcaatgggtg
gagtatttacggtaaactgcccacttggcagtacatcaagtgtat
catatgccaagtacgccccctattgacgtcaatgacggtaaatgg
cccgcctggcattatgcccagtacatgaccttatgggactttcct
acttggcagtacatctacgtattagtcatcgctattaccatggtg
atgcggttttggcagtacatcaatgggcgtggatagcggtttgac
tcacggggatttccaagtctccaccccattgacgtcaatgggagt
ttgttttggcaccaaaatcaacgggactttccaaaatgtcgtaac
aactccgccccattgacgcaaatgggcggtaggcgtgtacggtgg
gaggtctatataagcagagcttaatacgactcactatagggggcc
ggccattaaaggtttataccttcccaggtaacaaaccaaccaact
ttcgatctcttgtagatctgttctctaaacgaactttaaaatctg
tgtggctgtcactcggctgcatgcttagtgcactcacgcagtata
attaataactaattactgtcgttgacaggacacgagtaactcgtc
tatcttctgcaggctgcttacggtttcgtccgtgttgcagccgat
catcagcacatctaggtttcgtccgggtgtgaccgaaaggtaaga
tggagagccttgtccctggtttcaacgagaaaacacacgtccaac
tcagtttgcctgttttacaggttcgcgacgtgctcgtacgtggct
ttggagactccgtggaggaggtcttatcagaggcacgtcaacatc
ttaaagatggcacttgtggcttagtagaagttgaaaaaggcgttt
tgcctcaacttgaacagccctatgtgttcatcaaacgttcggatg
ctcgaactgcacctcatggtcatgttatggttgagctggtagcag
aactcgaaggcattcagtacggtcgtagtggtgagacacttggtg
tccttgtccctcatgtgggcgaaataccagtggcttaccgcaagg
ttcttcttcgtaagaacggtaataaaggagctggtggccatagtt
acggcgccgatctaaagtcatttgacttaggcgacgagcttggca
ctgatccttatgaagattttcaagaaaactggaacactaaacata
gcagtggtgttacccgtgaactcatgcgtgagcttaacggagggg
caactttacaaagtggttttagaaaaatggcattcccatctggta
aagttgagggttgtatggtacaagtaacttgtggtacaactacac
ttaacggtctttggcttgatgacgtagtttactgtccaagacatg
tgatctgcacctctgaagacatgcttaaccctaattatgaagatt
tactcattcgtaagtctaatcataatttcttggtacaggctggta
atgttcaactcagggttattggacattctatgcaaaattgtgtac
ttaagcttaaggttgatacagccaatcctaagacacctaagtata
agtttgttcgcattcaaccaggacagactttttcagtgttagctt
gttacaatggttcaccatctggtgtttaccaatgtgctatgaggc
ccaatttcactattaagggttcattccttaatggttcatgtggta
gtgttggttttaacatagattatgactgtgtctctttttgttaca
tgcaccatatggaattaccaactggagttcatgctggcacagact
tagaaggtaacttttatggaccttttgttgacaggcaaacagcac
aagcagctggtacggacacaactattacagttaatgttttagctt
ggttgtacgctgctgttataaatggagacaggtggtttctcaatc
gatttaccacaactcttaatgactttaaccttgtggctatgaagt
acaattatgaacctctaacacaagaccatgttgacatactaggac
ctctttctgctcaaactggaattgccgttttagatatgtgtgctt
cattaaaagaattactgcaaaatggtatgaatggacgtaccatat
tgggtagtgctttattagaagatgaatttacaccttttgatgttg
ttagacaatgctcaggtgttactttccaaagtgcagtgaaaagaa
caatcaagggtacacaccactggttgttactcacaattttgactt
cacttttagttttagtccagagtactcaatggtctttgttctttt
ttttgtatgaaaatgcctttttaccttttgctatgggtattattg
ctatgtctgcttttgcaatgatgtttgtcaaacataagcatgcat
ttctctgtttgtttttgttaccttctcttgccactgtagcttatt
ttaatatggtctatatgcctgctagttgggtgatgcgtattatga
catggttggatatggttgatactagtttgtctggttttaagctaa
aagactgtgttatgtatgcatcagctgtagtgttactaatcctta
tgacagcaagaactgtgtatgatgatggtgctaggagagtgtgga
cacttatgaatgtcttgacactcgtttataaagtttattatggta
atgctttagatcaagccatttccatgtgggctcttataatctctg
ttacttctaactactcaggtgtagttacaactgtcatgtttttgg
ccagaggtattgtttttatgtgtgttgagtattgccctattttct
tcataactggtaatacacttcagtgtataatgctagtttattgtt
tcttaggctatttttgtacttgttactttggcctcttttgtttac
tcaaccgctactttagactgactcttggtgtttatgattacttag
tttctacacaggagtttagatatatgaattcacagggactactcc
cacccaagaatagcatagatgccttcaaactcaacattaaattgt
tgggtgttggtggcaaaccttgtatcaaagtagccactgtacagt
ctaaaatgtcagatgtaaagtgcacatcagtagtcttactctcag
ttttgcaacaactcagagtagaatcatcatctaaattgtgggctc
aatgtgtccagttacacaatgacattctcttagctaaagatacta
ctgaagcctttgaaaaaatggtttcactactttctgttttgcttt
ccatgcagggtgctgtagacataaacaagctttgtgaagaaatgc
tggacaacagggcaaccttacaagctatagcctcagagtttagtt
cccttccatcatatgcagcttttgctactgctcaagaagcttatg
agcaggctgttgctaatggtgattctgaagttgttcttaaaaagt
tgaagaagtctttgaatgtggctaaatctgaatttgaccgtgatg
cagccatgcaacgtaagttggaaaagatggctgatcaagctatga
cccaaatgtataaacaggctagatctgaggacaagagggcaaaag
ttactagtgctatgcagacaatgcttttcactatgcttagaaagt
tggataatgatgcactcaacaacattatcaacaatgcaagagatg
gttgtgttcccttgaacataatacctcttacaacagcagccaaac
taatggttgtcataccagactataacacatataaaaatacgtgtg
atggtacaacatttacttatgcatcagcattgtgggaaatccaac
aggttgtagatgcagatagtaaaattgttcaacttagtgaaatta
gtatggacaattcacctaatttagcatggcctcttattgtaacag
ctttaagggccaattctgctgtcaaattacagaataatgagctta
gtcctgttgcactacgacagatgtcttgtgctgccggtactacac
aaactgcttgcactgatgacaatgcgttagcttactacaacacaa
caaagggaggtaggtttgtacttgcactgttatccgatttacagg
atttgaaatgggctagattccctaagagtgatggaactggtacta
tctatacagaactggaaccaccttgtaggtttgttacagacacac
ctaaaggtcctaaagtgaagtatttatactttattaaaggattaa
acaacctaaatagaggtatggtacttggtagtttagctgccacag
tacgtctacaagctggtaatgcaacagaagtgcctgccaattcaa
ctgtattatctttctgtgcttttgctgtagatgctgctaaagctt
acaaagattatctagctagtgggggacaaccaatcactaattgtg
ttaagatgttgtgtacacacactggtactggtcaggcaataacag
ttacaccggaagccaatatggatcaagaatcctttggtggtgcat
cgtgttgtctgtactgccgttgccacatagatcatccaaatccta
aaggattttgtgacttaaaaggtaagtatgtacaaatacctacaa
cttgtgctaatgaccctgtgggttttacacttaaaaacacagtct
gtaccgtctgcggtatgtggaaaggttatggctgtagttgtgatc
aactccgcgaacccatgcttcagtcagctgatgcacaatcgtttt
taaacgggtttgcggtgtaagtgcagcccgtcttacaccgtgcgg
cacaggcactagtactgatgtcgtatacagggcttttgacatcta
caatgataaagtagctggttttgctaaattcctaaaaactaattg
ttgtcgcttccaagaaaaggacgaagatgacaatttaattgattc
ttactttgtagttaagagacacactttctctaactaccaacatga
agaaacaatttataatttacttaaggattgtccagctgttgctaa
acatgacttctttaagtttagaatagacggtgacatggtaccaca
tatatcacgtcaacgtcttactaaatacacaatggcagacctcgt
ctatgctttaaggcattttgatgaaggtaattgtgacacattaaa
agaaatacttgtcacatacaattgttgtgatgatgattatttcaa
taaaaaggactggtatgattttgtagaaaacccagatatattacg
cgtatacgccaacttaggtgaacgtgtacgccaagctttgttaaa
aacagtacaattctgtgatgccatgcgaaatgctggtattgttgg
tgtactgacattagataatcaagatctcaatggtaactggtatga
tttcggtgatttcatacaaaccacgccaggtagtggagttcctgt
tgtagattcttattattcattgttaatgcctatattaaccttgac
cagggctttaactgcagagtcacatgttgacactgacttaacaaa
gccttacattaagtgggatttgttaaaatatgacttcacggaaga
gaggttaaaactctttgaccgttattttaaatattgggatcagac
ataccacccaaattgtgttaactgtttggatgacagatgcattct
gcattgtgcaaactttaatgttttattctctacagtgttcccacc
tacaagttttggaccactagtgagaaaaatatttgttgatggtgt
tccatttgtagtttcaactggataccacttcagagagctaggtgt
tgtacataatcaggatgtaaacttacatagctccagacttagttt
taaggaattacttgtgtatgctgctgaccctgctatgcacgctgc
ttctggtaatctattactagataaacgcactacgtgcttttcagt
agctgcacttactaacaatgttgcttttcaaactgtcaaacccgg
taattttaacaaagacttctatgactttgctgtgtctaagggttt
ctttaaggaaggaagttctgttgaattaaaacacttcttctttgc
tcaggatggtaatgctgctatcagcgattatgactactatcgtta
taatctaccaacaatgtgtgatatcagacaactactatttgtagt
tgaagttgttgataagtactttgattgttacgatggtggctgtat
taatgctaaccaagtcatcgtcaacaacctagacaaatcagctgg
ttttccatttaataaatggggtaaggctagactttattatgattc
aatgagttatgaggatcaagatgcacttttcgcatatacaaaacg
taatgtcatccctactataactcaaatgaatcttaagtatgccat
tagtgcaaagaatagagctcgcaccgtagctggtgtctctatctg
tagtactatgaccaatagacagtttcatcaaaaattattgaaatc
aatagccgccactagaggagctactgtagtaattggaacaagcaa
attctatggtggttggcacaacatgttaaaaactgtttatagtga
tgtagaaaaccctcaccttatgggttgggattatcctaaatgtga
tagagccatgcctaacatgcttagaattatggcctcacttgttct
tgctcgcaaacatacaacgtgttgtagcttgtcacaccgtttcta
tagattagctaatgagtgtgctcaagtattgagtgaaatggtcat
gtgtggcggttcactatatgttaaaccaggtggaacctcatcagg
agatgccacaactgcttatgctaatagtgtttttaacatttgtca
agctgtcacggccaatgttaatgcacttttatctactgatggtaa
caaaattgccgataagtatgtccgcaatttacaacacagacttta
tgagtgtctctatagaaatagagatgttgacacagactttgtgaa
tgagttttacgcatatttgcgtaaacatttctcaatgatgatact
ctctgacgatgctgttgtgtgtttcaatagcacttatgcatctca
aggtctagtggctagcataaagaactttaagtcagttctttatta
tcaaaacaatgtttttatgtctgaagcaaaatgttggactgagac
tgaccttactaaaggacctcatgaattttgctctcaacatacaat
gctagttaaacagggtgatgattatgtgtaccttccttacccaga
tccatcaagaatcctaggggccggctgttttgtagatgatatcgt
aaaaacagatggtacacttatgattgaacggttcgtgtctttagc
tatagatgcttacccacttactaaacatcctaatcaggagtatgc
tgatgtctttcatttgtacttacaatacataagaaagctacatga
tgagttaacaggacacatgttagacatgtattctgttatgcttac
taatgataacacttcaaggtattgggaacctgagttttatgaggc
tatgtacacaccgcatacagtcttacaggctgttggggcttgtgt
tctttgcaattcacagacttcattaagatgtggtgcttgcatacg
tagaccattcttatgttgtaaatgctgttacgaccatgtcatatc
aacatcacataaattagtcttgtctgttaatccgtatgtttgcaa
tgctccaggttgtgatgtcacagatgtgactcaactttacttagg
aggtatgagctattattgtaaatcacataaaccacccattagttt
tccattgtgtgctaatggacaagtttttggtttatataaaaatac
atgtgttggtagcgataatgttactgactttaatgcaattgcaac
atgtgactggacaaatgctggtgattacattttagctaacacctg
tactgaaagactcaagctttttgcagcagaaacgctcaaagctac
tgaggagacatttaaactgtcttatggtattgctactgtacgtga
agtgctgtctgacagagaattacatctttcatgggaagttggtaa
acctagaccaccacttaaccgaaattatgtctttactggttatcg
tgtaactaaaaacagtaaagtacaaataggagagtacacctttga
aaaaggtgactatggtgatgctgttgtttaccgaggtacaacaac
ttacaaattaaatgttggtgattattttgtgctgacatcacatac
agtaatgccattaagtgcacctacactagtgccacaagagcacta
tgttagaattactggcttatacccaacactcaatatctcagatga
gttttctagcaatgttgcaaattatcaaaaggttggtatgcaaaa
gtattctacactccagggaccacctggtactggtaagagtcattt
tgctattggcctagctctctactacccttctgctcgcatagtgta
tacagcttgctctcatgccgctgttgatgcactatgtgagaaggc
attaaaatatttgcctatagataaatgtagtagaattatacctgc
acgtgctcgtgtagagtgttttgataaattcaaagtgaattcaac
attagaacagtatgtcttttgtactgtaaatgcattgcctgagac
tacagcagatatagttgtctttgatgaaatttcaatggccacaaa
ttatgatttgagtgttgtcaatgccagattacgtgctaagcacta
tgtgtacattggcgaccctgctcaattacctgcaccacgcacatt
gctaactaagggcacactagaaccagaatatttcaattcagtgtg
tagacttatgaaaactataggtccagacatgttcctcggaacttg
tcggcgttgtcctgctgaaattgttgacactgtgagtgctttggt
ttatgataataagcttaaagcacataaagacaaatcagctcaatg
ctttaaaatgttttataagggtgttatcacgcatgatgtttcatc
tgcaattaacaggccacaaataggcgtggtaagagaattccttac
acgtaaccctgcttggagaaaagctgtctttatttcaccttataa
ttcacagaatgctgtagcctcaaagattttgggactaccaactca
aactgttgattcatcacagggctcagaatatgactatgtcatatt
cactcaaaccactgaaacagctcactcttgtaatgtaaacagatt
taatgttgctattaccagagcaaaagtaggcatactttgcataat
gtctgatagagacttatatgacaagttgcaatttacaagtcttga
aattccacgtaggaatgtggcaactttacaagctgaaaatgtaac
aggactctttaaagattgtagtaaggtaatcactgggttacatcc
tacacaggcacctacacacctcagtgttgacactaaattcaaaac
tgaaggtttatgtgttgacatacctggcatacctaaggacatgac
ctatagaagactcatctctatgatgggttttaaaatgaattatca
agttaatggttaccctaacatgtttatcacccgcgaagaagctat
aagacatgtacgtgcatggattggcttcgatgtcgaggggtgtca
tgctactagagaagctgttggtaccaatttacctttacagctagg
tttttctacaggtgttaacctagttgctgtacctacaggttatgt
tgatacacctaataatacagatttttccagagttagtgctaaacc
accgcctggagatcaatttaaacacctcataccacttatgtacaa
aggacttccttggaatgtagtgcgtataaagattgtacaaatgtt
aagtgacacacttaaaaatctctctgacagagtcgtatttgtctt
atgggcacatggctttgagttgacatctatgaagtattttgtgaa
aataggacctgagcgcacctgttgtctatgtgatagacgtgccac
atgcttttccactgcttcagacacttatgcctgttggcatcattc
tattggatttgattacgtctataatccgtttatgattgatgttca
acaatggggttttacaggtaacctacaaagcaaccatgatctgta
ttgtcaagtccatggtaatgcacatgtagctagttgtgatgcaat
catgactaggtgtctagctgtccacgagtgctttgttaagcgtgt
tgactggactattgaatatcctataattggtgatgaactgaagat
taatgcggcttgtagaaaggttcaacacatggttgttaaagctgc
attattagcagacaaattcccagttcttcacgacattggtaaccc
taaagctattaagtgtgtacctcaagctgatgtagaatggaagtt
ctatgatgcacagccttgtagtgacaaagcttataaaatagaaga
attattctattcttatgccacacattctgacaaattcacagatgg
tgtatgcctattttggaattgcaatgtcgatagatatcctgctaa
ttccattgtttgtagatttgacactagagtgctatctaaccttaa
cttgcctggttgtgatggtggcagtttgtatgtaaataaacatgc
attccacacaccagcttttgataaaagtgcttttgttaatttaaa
acaattaccatttttctattactctgacagtccatgtgagtctca
tggaaaacaagtagtgtcagatatagattatgtaccactaaagtc
tgctacgtgtataacacgttgcaatttaggtggtgctgtctgtag
acatcatgctaatgagtacagattgtatctcgatgcttataacat
gatgatctcagctggctttagcttgtgggtttacaaacaatttga
tacttataacctctggaacacttttacaagacttcagagtttaga
aaatgtggcttttaatgttgtaaataagggacactttgatggaca
acagggtgaagtaccagtttctatcattaataacactgtttacac
aaaagttgatggtgttgatgtagaattgtttgaaaataaaacaac
attacctgttaatgtagcatttgagctttgggctaagcgcaacat
taaaccagtaccagaggtgaaaatactcaataatttgggtgtgga
cattgctgctaatactgtgatctgggactacaaaagagatgctcc
agcacatatatctactattggtgtttgttctatgactgacatagc
caagaaaccaactgaaacgatttgtgcaccactcactgtcttttt
tgatggtagagttgatggtcaagtagacttatttagaaatgcccg
taatggtgttcttattacagaaggtagtgttaaaggtttacaacc
atctgtaggtcccaaacaagctagtcttaatggagtcacattaat
tggagaagccgtaaaaacacagttcaattattataagaaagttga
tggtgttgtccaacaattacctgaaacttactttactcagagtag
aaatttacaagaatttaaacccaggagtcaaatggaaattgattt
cttagaattagctatggatgaattcattgaacggtataaattaga
aggctatgccttcgaacatatcgtttatggagattttagtcatag
tcagttaggtggtttacatctactgattggactagctaaacgttt
taaggaatcaccttttgaattagaagattttattcctatggacag
tacagttaaaaactatttcataacagatgcgcaaacaggttcatc
taagtgtgtgtgttctgttattgatttattacttgatgattttgt
tgaaataataaaatcccaagatttatctgtagtttctaaggttgt
caaagtgactattgactatacagaaatttcatttatgctttggtg
taaagatggccatgtagaaacattttacccaaaattacaatctag
tcaagcgtggcaaccgggtgttgctatgcctaatctttacaaaat
gcaaagaatgctattagaaaagtgtgaccttcaaaattatggtga
tagtgcaacattacctaaaggcataatgatgaatgtcgcaaaata
tactcaactgtgtcaatatttaaacacattaacattagctgtacc
ctataatatgagagttatacattttggtgctggttctgataaagg
agttgcaccaggtacagctgttttaagacagtggttgcctacggg
tacgctgcttgtcgattcagatcttaatgactttgtctctgatgc
agattcaactttgattggtgattgtgcaactgtacatacagctaa
taaatgggatctcattattagtgatatgtacgaccctaagactaa
aaatgttacaaaagaaaatgactctaaagagggttttttcactta
catttgtgggtttatacaacaaaagctagctcttggaggttccgt
ggctataaagataacagaacattcttggaatgctgatctttataa
gctcatgggacacttcgcatggtggacagcctttgttactaatgt
gaatgcgtcatcatctgaagcatttttaattggatgtaattatct
tggcaaaccacgcgaacaaatagatggttatgtcatgcatgcaaa
ttacatattttggaggaatacaaatccaattcagttgtcttccta
ttctttatttgacatgagtaaatttccccttaaattaaggggtac
tgctgttatgtctttaaaagaaggtcaaatcaatgatatgatttt
atctcttcttagtaaaggtagacttataattagagaaaacaacag
agttgttatttctagtgatgttcttgttaacaactaaACGAACgg
cgcgccagaatttatacgtctcaatgctctagactcctgcaggat
gggctatataaacgttttcgcttttccgtttacgatatatagtct
actcttgtgcagaatgaattctcgtaactacatagcacaagtaga
tgtagttaactttaatctcacatagcaatctttaatcagtgtgta
acattagggaggacttgaaagagccaccacattttcaccgaggcc
acgcggagtacgatcgagtgtacagtgaacaatgctagggagagc
tgcctatatggaagagccctaatgtgtaaaattaattttagtagt
gctatccccatgtgattttaatagcttcttaggagaatgacaaaa
aaaaaaaaaaaaaaaaaaaaaaaaaaaaacgcggaacccctattt
gtttatttttctaaatacattcaaatatgtatccgctcatgagac
aataaccctgataaatgcttcaataatattgaaaaaggaagagta
tgagtattcaacatttccgtgtcgcccttattcccttttttgcgg
cattttgccttcctgtttttgctcacccagaaacgctggtgaaag
taaaagatgctgaagatcagttgggtgcacgagtgggttacatcg
aactggatctcaacagcggtaagatccttgagagttttcgccccg
aagaacgttttccaatgatgagcacttttaaagttctgctatgtg
gcgcggtattatcccgtgttgacgccgggcaagagcaactcggtc
gccgcatacactattctcagaatgacttggttgagtactcaccag
tcacagaaaagcatcttacggatggcatgacagtaagagaattat
gcagtgctgccataaccatgagtgataacactgcggccaacttac
ttctgacaacgatcggaggaccgaaggagctaaccgcttttttgc
acaacatgggggatcatgtaactcgccttgatcgttgggaaccgg
agctgaatgaagccataccaaacgacgagcgtgacaccacgatgc
ctgtagcaatggcaacaacgttgcgcaaactattaactggcgaac
tacttactctagcttcccggcaacaattaatagactggatggagg
cggataaagttgcaggaccacttctgcgctcggcccttccggctg
gctggtttattgctgataaatctggagccggtgagcgtggctctc
gcggtatcattgcagcactggggccagatggtaagccctcccgta
tcgtagttatctacacgacggggagtcaggcaactatggatgaac
gaaatagacagatcgctgagataggtgcctcactgattaagcatt
ggtaactgtcagaccaagtttactcatatatactttagattgatt
taaaacttcatttttaatttaaaaggatctaggtgaagatccttt
ttgataatctcatgaccaaaatcccttaacgtgagttttcgttcc
actgagcgtcagaccccgtagaaaagatcaaaggatcttcttgag
atcctttttttctgcgcgtaatctgctgcttgcaaacaaaaaaac
caccgctaccagcggtggtttgtttgccggatcaagagctaccaa
ctctttttccgaaggtaactggcttcagcagagcgcagataccaa
atactgtccttctagtgtagccgtagttaggccaccacttcaaga
actctgtagcaccgcctacatacctcgctctgctaatcctgttac
cagtggctgctgccagtggcgataagtcgtgtcttaccgggttgg
actcaagacgatagttaccggataaggcgcagcggtcgggctgaa
cggggggttcgtgcacacagcccagcttggagcgaacgacctaca
ccgaactgagatacctacagcgtgagctatgagaaagcgccacgc
ttcccgaagggagaaaggcggacaggtatccggtaagcggcaggg
tcggaacaggagagcgcacgagggagcttccagggggaaacgcct
ggtatctttatagtcctgtcgggtttcgccacctctgacttgagc
gtcgatttttgtgatgctcgtcaggggggcggagcctatggaaag
cggccgcaacgccagcaacgcgagctcattaaggggtactgctgt
tatgtctttaaaagaaggtcaaatcaatgatatgattttatctct
tcttagtaaaggtagacttataattagagaaaacaacagagttgt
tatttctagtgatgttcttgttaacaactaact
DNA sequence of CMV(map set forth in FIG. 5)
gcgatcgcgacattgattattgactagttattaatagtaatcaat
tacggggtcattagttcatagcccatatatggagttccgcgttac
ataacttacggtaaatggcccgcctggctgaccgcccaacgaccc
ccgcccattgacgtcaataatgacgtatgttcccatagtaacgcc
aatagggactttccattgacgtcaatgggtggagtatttacggta
aactgcccacttggcagtacatcaagtgtatcatatgccaagtac
gccccctattgacgtcaatgacggtaaatggcccgcctggcatta
tgcccagtacatgaccttatgggactttcctacttggcagtacat
ctacgtattagtcatcgctattaccatggtcgaggtgagccccac
gttctgcttcactctccccatctcccccccctccccacccccaat
tttgtatttatttattttttaattattttgtgcagcgatgggggc
ggggggggggggggcgcgcgccagggggggggggggggggggggg
gggggggggggggggggggggggggggcggcggcagccaatcaga
gcggcgcgctccgaaagtttccttttatggcgaggcggcggcggc
ggcggccctataaaaagcgaagcgcgcggggggggagtcgctgcg
cgctgccttcgccccgtgccccgctccgccgccgcctcgcgccgc
ccgccccggctctgactgaccgcgttactcccacaggtgagcggg
gggacggcccttctcctccgggctgtaattagcgcttggtttaat
gacggcttgtttcttttctgtggctgcgtgaaagccttgaggggc
tccgggagggccctttgtgcggggggagcggctcggggggtgcgt
gcgtgtgtgtgtgcgtggggagcgccgcgtgcggctccgcgctgc
ccggcggctgtgagcgctgcgggcgcggcgcggggctttgtgcgc
tccgcagtgtgcgcgaggggagcgcggccgggggcggtgccccgc
ggtgcggggggggctgcgaggggaacaaaggctgcgtgcggggtg
tgtgcgtgggggggtgagcagggggtgtgggcgcgtcggtcgggc
tgcaaccccccctgcacccccctccccgagttgctgagcacggcc
cggcttcgggtgcggggctccgtacggggcgtggcgcggggctcg
ccgtgccgggcggggggggcggcaggtgggggtgccgggcggggg
gggccgcctcgggccggggggggctcggggggggggcgcggcggc
ccccggagcgccggcggctgtcgaggcgcggcgagccgcagccat
tgccttttatggtaatcgtgcgagagggcgcagggacttcctttg
tcccaaatctgtgcggagccgaaatctgggaggcgccgccgcacc
ccctctagcgggcgcggggcgaagcggtgcggcgccggcaggaag
gaaatgggcggggagggccttcgtgcgtcgccgcgccgccgtccc
cttctccctctccagcctcggggctgtccgcggggggacggctgc
cttcgggggggacggggcagggcggggttcggcttctggcgtgtg
accggcggctctagagcctctgctaaccatgttcatgccttcttc
tttttcctacagtaatacgactcactatagggggccggccattaa
aggtttataccttcccaggtaacaaaccaaccaactttcgatctc
ttgtagatctgttctctaaacgaactttaaaatctgtgtggctgt
cactcggctgcatgcttagtgcactcacgcagtataattaataac
taattactgtcgttgacaggacacgagtaactcgtctatcttctg
caggctgcttacggtttcgtccgtgttgcagccgatcatcagcac
atctaggtttcgtccgggtgtgaccgaaaggtaagatggagagcc
ttgtccctggtttcaacgagaaaacacacgtccaactcagtttgc
ctgttttacaggttcgcgacgtgctcgtacgtggctttggagact
ccgtggaggaggtcttatcagaggcacgtcaacatcttaaagatg
gcacttgtggcttagtagaagttgaaaaaggcgttttgcctcaac
ttgaacagccctatgtgttcatcaaacgttcggatgctcgaactg
cacctcatggtcatgttatggttgagctggtagcagaactcgaag
gcattcagtacggtcgtagtggtgagacacttggtgtccttgtcc
ctcatgtgggcgaaataccagtggcttaccgcaaggttcttcttc
gtaagaacggtaataaaggagctggtggccatagttacggcgccg
atctaaagtcatttgacttaggcgacgagcttggcactgatcctt
atgaagattttcaagaaaactggaacactaaacatagcagtggtg
ttacccgtgaactcatgcgtgagcttaacggaggggcaactttac
aaagtggttttagaaaaatggcattcccatctggtaaagttgagg
gttgtatggtacaagtaacttgtggtacaactacacttaacggtc
tttggcttgatgacgtagtttactgtccaagacatgtgatctgca
cctctgaagacatgcttaaccctaattatgaagatttactcattc
gtaagtctaatcataatttcttggtacaggctggtaatgttcaac
tcagggttattggacattctatgcaaaattgtgtacttaagctta
aggttgatacagccaatcctaagacacctaagtataagtttgttc
gcattcaaccaggacagactttttcagtgttagcttgttacaatg
gttcaccatctggtgtttaccaatgtgctatgaggcccaatttca
ctattaagggttcattccttaatggttcatgtggtagtgttggtt
ttaacatagattatgactgtgtctctttttgttacatgcaccata
tggaattaccaactggagttcatgctggcacagacttagaaggta
acttttatggaccttttgttgacaggcaaacagcacaagcagctg
gtacggacacaactattacagttaatgttttagcttggttgtacg
ctgctgttataaatggagacaggtggtttctcaatcgatttacca
caactcttaatgactttaaccttgtggctatgaagtacaattatg
aacctctaacacaagaccatgttgacatactaggacctctttctg
ctcaaactggaattgccgttttagatatgtgtgcttcattaaaag
aattactgcaaaatggtatgaatggacgtaccatattgggtagtg
ctttattagaagatgaatttacaccttttgatgttgttagacaat
gctcaggtgttactttccaaagtgcagtgaaaagaacaatcaagg
gtacacaccactggttgttactcacaattttgacttcacttttag
ttttagtccagagtactcaatggtctttgttcttttttttgtatg
aaaatgcctttttaccttttgctatgggtattattgctatgtctg
cttttgcaatgatgtttgtcaaacataagcatgcatttctctgtt
tgtttttgttaccttctcttgccactgtagcttattttaatatgg
tctatatgcctgctagttgggtgatgcgtattatgacatggttgg
atatggttgatactagtttgtctggttttaagctaaaagactgtg
ttatgtatgcatcagctgtagtgttactaatccttatgacagcaa
gaactgtgtatgatgatggtgctaggagagtgtggacacttatga
atgtcttgacactcgtttataaagtttattatggtaatgctttag
atcaagccatttccatgtgggctcttataatctctgttacttcta
actactcaggtgtagttacaactgtcatgtttttggccagaggta
ttgtttttatgtgtgttgagtattgccctattttcttcataactg
gtaatacacttcagtgtataatgctagtttattgtttcttaggct
atttttgtacttgttactttggcctcttttgtttactcaaccgct
actttagactgactcttggtgtttatgattacttagtttctacac
aggagtttagatatatgaattcacagggactactcccacccaaga
atagcatagatgccttcaaactcaacattaaattgttgggtgttg
gtggcaaaccttgtatcaaagtagccactgtacagtctaaaatgt
cagatgtaaagtgcacatcagtagtcttactctcagttttgcaac
aactcagagtagaatcatcatctaaattgtgggctcaatgtgtcc
agttacacaatgacattctcttagctaaagatactactgaagcct
ttgaaaaaatggtttcactactttctgttttgctttccatgcagg
gtgctgtagacataaacaagctttgtgaagaaatgctggacaaca
gggcaaccttacaagctatagcctcagagtttagttcccttccat
catatgcagcttttgctactgctcaagaagcttatgagcaggctg
ttgctaatggtgattctgaagttgttcttaaaaagttgaagaagt
ctttgaatgtggctaaatctgaatttgaccgtgatgcagccatgc
aacgtaagttggaaaagatggctgatcaagctatgacccaaatgt
ataaacaggctagatctgaggacaagagggcaaaagttactagtg
ctatgcagacaatgcttttcactatgcttagaaagttggataatg
atgcactcaacaacattatcaacaatgcaagagatggttgtgttc
ccttgaacataatacctcttacaacagcagccaaactaatggttg
tcataccagactataacacatataaaaatacgtgtgatggtacaa
catttacttatgcatcagcattgtgggaaatccaacaggttgtag
atgcagatagtaaaattgttcaacttagtgaaattagtatggaca
attcacctaatttagcatggcctcttattgtaacagctttaaggg
ccaattctgctgtcaaattacagaataatgagcttagtcctgttg
cactacgacagatgtcttgtgctgccggtactacacaaactgctt
gcactgatgacaatgcgttagcttactacaacacaacaaagggag
gtaggtttgtacttgcactgttatccgatttacaggatttgaaat
gggctagattccctaagagtgatggaactggtactatctatacag
aactggaaccaccttgtaggtttgttacagacacacctaaaggtc
ctaaagtgaagtatttatactttattaaaggattaaacaacctaa
atagaggtatggtacttggtagtttagctgccacagtacgtctac
aagctggtaatgcaacagaagtgcctgccaattcaactgtattat
ctttctgtgcttttgctgtagatgctgctaaagcttacaaagatt
atctagctagtgggggacaaccaatcactaattgtgttaagatgt
tgtgtacacacactggtactggtcaggcaataacagttacaccgg
aagccaatatggatcaagaatcctttggtggtgcatcgtgttgtc
tgtactgccgttgccacatagatcatccaaatcctaaaggatttt
gtgacttaaaaggtaagtatgtacaaatacctacaacttgtgcta
atgaccctgtgggttttacacttaaaaacacagtctgtaccgtct
gcggtatgtggaaaggttatggctgtagttgtgatcaactccgcg
aacccatgcttcagtcagctgatgcacaatcgtttttaaacgggt
ttgcggtgtaagtgcagcccgtcttacaccgtgcggcacaggcac
tagtactgatgtcgtatacagggcttttgacatctacaatgataa
agtagctggttttgctaaattcctaaaaactaattgttgtcgctt
ccaagaaaaggacgaagatgacaatttaattgattcttactttgt
agttaagagacacactttctctaactaccaacatgaagaaacaat
ttataatttacttaaggattgtccagctgttgctaaacatgactt
ctttaagtttagaatagacggtgacatggtaccacatatatcacg
tcaacgtcttactaaatacacaatggcagacctcgtctatgcttt
aaggcattttgatgaaggtaattgtgacacattaaaagaaatact
tgtcacatacaattgttgtgatgatgattatttcaataaaaagga
ctggtatgattttgtagaaaacccagatatattacgcgtatacgc
caacttaggtgaacgtgtacgccaagctttgttaaaaacagtaca
attctgtgatgccatgcgaaatgctggtattgttggtgtactgac
attagataatcaagatctcaatggtaactggtatgatttcggtga
tttcatacaaaccacgccaggtagtggagttcctgttgtagattc
ttattattcattgttaatgcctatattaaccttgaccagggcttt
aactgcagagtcacatgttgacactgacttaacaaagccttacat
taagtgggatttgttaaaatatgacttcacggaagagaggttaaa
actctttgaccgttattttaaatattgggatcagacataccaccc
aaattgtgttaactgtttggatgacagatgcattctgcattgtgc
aaactttaatgttttattctctacagtgttcccacctacaagttt
tggaccactagtgagaaaaatatttgttgatggtgttccatttgt
agtttcaactggataccacttcagagagctaggtgttgtacataa
tcaggatgtaaacttacatagctccagacttagttttaaggaatt
acttgtgtatgctgctgaccctgctatgcacgctgcttctggtaa
tctattactagataaacgcactacgtgcttttcagtagctgcact
tactaacaatgttgcttttcaaactgtcaaacccggtaattttaa
caaagacttctatgactttgctgtgtctaagggtttctttaagga
aggaagttctgttgaattaaaacacttcttctttgctcaggatgg
taatgctgctatcagcgattatgactactatcgttataatctacc
aacaatgtgtgatatcagacaactactatttgtagttgaagttgt
tgataagtactttgattgttacgatggtggctgtattaatgctaa
ccaagtcatcgtcaacaacctagacaaatcagctggttttccatt
taataaatggggtaaggctagactttattatgattcaatgagtta
tgaggatcaagatgcacttttcgcatatacaaaacgtaatgtcat
ccctactataactcaaatgaatcttaagtatgccattagtgcaaa
gaatagagctcgcaccgtagctggtgtctctatctgtagtactat
gaccaatagacagtttcatcaaaaattattgaaatcaatagccgc
cactagaggagctactgtagtaattggaacaagcaaattctatgg
tggttggcacaacatgttaaaaactgtttatagtgatgtagaaaa
ccctcaccttatgggttgggattatcctaaatgtgatagagccat
gcctaacatgcttagaattatggcctcacttgttcttgctcgcaa
acatacaacgtgttgtagcttgtcacaccgtttctatagattagc
taatgagtgtgctcaagtattgagtgaaatggtcatgtgtggcgg
ttcactatatgttaaaccaggtggaacctcatcaggagatgccac
aactgcttatgctaatagtgtttttaacatttgtcaagctgtcac
ggccaatgttaatgcacttttatctactgatggtaacaaaattgc
cgataagtatgtccgcaatttacaacacagactttatgagtgtct
ctatagaaatagagatgttgacacagactttgtgaatgagtttta
cgcatatttgcgtaaacatttctcaatgatgatactctctgacga
tgctgttgtgtgtttcaatagcacttatgcatctcaaggtctagt
ggctagcataaagaactttaagtcagttctttattatcaaaacaa
tgtttttatgtctgaagcaaaatgttggactgagactgaccttac
taaaggacctcatgaattttgctctcaacatacaatgctagttaa
acagggtgatgattatgtgtaccttccttacccagatccatcaag
aatcctaggggccggctgttttgtagatgatatcgtaaaaacaga
tggtacacttatgattgaacggttcgtgtctttagctatagatgc
ttacccacttactaaacatcctaatcaggagtatgctgatgtctt
tcatttgtacttacaatacataagaaagctacatgatgagttaac
aggacacatgttagacatgtattctgttatgcttactaatgataa
cacttcaaggtattgggaacctgagttttatgaggctatgtacac
accgcatacagtcttacaggctgttggggcttgtgttctttgcaa
ttcacagacttcattaagatgtggtgcttgcatacgtagaccatt
cttatgttgtaaatgctgttacgaccatgtcatatcaacatcaca
taaattagtcttgtctgttaatccgtatgtttgcaatgctccagg
ttgtgatgtcacagatgtgactcaactttacttaggaggtatgag
ctattattgtaaatcacataaaccacccattagttttccattgtg
tgctaatggacaagtttttggtttatataaaaatacatgtgttgg
tagcgataatgttactgactttaatgcaattgcaacatgtgactg
gacaaatgctggtgattacattttagctaacacctgtactgaaag
actcaagctttttgcagcagaaacgctcaaagctactgaggagac
atttaaactgtcttatggtattgctactgtacgtgaagtgctgtc
tgacagagaattacatctttcatgggaagttggtaaacctagacc
accacttaaccgaaattatgtctttactggttatcgtgtaactaa
aaacagtaaagtacaaataggagagtacacctttgaaaaaggtga
ctatggtgatgctgttgtttaccgaggtacaacaacttacaaatt
aaatgttggtgattattttgtgctgacatcacatacagtaatgcc
attaagtgcacctacactagtgccacaagagcactatgttagaat
tactggcttatacccaacactcaatatctcagatgagttttctag
caatgttgcaaattatcaaaaggttggtatgcaaaagtattctac
actccagggaccacctggtactggtaagagtcattttgctattgg
cctagctctctactacccttctgctcgcatagtgtatacagcttg
ctctcatgccgctgttgatgcactatgtgagaaggcattaaaata
tttgcctatagataaatgtagtagaattatacctgcacgtgctcg
tgtagagtgttttgataaattcaaagtgaattcaacattagaaca
gtatgtcttttgtactgtaaatgcattgcctgagactacagcaga
tatagttgtctttgatgaaatttcaatggccacaaattatgattt
gagtgttgtcaatgccagattacgtgctaagcactatgtgtacat
tggcgaccctgctcaattacctgcaccacgcacattgctaactaa
gggcacactagaaccagaatatttcaattcagtgtgtagacttat
gaaaactataggtccagacatgttcctcggaacttgtcggcgttg
tcctgctgaaattgttgacactgtgagtgctttggtttatgataa
taagcttaaagcacataaagacaaatcagctcaatgctttaaaat
gttttataagggtgttatcacgcatgatgtttcatctgcaattaa
caggccacaaataggcgtggtaagagaattccttacacgtaaccc
tgcttggagaaaagctgtctttatttcaccttataattcacagaa
tgctgtagcctcaaagattttgggactaccaactcaaactgttga
ttcatcacagggctcagaatatgactatgtcatattcactcaaac
cactgaaacagctcactcttgtaatgtaaacagatttaatgttgc
tattaccagagcaaaagtaggcatactttgcataatgtctgatag
agacttatatgacaagttgcaatttacaagtcttgaaattccacg
taggaatgtggcaactttacaagctgaaaatgtaacaggactctt
taaagattgtagtaaggtaatcactgggttacatcctacacaggc
acctacacacctcagtgttgacactaaattcaaaactgaaggttt
atgtgttgacatacctggcatacctaaggacatgacctatagaag
actcatctctatgatgggttttaaaatgaattatcaagttaatgg
ttaccctaacatgtttatcacccgcgaagaagctataagacatgt
acgtgcatggattggcttcgatgtcgaggggtgtcatgctactag
agaagctgttggtaccaatttacctttacagctaggtttttctac
aggtgttaacctagttgctgtacctacaggttatgttgatacacc
taataatacagatttttccagagttagtgctaaaccaccgcctgg
agatcaatttaaacacctcataccacttatgtacaaaggacttcc
ttggaatgtagtgcgtataaagattgtacaaatgttaagtgacac
acttaaaaatctctctgacagagtcgtatttgtcttatgggcaca
tggctttgagttgacatctatgaagtattttgtgaaaataggacc
tgagcgcacctgttgtctatgtgatagacgtgccacatgcttttc
cactgcttcagacacttatgcctgttggcatcattctattggatt
tgattacgtctataatccgtttatgattgatgttcaacaatgggg
ttttacaggtaacctacaaagcaaccatgatctgtattgtcaagt
ccatggtaatgcacatgtagctagttgtgatgcaatcatgactag
gtgtctagctgtccacgagtgctttgttaagcgtgttgactggac
tattgaatatcctataattggtgatgaactgaagattaatgcggc
ttgtagaaaggttcaacacatggttgttaaagctgcattattagc
agacaaattcccagttcttcacgacattggtaaccctaaagctat
taagtgtgtacctcaagctgatgtagaatggaagttctatgatgc
acagccttgtagtgacaaagcttataaaatagaagaattattcta
ttcttatgccacacattctgacaaattcacagatggtgtatgcct
attttggaattgcaatgtcgatagatatcctgctaattccattgt
ttgtagatttgacactagagtgctatctaaccttaacttgcctgg
ttgtgatggtggcagtttgtatgtaaataaacatgcattccacac
accagcttttgataaaagtgcttttgttaatttaaaacaattacc
atttttctattactctgacagtccatgtgagtctcatggaaaaca
agtagtgtcagatatagattatgtaccactaaagtctgctacgtg
tataacacgttgcaatttaggtggtgctgtctgtagacatcatgc
taatgagtacagattgtatctcgatgcttataacatgatgatctc
agctggctttagcttgtgggtttacaaacaatttgatacttataa
cctctggaacacttttacaagacttcagagtttagaaaatgtggc
ttttaatgttgtaaataagggacactttgatggacaacagggtga
agtaccagtttctatcattaataacactgtttacacaaaagttga
tggtgttgatgtagaattgtttgaaaataaaacaacattacctgt
taatgtagcatttgagctttgggctaagcgcaacattaaaccagt
accagaggtgaaaatactcaataatttgggtgtggacattgctgc
taatactgtgatctgggactacaaaagagatgctccagcacatat
atctactattggtgtttgttctatgactgacatagccaagaaacc
aactgaaacgatttgtgcaccactcactgtcttttttgatggtag
agttgatggtcaagtagacttatttagaaatgcccgtaatggtgt
tcttattacagaaggtagtgttaaaggtttacaaccatctgtagg
tcccaaacaagctagtcttaatggagtcacattaattggagaagc
cgtaaaaacacagttcaattattataagaaagttgatggtgttgt
ccaacaattacctgaaacttactttactcagagtagaaatttaca
agaatttaaacccaggagtcaaatggaaattgatttcttagaatt
agctatggatgaattcattgaacggtataaattagaaggctatgc
cttcgaacatatcgtttatggagattttagtcatagtcagttagg
tggtttacatctactgattggactagctaaacgttttaaggaatc
accttttgaattagaagattttattcctatggacagtacagttaa
aaactatttcataacagatgcgcaaacaggttcatctaagtgtgt
gtgttctgttattgatttattacttgatgattttgttgaaataat
aaaatcccaagatttatctgtagtttctaaggttgtcaaagtgac
tattgactatacagaaatttcatttatgctttggtgtaaagatgg
ccatgtagaaacattttacccaaaattacaatctagtcaagcgtg
gcaaccgggtgttgctatgcctaatctttacaaaatgcaaagaat
gctattagaaaagtgtgaccttcaaaattatggtgatagtgcaac
attacctaaaggcataatgatgaatgtcgcaaaatatactcaact
gtgtcaatatttaaacacattaacattagctgtaccctataatat
gagagttatacattttggtgctggttctgataaaggagttgcacc
aggtacagctgttttaagacagtggttgcctacgggtacgctgct
tgtcgattcagatcttaatgactttgtctctgatgcagattcaac
tttgattggtgattgtgcaactgtacatacagctaataaatggga
tctcattattagtgatatgtacgaccctaagactaaaaatgttac
aaaagaaaatgactctaaagagggttttttcacttacatttgtgg
gtttatacaacaaaagctagctcttggaggttccgtggctataaa
gataacagaacattcttggaatgctgatctttataagctcatggg
acacttcgcatggtggacagcctttgttactaatgtgaatgcgtc
atcatctgaagcatttttaattggatgtaattatcttggcaaacc
acgcgaacaaatagatggttatgtcatgcatgcaaattacatatt
ttggaggaatacaaatccaattcagttgtcttcctattctttatt
tgacatgagtaaatttccccttaaattaaggggtactgctgttat
gtctttaaaagaaggtcaaatcaatgatatgattttatctcttct
tagtaaaggtagacttataattagagaaaacaacagagttgttat
ttctagtgatgttcttgttaacaactaaACGAACggcgcgccACC
ATGGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATCTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGGACGGCAGCGTGCAGCTCGCC
GACCACTACCAGCAGAACACCCCCATCGGCGACGGCCCCGTGCTG
CTGCCCGACAACCACTACCTGAGCACCCAGTCCGCCCTGAGCAAA
GACCCCAACGAGAAGCGCGATCACATGGTCCTGCTGGAGTTCGTG
ACCGCCGCCGGGATCACTCTCGGCATGGACGAGCTGTACAAGTAG
tctagactcctgcaggatgggctatataaacgttttcgcttttcc
gtttacgatatatagtctactcttgtgcagaatgaattctcgtaa
ctacatagcacaagtagatgtagttaactttaatctcacatagca
atctttaatcagtgtgtaacattagggaggacttgaaagagccac
cacattttcaccgaggccacgcggagtacgatcgagtgtacagtg
aacaatgctagggagagctgcctatatggaagagccctaatgtgt
aaaattaattttagtagtgctatccccatgtgattttaatagctt
cttaggagaatgacaaaaaaaaaaaaaaaaaaaaaaaaaaaaaaa
aacgcggaacccctatttgtttatttttctaaatacattcaaata
tgtatccgctcatgagacaataaccctgataaatgcttcaataat
attgaaaaaggaagagtatgagtattcaacatttccgtgtcgccc
ttattcccttttttgcggcattttgccttcctgtttttgctcacc
cagaaacgctggtgaaagtaaaagatgctgaagatcagttgggtg
cacgagtgggttacatcgaactggatctcaacagcggtaagatcc
ttgagagttttcgccccgaagaacgttttccaatgatgagcactt
ttaaagttctgctatgtggcgcggtattatcccgtgttgacgccg
ggcaagagcaactcggtcgccgcatacactattctcagaatgact
tggttgagtactcaccagtcacagaaaagcatcttacggatggca
tgacagtaagagaattatgcagtgctgccataaccatgagtgata
acactgcggccaacttacttctgacaacgatcggaggaccgaagg
agctaaccgcttttttgcacaacatgggggatcatgtaactcgcc
ttgatcgttgggaaccggagctgaatgaagccataccaaacgacg
agcgtgacaccacgatgcctgtagcaatggcaacaacgttgcgca
aactattaactggcgaactacttactctagcttcccggcaacaat
taatagactggatggaggcggataaagttgcaggaccacttctgc
gctcggcccttccggctggctggtttattgctgataaatctggag
ccggtgagcgtggctctcgcggtatcattgcagcactggggccag
atggtaagccctcccgtatcgtagttatctacacgacggggagtc
aggcaactatggatgaacgaaatagacagatcgctgagataggtg
cctcactgattaagcattggtaactgtcagaccaagtttactcat
atatactttagattgatttaaaacttcatttttaatttaaaagga
tctaggtgaagatcctttttgataatctcatgaccaaaatccctt
aacgtgagttttcgttccactgagcgtcagaccccgtagaaaaga
tcaaaggatcttcttgagatcctttttttctgcgcgtaatctgct
gcttgcaaacaaaaaaaccaccgctaccagcggtggtttgtttgc
cggatcaagagctaccaactctttttccgaaggtaactggcttca
gcagagcgcagataccaaatactgtccttctagtgtagccgtagt
taggccaccacttcaagaactctgtagcaccgcctacatacctcg
ctctgctaatcctgttaccagtggctgctgccagtggcgataagt
cgtgtcttaccgggttggactcaagacgatagttaccggataagg
cgcagcggtcgggctgaacggggggttcgtgcacacagcccagct
tggagcgaacgacctacaccgaactgagatacctacagcgtgagc
tatgagaaagcgccacgcttcccgaagggagaaaggcggacaggt
atccggtaagcggcagggtcggaacaggagagcgcacgagggagc
ttccagggggaaacgcctggtatctttatagtcctgtcgggtttc
gccacctctgacttgagcgtcgatttttgtgatgctcgtcagggg
ggggagcctatggaaagcggccgcaacgccagcaacgcgagctca
ttaaggggtactgctgttatgtctttaaaagaaggtcaaatcaat
gatatgattttatctcttcttagtaaaggtagacttataattaga
gaaaacaacagagttgttatttctagtgatgttcttgttaacaac
taact

### Example 2: Vectors Based on VEEV Replicon or Partial Replicon

FIG. 3 illustrates a vector based on a full VEEV replicon, with the EGFP gene as payload. The vector consists of the NS gene, and it has been designed according to the description in Table 9. The exemplary payload consists of the EGFP gene, and it has been designed according to the description in Table 14. In addition to the vector and the payload, the construct contains an origin of replication, a bacterial promoter, and a NeoR/KanR gene acting as a selection marker, useful when the construct is used as a plasmid; and a human CMV enhancer/promoter, useful when the construct is used as a DNA/RNA vector in humans.

The features present in the construct are listed in the following table:

TABLE 5
Sequence of a vector based on the full
VEEV replicon (in GenBank format)

LOCUS Exported 11131 bp ds-DNA circular SYN 11 MAR. 2021
DEFINITION construct.
ACCESSION
VERSION
KEYWORDS
SOURCE synthetic DNA construct
ORGANISM synthetic DNA construct
REFERENCE 1 (bases 1 to 11131)
AUTHORS
TITLE Direct Submission
JOURNAL Exported Thursday, May 20, 2021 from SnapGene Viewer 5.2.4
https://www.snapgene.com

| FEATURES | Location/Qualifiers |
|---|---|
| source | 1..11131 |

/organism=“synthetic DNA construct”

| | |
|---|---|
| source | 8903..8910 |
| source | 9618..9625 |
| source | 9626..10542 |
| source | 10563..10570 |
| source | 11079..11086 |
| gene | 1..8133 |

/gene=“NS”
/locus_tag=“VEEVgp2”
/label=NS
/db_xref=“GeneID:2652923”

| | |
|---|---|
| 5′UTR | 1..44 |

/gene=“NS”
/locus_tag=“VEEVgp2”

| | |
|---|---|
| misc_feature | 45..645 |

/label=IRES
/note=“internal ribosome entry site (IRES) of the
encephalomyocarditis virus (EMCV)”

| | |
|---|---|
| regulatory | 646..651 |

/regulatory_class=“other”
/label=Kozak sequence
/note=“vertebrate consensus sequence for strong initiation
of translation (Kozak, 1987)”

| | |
|---|---|
| CDS | 652..8133 |

/codon_start=1
/gene=“NS”
/locus_tag=“VEEVgp2”
/product=“non-structural polyprotein precursor P1234”
/label=NS
/note=“possible incorporation of arginine, cysteine or
tryptophan at read through of UGA codon”
/db_xref=“GeneID:2652923”
/protein_id=“NP_040822.1”
/translation=“MEKVHVDIEEDSPFLRALQRSFPQFEVEAKQVTDNDHANARAFSH
LASKLIETEVDPSDTILDIGSAPARRMYSKHKYHCICPMRCAEDPDRLYKYATKLKKNC
KEITDKELDKKMKELAAVMSDPDLETETMCLHDDESCRYEGQVAVYQDVYAVDGPTSLY
HQANKGVRVAYWIGFDTTPFMFKNLAGAYPSYSTNWADETVLTARNIGLCSSDVMERSR
RGMSILRKKYLKPSNNVLFSVGSTIYHEKRDLLRSWHLPSVFHLRGKQNYTCRCETIVS
CDGYVVKRIAISPGLYGKPSGYAATMHREGFLCCKVTDTLNGERVSFPVCTYVPATLCD
QMTGILATDVSADDAQKLLVGLNQRIVVNGRTQRNTNTMKNYLLPVVAQAFARWAKEYK
EDQEDERPLGLRDRQLVMGCCWAFRRHKITSIYKRPDTQTIIKVNSDFHSFVLPRIGSN
TLEIGLRTRIRKMLEEHKEPSPLITAEDIQEAKCAADEAKEVREAEELRAALPPLAADF
EEPTLEADVDLMLQEAGAGSVETPRGLIKVTSYAGEDKIGSYAVLSPQAVLKSEKLSCI
HPLAEQVIVITHSGRKGRYAVEPYHGKVVVPEGHAIPVQDFQALSESATIVYNEREFVN
RYLHHIATHGGALNTDEEYYKTVKPSEHDGEYLYDIDRKQCVKKELVTGLGLTGELVDP
PFHEFAYESLRTRPAAPYQVPTIGVYGVPGSGKSGIIKSAVTKKDLVVSAKKENCAEII
RDVKKMKGLDVNARTVDSVLLNGCKHPVETLYIDEAFACHAGTLRALIAIIRPKKAVLC
GDPKQCGFFNMMCLKVHFNHEICTQVFHKSISRRCTKSVTSVVSTLFYDKRMRTTNPKE
TKIVIDTTGSTKPKQDDLILTCFRGWVKQLQIDYKGNEIMTAAASQGLTRKGVYAVRYK
VNENPLYAPTSEHVNVLLTRTEDRIVWKTLAGDPWIKILTAKYPGNFTATIEEWQAEHD
AIMRHILERPDPTDVFQNKANVCWAKALVPVLKTAGIDMTTEQWNTVDYFETDKAHSAE
IVLNQLCVRFFGLDLDSGLFSAPTVPLSIRNNHWDNSPSPNMYGLNKEVVRQLSRRYPQ
LPRAVATGRVYDMNTGTLRNYDPRINLVPVNRRLPHALVLHHNEHPQSDFSSFVSKLKG
RTVLVVGEKLSVPGKKVDWLSDQPEATFRARLDLGIPGDVPKYDIVFINVRTPYKYHHY
QQCEDHAIKLSMLTKKACLHLNPGGTCVSIGYGYADRASESIIGAIARQFKFSRVCKPK
SSHEETEVLFVFIGYDRKARTHNPYKLSSTLTNIYTGSRLHEAGCAPSYHVVRGDIATA
TEGVIINAANSKGQPGGGVCGALYKKFPESFDLQPIEVGKARLVKGAAKHIIHAVGPNF
NKVSEVEGDKQLAEAYESIAKIVNDNNYKSVAIPLLSTGIFSGNKDRLTQSLNHLLTAL
DTTDADVAIYCRDKKWEMTLKEAVARREAVEEICISDDSSVTEPDAELVRVHPKSSLAG
RKGYSTSDGKTFSYLEGTKFHQAAKDIAEINAMWPVATEANEQVCMYILGESMSSIRSK
CPVEESEASTPPSTLPCLCIHAMTPERVQRLKASRPEQITVCSSFPLPKYRITGVQKIQ
CSQPILFSPKVPAYIHPRKYLVETPPVEETPESPAENQSTEGTPEQPALVNVDATRTRM
PEPIIIEEEEEDSISLLSDGPTHQVLQVEADIHGSPSVSSSSWSIPHASDFDVDSLSIL
DTLDGASVTSGAVSAETNSYFARSMEFRARPVPAPRTVFRNPPHPAPRTRTPPLAHSRA
SSRTSLVSTPPGVNRVITREELEALTPSRAPSRSASRTSLVSNPPGVNRVITREEFEAF
VAQQQ*RFDAGAYIFSSDTGQGHLQQKSVRQTVLSEVVLERTELEISYAPRLDQEKEEL
LRKKLQLNPTPANRSRYQSRRVENMKAITARRILQGLGHYLKAEGKVECYRTLHPVPLY
SSSVNRAFSSPKVAVEACNAMLKENFPTVASYCIIPEYDAYLDMVDGASCCLDTASFCP
AKLRSFPKKHSYLEPTIRSAVPSAIQNTLQNVLAAATKRNCNVTQMRELPVLDSAAFNV
ECFKKYACNNEYWETFKENPIRLTEENVVNYITKLKGPKAAALFAKTHNLNMLQDIPMD
RFVMDLKRDVKVTPGTKHTEERPKVQVIQAADPLATADLCGIHRELVRRLNAVLLPNIH
TLFDMSAEDFDAIIAEHFQPGDCVLETDIASFDKSEDDAMALTALMILEDLGVDAELLT
LIEAAFGEISSIHLPTKTKFKFGAMMKSGMFLTLFVNTVINIVIASRVLRERLTGSPCA
AFIGDDNIVKGVKSDKLMADRCATWLNMEVKIIDAVVGEKAPYFCGGFILCDSVTGTAC
RVADPLKRLFKLGKPLAVDDEHDDDRRRALHEESTRWNRVGILPELCKAVESRYETVGT
SIIVMAMTTLASSVKSFSYLRGAPITLYG”

| | |
|---|---|
| CDS | 652..6291 |

/codon_start=1
/gene=“NS”
/locus_tag=“VEEVgp2”
/product=“non-structural polyprotein precursor P123”
/label=NS
/db_xref=“GenelD:2652923”
/protein_id=“NP_040823.1”
/translation=“MEKVHVDIEEDSPFLRALQRSFPQFEVEAKQVTDNDHANARAFSH
LASKLIETEVDPSDTILDIGSAPARRMYSKHKYHCICPMRCAEDPDRLYKYATKLKKNC
KEITDKELDKKMKELAAVMSDPDLETETMCLHDDESCRYEGQVAVYQDVYAVDGPTSLY
HQANKGVRVAYWIGFDTTPFMFKNLAGAYPSYSTNWADETVLTARNIGLCSSDVMERSR
RGMSILRKKYLKPSNNVLFSVGSTIYHEKRDLLRSWHLPSVFHLRGKQNYTCRCETIVS
CDGYVVKRIAISPGLYGKPSGYAATMHREGFLCCKVTDTLNGERVSFPVCTYVPATLCD
QMTGILATDVSADDAQKLLVGLNQRIVVNGRTQRNTNTMKNYLLPVVAQAFARWAKEYK
EDQEDERPLGLRDRQLVMGCCWAFRRHKITSIYKRPDTQTIIKVNSDFHSFVLPRIGSN
TLEIGLRTRIRKMLEEHKEPSPLITAEDIQEAKCAADEAKEVREAEELRAALPPLAADF
EEPTLEADVDLMLQEAGAGSVETPRGLIKVTSYAGEDKIGSYAVLSPQAVLKSEKLSCI
HPLAEQVIVITHSGRKGRYAVEPYHGKVVVPEGHAIPVQDFQALSESATIVYNEREFVN
RYLHHIATHGGALNTDEEYYKTVKPSEHDGEYLYDIDRKQCVKKELVTGLGLTGELVDP
PFHEFAYESLRTRPAAPYQVPTIGVYGVPGSGKSGIIKSAVTKKDLVVSAKKENCAEII
RDVKKMKGLDVNARTVDSVLLNGCKHPVETLYIDEAFACHAGTLRALIAIIRPKKAVLC
GDPKQCGFFNMMCLKVHFNHEICTQVFHKSISRRCTKSVTSVVSTLFYDKRMRTTNPKE
TKIVIDTTGSTKPKQDDLILTCFRGWVKQLQIDYKGNEIMTAAASQGLTRKGVYAVRYK
VNENPLYAPTSEHVNVLLTRTEDRIVWKTLAGDPWIKILTAKYPGNFTATIEEWQAEHD
AIMRHILERPDPTDVFQNKANVCWAKALVPVLKTAGIDMTTEQWNTVDYFETDKAHSAE
IVLNQLCVRFFGLDLDSGLFSAPTVPLSIRNNHWDNSPSPNMYGLNKEVVRQLSRRYPQ
LPRAVATGRVYDMNTGTLRNYDPRINLVPVNRRLPHALVLHHNEHPQSDFSSFVSKLKG
RTVLVVGEKLSVPGKKVDWLSDQPEATFRARLDLGIPGDVPKYDIVFINVRTPYKYHHY
QQCEDHAIKLSMLTKKACLHLNPGGTCVSIGYGYADRASESIIGAIARQFKFSRVCKPK
SSHEETEVLFVFIGYDRKARTHNPYKLSSTLTNIYTGSRLHEAGCAPSYHVVRGDIATA
TEGVIINAANSKGQPGGGVCGALYKKFPESFDLQPIEVGKARLVKGAAKHIIHAVGPNF
NKVSEVEGDKQLAEAYESIAKIVNDNNYKSVAIPLLSTGIFSGNKDRLTQSLNHLLTAL
DTTDADVAIYCRDKKWEMTLKEAVARREAVEEICISDDSSVTEPDAELVRVHPKSSLAG
RKGYSTSDGKTFSYLEGTKFHQAAKDIAEINAMWPVATEANEQVCMYILGESMSSIRSK
CPVEESEASTPPSTLPCLCIHAMTPERVQRLKASRPEQITVCSSFPLPKYRITGVQKIQ
CSQPILFSPKVPAYIHPRKYLVETPPVEETPESPAENQSTEGTPEQPALVNVDATRTRM
PEPIIIEEEEEDSISLLSDGPTHQVLQVEADIHGSPSVSSSSWSIPHASDFDVDSLSIL
DTLDGASVTSGAVSAETNSYFARSMEFRARPVPAPRTVFRNPPHPAPRTRTPPLAHSRA
SSRTSLVSTPPGVNRVITREELEALTPSRAPSRSASRTSLVSNPPGVNRVITREEFEAF
VAQQQ”

| | |
|---|---|
| mat_peptide | 652..2256 |

/gene=“NS”
/locus_tag=“VEEVgp2”
/product=“mRNA-capping enzyme nsP1”
/function=“minus strand RNA synthesis; methyltransferase;
guanyltransferase”
/note=“/protein_id=NP_740696.1”

| | |
|---|---|
| mat_peptide | 2257..4638 |

/gene=“NS”
/locus_tag=“VEEVgp2”
/product=“protease nsP2”
/function=“RNA helicase; nonstructural proteinase; necessary
for subgenomic 26S mRNA synthesis”
/function=“replication”
/note=“/protein_id=NP_740697.1”

| | |
|---|---|
| mat_peptide | 4639..6309 |

/gene=“NS”
/locus_tag=“VEEVgp2”
/product=“non-structural protein nsp3”
/function=“replication”
/note=“/protein_id=NP_740698.1”

| | |
|---|---|
| mat_peptide | 6310..8127 |

/gene=“NS”
/locus_tag=“VEEVgp2”
/product=“RNA-directed RNA polymerase nsP4”
/function=“RNA polymerase”
/function=“replication”
/note=“/protein_id=NP_740699.1”

| | |
|---|---|
| misc_feature | 8131..8168 |

/locus_tag=“VEEVgp1”
/function=“noncoding segment between nonstructural
and structural genes”
/label=putative
/note=“putative”

| | |
|---|---|
| misc_feature | 8169..8176 |

/label=AsiSI restriction site
/note=“AsiSI restriction site”

| | |
|---|---|
| regulatory | 8177..8186 |

/regulatory_class=“other”
/note=“vertebrate consensus sequence for strong
initiation of translation (Kozak, 1987)”

| | |
|---|---|
| CDS | 8183..8902 |

/codon_start=1
/product=“enhanced GFP”
/label=enhanced GFP
/note=“mammalian codon-optimized”
/translation=“MVSKGEELFTGVVPILVELDGDVNGHKFSVSGEGEGDATYGKLTL
KFICTTGKLPVPWPTLVTTLTYGVQCFSRYPDHMKQHDFFKSAMPEGYVQERTIFFKDD
GNYKTRAEVKFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPNEKRDHMVLL
EFVTAAGITLGMDELYK”

| | |
|---|---|
| primer_bind | complement(8228..8249) |

/label=EGFP, reverse primer
/note=“EGFP, reverse primer”

| | |
|---|---|
| primer_bind | complement(8489..8508) |

/label=For distinguishing EGFP variants, reverse primer
/note=“For distinguishing EGFP variants, reverse primer”

| | |
|---|---|
| primer_bind | 8836..8857 |

/label=EGFP, forward primer
/note=“EGFP, forward primer”

| | |
|---|---|
| misc_feature | 8903..8910 |

/label=SbfI restriction site
/note=“SbfI restriction site”

| | |
|---|---|
| 3′UTR | 8911..9028 |

/gene=“26S mRNA”
/locus_tag=“VEEVgp3”

| | |
|---|---|
| polyA_site | 9028 |

/gene=“26S mRNA”
/locus_tag=“VEEVgp3”

| | |
|---|---|
| rep_origin | 9029..9617 |

/direction=RIGHT
/note=“high-copy-number ColE1/pMB1/pBR322/pUC origin of
replication”

| | |
|---|---|
| primer_bind | 9518..9537 |

/label=pBR322 origin, forward primer
/note=“pBR322 origin, forward primer”

| | |
|---|---|
| misc_feature | 9618..9625 |

/label=NotI restriction site
/note=“NotI restriction site”

| | |
|---|---|
| promoter | 9626..9730 |

/gene=“bla”
/label=AmpR promoter

| | |
|---|---|
| CDS | 9731..10525 |

/codon_start=1
/gene=“aph(3′)-II (or nptII)”
/product=“aminoglycoside phosphotransferase from Tn5”
/label=NeoR/KanR
/note=“confers resistance to neomycin, kanamycin, and G418
(Geneticin(R))”
/translation=“MIEQDGLHAGSPAAWVERLFGYDWAQQTIGCSDAAVFRLSAQGRP
VLFVKTDLSGALNELQDEAARLSWLATTGVPCAAVLDVVTEAGRDWLLLGEVPGQDLLS
SHLAPAEKVSIMADAMRRLHTLDPATCPFDHQAKHRIERARTRMEAGLVDQDDLDEEHQ
GLAPAELFARLKARMPDGEDLVVTHGDACLPNIMVENGRFSGFIDCGRLGVADRYQDIA
LATRDIAEELGGEWADRFLVLYGIAAPDSQRIAFYRLLDEFF”

| | |
|---|---|
| primer_bind | complement(9785..9804) |

/label=Neo-R
/note=“Neomycin resistance gene, reverse primer”

| | |
|---|---|
| primer_bind | 10395..10414 |

/label=Neo-F
/note=“Neomycin resistance gene, forward primer”

| | |
|---|---|
| misc_feature | 10563..10570 |

/label=Fsel restriction site
/note=“Fsel restriction site”

| | |
|---|---|
| enhancer | 10571..10874 |

/note=“human cytomegalovirus immediate early enhancer”

| | |
|---|---|
| promoter | 10875..11078 |

/note=“human cytomegalovirus (CMV) immediate earlypromoter”

| | |
|---|---|
| misc_feature | 11079..11086 |

/label=Fsel restriction site
/note=“Fsel restriction site”

| | |
|---|---|
| promoter | 11113..11131 |

/note=“T7 promoter”

The DNA sequence of the construct is listed in the following table:

TABLE 6
Sequence of a vector based on the full VEEV replicon

| | Nucleic Acid Sequence (Description of what is encoded. Sequence |
|---|---|
| Sequence | represented as DNA for corresponding RNA sequence T is replaced with |
| ID | U). The number at the beginning of each line represents the coordinate |
| number | (1-based) of the first nucleotide in that line |
| 3 | CMV+T7-IRES_VEEV |
| | 1 atgggcggcg caagagagaa gcccaaacca attacctacc caaacctctc cccccccccc |
| | 61 ctctccctcc ccccccccta acgttactgg ccgaagccgc ttggaataag gccggtgtgc |
| | 121 gtttgtctat atgttatttt ccaccatatt gccgtctttt ggcaatgtga gggcccggaa |
| | 181 acctggccct gtcttcttga cgagcattcc taggggtctt tcccctctcg ccaaaggaat |
| | 241 gcaaggtctg ttgaatgtcg tgaaggaagc agttcctctg gaagcttctt gaagacaaac |
| | 301 aacgtctgta gcgacccttt gcaggcagcg gaacccccca cctggcgaca ggtgcctctg |
| | 361 cggccaaaag ccacgtgtat aagatacacc tgcaaaggcg gcacaacccc agtgccacgt |
| | 421 tgtgagttgg atagttgtgg aaagagtcaa atggctctcc tcaagcgtat tcaacaaggg |
| | 481 gctgaaggat gcccagaagg taccccattg tatgggatct gatctggggc ctcggtgcac |
| | 541 atgctttaca tgtgtttagt cgaggttaaa aaaacgtcta ggccccccga accacgggga |
| | 601 cgtggttttc ctttgaaaaa cacgatgata agcttgccac aacccgccac catggagaaa |
| | 661 gttcacgttg acatcgagga agacagccca ttcctcagag ctttacaacg gagcttcccg |
| | 721 cagtttgagg tagaagccaa gcaggtcact gataatgacc atgctaatgc cagagcgttt |
| | 781 tcgcatctgg cttcaaaact gatcgaaacg gaggtggacc catccgacac gatccttgac |
| | 841 attggaagtg cgcccgcccg cagaatgtat tctaagcata agtatcattg catctgtccg |
| | 901 atgagatgtg cggaagatcc ggacagattg tacaagtatg caactaagct gaagaaaaat |
| | 961 tgcaaggaaa taactgacaa ggaattggac aagaaaatga aggagctcgc cgccgtcatg |
| | 1021 agcgaccctg acctggaaac tgagactatg tgcctccacg acgatgagtc atgtcgctac |
| | 1081 gaggggcaag tcgctgttta ccaggatgta tacgcagttg acggaccgac aagtctctat |
| | 1141 caccaagcca acaagggagt tagagtcgcc tactggatag gctttgacac cacccctttt |
| | 1201 atgtttaaga acttggctgg agcatatcca tcatactcta ccaactgggc cgacgaaacc |
| | 1261 gtgttaacgg ctcgtaacat aggcctatgc agctccgacg tcatggagcg gtcacgtaga |
| | 1321 gggatgtcca ttcttaggaa gaagtatttg aaaccatcca ataatgtcct attctctgtt |
| | 1381 ggctcgacca tctaccacga gaagagggac ttactgagga gctggcacct gccgtctgta |
| | 1441 tttcacttac gtggcaagca aaattacaca tgtcggtgtg agactatagt tagttgcgac |
| | 1501 gggtacgtcg ttaaaagaat agctatcagt ccaggcctgt atgggaagcc ttcaggctat |
| | 1561 gctgctacga tgcaccgcga gggattcttg tgctgcaaag tgacagacac attgaacggg |
| | 1621 gagagggtct cttttcccgt gtgcacgtat gtgccagcta cattgtgtga ccaaatgact |
| | 1681 ggcatactgg caacagatgt cagtgcggac gacgcgcaaa aactgctggt tgggctcaac |
| | 1741 cagcgcatag tcgtcaacgg tcgcacccaa agaaacacca ataccatgaa gaattatott |
| | 1801 ttgcccgtag tggcccaggc atttgctagg tgggcaaagg aatataagga agatcaagaa |
| | 1861 gatgagaggc cactaggact acgagataga cagttagtca tggggtgctg ctgggctttt |
| | 1921 agaaggcaca agataacatc tatttataag cgcccagata cccaaaccat catcaaagtg |
| | 1981 aacagcgatt tccactcatt cgtgctgccc aggataggca gtaacacact ggagatcggg |
| | 2041 ctgagaacga gaatcaggaa aatgctagaa gagcacaagg agccgtcacc tctcattact |
| | 2101 gccgaggaca tacaagaggc taagtgcgca gccgatgagg ctaaggaagt gcgtgaagcc |
| | 2161 gaggagctgc gcgctgctct accacctttg gcagctgatt ttgaggagcc cactctggaa |
| | 2221 gccgatgtcg acttgatgtt acaagaggct ggggccggct cagtggagac acctcgtggc |
| | 2281 ttgataaagg ttaccagcta tgccggcgag gacaagatcg gctcttacgc agtgctttct |
| | 2341 ccacaggctg tactcaagag tgagaaacta tottgcattc accctctcgc tgaacaagtc |
| | 2401 atagtgataa cacactctgg ccgaaaaggg cgttatgccg tggaacccta ccatggaaaa |
| | 2461 gtagtggtgc cagagggaca tgcaataccc gtccaggact ttcaagctct gagtgaaagt |
| | 2521 gccaccatcg tgtacaacga acgagagttc gtaaacaggt acctgcacca tattgccaca |
| | 2581 catggaggag cgctgaacac agatgaagaa tattacaaaa ctgtcaagcc cagcgagcac |
| | 2641 gacggcgaat acctgtacga catcgacagg aaacaatgcg tcaagaaaga attagtcact |
| | 2701 gggctagggc ttacaggcga gctggtggat cctcccttcc atgaatttgc ctacgagagt |
| | 2761 ctgagaacac gtccggccgc tccttaccaa gtaccaacca taggggtgta tggcgtgccg |
| | 2821 gggtcaggca agtctggcat cattaaaagc gcagtcacca aaaaagatct ggtggtgagc |
| | 2881 gccaagaaag aaaactgcgc agaaataata agggacgtca agaaaatgaa agggctggac |
| | 2941 gtcaatgcca gaactgtgga ctcagtgctc ttgaatggat gcaaacaccc cgtagagacc |
| | 3001 ctgtatattg acgaagcttt tgcttgtcat gcaggcactc tcagagcgct catagccatc |
| | 3061 ataagaccta aaaaggcagt gctctgcggg gatccaaaac agtgtggctt tttcaatatg |
| | 3121 atgtgcctga aagtgcattt taaccacgag atttgcacgc aggtcttcca caaaagcatc |
| | 3181 tctcgccgtt gcactaaatc cgtgacttcg gtcgtctcaa ccttgtttta cgacaaaagg |
| | 3241 atgagaacga cgaacccgaa agagactaag attgtgattg acactactgg cagtaccaaa |
| | 3301 ccgaagcagg acgatctcat tctcacttgt ttcagagggt gggtgaagca gttgcaaata |
| | 3361 gattacaaag gcaacgaaat aatgacggca gctgcctctc aagggctgac ccgtaaaggc |
| | 3421 gtgtatgccg ttcggtacaa ggtgaatgaa aatcccctgt acgcacccac ctcagaacat |
| | 3481 gtgaacgtcc tactgacccg cacggaggac cgtatcgtgt ggaaaacact agccggtgat |
| | 3541 ccatggataa aaatactgac ggccaagtat cctgggaact tcactgccac gatagaggaa |
| | 3601 tggcaagcag agcatgatgc catcatgagg cacatcttgg agagaccgga ccctaccgac |
| | 3661 gttttccaaa ataaggcgaa cgtgtgttgg gccaaggctt tggtgccggt actgaagact |
| | 3721 gcaggcatag acatgaccac tgaacaatgg aacactgtgg attacttcga aacggacaaa |
| | 3781 gctcactcag cagagatagt attgaaccaa ctatgcgtga ggttctttgg actcgacctg |
| | 3841 gactccggtc tattttctgc acccactgtt ccgttatcca ttaggaataa tcactgggat |
| | 3901 aattccccgt cgcctaacat gtacgggttg aataaagaag tggtccgcca gctctcccgc |
| | 3961 aggtacccac aactgcctcg agcagttgcc accggaagag tctatgacat gaacactggc |
| | 4021 acgctgcgca attatgatcc gcgcataaat ctagtacctg tgaacagaag actgcctcat |
| | 4081 gctttagtcc tccaccataa tgaacaccca cagagtgact tttcttcatt cgtcagcaaa |
| | 4141 ctgaagggca gaactgtctt ggtggtcggg gagaagttgt ccgtcccagg caaaaaggtc |
| | 4201 gactggttgt cagaccagcc tgaggctacc tttagagctc ggctggattt aggtatccca |
| | 4261 ggtgacgtgc ccaaatacga cattgtattt attaacgtga ggactccata taaataccat |
| | 4321 cattatcagc agtgtgaaga ccacgccatt aagcttagta tgttgaccaa gaaagcttgt |
| | 4381 ctgcatttga atcccggcgg aacctgcgtc agcataggtt atggttacgc tgacagggcc |
| | 4441 agcgagagca tcattggtgc tatagcgcgg cagttcaagt tctcccgggt atgcaaaccg |
| | 4501 aaatcctcac atgaagagac agaagtactg tttgtattca ttgggtacga tcgcaaggcc |
| | 4561 cgtacgcaca atccttacaa gotttcatct accttgacca acatctatac aggttccaga |
| | 4621 ctccacgaag ccggatgcgc accctcatat catgtggtgc gaggggatat tgccacggcc |
| | 4681 accgaaggag tgatcataaa tgctgctaac agcaaaggac aacctggcgg aggggtgtgc |
| | 4741 ggagcgctgt ataagaaatt cccggaaagc ttcgatttac agccgatcga agtaggaaaa |
| | 4801 gcgcgactgg tcaaaggtgc agctaaacat atcattcatg ccgtaggacc aaacttcaac |
| | 4861 aaagtttcgg aagttgaagg ggacaaacag ttggcagagg cttatgagtc catcgctaaa |
| | 4921 attgtcaacg ataacaatta caagtcagta gcgattccac tgttgtccac cggcatcttt |
| | 4981 tccgggaaca aagatcgact aacccaatca ttgaaccatt tgctgacagc tttagacacc |
| | 5041 actgatgcag atgtagccat atactgcagg gacaagaaat gggaaatgac tctcaaggaa |
| | 5101 gcagtggcta ggagagaagc agtggaggag atatgcatat cagacgactc ttcggtgaca |
| | 5161 gaaccggatg cagagctggt gagggtacat ccgaagagtt ctttggctgg aaggaagggc |
| | 5221 tacagcacaa gtgatggcaa gactttctca tatttggaag ggaccaaatt tcaccaggcg |
| | 5281 gccaaggata tagcagaaat taatgccatg tggccagttg caacggaggc caatgagcaa |
| | 5341 gtatgcatgt atatcctcgg tgaaagcatg agcagcatta ggtcgaaatg ccccgtcgag |
| | 5401 gagtcggaag cctccacacc acctagcacg ctgccttgct tgtgcatcca tgctatgact |
| | 5461 ccagaaagag tacaacgcct aaaagcctca cgtccagaac aaattactgt gtgctcatcc |
| | 5521 tttccattgc cgaagtatag aatcactggt gtgcagaaga tccagtgctc ccagcctata |
| | 5581 ctgttctcac cgaaggtgcc tgcgtacatt catccacgga agtacctcgt ggaaacacca |
| | 5641 ccggtagaag agactccgga gtcgccggca gagaaccaat ccacagaggg gacacctgaa |
| | 5701 caaccagcac ttgtaaacgt ggatgcaacc aggactagaa tgcctgaacc gatcatcatt |
| | 5761 gaagaggaag aagaggatag tataagtttg ctgtcagacg gcccgaccca ccaggtgctg |
| | 5821 caagtcgagg cagacattca cgggtcgcct tctgtatcca gctcatcctg gtccattcct |
| | 5881 catgcatccg actttgatgt ggacagctta tccatccttg acaccctgga tggagctagc |
| | 5941 gtgaccagcg gggcagtgtc agccgagact aactcctact tcgcaaggag catggagttt |
| | 6001 cgggcgcgac cggtgcctgc gcctcgaacc gtattcagga accctccaca tcccgcaccg |
| | 6061 cgcacaagaa caccgccact tgcacacagc agggccagct cgagaactag cctagtttcc |
| | 6121 accccgccag gcgtgaatag ggtgattact agagaggagc tegaggcgct taccccgtcc |
| | 6181 cgcgctccta gcaggteggc ctcaagaact agcctggtct ctaacccgcc aggcgtaaat |
| | 6241 agggtgatta caagagagga gtttgaggcg ttcgtagcac aacaacaatg acggtttgac |
| | 6301 gcgggtgcat acatcttttc ctccgatacc ggtcaagggc atttacaaca aaaatcagta |
| | 6361 aggcaaacgg tgttatccga agtggtgttg gagaggaccg aattggagat ttcgtatgcc |
| | 6421 ccgcgcctcg accaggaaaa agaagaacta ctacgcaaga aattacagct gaatcccaca |
| | 6481 cctgctaaca gaagcagata ccagtccagg agggtggaga atatgaaagc cataacagct |
| | 6541 agacgtattc tgcaaggcct agggcattat ttgaaggcag aaggaaaagt ggagtgctat |
| | 6601 cgaaccctgc atcctgttcc tttgtattca tctagtgtga atcgtgcttt ttcaagcccc |
| | 6661 aaggtcgcag tggaagcctg caatgccatg ctgaaagaaa attttccgac tgtagcttcc |
| | 6721 tactgtatta ttccagagta cgatgcctat ctggacatgg ttgacggcgc ttcttgttgc |
| | 6781 ttagacactg ccagtttttg ccctgcgaag ctgcgcagct ttccaaagaa acactcctat |
| | 6841 ttggaaccca caatacggtc ggcagtgcca tcagcgattc agaacacgct ccagaacgtc |
| | 6901 ctggcagctg ccacaaaaag aaattgcaac gtcacgcaaa tgagagaatt gcccgtattg |
| | 6961 gattcggctg cctttaatgt ggaatgcttc aagaaatatg cgtgcaataa tgaatattgg |
| | 7021 gaaacgttta aagaaaaccc catcaggctt actgaagaaa atgtggtaaa ttacattact |
| | 7081 aaattaaaag gaccaaaagc tgctgctctt tttgcgaaga cacataattt gaatatgtta |
| | 7141 caggacatac caatggacag gtttgtaatg gacttaaaga gggacgtgaa agtgactcca |
| | 7201 ggaacaaaac atactgaaga acggcccaag gtacaggtga ttcaggctgc cgatccacta |
| | 7261 gcgacagcgg atctgtgcgg aatccaccgg gagttggtta ggagattaaa tgctgtcctg |
| | 7321 cttccgaaca tccatacact gtttgacatg tcggctgaag actttgacgc tattattgcc |
| | 7381 gagcatttcc agcctgggga ctgtgtactg gaaactgaca ttgcgtcgtt tgataaaagt |
| | 7441 gaggacgacg ccatggctct gaccgcgtta atgattctgg aagacctagg agtggacgca |
| | 7501 gagctgttga cgctgattga ggcggctttc ggcgaaatat catcaataca tttgcccacc |
| | 7561 aaaactaaat ttaaattcgg agccatgatg aaatccggaa tgttcctcac actgtttgtg |
| | 7621 aacacagtca tcaacatcgt aatcgcaagc agagtgttaa gagagcggct aaccggatca |
| | 7681 ccatgtgcag cattcattgg agatgacaat atcgtgaaag gagtcaaatc tgacaaatta |
| | 7741 atggcagaca ggtgcgccac ttggttgaac atggaagtca agatcataga cgccgtggtg |
| | 7801 ggcgagaaag cgccctattt ttgtggaggg tttatcttgt gtgactccgt gaccggcaca |
| | 7861 gcgtgccgtg tggcagaccc cctaaaaagg ctgtttaagc ttggcaaacc cctggcagta |
| | 7921 gacgatgaac atgacgatga caggagaagg gcattacacg aagagtcaac acgctggaat |
| | 7981 cgagtgggaa ttcttccaga gctgtgtaag gcagtagaat caaggtatga aaccgtagga |
| | 8041 acttccatca tagttatggc catgactact ctagctagca gtgttaaatc attcagctac |
| | 8101 ctgagagggg cccctataac tctctacggc taacctgaat ggactacgac atagtctagt |
| | 8161 ccgccaaggc gatcgcgcca ccatggtgag caagggcgag gagctgttca ccggggtggt |
| | 8221 gcccatcctg gtcgagctgg acggcgacgt aaacggccac aagttcagcg tgtccggcga |
| | 8281 gggcgagggc gatgccacct acggcaagct gaccctgaag ttcatctgca ccaccggcaa |
| | 8341 gctgcccgtg ccctggccca ccctcgtgac caccctgacc tacggcgtgc agtgcttcag |
| | 8401 ccgctacccc gaccacatga agcagcacga cttcttcaag tccgccatgc ccgaaggcta |
| | 8461 cgtccaggag cgcaccatct tottcaagga cgacggcaac tacaagaccc gcgccgaggt |
| | 8521 gaagttcgag ggcgacaccc tggtgaaccg catcgagctg aagggcatcg acttcaagga |
| | 8581 ggacggcaac atcctggggc acaagctgga gtacaactac aacagccaca acgtctatat |
| | 8641 catggccgac aagcagaaga acggcatcaa ggtgaacttc aagatccgcc acaacatcga |
| | 8701 ggacggcagc gtgcagctcg ccgaccacta ccagcagaac acccccatcg gcgacggccc |
| | 8761 cgtgctgctg cccgacaacc actacctgag cacccagtcc gccctgagca aagaccccaa |
| | 8821 cgagaagcgc gatcacatgg tcctgctgga gttcgtgacc gccgccggga tcactctcgg |
| | 8881 catggacgag ctgtacaagt agcctgcagg tgaacatagc agcaattggc aagctgctta |
| | 8941 tatagaactt gcggcgattg gcatgccgct ttaaaatttt attttatttt cttttctttt |
| | 9001 ccgaatcgga ttttgttttt aatatttett gagatccttt ttttctgcgc gtaatctgct |
| | 9061 gcttgcaaac aaaaaaacca ccgctaccag cggtggtttg tttgccggat caagagctac |
| | 9121 caactctttt tccgaaggta actggcttca gcagagcgca gataccaaat actgtccttc |
| | 9181 tagtgtagcc gtagttaggc caccacttca agaactctgt agcaccgcct acatacctcg |
| | 9241 ctctgctaat cctgttacca gtggctgctg ccagtggcga taagtcgtgt cttaccgggt |
| | 9301 tggactcaag acgatagtta ccggataagg cgcagcggtc gggctgaacg gggggttcgt |
| | 9361 gcacacagcc cagcttggag cgaacgacct acaccgaact gagataccta cagcgtgagc |
| | 9421 tatgagaaag cgccacgctt cccgaaggga gaaaggcgga caggtatccg gtaagcggca |
| | 9481 gggtcggaac aggagagcgc acgagggagc ttccaggggg aaacgcctgg tatctttata |
| | 9541 gtcctgtcgg gtttcgccac ctctgacttg agcgtcgatt tttgtgatgc tcgtcagggg |
| | 9601 ggcggagcct atggaaagcg gccgccgcgg aacccctatt tgtttatttt tctaaataca |
| | 9661 ttcaaatatg tatccgctca tgagacaata accctgataa atgcttcaat aatattgaaa |
| | 9721 aaggaagagt atgattgaac aagatggatt gcacgcaggt tctccggccg cttgggtgga |
| | 9781 gaggctattc ggctatgact gggcacaaca gacaatcggc tgctctgatg ccgccgtgtt |
| | 9841 ccggctgtca gcgcaggggc gcccggttct ttttgtcaag accgacctgt ccggtgccct |
| | 9901 gaatgaactg caggacgagg cagcgcggct atcgtggctg gccacgacgg gcgttccttg |
| | 9961 cgcagctgtg ctcgacgttg tcactgaagc gggaagggac tggctgctat tgggcgaagt |
| | 10021 gccggggcag gatctcctgt catctcacct tgctcctgcc gagaaagtat ccatcatggc |
| | 10081 tgatgcaatg cggcggctgc atacgcttga tccggctacc tgcccattcg accaccaagc |
| | 10141 gaaacatcgc atcgagcgag cacgtactcg gatggaagcc ggtcttgtcg atcaggatga |
| | 10201 tctggacgaa gagcatcagg ggctcgcgcc agccgaactg ttcgccaggc tcaaggcgcg |
| | 10261 catgcccgac ggcgaggatc tcgtcgtgac ccatggcgat gcctgcttgc cgaatatcat |
| | 10321 ggtggaaaat ggccgctttt ctggattcat cgactgtggc cggctgggtg tggcggaccg |
| | 10381 ctatcaggac atagcgttgg ctacccgtga tattgctgaa gagcttggcg gcgaatgggc |
| | 10441 tgaccgcttc ctcgtgcttt acggtatcgc cgctcccgat tcgcagcgca tcgccttcta |
| | 10501 tcgccttctt gacgagttct tctgaattga aaaaggaaga gtaacgccag caacgcgagc |
| | 10561 tcggccggcc cgttacataa cttacggtaa atggcccgcc tggctgaccg cccaacgacc |
| | 10621 cccgcccatt gacgtcaata atgacgtatg ttcccatagt aacgccaata gggactttcc |
| | 10681 attgacgtca atgggtggag tatttacggt aaactgccca cttggcagta catcaagtgt |
| | 10741 atcatatgcc aagtacgccc cctattgacg tcaatgacgg taaatggccc gcctggcatt |
| | 10801 atgcccagta catgacctta tgggactttc ctacttggca gtacatctac gtattagtca |
| | 10861 tcgctattac catggtgatg cggttttggc agtacatcaa tgggcgtgga tagcggtttg |
| | 10921 actcacgggg atttccaagt ctccacccca ttgacgtcaa tgggagtttg ttttggcacc |
| | 10981 aaaatcaacg ggactttcca aaatgtcgta acaactccgc cccattgacg caaatgggcg |
| | 11041 gtaggcgtgt acggtgggag gtctatataa gcagagctgg ccggcccgtt tagtgaaccg |
| | 11101 tcagaatttt gtaatacgac tcactatagg g |

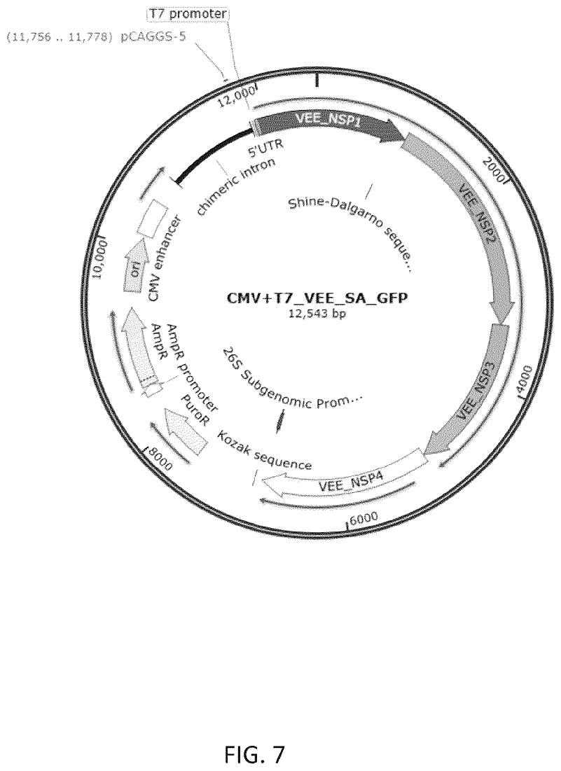

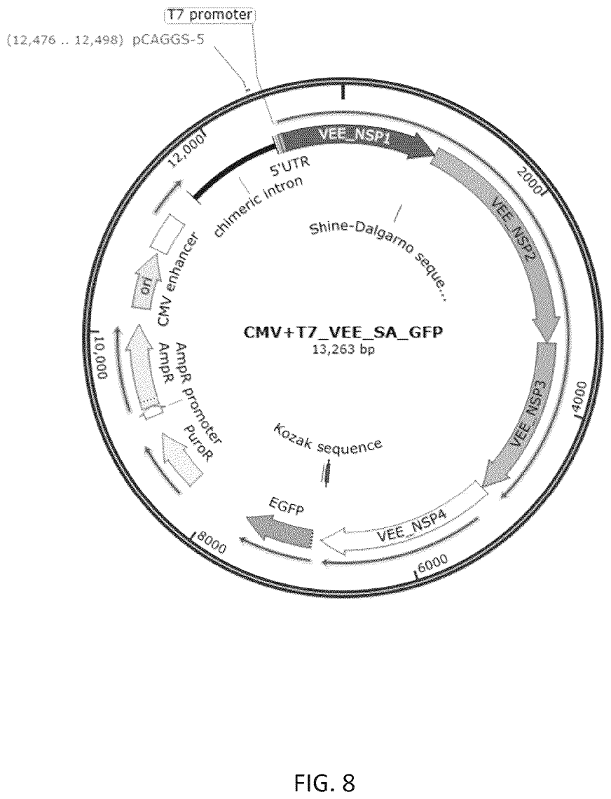

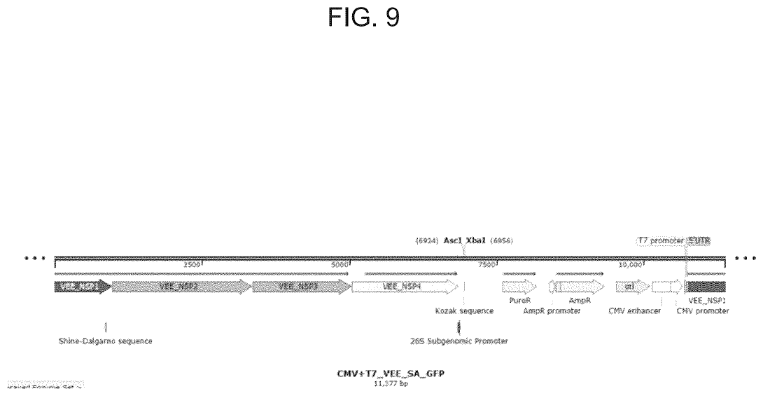

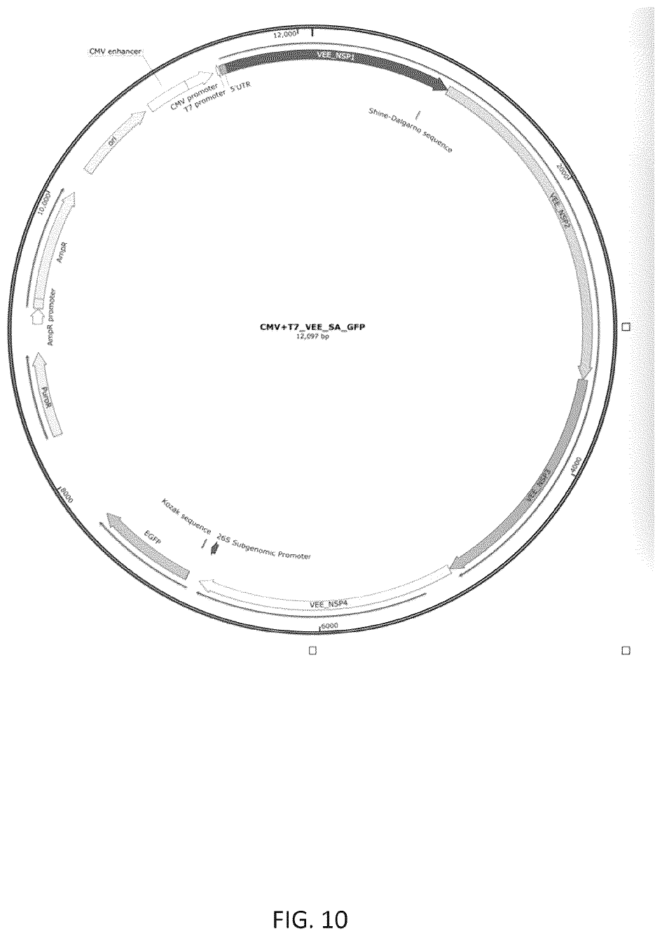

DNA sequence of CMV+T7_VEE_SA_GFP
(map set forth in FIG. 7)
AACGGCTCGTAACATAGGCCTATGCAGCTCTGACGTTATGGAGCG
GTCACGTAGAGGGATGTCCATTCTTAGAAAGAAGTATTTGAAACC
ATCCAACAATGTTCTATTCTCTGTTGGCTCGACCATCTACCACGA
GAAGAGGGACTTACTGAGGAGCTGGCACCTGCCGTCTGTATTTCA
CTTACGTGGCAAGCAAAATTACACATGTCGGTGTGAGACTATAGT
TAGTTGCGACGGGTACGTCGTTAAAAGAATAGCTATCAGTCCAGG
CCTGTATGGGAAGCCTTCAGGCTATGCTGCTACGATGCACCGCGA
GGGATTCTTGTGCTGCAAAGTGACAGACACATTGAACGGGGAGAG
GGTCTCTTTTCCCGTGTGCACGTATGTGCCAGCTACATTGTGTGA
CCAAATGACTGGCATACTGGCAACAGATGTCAGTGCGGACGACGC
GCAAAAACTGCTGGTTGGGCTCAACCAGCGTATAGTCGTCAACGG
TCGCACCCAGAGAAACACCAATACCATGAAAAATTACCTTTTGCC
CGTAGTGGCCCAGGCATTTGCTAGGTGGGCAAAGGAATATAAGGA
AGATCAAGAAGATGAAAGGCCACTAGGACTACGAGATAGACAGTT
AGTCATGGGGTGTTGTTGGGCTTTTAGAAGGCACAAGATAACATC
TATTTATAAGCGCCCGGATACCCAAACCATCATCAAAGTGAACAG
CGATTTCCACTCATTCGTGCTGCCCAGGATAGGCAGTAACACATT
GGAGATCGGGCTGAGAACAAGAATCAGGAAAATGTTAGAGGAGCA
CAAGGAGCCGTCACCTCTCATTACCGCCGAGGACGTACAAGAAGC
TAAGTGCGCAGCCGATGAGGCTAAGGAGGTGCGTGAAGCCGAGGA
GTTGCGCGCAGCTCTACCACCTTTGGCAGCTGATGTTGAGGAGCC
CACTCTGGAGGCAGACGTCGACTTGATGTTACAAGAGGCTGGGGC
CGGCTCAGTGGAGACACCTCGTGGCTTGATAAAGGTTACCAGCTA
CGATGGCGAGGACAAGATCGGCTCTTACGCTGTGCTTTCTCCGCA
GGCTGTACTCAAGAGTGAAAAATTATCTTGCATCCACCCTCTCGC
TGAACAAGTCATAGTGATAACACACTCTGGCCGAAAAGGGCGTTA
TGCCGTGGAACCATACCATGGTAAAGTAGTGGTGCCAGAGGGACA
TGCAATACCCGTCCAGGACTTTCAAGCTCTGAGTGAAAGTGCCAC
CATTGTGTACAACGAACGTGAGTTCGTAAACAGGTACCTGCACCA
TATTGCCACACATGGAGGAGCGCTGAACACTGATGAAGAATATTA
CAAAACTGTCAAGCCCAGCGAGCACGACGGCGAATACCTGTACGA
CATCGACAGGAAACAGTGCGTCAAGAAAGAACTAGTCACTGGGCT
AGGGCTCACAGGCGAGCTGGTGGATCCTCCCTTCCATGAATTCGC
CTACGAGAGTCTGAGAACACGACCAGCCGCTCCTTACCAAGTACC
AACCATAGGGGTGTATGGCGTGCCAGGATCAGGCAAGTCTGGCAT
CATTAAAAGCGCAGTCACCAAAAAAGATCTAGTGGTGAGCGCCAA
GAAAGAAAACTGTGCAGAAATTATAAGGGACGTCAAGAAAATGAA
AGGGCTGGACGTCAATGCCAGAACTGTGGACTCAGTGCTCTTGAA
TGGATGCAAACACCCCGTAGAGACCCTGTATATTGACGAAGCTTT
TGCTTGTCATGCAGGTACTCTCAGAGCGCTCATAGCCATTATAAG
ACCTAAAAAGGCAGTGCTCTGCGGGGATCCCAAACAGTGCGGTTT
TTTTAACATGATGTGCCTGAAAGTGCATTTTAACCACGAGATTTG
CACACAAGTCTTCCACAAAAGCATCTCTCGCCGTTGCACTAAATC
TGTGACTTCGGTCGTCTCAACCTTGTTTTACGACAAAAAAATGAG
AACGACGAATCCGAAAGAGACTAAGATTGTGATTGACACTACCGG
CAGTACCAAACCTAAGCAGGACGATCTCATTCTCACTTGTTTCAG
AGGGTGGGTGAAGCAGTTGCAAATAGATTACAAAGGCAACGAAAT
AATGACGGCAGCTGCCTCTCAAGGGCTGACCCGTAAAGGTGTGTA
TGCCGTTCGGTACAAGGTGAATGAAAATCCTCTGTACGCACCCAC
CTCAGAACATGTGAACGTCCTACTGACCCGCACGGAGGACCGCAT
CGTGTGGAAAACACTAGCCGGCGACCCATGGATAAAAACACTGAC
TGCCAAGTACCCTGGGAATTTCACTGCCACGATAGAGGAGTGGCA
AGCAGAGCATGATGCCATCATGAGGCACATCTTGGAGAGACCGGA
CCCTACCGACGTCTTCCAGAATAAGGCAAACGTGTGTTGGGCCAA
GGCTTTAGTGCCGGTGCTGAAGACCGCTGGCATAGACATGACCAC
TGAACAATGGAACACTGTGGATTATTTTGAAACGGACAAAGCTCA
CTCAGCAGAGATAGTATTGAACCAACTATGCGTGAGGTTCTTTGG
ACTCGATCTGGACTCCGGTCTATTTTCTGCACCCACTGTTCCGTT
ATCCATTAGGAATAATCACTGGGATAACTCCCCGTCGCCTAACAT
GTACGGGCTGAATAAAGAAGTGGTCCGTCAGCTCTCTCGCAGGTA
CCCACAACTGCCTCGGGCAGTTGCCACTGGAAGAGTCTATGACAT
GAACACTGGTACACTGCGCAATTATGATCCGCGCATAAACCTAGT
ACCTGTAAACAGAAGACTGCCTCATGCTTTAGTCCTCCACCATAA
TGAACACCCACAGAGTGACTTTTCTTCATTCGTCAGCAAATTGAA
GGGCAGAACTGTCCTGGTGGTCGGGGAAAAGTTGTCCGTCCCAGG
CAAAATGGTTGACTGGTTGTCAGACCGGCCTGAGGCTACCTTCAG
AGCTCGGCTGGATTTAGGCATCCCAGGTGATGTGCCCAAATATGA
CATAATATTTGTTAATGTGAGGACCCCATATAAATACCATCACTA
TCAGCAGTGTGAAGACCATGCCATTAAGCTTAGCATGTTGACCAA
GAAAGCTTGTCTGCATCTGAATCCCGGCGGAACCTGTGTCAGCAT
AGGTTATGGTTACGCTGACAGGGCCAGCGAAAGCATCATTGGTGC
TATAGCGCGGCAGTTCAAGTTTTCCCGGGTATGCAAACCGAAATC
CTCACTTGAAGAGACGGAAGTTCTGTTTGTATTCATTGGGTACGA
TCGCAAGGCCCGTACGCACAATTCTTACAAGCTTTCATCAACCTT
GACCAACATTTATACAGGTTCCAGACTCCACGAAGCCGGATGTGC
ACCCTCATATCATGTGGTGCGAGGGGATATTGCCACGGCCACCGA
AGGAGTGATTATAAATGCTGCTAACAGCAAAGGACAACCTGGCGG
AGGGGTGTGCGGAGCGCTGTATAAGAAATTCCCGGAAAGCTTCGA
TTTACAGCCGATCGAAGTAGGAAAAGCGCGACTGGTCAAAGGTGC
AGCTAAACATATCATTCATGCCGTAGGACCAAACTTCAACAAAGT
TTCGGAGGTTGAAGGTGACAAACAGTTGGCAGAGGCTTATGAGTC
CATCGCTAAGATTGTCAACGATAACAATTACAAGTCAGTAGCGAT
TCCACTGTTGTCCACCGGCATCTTTTCCGGGAACAAAGATCGACT
AACCCAATCATTGAACCATTTGCTGACAGCTTTAGACACCACTGA
TGCAGATGTAGCCATATACTGCAGGGACAAGAAATGGGAAATGAC
TCTCAAGGAAGCAGTGGCTAGGAGAGAAGCAGTGGAGGAGATATG
CATATCCGACGACTCTTCAGTGACAGAACCTGATGCAGAGCTGGT
GAGGGTGCATCCGAAGAGTTCTTTGGCTGGAAGGAAGGGCTACAG
CACAAGCGATGGCAAAACTTTCTCATATTTGGAAGGGACCAAGTT
TCACCAGGCGGCCAAGGATATAGCAGAAATTAATGCCATGTGGCC
CGTTGCAACGGAGGCCAATGAGCAGGTATGCATGTATATCCTCGG
AGAAAGCATGAGCAGTATTAGGTCGAAATGCCCCGTCGAAGAGTC
GGAAGCCTCCACACCACCTAGCACGCTGCCTTGCTTGTGCATCCA
TGCCATGACTCCAGAAAGAGTACAGCGCCTAAAAGCCTCACGTCC
AGAACAAATTACTGTGTGCTCATCCTTTCCATTGCCGAAGTATAG
AATCACTGGTGTGCAGAAGATCCAATGCTCCCAGCCTATATTGTT
CTCACCGAAAGTGCCTGCGTATATTCATCCAAGGAAGTATCTCGT
GGAAACACCACCGGTAGACGAGACTCCGGAGCCATCGGCAGAGAA
CCAATCCACAGAGGGGACACCTGAACAACCACCACTTATAACCGA
GGATGAGACCAGGACTAGAACGCCTGAGCCGATCATCATCGAAGA
GGAAGAAGAGGATAGCATAAGTTTGCTGTCAGATGGCCCGACCCA
CCAGGTGCTGCAAGTCGAGGCAGACATTCACGGGCCGCCCTCTGT
ATCTAGCTCATCCTGGTCCATTCCTCATGCATCCGACTTTGATGT
GGACAGTTTATCCATACTTGACACCCTGGAGGGAGCTAGCGTGAC
CAGCGGGGCAACGTCAGCCGAGACTAACTCTTACTTCGCAAAGAG
TATGGAGTTTCTGGCGCGACCGGTGCCTGCGCCTCGAACAGTATT
CAGGAACCCTCCACATCCCGCTCCGCGCACAAGAACACCGTCACT
TGCACCCAGCAGGGCCTGCTCGAGAACCAGCCTAGTTTCCACCCC
GCCAGGCGTGAATAGGGTGATCACTAGAGAGGAGCTCGAGGCGCT
TACCCCGTCACGCACTCCTAGCAGGTCGGTCTCGAGAACCAGCCT
GGTCTCCAACCCGCCAGGCGTAAATAGGGTGATTACAAGAGAGGA
GTTTGAGGCGTTCGTAGCACAACAACAATGACGGTTTGATGCGGG
TGCATACATCTTTTCCTCCGACACCGGTCAAGGGCATTTACAACA
AAAATCAGTAAGGCAAACGGTGCTATCCGAAGTGGTGTTGGAGAG
GACCGAATTGGAGATTTCGTATGCCCCGCGCCTCGACCAAGAAAA
AGAAGAATTACTACGCAAGAAATTACAGTTAAATCCCACACCTGC
TAACAGAAGCAGATACCAGTCCAGGAAGGTGGAGAACATGAAAGC
CATAACAGCTAGACGTATTCTGCAAGGCCTAGGGCATTATTTGAA
GGCAGAAGGAAAAGTGGAGTGCTACCGAACCCTGCATCCTGTTCC
TTTGTATTCATCTAGTGTGAACCGTGCCTTTTCAAGCCCCAAGGT
CGCAGTGGAAGCCTGTAACGCCATGTTGAAAGAGAACTTTCCGAC
TGTGGCTTCTTACTGTATTATTCCAGAGTACGATGCCTATTTGGA
CATGGTTGACGGAGCTTCATGCTGCTTAGACACTGCCAGTTTTTG
CCCTGCAAAGCTGCGCAGCTTTCCAAAGAAACACTCCTATTTGGA
ACCCACAATACGATCGGCAGTGCCTTCAGCGATCCAGAACACGCT
CCAGAACGTCCTGGCAGCTGCCACAAAAAGAAATTGCAATGTCAC
GCAAATGAGAGAATTGCCCGTATTGGATTCGGCGGCCTTTAATGT
GGAATGCTTCAAGAAATATGCGTGTAATAATGAATATTGGGAAAC
GTTTAAAGAAAACCCCATCAGGCTTACTGAAGAAAACGTGGTAAA
TTACATTACCAAATTAAAAGGACCAAAAGCTGCTGCTCTTTTTGC
GAAGACACATAATTTGAATATGTTGCAGGACATACCAATGGACAG
GTTTGTAATGGACTTAAAGAGAGACGTGAAAGTGACTCCAGGAAC
AAAACATACTGAAGAACGGCCCAAGGTACAGGTGATCCAGGCTGC
CGATCCGCTAGCAACAGCGTATCTGTGCGGAATCCACCGAGAGCT
GGTTAGGAGATTAAATGCGGTCCTGCTTCCGAACATTCATACACT
GTTTGATATGTCGGCTGAAGACTTTGACGCTATTATAGCCGAGCA
CTTCCAGCCTGGGGATTGTGTTCTGGAAACTGACATCGCGTCGTT
TGATAAAAGTGAGGACGACGCCATGGCTCTGACCGCGTTAATGAT
TCTGGAAGACTTAGGTGTGGACGCAGAGCTGTTGACGCTGATTGA
GGCGGCTTTCGGCGAAATTTCATCAATACATTTGCCCACTAAAAC
TAAATTTAAATTCGGAGCCATGATGAAATCTGGAATGTTCCTCAC
ACTGTTTGTGAACACAGTCATTAACATTGTAATCGCAAGCAGAGT
GTTGAGAGAACGGCTAACCGGATCACCATGTGCAGCATTCATTGG
AGATGACAATATCGTGAAAGGAGTCAAATCGGACAAATTAATGGC
AGACAGGTGCGCCACCTGGTTGAATATGGAAGTCAAGATTATAGA
TGCTGTGGTGGGCGAGAAAGCGCCTTATTTCTGTGGAGGGTTTAT
TTTGTGTGACTCCGTGACCGGCACAGCGTGCCGTGTGGCAGACCC
CCTAAAAAGGCTGTTTAAGCTTGGCAAACCTCTGGCAGCAGACGA
TGAACATGATGATGACAGGAGAAGGGCATTGCATGAAGAGTCAAC
ACGCTGGAACCGAGTGGGTATTCTTTCAGAGCTGTGCAAGGCAGT
AGAATCAAGGTATGAAACCGTAGGAACTTCCATCATAGTTATGGC
CATGACTACTCTAGCTAGCAGTGTTAAATCATTCAGCTACCTGAG
AGGGGCCCCTATAACTCTCTACGGCTAACCTGAATGGACTACGAC
ATAGTCTAGTCCGCCAAGTCTGTTTAAACAGCATATGGGCGCGCC
CTCAGCATCGATTCAATTCGCCACCTCTAGAGTGTTTAAACCGAC
CCGGGCGGCCGCAACTAACTTAAGCTAGCAACGGTTTCCCTCTAG
CGGGATCAATTCCGCCCCCCCCCCCTAACGTTACTGGCCGAAGCC
GCTTGGAATAAGGCCGGTGTGCGTTTGTCTATATGTTATTTTCCA
CCATATTGCCGTCTTTTGGCAATGTGAGGGCCCGGAAACCTGGCC
CTGTCTTCTTGACGAGCATTCCTAGGGGTCTTTCCCCTCTCGCCA
AAGGAATGCAAGGTCTGTTGAATGTCGTGAAGGAAGCAGTTCCTC
TGGAAGCTTCTTGAAGACAAACAACGTCTGTAGCGACCCTTTGCA
GGCAGCGGAACCCCCCACCTGGCGACAGGTGCCTCTGCGGCCAAA
AGCCACGTGTATAAGATACACCTGCAAAGGCGGCACAACCCCAGT
GCCACGTTGTGAGTTGGATAGTTGTGGAAAGAGTCAAATGGCTCT
CCTCAAGCGTATTCAACAAGGGGCTGAAGGATGCCCAGAAGGTAC
CCCATTGTATGGGATCTGATCTGGGGCCTCGGTGCACATGCTTTA
CATGTGTTTAGTCGAGGTTAAAAAAACGTCTAGGCCCCCCGAACC
ACGGGGACGTGGTTTTCCTTTGAAAAACACGATAATACCATGACC
GAGTACAAGCCCACGGTGCGCCTCGCCACCCGCGACGACGTCCCC
AGGGCCGTACGCACCCTCGCCGCCGCGTTCGCCGACTACCCCGCC
ACGCGCCACACCGTCGATCCGGACCGCCACATCGAGCGGGTCACC
GAGCTGCAAGAACTCTTCCTCACGCGCGTCGGGCTCGACATCGGC
AAGGTGTGGGTCGCGGACGACGGCGCCGCGGTGGCGGTCTGGACC
ACGCCGGAGAGCGTCGAAGCGGGGGCGGTGTTCGCCGAGATCGGC
CCGCGCATGGCCGAGTTGAGCGGTTCCCGGCTGGCCGCGCAGCAA
CAGATGGAAGGCCTCCTGGCGCCGCACCGGCCCAAGGAGCCCGCG
TGGTTCCTGGCCACCGTCGGCGTCTCGCCCGACCACCAGGGCAAG
GGTCTGGGCAGCGCCGTCGTGCTCCCCGGAGTGGAGGCGGCCGAG
CGCGCCGGGGTGCCCGCCTTCCTGGAGACCTCCGCGCCCCGCAAC
CTCCCCTTCTACGAGCGGCTCGGCTTCACCGTCACCGCCGACGTC
GAGGTGCCCGAAGGACCGCGCACCTGGTGCATGACCCGCAAGCCC
GGTGCCTGAGAATTGGCAAGCTGCTTACATAGAACTCGCGGCGAT
TGGCATGCCGCCTTAAAATTTTTATTTTATTTTTTCTTTTCTTTT
CCGAATCGGATTTTGTTTTTAATATTTCAAAAAAAAAAAAAAAAA
AAAAAAAAACGCGTCGAGGGGAATTAATTCTTGAAGACGAAAGGG
CCAGGTGGCACTTTTCGGGGAAATGTGCGCGGAACCCCTATTTGT
TTATTTTTCTAAATACATTCAAATATGTATCCGCTCATGAGACAA
TAACCCTGATAAATGCTTCAATAATATTGAAAAAGGAAGAGTATG
AGTATTCAACATTTCCGTGTCGCCCTTATTCCCTTTTTTGCGGCA
TTTTGCCTTCCTGTTTTTGCTCACCCAGAAACGCTGGTGAAAGTA
AAAGATGCTGAAGATCAGTTGGGTGCACGAGTGGGTTACATCGAA
CTGGATCTCAACAGCGGTAAGATCCTTGAGAGTTTTCGCCCCGAA
GAACGTTTTCCAATGATGAGCACTTTTAAAGTTCTGCTATGTGGC
GCGGTATTATCCCGTGTTGACGCCGGGCAAGAGCAACTCGGTCGC
CGCATACACTATTCTCAGAATGACTTGGTTGAGTACTCACCAGTC
ACAGAAAAGCATCTTACGGATGGCATGACAGTAAGAGAATTATGC
AGTGCTGCCATAACCATGAGTGATAACACTGCGGCCAACTTACTT
CTGACAACGATCGGAGGACCGAAGGAGCTAACCGCTTTTTTGCAC
AACATGGGGGATCATGTAACTCGCCTTGATCGTTGGGAACCGGAG
CTGAATGAAGCCATACCAAACGACGAGCGTGACACCACGATGCCT
GTAGCAATGGCAACAACGTTGCGCAAACTATTAACTGGCGAACTA
CTTACTCTAGCTTCCCGGCAACAATTAATAGACTGGATGGAGGCG
GATAAAGTTGCAGGACCACTTCTGCGCTCGGCCCTTCCGGCTGGC
TGGTTTATTGCTGATAAATCTGGAGCCGGTGAGCGTGGGTCTCGC
GGTATCATTGCAGCACTGGGGCCAGATGGTAAGCCCTCCCGTATC
GTAGTTATCTACACGACGGGGAGTCAGGCAACTATGGATGAACGA
AATAGACAGATCGCTGAGATAGGTGCCTCACTGATTAAGCATTGG
TAACTGTCAGACCAAGTTTACTCATATATACTTTAGATTGATTTA
AAACTTCATTTTTAATTTAAAAGGATCTAGGTGAAGATCCTTTTT
GATAATCTCATGACCAAAATCCCTTAACGTGAGTTTTCGTTCCAC
TGAGCGTCAGACCCCGTAGAAAAGATCAAAGGATCTTCTTGAGAT
CCTTTTTTTCTGCGCGTAATCTGCTGCTTGCAAACAAAAAAACCA
CCGCTACCAGCGGTGGTTTGTTTGCCGGATCAAGAGCTACCAACT
CTTTTTCCGAAGGTAACTGGCTTCAGCAGAGCGCAGATACCAAAT
ACTGTCCTTCTAGTGTAGCCGTAGTTAGGCCACCACTTCAAGAAC
TCTGTAGCACCGCCTACATACCTCGCTCTGCTAATCCTGTTACCA
GTGGCTGCTGCCAGTGGCGATAAGTCGTGTCTTACCGGGTTGGAC
TCAAGACGATAGTTACCGGATAAGGCGCAGCGGTCGGGCTGAACG
GGGGGTTCGTGCACACAGCCCAGCTTGGAGCGAACGACCTACACC
GAACTGAGATACCTACAGCGTGAGCTATGAGAAAGCGCCACGCTT
CCCGAAGGGAGAAAGGCGGACAGGTATCCGGTAAGCGGCAGGGTC
GGAACAGGAGAGCGCACGAGGGAGCTTCCAGGGGGAAACGCCTGG
TATCTTTATAGTCCTGTCGGGTTTCGCCACCTCTGACTTGAGCGT
CGATTTTTGTGATGCTCGTCAGGGGGGCGGAGCCTATGGAAAAAC
GCCAGCAACGCGAGCTCGCGATCGCTTAATTAAgacattgattat
tgactagttattaatagtaatcaattacggggtcattagttcata
gcccatatatggagttccgcgttacataacttacggtaaatggcc
cgcctggctgaccgcccaacgacccccgcccattgacgtcaataa
tgacgtatgttcccatagtaacgccaatagggactttccattgac
gtcaatgggggagtatttacggtaaactgcccacttggcagtaca
tcaagtgtatcatatgccaagtacgccccctattgacgtcaatga
cggtaaatggcccgcctggcattatgcccagtacatgaccttatg
ggactttcctacttggcagtacatctacgtattagtcatcgctat
taccatggtcgaggtgagccccacgttctgcttcactctccccat
ctcccccccctccccacccccaattttgtatttatttatttttta
attattttgtgcagcgatgggggcggggggggggggggcgcgcgc
caggggggggggggggggggggggggggggggggggggggggggg
ggggggcggcggcagccaatcagagcggcgcgctccgaaagtttc
cttttatggcgaggcggcggcggcggcggccctataaaaagcgaa
gcgcgcggcgggcgggagtcgctgcgcgctgccttcgccccgtgc
cccgctccgccgccgcctcgcgccgcccgccccggctctgactga
ccgcgttactcccacaggtgagcggggggacggcccttctcctcc
gggctgtaattagcgcttggtttaatgacggcttgtttcttttct
gtggctgcgtgaaagccttgaggggctccgggagggccctttgtg
cggggggagcggctcggggggtgcgtgcgtgtgtgtgtgcgtggg
gagcgccgcgtgcggctccgcgctgcccggcggctgtgagcgctg
cgggcgcggcgcggggctttgtgcgctccgcagtgtgcgcgaggg
gagcgcggccgggggcggtgccccgcggtgcggggggggctgcga
ggggaacaaaggctgcgtgcggggtgtgtgcgtgggggggtgagc
agggggtgtgggcgcgtcggtcgggctgcaaccccccctgcaccc
ccctccccgagttgctgagcacggcccggcttcgggtgcggggct
ccgtacggggcgtggcgcggggctcgccgtgccgggcgggggggg
cggcaggtgggggtgccgggcggggcggggccgcctcgggccggg
gggggctcggggggggggcgcggcggcccccggagcgccggcggc
tgtcgaggcgcggcgagccgcagccattgccttttatggtaatcg
tgcgagagggcgcagggacttcctttgtcccaaatctgtgcggag
ccgaaatctgggaggcgccgccgcaccccctctagcgggcgcggg
gcgaagcggtgcggcgccggcaggaaggaaatgggcggggagggc
cttcgtgcgtcgccgcgccgccgtccccttctccctctccagcct
cggggctgtccgcggggggacggctgccttcgggggggacggggc
agggcggggttcggcttctggcgtgtgaccggcggctctagagcc
tctgctaaccatgttcatgccttcttctttttcctacagGGTTTA
GTGAACCGTCAGATCCGCTAGTAATACGACTCACTATAGGGCCGG
CCATAGGCGGCGCATGAGAGAAGCCCAGACCAATTACCTACCCAA
AATGGAGAAAGTTCACGTTGACATCGAGGAAGACAGCCCATTCCT
CAGAGCTTTGCAGCGGAGCTTCCCGCAGTTTGAGGTAGAAGCCAA
GCAGGTCACTGATAATGACCATGCTAATGCCAGAGCGTTTTCGCA
TCTGGCTTCAAAACTGATCGAAACGGAGGTGGACCCATCCGACAC
GATCCTTGACATTGGAAGTGCGCCCGCCCGCAGAATGTATTCTAA
GCACAAGTATCATTGTATCTGTCCGATGAGATGTGCGGAAGATCC
GGACAGATTGTATAAGTATGCAACTAAGCTGAAGAAAAACTGTAA
GGAAATAACTGATAAGGAATTGGACAAGAAAATGAAGGAGCTGGC
CGCCGTCATGAGCGACCCTGACCTGGAAACTGAGACTATGTGCCT
CCACGACGACGAGTCGTGTCGCTACGAAGGGCAAGTCGCTGTTTA
CCAGGATGTATACGCGGTTGACGGACCGACAAGTCTCTATCACCA
AGCCAATAAGGGAGTTAGAGTCGCCTACTGGATAGGCTTTGACAC
CACCCCTTTTATGTTTAAGAACTTGGCTGGAGCATATCCATCATA
CTCTACCAACTGGGCCGACGAAACCGTGTT
DNA sequence of CMV+T7_VEE_SA_GFP
(map set forth in FIG. 8)
AACGGCTCGTAACATAGGCCTATGCAGCTCTGACGTTATGGAGCG
GTCACGTAGAGGGATGTCCATTCTTAGAAAGAAGTATTTGAAACC
ATCCAACAATGTTCTATTCTCTGTTGGCTCGACCATCTACCACGA
GAAGAGGGACTTACTGAGGAGCTGGCACCTGCCGTCTGTATTTCA
CTTACGTGGCAAGCAAAATTACACATGTCGGTGTGAGACTATAGT
TAGTTGCGACGGGTACGTCGTTAAAAGAATAGCTATCAGTCCAGG
CCTGTATGGGAAGCCTTCAGGCTATGCTGCTACGATGCACCGCGA
GGGATTCTTGTGCTGCAAAGTGACAGACACATTGAACGGGGAGAG
GGTCTCTTTTCCCGTGTGCACGTATGTGCCAGCTACATTGTGTGA
CCAAATGACTGGCATACTGGCAACAGATGTCAGTGCGGACGACGC
GCAAAAACTGCTGGTTGGGCTCAACCAGCGTATAGTCGTCAACGG
TCGCACCCAGAGAAACACCAATACCATGAAAAATTACCTTTTGCC
CGTAGTGGCCCAGGCATTTGCTAGGTGGGCAAAGGAATATAAGGA
AGATCAAGAAGATGAAAGGCCACTAGGACTACGAGATAGACAGTT
AGTCATGGGGTGTTGTTGGGCTTTTAGAAGGCACAAGATAACATC
TATTTATAAGCGCCCGGATACCCAAACCATCATCAAAGTGAACAG
CGATTTCCACTCATTCGTGCTGCCCAGGATAGGCAGTAACACATT
GGAGATCGGGCTGAGAACAAGAATCAGGAAAATGTTAGAGGAGCA
CAAGGAGCCGTCACCTCTCATTACCGCCGAGGACGTACAAGAAGC
TAAGTGCGCAGCCGATGAGGCTAAGGAGGTGCGTGAAGCCGAGGA
GTTGCGCGCAGCTCTACCACCTTTGGCAGCTGATGTTGAGGAGCC
CACTCTGGAGGCAGACGTCGACTTGATGTTACAAGAGGCTGGGGC
CGGCTCAGTGGAGACACCTCGTGGCTTGATAAAGGTTACCAGCTA
CGATGGCGAGGACAAGATCGGCTCTTACGCTGTGCTTTCTCCGCA
GGCTGTACTCAAGAGTGAAAAATTATCTTGCATCCACCCTCTCGC
TGAACAAGTCATAGTGATAACACACTCTGGCCGAAAAGGGCGTTA
TGCCGTGGAACCATACCATGGTAAAGTAGTGGTGCCAGAGGGACA
TGCAATACCCGTCCAGGACTTTCAAGCTCTGAGTGAAAGTGCCAC
CATTGTGTACAACGAACGTGAGTTCGTAAACAGGTACCTGCACCA
TATTGCCACACATGGAGGAGCGCTGAACACTGATGAAGAATATTA
CAAAACTGTCAAGCCCAGCGAGCACGACGGCGAATACCTGTACGA
CATCGACAGGAAACAGTGCGTCAAGAAAGAACTAGTCACTGGGCT
AGGGCTCACAGGCGAGCTGGTGGATCCTCCCTTCCATGAATTCGC
CTACGAGAGTCTGAGAACACGACCAGCCGCTCCTTACCAAGTACC
AACCATAGGGGTGTATGGCGTGCCAGGATCAGGCAAGTCTGGCAT
CATTAAAAGCGCAGTCACCAAAAAAGATCTAGTGGTGAGCGCCAA
GAAAGAAAACTGTGCAGAAATTATAAGGGACGTCAAGAAAATGAA
AGGGCTGGACGTCAATGCCAGAACTGTGGACTCAGTGCTCTTGAA
TGGATGCAAACACCCCGTAGAGACCCTGTATATTGACGAAGCTTT
TGCTTGTCATGCAGGTACTCTCAGAGCGCTCATAGCCATTATAAG
ACCTAAAAAGGCAGTGCTCTGCGGGGATCCCAAACAGTGCGGTTT
TTTTAACATGATGTGCCTGAAAGTGCATTTTAACCACGAGATTTG
CACACAAGTCTTCCACAAAAGCATCTCTCGCCGTTGCACTAAATC
TGTGACTTCGGTCGTCTCAACCTTGTTTTACGACAAAAAAATGAG
AACGACGAATCCGAAAGAGACTAAGATTGTGATTGACACTACCGG
CAGTACCAAACCTAAGCAGGACGATCTCATTCTCACTTGTTTCAG
AGGGTGGGTGAAGCAGTTGCAAATAGATTACAAAGGCAACGAAAT
AATGACGGCAGCTGCCTCTCAAGGGCTGACCCGTAAAGGTGTGTA
TGCCGTTCGGTACAAGGTGAATGAAAATCCTCTGTACGCACCCAC
CTCAGAACATGTGAACGTCCTACTGACCCGCACGGAGGACCGCAT
CGTGTGGAAAACACTAGCCGGCGACCCATGGATAAAAACACTGAC
TGCCAAGTACCCTGGGAATTTCACTGCCACGATAGAGGAGTGGCA
AGCAGAGCATGATGCCATCATGAGGCACATCTTGGAGAGACCGGA
CCCTACCGACGTCTTCCAGAATAAGGCAAACGTGTGTTGGGCCAA
GGCTTTAGTGCCGGTGCTGAAGACCGCTGGCATAGACATGACCAC
TGAACAATGGAACACTGTGGATTATTTTGAAACGGACAAAGCTCA
CTCAGCAGAGATAGTATTGAACCAACTATGCGTGAGGTTCTTTGG
ACTCGATCTGGACTCCGGTCTATTTTCTGCACCCACTGTTCCGTT
ATCCATTAGGAATAATCACTGGGATAACTCCCCGTCGCCTAACAT
GTACGGGCTGAATAAAGAAGTGGTCCGTCAGCTCTCTCGCAGGTA
CCCACAACTGCCTCGGGCAGTTGCCACTGGAAGAGTCTATGACAT
GAACACTGGTACACTGCGCAATTATGATCCGCGCATAAACCTAGT
ACCTGTAAACAGAAGACTGCCTCATGCTTTAGTCCTCCACCATAA
TGAACACCCACAGAGTGACTTTTCTTCATTCGTCAGCAAATTGAA
GGGCAGAACTGTCCTGGTGGTCGGGGAAAAGTTGTCCGTCCCAGG
CAAAATGGTTGACTGGTTGTCAGACCGGCCTGAGGCTACCTTCAG
AGCTCGGCTGGATTTAGGCATCCCAGGTGATGTGCCCAAATATGA
CATAATATTTGTTAATGTGAGGACCCCATATAAATACCATCACTA
TCAGCAGTGTGAAGACCATGCCATTAAGCTTAGCATGTTGACCAA
GAAAGCTTGTCTGCATCTGAATCCCGGCGGAACCTGTGTCAGCAT
AGGTTATGGTTACGCTGACAGGGCCAGCGAAAGCATCATTGGTGC
TATAGCGCGGCAGTTCAAGTTTTCCCGGGTATGCAAACCGAAATC
CTCACTTGAAGAGACGGAAGTTCTGTTTGTATTCATTGGGTACGA
TCGCAAGGCCCGTACGCACAATTCTTACAAGCTTTCATCAACCTT
GACCAACATTTATACAGGTTCCAGACTCCACGAAGCCGGATGTGC
ACCCTCATATCATGTGGTGCGAGGGGATATTGCCACGGCCACCGA
AGGAGTGATTATAAATGCTGCTAACAGCAAAGGACAACCTGGCGG
AGGGGTGTGCGGAGCGCTGTATAAGAAATTCCCGGAAAGCTTCGA
TTTACAGCCGATCGAAGTAGGAAAAGCGCGACTGGTCAAAGGTGC
AGCTAAACATATCATTCATGCCGTAGGACCAAACTTCAACAAAGT
TTCGGAGGTTGAAGGTGACAAACAGTTGGCAGAGGCTTATGAGTC
CATCGCTAAGATTGTCAACGATAACAATTACAAGTCAGTAGCGAT
TCCACTGTTGTCCACCGGCATCTTTTCCGGGAACAAAGATCGACT
AACCCAATCATTGAACCATTTGCTGACAGCTTTAGACACCACTGA
TGCAGATGTAGCCATATACTGCAGGGACAAGAAATGGGAAATGAC
TCTCAAGGAAGCAGTGGCTAGGAGAGAAGCAGTGGAGGAGATATG
CATATCCGACGACTCTTCAGTGACAGAACCTGATGCAGAGCTGGT
GAGGGTGCATCCGAAGAGTTCTTTGGCTGGAAGGAAGGGCTACAG
CACAAGCGATGGCAAAACTTTCTCATATTTGGAAGGGACCAAGTT
TCACCAGGCGGCCAAGGATATAGCAGAAATTAATGCCATGTGGCC
CGTTGCAACGGAGGCCAATGAGCAGGTATGCATGTATATCCTCGG
AGAAAGCATGAGCAGTATTAGGTCGAAATGCCCCGTCGAAGAGTC
GGAAGCCTCCACACCACCTAGCACGCTGCCTTGCTTGTGCATCCA
TGCCATGACTCCAGAAAGAGTACAGCGCCTAAAAGCCTCACGTCC
AGAACAAATTACTGTGTGCTCATCCTTTCCATTGCCGAAGTATAG
AATCACTGGTGTGCAGAAGATCCAATGCTCCCAGCCTATATTGTT
CTCACCGAAAGTGCCTGCGTATATTCATCCAAGGAAGTATCTCGT
GGAAACACCACCGGTAGACGAGACTCCGGAGCCATCGGCAGAGAA
CCAATCCACAGAGGGGACACCTGAACAACCACCACTTATAACCGA
GGATGAGACCAGGACTAGAACGCCTGAGCCGATCATCATCGAAGA
GGAAGAAGAGGATAGCATAAGTTTGCTGTCAGATGGCCCGACCCA
CCAGGTGCTGCAAGTCGAGGCAGACATTCACGGGCCGCCCTCTGT
ATCTAGCTCATCCTGGTCCATTCCTCATGCATCCGACTTTGATGT
GGACAGTTTATCCATACTTGACACCCTGGAGGGAGCTAGCGTGAC
CAGCGGGGCAACGTCAGCCGAGACTAACTCTTACTTCGCAAAGAG
TATGGAGTTTCTGGCGCGACCGGTGCCTGCGCCTCGAACAGTATT
CAGGAACCCTCCACATCCCGCTCCGCGCACAAGAACACCGTCACT
TGCACCCAGCAGGGCCTGCTCGAGAACCAGCCTAGTTTCCACCCC
GCCAGGCGTGAATAGGGTGATCACTAGAGAGGAGCTCGAGGCGCT
TACCCCGTCACGCACTCCTAGCAGGTCGGTCTCGAGAACCAGCCT
GGTCTCCAACCCGCCAGGCGTAAATAGGGTGATTACAAGAGAGGA
GTTTGAGGCGTTCGTAGCACAACAACAATGACGGTTTGATGCGGG
TGCATACATCTTTTCCTCCGACACCGGTCAAGGGCATTTACAACA
AAAATCAGTAAGGCAAACGGTGCTATCCGAAGTGGTGTTGGAGAG
GACCGAATTGGAGATTTCGTATGCCCCGCGCCTCGACCAAGAAAA
AGAAGAATTACTACGCAAGAAATTACAGTTAAATCCCACACCTGC
TAACAGAAGCAGATACCAGTCCAGGAAGGTGGAGAACATGAAAGC
CATAACAGCTAGACGTATTCTGCAAGGCCTAGGGCATTATTTGAA
GGCAGAAGGAAAAGTGGAGTGCTACCGAACCCTGCATCCTGTTCC
TTTGTATTCATCTAGTGTGAACCGTGCCTTTTCAAGCCCCAAGGT
CGCAGTGGAAGCCTGTAACGCCATGTTGAAAGAGAACTTTCCGAC
TGTGGCTTCTTACTGTATTATTCCAGAGTACGATGCCTATTTGGA
CATGGTTGACGGAGCTTCATGCTGCTTAGACACTGCCAGTTTTTG
CCCTGCAAAGCTGCGCAGCTTTCCAAAGAAACACTCCTATTTGGA
ACCCACAATACGATCGGCAGTGCCTTCAGCGATCCAGAACACGCT
CCAGAACGTCCTGGCAGCTGCCACAAAAAGAAATTGCAATGTCAC
GCAAATGAGAGAATTGCCCGTATTGGATTCGGCGGCCTTTAATGT
GGAATGCTTCAAGAAATATGCGTGTAATAATGAATATTGGGAAAC
GTTTAAAGAAAACCCCATCAGGCTTACTGAAGAAAACGTGGTAAA
TTACATTACCAAATTAAAAGGACCAAAAGCTGCTGCTCTTTTTGC
GAAGACACATAATTTGAATATGTTGCAGGACATACCAATGGACAG
GTTTGTAATGGACTTAAAGAGAGACGTGAAAGTGACTCCAGGAAC
AAAACATACTGAAGAACGGCCCAAGGTACAGGTGATCCAGGCTGC
CGATCCGCTAGCAACAGCGTATCTGTGCGGAATCCACCGAGAGCT
GGTTAGGAGATTAAATGCGGTCCTGCTTCCGAACATTCATACACT
GTTTGATATGTCGGCTGAAGACTTTGACGCTATTATAGCCGAGCA
CTTCCAGCCTGGGGATTGTGTTCTGGAAACTGACATCGCGTCGTT
TGATAAAAGTGAGGACGACGCCATGGCTCTGACCGCGTTAATGAT
TCTGGAAGACTTAGGTGTGGACGCAGAGCTGTTGACGCTGATTGA
GGCGGCTTTCGGCGAAATTTCATCAATACATTTGCCCACTAAAAC
TAAATTTAAATTCGGAGCCATGATGAAATCTGGAATGTTCCTCAC
ACTGTTTGTGAACACAGTCATTAACATTGTAATCGCAAGCAGAGT
GTTGAGAGAACGGCTAACCGGATCACCATGTGCAGCATTCATTGG
AGATGACAATATCGTGAAAGGAGTCAAATCGGACAAATTAATGGC
AGACAGGTGCGCCACCTGGTTGAATATGGAAGTCAAGATTATAGA
TGCTGTGGTGGGCGAGAAAGCGCCTTATTTCTGTGGAGGGTTTAT
TTTGTGTGACTCCGTGACCGGCACAGCGTGCCGTGTGGCAGACCC
CCTAAAAAGGCTGTTTAAGCTTGGCAAACCTCTGGCAGCAGACGA
TGAACATGATGATGACAGGAGAAGGGCATTGCATGAAGAGTCAAC
ACGCTGGAACCGAGTGGGTATTCTTTCAGAGCTGTGCAAGGCAGT
AGAATCAAGGTATGAAACCGTAGGAACTTCCATCATAGTTATGGC
CATGACTACTCTAGCTAGCAGTGTTAAATCATTCAGCTACCTGAG
AGGGGCCCCTATAACTCTCTACGGCTAACCTGAATGGACTACGAC
ATAGTCTAGTCCGCCAAGTCTGTTTAAACAGCATATGGGCGCGCC
CTCAGCATCGATTCAATTCGCCACCATGGTGAGCAAGGGCGAGGA
GCTGTTCACCGGGGTGGTGCCCATCCTGGTCGAGCTGGACGGCGA
CGTAAACGGCCACAAGTTCAGCGTGTCCGGCGAGGGCGAGGGCGA
TGCCACCTACGGCAAGCTGACCCTGAAGTTCATCTGCACCACCGG
CAAGCTGCCCGTGCCCTGGCCCACCCTCGTGACCACCCTGACCTA
CGGCGTGCAGTGCTTCAGCCGCTACCCCGACCACATGAAGCAGCA
CGACTTCTTCAAGTCCGCCATGCCCGAAGGCTACGTCCAGGAGCG
CACCATCTTCTTCAAGGACGACGGCAACTACAAGACCCGCGCCGA
GGTGAAGTTCGAGGGCGACACCCTGGTGAACCGCATCGAGCTGAA
GGGCATCGACTTCAAGGAGGACGGCAACATCCTGGGGCACAAGCT
GGAGTACAACTACAACAGCCACAACGTCTATATCATGGCCGACAA
GCAGAAGAACGGCATCAAGGTGAACTTCAAGATCCGCCACAACAT
CGAGGACGGCAGCGTGCAGCTCGCCGACCACTACCAGCAGAACAC
CCCCATCGGCGACGGCCCCGTGCTGCTGCCCGACAACCACTACCT
GAGCACCCAGTCCGCCCTGAGCAAAGACCCCAACGAGAAGCGCGA
TCACATGGTCCTGCTGGAGTTCGTGACCGCCGCCGGGATCACTCT
CGGCATGGACGAGCTGTACAAGTAGTCTAGAGTGTTTAAACCGAC
CCGGGCGGCCGCAACTAACTTAAGCTAGCAACGGTTTCCCTCTAG
CGGGATCAATTCCGCCCCCCCCCCCTAACGTTACTGGCCGAAGCC
GCTTGGAATAAGGCCGGTGTGCGTTTGTCTATATGTTATTTTCCA
CCATATTGCCGTCTTTTGGCAATGTGAGGGCCCGGAAACCTGGCC
CTGTCTTCTTGACGAGCATTCCTAGGGGTCTTTCCCCTCTCGCCA
AAGGAATGCAAGGTCTGTTGAATGTCGTGAAGGAAGCAGTTCCTC
TGGAAGCTTCTTGAAGACAAACAACGTCTGTAGCGACCCTTTGCA
GGCAGCGGAACCCCCCACCTGGCGACAGGTGCCTCTGCGGCCAAA
AGCCACGTGTATAAGATACACCTGCAAAGGCGGCACAACCCCAGT
GCCACGTTGTGAGTTGGATAGTTGTGGAAAGAGTCAAATGGCTCT
CCTCAAGCGTATTCAACAAGGGGCTGAAGGATGCCCAGAAGGTAC
CCCATTGTATGGGATCTGATCTGGGGCCTCGGTGCACATGCTTTA
CATGTGTTTAGTCGAGGTTAAAAAAACGTCTAGGCCCCCCGAACC
ACGGGGACGTGGTTTTCCTTTGAAAAACACGATAATACCATGACC
GAGTACAAGCCCACGGTGCGCCTCGCCACCCGCGACGACGTCCCC
AGGGCCGTACGCACCCTCGCCGCCGCGTTCGCCGACTACCCCGCC
ACGCGCCACACCGTCGATCCGGACCGCCACATCGAGCGGGTCACC
GAGCTGCAAGAACTCTTCCTCACGCGCGTCGGGCTCGACATCGGC
AAGGTGTGGGTCGCGGACGACGGCGCCGCGGTGGCGGTCTGGACC
ACGCCGGAGAGCGTCGAAGCGGGGGCGGTGTTCGCCGAGATCGGC
CCGCGCATGGCCGAGTTGAGCGGTTCCCGGCTGGCCGCGCAGCAA
CAGATGGAAGGCCTCCTGGCGCCGCACCGGCCCAAGGAGCCCGCG
TGGTTCCTGGCCACCGTCGGCGTCTCGCCCGACCACCAGGGCAAG
GGTCTGGGCAGCGCCGTCGTGCTCCCCGGAGTGGAGGCGGCCGAG
CGCGCCGGGGTGCCCGCCTTCCTGGAGACCTCCGCGCCCCGCAAC
CTCCCCTTCTACGAGCGGCTCGGCTTCACCGTCACCGCCGACGTC
GAGGTGCCCGAAGGACCGCGCACCTGGTGCATGACCCGCAAGCCC
GGTGCCTGAGAATTGGCAAGCTGCTTACATAGAACTCGCGGCGAT
TGGCATGCCGCCTTAAAATTTTTATTTTATTTTTTCTTTTCTTTT
CCGAATCGGATTTTGTTTTTAATATTTCAAAAAAAAAAAAAAAAA
AAAAAAAAACGCGTCGAGGGGAATTAATTCTTGAAGACGAAAGGG
CCAGGTGGCACTTTTCGGGGAAATGTGCGCGGAACCCCTATTTGT
TTATTTTTCTAAATACATTCAAATATGTATCCGCTCATGAGACAA
TAACCCTGATAAATGCTTCAATAATATTGAAAAAGGAAGAGTATG
AGTATTCAACATTTCCGTGTCGCCCTTATTCCCTTTTTTGCGGCA
TTTTGCCTTCCTGTTTTTGCTCACCCAGAAACGCTGGTGAAAGTA
AAAGATGCTGAAGATCAGTTGGGTGCACGAGTGGGTTACATCGAA
CTGGATCTCAACAGCGGTAAGATCCTTGAGAGTTTTCGCCCCGAA
GAACGTTTTCCAATGATGAGCACTTTTAAAGTTCTGCTATGTGGC
GCGGTATTATCCCGTGTTGACGCCGGGCAAGAGCAACTCGGTCGC
CGCATACACTATTCTCAGAATGACTTGGTTGAGTACTCACCAGTC
ACAGAAAAGCATCTTACGGATGGCATGACAGTAAGAGAATTATGC
AGTGCTGCCATAACCATGAGTGATAACACTGCGGCCAACTTACTT
CTGACAACGATCGGAGGACCGAAGGAGCTAACCGCTTTTTTGCAC
AACATGGGGGATCATGTAACTCGCCTTGATCGTTGGGAACCGGAG
CTGAATGAAGCCATACCAAACGACGAGCGTGACACCACGATGCCT
GTAGCAATGGCAACAACGTTGCGCAAACTATTAACTGGCGAACTA
CTTACTCTAGCTTCCCGGCAACAATTAATAGACTGGATGGAGGCG
GATAAAGTTGCAGGACCACTTCTGCGCTCGGCCCTTCCGGCTGGC
TGGTTTATTGCTGATAAATCTGGAGCCGGTGAGCGTGGGTCTCGC
GGTATCATTGCAGCACTGGGGCCAGATGGTAAGCCCTCCCGTATC
GTAGTTATCTACACGACGGGGAGTCAGGCAACTATGGATGAACGA
AATAGACAGATCGCTGAGATAGGTGCCTCACTGATTAAGCATTGG
TAACTGTCAGACCAAGTTTACTCATATATACTTTAGATTGATTTA
AAACTTCATTTTTAATTTAAAAGGATCTAGGTGAAGATCCTTTTT
GATAATCTCATGACCAAAATCCCTTAACGTGAGTTTTCGTTCCAC
TGAGCGTCAGACCCCGTAGAAAAGATCAAAGGATCTTCTTGAGAT
CCTTTTTTTCTGCGCGTAATCTGCTGCTTGCAAACAAAAAAACCA
CCGCTACCAGCGGTGGTTTGTTTGCCGGATCAAGAGCTACCAACT
CTTTTTCCGAAGGTAACTGGCTTCAGCAGAGCGCAGATACCAAAT
ACTGTCCTTCTAGTGTAGCCGTAGTTAGGCCACCACTTCAAGAAC
TCTGTAGCACCGCCTACATACCTCGCTCTGCTAATCCTGTTACCA
GTGGCTGCTGCCAGTGGCGATAAGTCGTGTCTTACCGGGTTGGAC
TCAAGACGATAGTTACCGGATAAGGCGCAGCGGTCGGGCTGAACG
GGGGGTTCGTGCACACAGCCCAGCTTGGAGCGAACGACCTACACC
GAACTGAGATACCTACAGCGTGAGCTATGAGAAAGCGCCACGCTT
CCCGAAGGGAGAAAGGCGGACAGGTATCCGGTAAGCGGCAGGGTC
GGAACAGGAGAGCGCACGAGGGAGCTTCCAGGGGGAAACGCCTGG
TATCTTTATAGTCCTGTCGGGTTTCGCCACCTCTGACTTGAGCGT
CGATTTTTGTGATGCTCGTCAGGGGGGCGGAGCCTATGGAAAAAC
GCCAGCAACGCGAGCTCGCGATCGCTTAATTAAgacattgattat
tgactagttattaatagtaatcaattacggggtcattagttcata
gcccatatatggagttccgcgttacataacttacggtaaatggcc
cgcctggctgaccgcccaacgacccccgcccattgacgtcaataa
tgacgtatgttcccatagtaacgccaatagggactttccattgac
gtcaatgggtggagtatttacggtaaactgcccacttggcagtac
atcaagtgtatcatatgccaagtacgccccctattgacgtcaatg
acggtaaatggcccgcctggcattatgcccagtacatgaccttat
gggactttcctacttggcagtacatctacgtattagtcatcgcta
ttaccatggtcgaggtgagccccacgttctgcttcactctcccca
tctcccccccctccccacccccaattttgtatttatttatttttt
aattattttgtgcagcgatgggggcggggggggggggggcgcgcg
ccagggggggggggggggggggggggggggggggggggggggggg
gggggggcggcggcagccaatcagagcggcgcgctccgaaagttt
ccttttatggcgaggcggcggcggcggcggccctataaaaagcga
agcgcgcggcgggcgggagtcgctgcgcgctgccttcgccccgtg
ccccgctccgccgccgcctcgcgccgcccgccccggctctgactg
accgcgttactcccacaggtgagcggggggacggcccttctcctc
cgggctgtaattagcgcttggtttaatgacggcttgtttcttttc
tgtggctgcgtgaaagccttgaggggctccgggagggccctttgt
gcggggggagcggctcggggggtgcgtgcgtgtgtgtgtgcgtgg
ggagcgccgcgtgcggctccgcgctgcccggcggctgtgagcgct
gcgggcgcggcgcggggctttgtgcgctccgcagtgtgcgcgagg
ggagcgcggccgggggcggtgccccgcggtgcggggggggctgcg
aggggaacaaaggctgcgtgcggggtgtgtgcgtgggggggtgag
cagggggtgtgggcgcgtcggtcgggctgcaaccccccctgcacc
cccctccccgagttgctgagcacggcccggcttcgggtgcggggc
tccgtacggggcgtggcgcggggctcgccgtgccgggcggggggg
gcggcaggtgggggtgccgggcggggggggccgcctcgggccggg
gggggctcggggggggggcgcggcggcccccggagcgccggcggc
tgtcgaggcgcggcgagccgcagccattgccttttatggtaatcg
tgcgagagggcgcagggacttcctttgtcccaaatctgtgcggag
ccgaaatctgggaggcgccgccgcaccccctctagcgggcgcggg
gcgaagcggtgcggcgccggcaggaaggaaatgggcggggagggc
cttcgtgcgtcgccgcgccgccgtccccttctccctctccagcct
cggggctgtccgcggggggacggctgccttcgggggggacggggc
agggcggggttcggcttctggcgtgtgaccggcggctctagagcc
tctgctaaccatgttcatgccttcttctttttcctacagGGTTTA
GTGAACCGTCAGATCCGCTAGTAATACGACTCACTATAGGGCCGG
CCATAGGCGGCGCATGAGAGAAGCCCAGACCAATTACCTACCCAA
AATGGAGAAAGTTCACGTTGACATCGAGGAAGACAGCCCATTCCT
CAGAGCTTTGCAGCGGAGCTTCCCGCAGTTTGAGGTAGAAGCCAA
GCAGGTCACTGATAATGACCATGCTAATGCCAGAGCGTTTTCGCA
TCTGGCTTCAAAACTGATCGAAACGGAGGTGGACCCATCCGACAC
GATCCTTGACATTGGAAGTGCGCCCGCCCGCAGAATGTATTCTAA
GCACAAGTATCATTGTATCTGTCCGATGAGATGTGCGGAAGATCC
GGACAGATTGTATAAGTATGCAACTAAGCTGAAGAAAAACTGTAA
GGAAATAACTGATAAGGAATTGGACAAGAAAATGAAGGAGCTGGC
CGCCGTCATGAGCGACCCTGACCTGGAAACTGAGACTATGTGCCT
CCACGACGACGAGTCGTGTCGCTACGAAGGGCAAGTCGCTGTTTA
CCAGGATGTATACGCGGTTGACGGACCGACAAGTCTCTATCACCA
AGCCAATAAGGGAGTTAGAGTCGCCTACTGGATAGGCTTTGACAC
CACCCCTTTTATGTTTAAGAACTTGGCTGGAGCATATCCATCATA
CTCTACCAACTGGGCCGACGAAACCGTGTT
DNA Sequence of Vector CMV+T7_VEE_SA_GFP
(Map set forth in FIG. 9)
AACGGCTCGTAACATAGGCCTATGCAGCTCTGACGTTATGGAGCG
GTCACGTAGAGGGATGTCCATTCTTAGAAAGAAGTATTTGAAACC
ATCCAACAATGTTCTATTCTCTGTTGGCTCGACCATCTACCACGA
GAAGAGGGACTTACTGAGGAGCTGGCACCTGCCGTCTGTATTTCA
CTTACGTGGCAAGCAAAATTACACATGTCGGTGTGAGACTATAGT
TAGTTGCGACGGGTACGTCGTTAAAAGAATAGCTATCAGTCCAGG
CCTGTATGGGAAGCCTTCAGGCTATGCTGCTACGATGCACCGCGA
GGGATTCTTGTGCTGCAAAGTGACAGACACATTGAACGGGGAGAG
GGTCTCTTTTCCCGTGTGCACGTATGTGCCAGCTACATTGTGTGA
CCAAATGACTGGCATACTGGCAACAGATGTCAGTGCGGACGACGC
GCAAAAACTGCTGGTTGGGCTCAACCAGCGTATAGTCGTCAACGG
TCGCACCCAGAGAAACACCAATACCATGAAAAATTACCTTTTGCC
CGTAGTGGCCCAGGCATTTGCTAGGTGGGCAAAGGAATATAAGGA
AGATCAAGAAGATGAAAGGCCACTAGGACTACGAGATAGACAGTT
AGTCATGGGGTGTTGTTGGGCTTTTAGAAGGCACAAGATAACATC
TATTTATAAGCGCCCGGATACCCAAACCATCATCAAAGTGAACAG
CGATTTCCACTCATTCGTGCTGCCCAGGATAGGCAGTAACACATT
GGAGATCGGGCTGAGAACAAGAATCAGGAAAATGTTAGAGGAGCA
CAAGGAGCCGTCACCTCTCATTACCGCCGAGGACGTACAAGAAGC
TAAGTGCGCAGCCGATGAGGCTAAGGAGGTGCGTGAAGCCGAGGA
GTTGCGCGCAGCTCTACCACCTTTGGCAGCTGATGTTGAGGAGCC
CACTCTGGAGGCAGACGTCGACTTGATGTTACAAGAGGCTGGGGC
CGGCTCAGTGGAGACACCTCGTGGCTTGATAAAGGTTACCAGCTA
CGATGGCGAGGACAAGATCGGCTCTTACGCTGTGCTTTCTCCGCA
GGCTGTACTCAAGAGTGAAAAATTATCTTGCATCCACCCTCTCGC
TGAACAAGTCATAGTGATAACACACTCTGGCCGAAAAGGGCGTTA
TGCCGTGGAACCATACCATGGTAAAGTAGTGGTGCCAGAGGGACA
TGCAATACCCGTCCAGGACTTTCAAGCTCTGAGTGAAAGTGCCAC
CATTGTGTACAACGAACGTGAGTTCGTAAACAGGTACCTGCACCA
TATTGCCACACATGGAGGAGCGCTGAACACTGATGAAGAATATTA
CAAAACTGTCAAGCCCAGCGAGCACGACGGCGAATACCTGTACGA
CATCGACAGGAAACAGTGCGTCAAGAAAGAACTAGTCACTGGGCT
AGGGCTCACAGGCGAGCTGGTGGATCCTCCCTTCCATGAATTCGC
CTACGAGAGTCTGAGAACACGACCAGCCGCTCCTTACCAAGTACC
AACCATAGGGGTGTATGGCGTGCCAGGATCAGGCAAGTCTGGCAT
CATTAAAAGCGCAGTCACCAAAAAAGATCTAGTGGTGAGCGCCAA
GAAAGAAAACTGTGCAGAAATTATAAGGGACGTCAAGAAAATGAA
AGGGCTGGACGTCAATGCCAGAACTGTGGACTCAGTGCTCTTGAA
TGGATGCAAACACCCCGTAGAGACCCTGTATATTGACGAAGCTTT
TGCTTGTCATGCAGGTACTCTCAGAGCGCTCATAGCCATTATAAG
ACCTAAAAAGGCAGTGCTCTGCGGGGATCCCAAACAGTGCGGTTT
TTTTAACATGATGTGCCTGAAAGTGCATTTTAACCACGAGATTTG
CACACAAGTCTTCCACAAAAGCATCTCTCGCCGTTGCACTAAATC
TGTGACTTCGGTCGTCTCAACCTTGTTTTACGACAAAAAAATGAG
AACGACGAATCCGAAAGAGACTAAGATTGTGATTGACACTACCGG
CAGTACCAAACCTAAGCAGGACGATCTCATTCTCACTTGTTTCAG
AGGGTGGGTGAAGCAGTTGCAAATAGATTACAAAGGCAACGAAAT
AATGACGGCAGCTGCCTCTCAAGGGCTGACCCGTAAAGGTGTGTA
TGCCGTTCGGTACAAGGTGAATGAAAATCCTCTGTACGCACCCAC
CTCAGAACATGTGAACGTCCTACTGACCCGCACGGAGGACCGCAT
CGTGTGGAAAACACTAGCCGGCGACCCATGGATAAAAACACTGAC
TGCCAAGTACCCTGGGAATTTCACTGCCACGATAGAGGAGTGGCA
AGCAGAGCATGATGCCATCATGAGGCACATCTTGGAGAGACCGGA
CCCTACCGACGTCTTCCAGAATAAGGCAAACGTGTGTTGGGCCAA
GGCTTTAGTGCCGGTGCTGAAGACCGCTGGCATAGACATGACCAC
TGAACAATGGAACACTGTGGATTATTTTGAAACGGACAAAGCTCA
CTCAGCAGAGATAGTATTGAACCAACTATGCGTGAGGTTCTTTGG
ACTCGATCTGGACTCCGGTCTATTTTCTGCACCCACTGTTCCGTT
ATCCATTAGGAATAATCACTGGGATAACTCCCCGTCGCCTAACAT
GTACGGGCTGAATAAAGAAGTGGTCCGTCAGCTCTCTCGCAGGTA
CCCACAACTGCCTCGGGCAGTTGCCACTGGAAGAGTCTATGACAT
GAACACTGGTACACTGCGCAATTATGATCCGCGCATAAACCTAGT
ACCTGTAAACAGAAGACTGCCTCATGCTTTAGTCCTCCACCATAA
TGAACACCCACAGAGTGACTTTTCTTCATTCGTCAGCAAATTGAA
GGGCAGAACTGTCCTGGTGGTCGGGGAAAAGTTGTCCGTCCCAGG
CAAAATGGTTGACTGGTTGTCAGACCGGCCTGAGGCTACCTTCAG
AGCTCGGCTGGATTTAGGCATCCCAGGTGATGTGCCCAAATATGA
CATAATATTTGTTAATGTGAGGACCCCATATAAATACCATCACTA
TCAGCAGTGTGAAGACCATGCCATTAAGCTTAGCATGTTGACCAA
GAAAGCTTGTCTGCATCTGAATCCCGGCGGAACCTGTGTCAGCAT
AGGTTATGGTTACGCTGACAGGGCCAGCGAAAGCATCATTGGTGC
TATAGCGCGGCAGTTCAAGTTTTCCCGGGTATGCAAACCGAAATC
CTCACTTGAAGAGACGGAAGTTCTGTTTGTATTCATTGGGTACGA
TCGCAAGGCCCGTACGCACAATTCTTACAAGCTTTCATCAACCTT
GACCAACATTTATACAGGTTCCAGACTCCACGAAGCCGGATGTGC
ACCCTCATATCATGTGGTGCGAGGGGATATTGCCACGGCCACCGA
AGGAGTGATTATAAATGCTGCTAACAGCAAAGGACAACCTGGCGG
AGGGGTGTGCGGAGCGCTGTATAAGAAATTCCCGGAAAGCTTCGA
TTTACAGCCGATCGAAGTAGGAAAAGCGCGACTGGTCAAAGGTGC
AGCTAAACATATCATTCATGCCGTAGGACCAAACTTCAACAAAGT
TTCGGAGGTTGAAGGTGACAAACAGTTGGCAGAGGCTTATGAGTC
CATCGCTAAGATTGTCAACGATAACAATTACAAGTCAGTAGCGAT
TCCACTGTTGTCCACCGGCATCTTTTCCGGGAACAAAGATCGACT
AACCCAATCATTGAACCATTTGCTGACAGCTTTAGACACCACTGA
TGCAGATGTAGCCATATACTGCAGGGACAAGAAATGGGAAATGAC
TCTCAAGGAAGCAGTGGCTAGGAGAGAAGCAGTGGAGGAGATATG
CATATCCGACGACTCTTCAGTGACAGAACCTGATGCAGAGCTGGT
GAGGGTGCATCCGAAGAGTTCTTTGGCTGGAAGGAAGGGCTACAG
CACAAGCGATGGCAAAACTTTCTCATATTTGGAAGGGACCAAGTT
TCACCAGGCGGCCAAGGATATAGCAGAAATTAATGCCATGTGGCC
CGTTGCAACGGAGGCCAATGAGCAGGTATGCATGTATATCCTCGG
AGAAAGCATGAGCAGTATTAGGTCGAAATGCCCCGTCGAAGAGTC
GGAAGCCTCCACACCACCTAGCACGCTGCCTTGCTTGTGCATCCA
TGCCATGACTCCAGAAAGAGTACAGCGCCTAAAAGCCTCACGTCC
AGAACAAATTACTGTGTGCTCATCCTTTCCATTGCCGAAGTATAG
AATCACTGGTGTGCAGAAGATCCAATGCTCCCAGCCTATATTGTT
CTCACCGAAAGTGCCTGCGTATATTCATCCAAGGAAGTATCTCGT
GGAAACACCACCGGTAGACGAGACTCCGGAGCCATCGGCAGAGAA
CCAATCCACAGAGGGGACACCTGAACAACCACCACTTATAACCGA
GGATGAGACCAGGACTAGAACGCCTGAGCCGATCATCATCGAAGA
GGAAGAAGAGGATAGCATAAGTTTGCTGTCAGATGGCCCGACCCA
CCAGGTGCTGCAAGTCGAGGCAGACATTCACGGGCCGCCCTCTGT
ATCTAGCTCATCCTGGTCCATTCCTCATGCATCCGACTTTGATGT
GGACAGTTTATCCATACTTGACACCCTGGAGGGAGCTAGCGTGAC
CAGCGGGGCAACGTCAGCCGAGACTAACTCTTACTTCGCAAAGAG
TATGGAGTTTCTGGCGCGACCGGTGCCTGCGCCTCGAACAGTATT
CAGGAACCCTCCACATCCCGCTCCGCGCACAAGAACACCGTCACT
TGCACCCAGCAGGGCCTGCTCGAGAACCAGCCTAGTTTCCACCCC
GCCAGGCGTGAATAGGGTGATCACTAGAGAGGAGCTCGAGGCGCT
TACCCCGTCACGCACTCCTAGCAGGTCGGTCTCGAGAACCAGCCT
GGTCTCCAACCCGCCAGGCGTAAATAGGGTGATTACAAGAGAGGA
GTTTGAGGCGTTCGTAGCACAACAACAATGACGGTTTGATGCGGG
TGCATACATCTTTTCCTCCGACACCGGTCAAGGGCATTTACAACA
AAAATCAGTAAGGCAAACGGTGCTATCCGAAGTGGTGTTGGAGAG
GACCGAATTGGAGATTTCGTATGCCCCGCGCCTCGACCAAGAAAA
AGAAGAATTACTACGCAAGAAATTACAGTTAAATCCCACACCTGC
TAACAGAAGCAGATACCAGTCCAGGAAGGTGGAGAACATGAAAGC
CATAACAGCTAGACGTATTCTGCAAGGCCTAGGGCATTATTTGAA
GGCAGAAGGAAAAGTGGAGTGCTACCGAACCCTGCATCCTGTTCC
TTTGTATTCATCTAGTGTGAACCGTGCCTTTTCAAGCCCCAAGGT
CGCAGTGGAAGCCTGTAACGCCATGTTGAAAGAGAACTTTCCGAC
TGTGGCTTCTTACTGTATTATTCCAGAGTACGATGCCTATTTGGA
CATGGTTGACGGAGCTTCATGCTGCTTAGACACTGCCAGTTTTTG
CCCTGCAAAGCTGCGCAGCTTTCCAAAGAAACACTCCTATTTGGA
ACCCACAATACGATCGGCAGTGCCTTCAGCGATCCAGAACACGCT
CCAGAACGTCCTGGCAGCTGCCACAAAAAGAAATTGCAATGTCAC
GCAAATGAGAGAATTGCCCGTATTGGATTCGGCGGCCTTTAATGT
GGAATGCTTCAAGAAATATGCGTGTAATAATGAATATTGGGAAAC
GTTTAAAGAAAACCCCATCAGGCTTACTGAAGAAAACGTGGTAAA
TTACATTACCAAATTAAAAGGACCAAAAGCTGCTGCTCTTTTTGC
GAAGACACATAATTTGAATATGTTGCAGGACATACCAATGGACAG
GTTTGTAATGGACTTAAAGAGAGACGTGAAAGTGACTCCAGGAAC
AAAACATACTGAAGAACGGCCCAAGGTACAGGTGATCCAGGCTGC
CGATCCGCTAGCAACAGCGTATCTGTGCGGAATCCACCGAGAGCT
GGTTAGGAGATTAAATGCGGTCCTGCTTCCGAACATTCATACACT
GTTTGATATGTCGGCTGAAGACTTTGACGCTATTATAGCCGAGCA
CTTCCAGCCTGGGGATTGTGTTCTGGAAACTGACATCGCGTCGTT
TGATAAAAGTGAGGACGACGCCATGGCTCTGACCGCGTTAATGAT
TCTGGAAGACTTAGGTGTGGACGCAGAGCTGTTGACGCTGATTGA
GGCGGCTTTCGGCGAAATTTCATCAATACATTTGCCCACTAAAAC
TAAATTTAAATTCGGAGCCATGATGAAATCTGGAATGTTCCTCAC
ACTGTTTGTGAACACAGTCATTAACATTGTAATCGCAAGCAGAGT
GTTGAGAGAACGGCTAACCGGATCACCATGTGCAGCATTCATTGG
AGATGACAATATCGTGAAAGGAGTCAAATCGGACAAATTAATGGC
AGACAGGTGCGCCACCTGGTTGAATATGGAAGTCAAGATTATAGA
TGCTGTGGTGGGCGAGAAAGCGCCTTATTTCTGTGGAGGGTTTAT
TTTGTGTGACTCCGTGACCGGCACAGCGTGCCGTGTGGCAGACCC
CCTAAAAAGGCTGTTTAAGCTTGGCAAACCTCTGGCAGCAGACGA
TGAACATGATGATGACAGGAGAAGGGCATTGCATGAAGAGTCAAC
ACGCTGGAACCGAGTGGGTATTCTTTCAGAGCTGTGCAAGGCAGT
AGAATCAAGGTATGAAACCGTAGGAACTTCCATCATAGTTATGGC
CATGACTACTCTAGCTAGCAGTGTTAAATCATTCAGCTACCTGAG
AGGGGCCCCTATAACTCTCTACGGCTAACCTGAATGGACTACGAC
ATAGTCTAGTCCGCCAAGTCTGTTTAAACAGCATATGGGCGCGCC
CTCAGCATCGATTCAATTCGCCACCTCTAGAGTGTTTAAACCGAC
CCGGGCGGCCGCAACTAACTTAAGCTAGCAACGGTTTCCCTCTAG
CGGGATCAATTCCGCCCCCCCCCCCTAACGTTACTGGCCGAAGCC
GCTTGGAATAAGGCCGGTGTGCGTTTGTCTATATGTTATTTTCCA
CCATATTGCCGTCTTTTGGCAATGTGAGGGCCCGGAAACCTGGCC
CTGTCTTCTTGACGAGCATTCCTAGGGGTCTTTCCCCTCTCGCCA
AAGGAATGCAAGGTCTGTTGAATGTCGTGAAGGAAGCAGTTCCTC
TGGAAGCTTCTTGAAGACAAACAACGTCTGTAGCGACCCTTTGCA
GGCAGCGGAACCCCCCACCTGGCGACAGGTGCCTCTGCGGCCAAA
AGCCACGTGTATAAGATACACCTGCAAAGGCGGCACAACCCCAGT
GCCACGTTGTGAGTTGGATAGTTGTGGAAAGAGTCAAATGGCTCT
CCTCAAGCGTATTCAACAAGGGGCTGAAGGATGCCCAGAAGGTAC
CCCATTGTATGGGATCTGATCTGGGGCCTCGGTGCACATGCTTTA
CATGTGTTTAGTCGAGGTTAAAAAAACGTCTAGGCCCCCCGAACC
ACGGGGACGTGGTTTTCCTTTGAAAAACACGATAATACCATGACC
GAGTACAAGCCCACGGTGCGCCTCGCCACCCGCGACGACGTCCCC
AGGGCCGTACGCACCCTCGCCGCCGCGTTCGCCGACTACCCCGCC
ACGCGCCACACCGTCGATCCGGACCGCCACATCGAGCGGGTCACC
GAGCTGCAAGAACTCTTCCTCACGCGCGTCGGGCTCGACATCGGC
AAGGTGTGGGTCGCGGACGACGGCGCCGCGGTGGCGGTCTGGACC
ACGCCGGAGAGCGTCGAAGCGGGGGCGGTGTTCGCCGAGATCGGC
CCGCGCATGGCCGAGTTGAGCGGTTCCCGGCTGGCCGCGCAGCAA
CAGATGGAAGGCCTCCTGGCGCCGCACCGGCCCAAGGAGCCCGCG
TGGTTCCTGGCCACCGTCGGCGTCTCGCCCGACCACCAGGGCAAG
GGTCTGGGCAGCGCCGTCGTGCTCCCCGGAGTGGAGGCGGCCGAG
CGCGCCGGGGTGCCCGCCTTCCTGGAGACCTCCGCGCCCCGCAAC
CTCCCCTTCTACGAGCGGCTCGGCTTCACCGTCACCGCCGACGTC
GAGGTGCCCGAAGGACCGCGCACCTGGTGCATGACCCGCAAGCCC
GGTGCCTGAGAATTGGCAAGCTGCTTACATAGAACTCGCGGCGAT
TGGCATGCCGCCTTAAAATTTTTATTTTATTTTTTCTTTTCTTTT
CCGAATCGGATTTTGTTTTTAATATTTCAAAAAAAAAAAAAAAAA
AAAAAAAAACGCGTCGAGGGGAATTAATTCTTGAAGACGAAAGGG
CCAGGTGGCACTTTTCGGGGAAATGTGCGCGGAACCCCTATTTGT
TTATTTTTCTAAATACATTCAAATATGTATCCGCTCATGAGACAA
TAACCCTGATAAATGCTTCAATAATATTGAAAAAGGAAGAGTATG
AGTATTCAACATTTCCGTGTCGCCCTTATTCCCTTTTTTGCGGCA
TTTTGCCTTCCTGTTTTTGCTCACCCAGAAACGCTGGTGAAAGTA
AAAGATGCTGAAGATCAGTTGGGTGCACGAGTGGGTTACATCGAA
CTGGATCTCAACAGCGGTAAGATCCTTGAGAGTTTTCGCCCCGAA
GAACGTTTTCCAATGATGAGCACTTTTAAAGTTCTGCTATGTGGC
GCGGTATTATCCCGTGTTGACGCCGGGCAAGAGCAACTCGGTCGC
CGCATACACTATTCTCAGAATGACTTGGTTGAGTACTCACCAGTC
ACAGAAAAGCATCTTACGGATGGCATGACAGTAAGAGAATTATGC
AGTGCTGCCATAACCATGAGTGATAACACTGCGGCCAACTTACTT
CTGACAACGATCGGAGGACCGAAGGAGCTAACCGCTTTTTTGCAC
AACATGGGGGATCATGTAACTCGCCTTGATCGTTGGGAACCGGAG
CTGAATGAAGCCATACCAAACGACGAGCGTGACACCACGATGCCT
GTAGCAATGGCAACAACGTTGCGCAAACTATTAACTGGCGAACTA
CTTACTCTAGCTTCCCGGCAACAATTAATAGACTGGATGGAGGCG
GATAAAGTTGCAGGACCACTTCTGCGCTCGGCCCTTCCGGCTGGC
TGGTTTATTGCTGATAAATCTGGAGCCGGTGAGCGTGGGTCTCGC
GGTATCATTGCAGCACTGGGGCCAGATGGTAAGCCCTCCCGTATC
GTAGTTATCTACACGACGGGGAGTCAGGCAACTATGGATGAACGA
AATAGACAGATCGCTGAGATAGGTGCCTCACTGATTAAGCATTGG
TAACTGTCAGACCAAGTTTACTCATATATACTTTAGATTGATTTA
AAACTTCATTTTTAATTTAAAAGGATCTAGGTGAAGATCCTTTTT
GATAATCTCATGACCAAAATCCCTTAACGTGAGTTTTCGTTCCAC
TGAGCGTCAGACCCCGTAGAAAAGATCAAAGGATCTTCTTGAGAT
CCTTTTTTTCTGCGCGTAATCTGCTGCTTGCAAACAAAAAAACCA
CCGCTACCAGCGGTGGTTTGTTTGCCGGATCAAGAGCTACCAACT
CTTTTTCCGAAGGTAACTGGCTTCAGCAGAGCGCAGATACCAAAT
ACTGTCCTTCTAGTGTAGCCGTAGTTAGGCCACCACTTCAAGAAC
TCTGTAGCACCGCCTACATACCTCGCTCTGCTAATCCTGTTACCA
GTGGCTGCTGCCAGTGGCGATAAGTCGTGTCTTACCGGGTTGGAC
TCAAGACGATAGTTACCGGATAAGGCGCAGCGGTCGGGCTGAACG
GGGGGTTCGTGCACACAGCCCAGCTTGGAGCGAACGACCTACACC
GAACTGAGATACCTACAGCGTGAGCTATGAGAAAGCGCCACGCTT
CCCGAAGGGAGAAAGGCGGACAGGTATCCGGTAAGCGGCAGGGTC
GGAACAGGAGAGCGCACGAGGGAGCTTCCAGGGGGAAACGCCTGG
TATCTTTATAGTCCTGTCGGGTTTCGCCACCTCTGACTTGAGCGT
CGATTTTTGTGATGCTCGTCAGGGGGGCGGAGCCTATGGAAAAAC
GCCAGCAACGCGAGCTCGCGATCGCTTAATTAACGTTACATAACT
TACGGTAAATGGCCCGCCTGGCTGACCGCCCAACGACCCCCGCCC
ATTGACGTCAATAATGACGTATGTTCCCATAGTAACGCCAATAGG
GACTTTCCATTGACGTCAATGGGTGGAGTATTTACGGTAAACTGC
CCACTTGGCAGTACATCAAGTGTATCATATGCCAAGTACGCCCCC
TATTGACGTCAATGACGGTAAATGGCCCGCCTGGCATTATGCCCA
GTACATGACCTTATGGGACTTTCCTACTTGGCAGTACATCTACGT
ATTAGTCATCGCTATTACCATGGTGATGCGGTTTTGGCAGTACAT
CAATGGGCGTGGATAGCGGTTTGACTCACGGGGATTTCCAAGTCT
CCACCCCATTGACGTCAATGGGAGTTTGTTTTGGCACCAAAATCA
ACGGGACTTTCCAAAATGTCGTAACAACTCCGCCCCATTGACGCA
AATGGGCGGTAGGCGTGTACGGTGGGAGGTCTATATAAGCAGAGC
TGGTTTAGTGAACCGTCAGATCCGCTAGTAATACGACTCACTATA
GGGCCGGCCATAGGCGGCGCATGAGAGAAGCCCAGACCAATTACC
TACCCAAAATGGAGAAAGTTCACGTTGACATCGAGGAAGACAGCC
CATTCCTCAGAGCTTTGCAGCGGAGCTTCCCGCAGTTTGAGGTAG
AAGCCAAGCAGGTCACTGATAATGACCATGCTAATGCCAGAGCGT
TTTCGCATCTGGCTTCAAAACTGATCGAAACGGAGGTGGACCCAT
CCGACACGATCCTTGACATTGGAAGTGCGCCCGCCCGCAGAATGT
ATTCTAAGCACAAGTATCATTGTATCTGTCCGATGAGATGTGCGG
AAGATCCGGACAGATTGTATAAGTATGCAACTAAGCTGAAGAAAA
ACTGTAAGGAAATAACTGATAAGGAATTGGACAAGAAAATGAAGG
AGCTGGCCGCCGTCATGAGCGACCCTGACCTGGAAACTGAGACTA
TGTGCCTCCACGACGACGAGTCGTGTCGCTACGAAGGGCAAGTCG
CTGTTTACCAGGATGTATACGCGGTTGACGGACCGACAAGTCTCT
ATCACCAAGCCAATAAGGGAGTTAGAGTCGCCTACTGGATAGGCT
TTGACACCACCCCTTTTATGTTTAAGAACTTGGCTGGAGCATATC
CATCATACTCTACCAACTGGGCCGACGAAACCGTGTT

### Example 3: Self Amplifying Constructs with Chicken Beta Actin and T7 Promoter

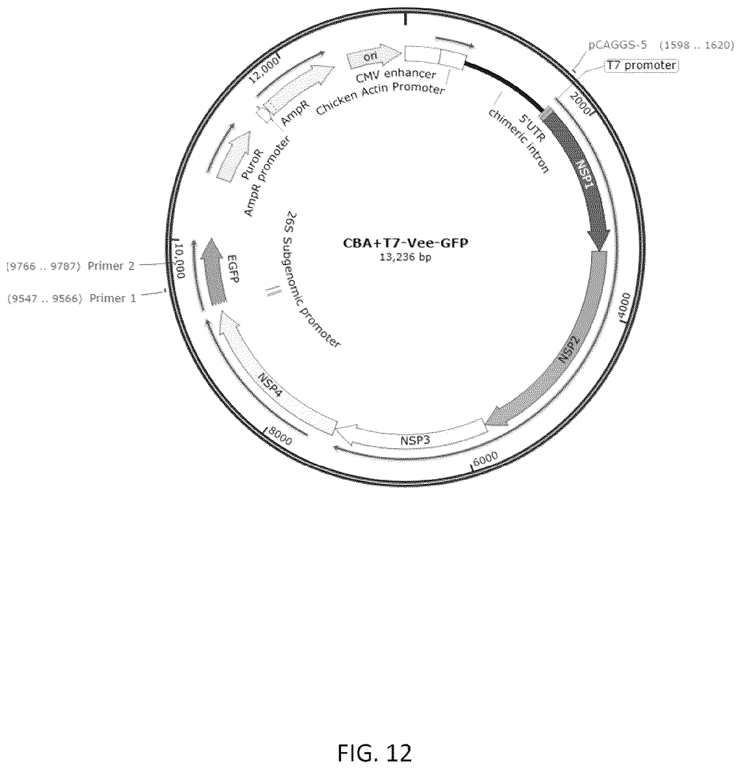

CBAVEE GFP: Variant construct with Chicken
BetaActin Promoter constructed in
CMVVEE GFP instead of CMV promoter (FIG. 12)

| | |
|---|---|
| CMV enhancer | 1 . . . 380 |
| Chicken Actin | 381 . . . 657 |
| Promoter | |
| chimeric intron | 658 . . . 1674 |
| T7 promoter | 1675 . . . 1693 |
| 5′ UTR | 1701 . . . 1744 |
| NSP1 | 1745 . . . 3349 |

MEKVHVDIEEDSPFLRALQRSFPQFEVEAKQVTDNDHANARAFSH
LASKLIETEVDPSDTILDIGSAPARRMYSKHKYHCICPMRCAEDP
DRLYKYATKLKKNCKEITDKELDKKMKELAAVMSDPDLETETMCL
HDDESCRYEGQVAVYQDVYAVDGPTSLYHQANKGVRVAYWIGFDT
TPFMFKNLAGAYPSYSTNWADETVLTARNIGLCSSDVMERSRRGM
SILRKKYLKPSNNVLFSVGSTIYHEKRDLLRSWHLPSVFHLRGKQ
NYTCRCETIVSCDGYVVKRIAISPGLYGKPSGYAATMHREGFLCC
KVTDTLNGERVSFPVCTYVPATLCDQMTGILATDVSADDAQKLLV
GLNQRIVVNGRTQRNTNTMKNYLLPVVAQAFARWAKEYKEDQEDE
RPLGLRDRQLVMGCCWAFRRHKITSIYKRPDTQTIIKVNSDFHSF
VLPRIGSNTLEIGLRTRIRKMLEEHKEPSPLITAEDVQEAKCAAD
EAKEVREAEELRAALPPLAADVEEPTLEADVDLMLQEAGA

| | |
|---|---|
| NSP2 | 3350 . . . 5731 |

GSVETPRGLIKVTSYDGEDKIGSYAVLSPQAVLKSEKLSCIHPLA
EQVIVITHSGRKGRYAVEPYHGKVVVPEGHAIPVQDFQALSESAT
IVYNEREFVNRYLHHIATHGGALNTDEEYYKTVKPSEHDGEYLYD
IDRKQCVKKELVTGLGLTGELVDPPFHEFAYESLRTRPAAPYQVP
TIGVYGVPGSGKSGIIKSAVTKKDLVVSAKKENCAEIIRDVKKMK
GLDVNARTVDSVLLNGCKHPVETLYIDEAFACHAGTLRALIAIIR
PKKAVLCGDPKQCGFFNMMCLKVHFNHEICTQVFHKSISRRCTKS
VTSVVSTLFYDKKMRTTNPKETKIVIDTTGSTKPKQDDLILTCFR
GWVKQLQIDYKGNEIMTAAASQGLTRKGVYAVRYKVNENPLYAPT
SEHVNVLLTRTEDRIVWKTLAGDPWIKTLTAKYPGNFTATIEEWQ
AEHDAIMRHILERPDPTDVFQNKANVCWAKALVPVLKTAGIDMTT
EQWNTVDYFETDKAHSAEIVLNQLCVRFFGLDLDSGLFSAPTVPL
SIRNNHWDNSPSPNMYGLNKEVVRQLSRRYPQLPRAVATGRVYDM
NTGTLRNYDPRINLVPVNRRLPHALVLHHNEHPQSDFSSFVSKLK
GRTVLVVGEKLSVPGKMVDWLSDRPEATFRARLDLGIPGDVPKYD
IIFVNVRTPYKYHHYQQCEDHAIKLSMLTKKACLHLNPGGTCVSI
GYGYADRASESIIGAIARQFKFSRVCKPKSSLEETEVLFVFIGYD
RKARTHNSYKLSSTLTNIYTGSRLHEAGC

| | |
|---|---|
| NSP3 | 5732 . . . 7402 |

APSYHVVRGDIATATEGVIINAANSKGQPGGGVCGALYKKEPESF
DLQPIEVGKARLVKGAAKHIIHAVGPNENKVSEVEGDKQLAEAYE
SIAKIVNDNNYKSVAIPLLSTGIFSGNKDRLTQSLNHLLTALDTT
DADVAIYCRDKKWEMTLKEAVARREAVEEICISDDSSVTEPDAEL
VRVHPKSSLAGRKGYSTSDGKTFSYLEGTKFHQAAKDIAEINAMW
PVATEANEQVCMYILGESMSSIRSKCPVEESEASTPPSTLPCLCI
HAMTPERVQRLKASRPEQITVCSSFPLPKYRITGVQKIQCSQPIL
FSPKVPAYIHPRKYLVETPPVDETPEPSAENQSTEGTPEQPPLIT
EDETRTRTPEPIIIEEEEEDSISLLSDGPTHQVLQVEADIHGPPS
VSSSSWSIPHASDFDVDSLSILDTLEGASVTSGATSAETNSYFAK
SMEFLARPVPAPRTVFRNPPHPAPRTRTPSLAPSRACSRTSLVST
PPGVNRVITREELEALTPSRTPSRSVSRTSLVSNPPGVNRVITRE
EFEAFVAQQQXRFDAGA

| | |
|---|---|
| NSP4 | 7403 . . . 9226 |

YIFSSDTGQGHLQQKSVRQTVLSEVVLERTELEISYAPRLDQEKE
ELLRKKLQLNPTPANRSRYQSRKVENMKAITARRILQGLGHYLKA
EGKVECYRTLHPVPLYSSSVNRAFSSPKVAVEACNAMLKENFPTV
ASYCIIPEYDAYLDMVDGASCCLDTASFCPAKLRSFPKKHSYLEP
TIRSAVPSAIQNTLQNVLAAATKRNCNVTQMRELPVLDSAAFNVE
CFKKYACNNEYWETFKENPIRLTEENVVNYITKLKGPKAAALFAK
THNLNMLQDIPMDRFVMDLKRDVKVTPGTKHTEERPKVQVIQAAD
PLATAYLCGIHRELVRRLNAVLLPNIHTLFDMSAEDFDAIIAEHF
QPGDCVLETDIASFDKSEDDAMALTALMILEDLGVDAELLTLIEA
AFGEISSIHLPTKTKFKFGAMMKSGMFLTLFVNTVINIVIASRVL
RERLTGSPCAAFIGDDNIVKGVKSDKLMADRCATWLNMEVKIIDA
VVGEKAPYFCGGFILCDSVTGTACRVADPLKRLFKLGKPLAADDE
HDDDRRRALHEESTRWNRVGILSELCKAVESRYETVGTSIIVMAM
TTLASSVKSFSYLRGAPITLYG*

| | |
|---|---|
| 26S Subgenomic | 9213 . . . 9236 |
| promoter | |
| Kozak sequence | 9308 . . . 9317 |
| EGFP | 9314 . . . 10,030 |
| PuroR | 10,678 . . . 11,277 |
| AmpRpromoter | 11,476 . . . 11,580 |
| AmpR | 11,581 . . . 12,441 |
| ori | 12,612 . . . 13,200 |

AATTACACATGTCGGTGTGAGACTATAGTTAGTTGCGACGGGTAC
GTCGTTAAAAGAATAGCTATCAGTCCAGGCCTGTATGGGAAGCCT
TCAGGCTATGCTGCTACGATGCACCGCGAGGGATTCTTGTGCTGC
AAAGTGACAGACACATTGAACGGGGAGAGGGTCTCTTTTCCCGTG
TGCACGTATGTGCCAGCTACATTGTGTGACCAAATGACTGGCATA
CTGGCAACAGATGTCAGTGCGGACGACGCGCAAAAACTGCTGGTT
GGGCTCAACCAGCGTATAGTCGTCAACGGTCGCACCCAGAGAAAC
ACCAATACCATGAAAAATTACCTTTTGCCCGTAGTGGCCCAGGCA
TTTGCTAGGTGGGCAAAGGAATATAAGGAAGATCAAGAAGATGAA
AGGCCACTAGGACTACGAGATAGACAGTTAGTCATGGGGTGTTGT
TGGGCTTTTAGAAGGCACAAGATAACATCTATTTATAAGCGCCCG
GATACCCAAACCATCATCAAAGTGAACAGCGATTTCCACTCATTC
GTGCTGCCCAGGATAGGCAGTAACACATTGGAGATCGGGCTGAGA
ACAAGAATCAGGAAAATGTTAGAGGAGCACAAGGAGCCGTCACCT
CTCATTACCGCCGAGGACGTACAAGAAGCTAAGTGCGCAGCCGAT
GAGGCTAAGGAGGTGCGTGAAGCCGAGGAGTTGCGCGCAGCTCTA
CCACCTTTGGCAGCTGATGTTGAGGAGCCCACTCTGGAGGCAGAC
GTCGACTTGATGTTACAAGAGGCTGGGGCCGGCTCAGTGGAGACA
CCTCGTGGCTTGATAAAGGTTACCAGCTACGATGGCGAGGACAAG
ATCGGCTCTTACGCTGTGCTTTCTCCGCAGGCTGTACTCAAGAGT
GAAAAATTATCTTGCATCCACCCTCTCGCTGAACAAGTCATAGTG
ATAACACACTCTGGCCGAAAAGGGCGTTATGCCGTGGAACCATAC
CATGGTAAAGTAGTGGTGCCAGAGGGACATGCAATACCCGTCCAG
GACTTTCAAGCTCTGAGTGAAAGTGCCACCATTGTGTACAACGAA
CGTGAGTTCGTAAACAGGTACCTGCACCATATTGCCACACATGGA
GGAGCGCTGAACACTGATGAAGAATATTACAAAACTGTCAAGCCC
AGCGAGCACGACGGCGAATACCTGTACGACATCGACAGGAAACAG
TGCGTCAAGAAAGAACTAGTCACTGGGCTAGGGCTCACAGGCGAG
CTGGTGGATCCTCCCTTCCATGAATTCGCCTACGAGAGTCTGAGA
ACACGACCAGCCGCTCCTTACCAAGTACCAACCATAGGGGTGTAT
GGCGTGCCAGGATCAGGCAAGTCTGGCATCATTAAAAGCGCAGTC
ACCAAAAAAGATCTAGTGGTGAGCGCCAAGAAAGAAAACTGTGCA
GAAATTATAAGGGACGTCAAGAAAATGAAAGGGCTGGACGTCAAT
GCCAGAACTGTGGACTCAGTGCTCTTGAATGGATGCAAACACCCC
GTAGAGACCCTGTATATTGACGAAGCTTTTGCTTGTCATGCAGGT
ACTCTCAGAGCGCTCATAGCCATTATAAGACCTAAAAAGGCAGTG
CTCTGCGGGGATCCCAAACAGTGCGGTTTTTTTAACATGATGTGC
CTGAAAGTGCATTTTAACCACGAGATTTGCACACAAGTCTTCCAC
AAAAGCATCTCTCGCCGTTGCACTAAATCTGTGACTTCGGTCGTC
TCAACCTTGTTTTACGACAAAAAAATGAGAACGACGAATCCGAAA
GAGACTAAGATTGTGATTGACACTACCGGCAGTACCAAACCTAAG
CAGGACGATCTCATTCTCACTTGTTTCAGAGGGTGGGTGAAGCAG
TTGCAAATAGATTACAAAGGCAACGAAATAATGACGGCAGCTGCC
TCTCAAGGGCTGACCCGTAAAGGTGTGTATGCCGTTCGGTACAAG
GTGAATGAAAATCCTCTGTACGCACCCACCTCAGAACATGTGAAC
GTCCTACTGACCCGCACGGAGGACCGCATCGTGTGGAAAACACTA
GCCGGCGACCCATGGATAAAAACACTGACTGCCAAGTACCCTGGG
AATTTCACTGCCACGATAGAGGAGTGGCAAGCAGAGCATGATGCC
ATCATGAGGCACATCTTGGAGAGACCGGACCCTACCGACGTCTTC
CAGAATAAGGCAAACGTGTGTTGGGCCAAGGCTTTAGTGCCGGTG
CTGAAGACCGCTGGCATAGACATGACCACTGAACAATGGAACACT
GTGGATTATTTTGAAACGGACAAAGCTCACTCAGCAGAGATAGTA
TTGAACCAACTATGCGTGAGGTTCTTTGGACTCGATCTGGACTCC
GGTCTATTTTCTGCACCCACTGTTCCGTTATCCATTAGGAATAAT
CACTGGGATAACTCCCCGTCGCCTAACATGTACGGGCTGAATAAA
GAAGTGGTCCGTCAGCTCTCTCGCAGGTACCCACAACTGCCTCGG
GCAGTTGCCACTGGAAGAGTCTATGACATGAACACTGGTACACTG
CGCAATTATGATCCGCGCATAAACCTAGTACCTGTAAACAGAAGA
CTGCCTCATGCTTTAGTCCTCCACCATAATGAACACCCACAGAGT
GACTTTTCTTCATTCGTCAGCAAATTGAAGGGCAGAACTGTCCTG
GTGGTCGGGGAAAAGTTGTCCGTCCCAGGCAAAATGGTTGACTGG
TTGTCAGACCGGCCTGAGGCTACCTTCAGAGCTCGGCTGGATTTA
GGCATCCCAGGTGATGTGCCCAAATATGACATAATATTTGTTAAT
GTGAGGACCCCATATAAATACCATCACTATCAGCAGTGTGAAGAC
CATGCCATTAAGCTTAGCATGTTGACCAAGAAAGCTTGTCTGCAT
CTGAATCCCGGCGGAACCTGTGTCAGCATAGGTTATGGTTACGCT
GACAGGGCCAGCGAAAGCATCATTGGTGCTATAGCGCGGCAGTTC
AAGTTTTCCCGGGTATGCAAACCGAAATCCTCACTTGAAGAGACG
GAAGTTCTGTTTGTATTCATTGGGTACGATCGCAAGGCCCGTACG
CACAATTCTTACAAGCTTTCATCAACCTTGACCAACATTTATACA
GGTTCCAGACTCCACGAAGCCGGATGTGCACCCTCATATCATGTG
GTGCGAGGGGATATTGCCACGGCCACCGAAGGAGTGATTATAAAT
GCTGCTAACAGCAAAGGACAACCTGGCGGAGGGGTGTGCGGAGCG
CTGTATAAGAAATTCCCGGAAAGCTTCGATTTACAGCCGATCGAA
GTAGGAAAAGCGCGACTGGTCAAAGGTGCAGCTAAACATATCATT
CATGCCGTAGGACCAAACTTCAACAAAGTTTCGGAGGTTGAAGGT
GACAAACAGTTGGCAGAGGCTTATGAGTCCATCGCTAAGATTGTC
AACGATAACAATTACAAGTCAGTAGCGATTCCACTGTTGTCCACC
GGCATCTTTTCCGGGAACAAAGATCGACTAACCCAATCATTGAAC
CATTTGCTGACAGCTTTAGACACCACTGATGCAGATGTAGCCATA
TACTGCAGGGACAAGAAATGGGAAATGACTCTCAAGGAAGCAGTG
GCTAGGAGAGAAGCAGTGGAGGAGATATGCATATCCGACGACTCT
TCAGTGACAGAACCTGATGCAGAGCTGGTGAGGGTGCATCCGAAG
AGTTCTTTGGCTGGAAGGAAGGGCTACAGCACAAGCGATGGCAAA
ACTTTCTCATATTTGGAAGGGACCAAGTTTCACCAGGCGGCCAAG
GATATAGCAGAAATTAATGCCATGTGGCCCGTTGCAACGGAGGCC
AATGAGCAGGTATGCATGTATATCCTCGGAGAAAGCATGAGCAGT
ATTAGGTCGAAATGCCCCGTCGAAGAGTCGGAAGCCTCCACACCA
CCTAGCACGCTGCCTTGCTTGTGCATCCATGCCATGACTCCAGAA
AGAGTACAGCGCCTAAAAGCCTCACGTCCAGAACAAATTACTGTG
TGCTCATCCTTTCCATTGCCGAAGTATAGAATCACTGGTGTGCAG
AAGATCCAATGCTCCCAGCCTATATTGTTCTCACCGAAAGTGCCT
GCGTATATTCATCCAAGGAAGTATCTCGTGGAAACACCACCGGTA
GACGAGACTCCGGAGCCATCGGCAGAGAACCAATCCACAGAGGGG
ACACCTGAACAACCACCACTTATAACCGAGGATGAGACCAGGACT
AGAACGCCTGAGCCGATCATCATCGAAGAGGAAGAAGAGGATAGC
ATAAGTTTGCTGTCAGATGGCCCGACCCACCAGGTGCTGCAAGTC
GAGGCAGACATTCACGGGCCGCCCTCTGTATCTAGCTCATCCTGG
TCCATTCCTCATGCATCCGACTTTGATGTGGACAGTTTATCCATA
CTTGACACCCTGGAGGGAGCTAGCGTGACCAGCGGGGCAACGTCA
GCCGAGACTAACTCTTACTTCGCAAAGAGTATGGAGTTTCTGGCG
CGACCGGTGCCTGCGCCTCGAACAGTATTCAGGAACCCTCCACAT
CCCGCTCCGCGCACAAGAACACCGTCACTTGCACCCAGCAGGGCC
TGCTCGAGAACCAGCCTAGTTTCCACCCCGCCAGGCGTGAATAGG
GTGATCACTAGAGAGGAGCTCGAGGCGCTTACCCCGTCACGCACT
CCTAGCAGGTCGGTCTCGAGAACCAGCCTGGTCTCCAACCCGCCA
GGCGTAAATAGGGTGATTACAAGAGAGGAGTTTGAGGCGTTCGTA
GCACAACAACAATGACGGTTTGATGCGGGTGCATACATCTTTTCC
TCCGACACCGGTCAAGGGCATTTACAACAAAAATCAGTAAGGCAA
ACGGTGCTATCCGAAGTGGTGTTGGAGAGGACCGAATTGGAGATT
TCGTATGCCCCGCGCCTCGACCAAGAAAAAGAAGAATTACTACGC
AAGAAATTACAGTTAAATCCCACACCTGCTAACAGAAGCAGATAC
CAGTCCAGGAAGGTGGAGAACATGAAAGCCATAACAGCTAGACGT
ATTCTGCAAGGCCTAGGGCATTATTTGAAGGCAGAAGGAAAAGTG
GAGTGCTACCGAACCCTGCATCCTGTTCCTTTGTATTCATCTAGT
GTGAACCGTGCCTTTTCAAGCCCCAAGGTCGCAGTGGAAGCCTGT
AACGCCATGTTGAAAGAGAACTTTCCGACTGTGGCTTCTTACTGT
ATTATTCCAGAGTACGATGCCTATTTGGACATGGTTGACGGAGCT
TCATGCTGCTTAGACACTGCCAGTTTTTGCCCTGCAAAGCTGCGC
AGCTTTCCAAAGAAACACTCCTATTTGGAACCCACAATACGATCG
GCAGTGCCTTCAGCGATCCAGAACACGCTCCAGAACGTCCTGGCA
GCTGCCACAAAAAGAAATTGCAATGTCACGCAAATGAGAGAATTG
CCCGTATTGGATTCGGCGGCCTTTAATGTGGAATGCTTCAAGAAA
TATGCGTGTAATAATGAATATTGGGAAACGTTTAAAGAAAACCCC
ATCAGGCTTACTGAAGAAAACGTGGTAAATTACATTACCAAATTA
AAAGGACCAAAAGCTGCTGCTCTTTTTGCGAAGACACATAATTTG
AATATGTTGCAGGACATACCAATGGACAGGTTTGTAATGGACTTA
AAGAGAGACGTGAAAGTGACTCCAGGAACAAAACATACTGAAGAA
CGGCCCAAGGTACAGGTGATCCAGGCTGCCGATCCGCTAGCAACA
GCGTATCTGTGCGGAATCCACCGAGAGCTGGTTAGGAGATTAAAT
GCGGTCCTGCTTCCGAACATTCATACACTGTTTGATATGTCGGCT
GAAGACTTTGACGCTATTATAGCCGAGCACTTCCAGCCTGGGGAT
TGTGTTCTGGAAACTGACATCGCGTCGTTTGATAAAAGTGAGGAC
GACGCCATGGCTCTGACCGCGTTAATGATTCTGGAAGACTTAGGT
GTGGACGCAGAGCTGTTGACGCTGATTGAGGCGGCTTTCGGCGAA
ATTTCATCAATACATTTGCCCACTAAAACTAAATTTAAATTCGGA
GCCATGATGAAATCTGGAATGTTCCTCACACTGTTTGTGAACACA
GTCATTAACATTGTAATCGCAAGCAGAGTGTTGAGAGAACGGCTA
ACCGGATCACCATGTGCAGCATTCATTGGAGATGACAATATCGTG
AAAGGAGTCAAATCGGACAAATTAATGGCAGACAGGTGCGCCACC
TGGTTGAATATGGAAGTCAAGATTATAGATGCTGTGGTGGGCGAG
AAAGCGCCTTATTTCTGTGGAGGGTTTATTTTGTGTGACTCCGTG
ACCGGCACAGCGTGCCGTGTGGCAGACCCCCTAAAAAGGCTGTTT
AAGCTTGGCAAACCTCTGGCAGCAGACGATGAACATGATGATGAC
AGGAGAAGGGCATTGCATGAAGAGTCAACACGCTGGAACCGAGTG
GGTATTCTTTCAGAGCTGTGCAAGGCAGTAGAATCAAGGTATGAA
ACCGTAGGAACTTCCATCATAGTTATGGCCATGACTACTCTAGCT
AGCAGTGTTAAATCATTCAGCTACCTGAGAGGGGCCCCTATAACT
CTCTACGGCTAACCTGAATGGACTACGACATAGTCTAGTCCGCCA
AGTCTGTTTAAACAGCATATGGGCGCGCCCTCAGCATCGATTCAA
TTCGCCACCATGGTGAGCAAGGGCGAGGAGCTGTTCACCGGGGTG
GTGCCCATCCTGGTCGAGCTGGACGGCGACGTAAACGGCCACAAG
TTCAGCGTGTCCGGCGAGGGCGAGGGCGATGCCACCTACGGCAAG
CTGACCCTGAAGTTCATCTGCACCACCGGCAAGCTGCCCGTGCCC
TGGCCCACCCTCGTGACCACCCTGACCTACGGCGTGCAGTGCTTC
AGCCGCTACCCCGACCACATGAAGCAGCACGACTTCTTCAAGTCC
GCCATGCCCGAAGGCTACGTCCAGGAGCGCACCATCTTCTTCAAG
GACGACGGCAACTACAAGACCCGCGCCGAGGTGAAGTTCGAGGGC
GACACCCTGGTGAACCGCATCGAGCTGAAGGGCATCGACTTCAAG
GAGGACGGCAACATCCTGGGGCACAAGCTGGAGTACAACTACAAC
AGCCACAACGTCTATATCATGGCCGACAAGCAGAAGAACGGCATC
AAGGTGAACTTCAAGATCCGCCACAACATCGAGGACGGCAGCGTG
CAGCTCGCCGACCACTACCAGCAGAACACCCCCATCGGCGACGGC
CCCGTGCTGCTGCCCGACAACCACTACCTGAGCACCCAGTCCGCC
CTGAGCAAAGACCCCAACGAGAAGCGCGATCACATGGTCCTGCTG
GAGTTCGTGACCGCCGCCGGGATCACTCTCGGCATGGACGAGCTG
TACAAGTAGTCTAGAGTGTTTAAACCGACCCGGGCGGCCGCAACT
AACTTAAGCTAGCAACGGTTTCCCTCTAGCGGGATCAATTCCGCC
CCCCCCCCCTAACGTTACTGGCCGAAGCCGCTTGGAATAAGGCCG
GTGTGCGTTTGTCTATATGTTATTTTCCACCATATTGCCGTCTTT
TGGCAATGTGAGGGCCCGGAAACCTGGCCCTGTCTTCTTGACGAG
CATTCCTAGGGGTCTTTCCCCTCTCGCCAAAGGAATGCAAGGTCT
GTTGAATGTCGTGAAGGAAGCAGTTCCTCTGGAAGCTTCTTGAAG
ACAAACAACGTCTGTAGCGACCCTTTGCAGGCAGCGGAACCCCCC
ACCTGGCGACAGGTGCCTCTGCGGCCAAAAGCCACGTGTATAAGA
TACACCTGCAAAGGCGGCACAACCCCAGTGCCACGTTGTGAGTTG
GATAGTTGTGGAAAGAGTCAAATGGCTCTCCTCAAGCGTATTCAA
CAAGGGGCTGAAGGATGCCCAGAAGGTACCCCATTGTATGGGATC
TGATCTGGGGCCTCGGTGCACATGCTTTACATGTGTTTAGTCGAG
GTTAAAAAAACGTCTAGGCCCCCCGAACCACGGGGACGTGGTTTT
CCTTTGAAAAACACGATAATACCATGACCGAGTACAAGCCCACGG
TGCGCCTCGCCACCCGCGACGACGTCCCCAGGGCCGTACGCACCC
TCGCCGCCGCGTTCGCCGACTACCCCGCCACGCGCCACACCGTCG
ATCCGGACCGCCACATCGAGCGGGTCACCGAGCTGCAAGAACTCT
TCCTCACGCGCGTCGGGCTCGACATCGGCAAGGTGTGGGTCGCGG
ACGACGGCGCCGCGGTGGCGGTCTGGACCACGCCGGAGAGCGTCG
AAGCGGGGGCGGTGTTCGCCGAGATCGGCCCGCGCATGGCCGAGT
TGAGCGGTTCCCGGCTGGCCGCGCAGCAACAGATGGAAGGCCTCC
TGGCGCCGCACCGGCCCAAGGAGCCCGCGTGGTTCCTGGCCACCG
TCGGCGTCTCGCCCGACCACCAGGGCAAGGGTCTGGGCAGCGCCG
TCGTGCTCCCCGGAGTGGAGGCGGCCGAGCGCGCCGGGGTGCCCG
CCTTCCTGGAGACCTCCGCGCCCCGCAACCTCCCCTTCTACGAGC
GGCTCGGCTTCACCGTCACCGCCGACGTCGAGGTGCCCGAAGGAC
CGCGCACCTGGTGCATGACCCGCAAGCCCGGTGCCTGAGAATTGG
CAAGCTGCTTACATAGAACTCGCGGCGATTGGCATGCCGCCTTAA
AATTTTTATTTTATTTTTTCTTTTCTTTTCCGAATCGGATTTTGT
TTTTAATATTTCAAAAAAAAAAAAAAAAAAAAAAAAAACGCGTCG
AGGGGAATTAATTCTTGAAGACGAAAGGGCCAGGTGGCACTTTTC
GGGGAAATGTGCGCGGAACCCCTATTTGTTTATTTTTCTAAATAC
ATTCAAATATGTATCCGCTCATGAGACAATAACCCTGATAAATGC
TTCAATAATATTGAAAAAGGAAGAGTATGAGTATTCAACATTTCC
GTGTCGCCCTTATTCCCTTTTTTGCGGCATTTTGCCTTCCTGTTT
TTGCTCACCCAGAAACGCTGGTGAAAGTAAAAGATGCTGAAGATC
AGTTGGGTGCACGAGTGGGTTACATCGAACTGGATCTCAACAGCG
GTAAGATCCTTGAGAGTTTTCGCCCCGAAGAACGTTTTCCAATGA
TGAGCACTTTTAAAGTTCTGCTATGTGGCGCGGTATTATCCCGTG
TTGACGCCGGGCAAGAGCAACTCGGTCGCCGCATACACTATTCTC
AGAATGACTTGGTTGAGTACTCACCAGTCACAGAAAAGCATCTTA
CGGATGGCATGACAGTAAGAGAATTATGCAGTGCTGCCATAACCA
TGAGTGATAACACTGCGGCCAACTTACTTCTGACAACGATCGGAG
GACCGAAGGAGCTAACCGCTTTTTTGCACAACATGGGGGATCATG
TAACTCGCCTTGATCGTTGGGAACCGGAGCTGAATGAAGCCATAC
CAAACGACGAGCGTGACACCACGATGCCTGTAGCAATGGCAACAA
CGTTGCGCAAACTATTAACTGGCGAACTACTTACTCTAGCTTCCC
GGCAACAATTAATAGACTGGATGGAGGCGGATAAAGTTGCAGGAC
CACTTCTGCGCTCGGCCCTTCCGGCTGGCTGGTTTATTGCTGATA
AATCTGGAGCCGGTGAGCGTGGGTCTCGCGGTATCATTGCAGCAC
TGGGGCCAGATGGTAAGCCCTCCCGTATCGTAGTTATCTACACGA
CGGGGAGTCAGGCAACTATGGATGAACGAAATAGACAGATCGCTG
AGATAGGTGCCTCACTGATTAAGCATTGGTAACTGTCAGACCAAG
TTTACTCATATATACTTTAGATTGATTTAAAACTTCATTTTTAAT
TTAAAAGGATCTAGGTGAAGATCCTTTTTGATAATCTCATGACCA
AAATCCCTTAACGTGAGTTTTCGTTCCACTGAGCGTCAGACCCCG
TAGAAAAGATCAAAGGATCTTCTTGAGATCCTTTTTTTCTGCGCG
TAATCTGCTGCTTGCAAACAAAAAAACCACCGCTACCAGCGGTGG
TTTGTTTGCCGGATCAAGAGCTACCAACTCTTTTTCCGAAGGTAA
CTGGCTTCAGCAGAGCGCAGATACCAAATACTGTCCTTCTAGTGT
AGCCGTAGTTAGGCCACCACTTCAAGAACTCTGTAGCACCGCCTA
CATACCTCGCTCTGCTAATCCTGTTACCAGTGGCTGCTGCCAGTG
GCGATAAGTCGTGTCTTACCGGGTTGGACTCAAGACGATAGTTAC
CGGATAAGGCGCAGCGGTCGGGCTGAACGGGGGGTTCGTGCACAC
AGCCCAGCTTGGAGCGAACGACCTACACCGAACTGAGATACCTAC
AGCGTGAGCTATGAGAAAGCGCCACGCTTCCCGAAGGGAGAAAGG
CGGACAGGTATCCGGTAAGCGGCAGGGTCGGAACAGGAGAGCGCA
CGAGGGAGCTTCCAGGGGGAAACGCCTGGTATCTTTATAGTCCTG
TCGGGTTTCGCCACCTCTGACTTGAGCGTCGATTTTTGTGATGCT
CGTCAGGGGGGCGGAGCCTATGGAAAAACGCCAGCAACGCGAGCT
CGCGATCGCTTAATTAAGACATTGATTATTGACTAGTTATTAATA
GTAATCAATTACGGGGTCATTAGTTCATAGCCCATATATGGAGTT
CCGCGTTACATAACTTACGGTAAATGGCCCGCCTGGCTGACCGCC
CAACGACCCCCGCCCATTGACGTCAATAATGACGTATGTTCCCAT
AGTAACGCCAATAGGGACTTTCCATTGACGTCAATGGGTGGAGTA
TTTACGGTAAACTGCCCACTTGGCAGTACATCAAGTGTATCATAT
GCCAAGTACGCCCCCTATTGACGTCAATGACGGTAAATGGCCCGC
CTGGCATTATGCCCAGTACATGACCTTATGGGACTTTCCTACTTG
GCAGTACATCTACGTATTAGTCATCGCTATTACCATGGTCGAGGT
GAGCCCCACGTTCTGCTTCACTCTCCCCATCTCCCCCCCCTCCCC
ACCCCCAATTTTGTATTTATTTATTTTTTAATTATTTTGTGCAGC
GATGGGGGCGGGGGGGGGGGGGGCGCGCGCCAGGGGGGGGGGGGG
GGGGGGGGGGGGGGGGGGGGGGGGGGGGGGGGGGGGCGGCGGCAG
CCAATCAGAGCGGCGCGCTCCGAAAGTTTCCTTTTATGGCGAGGC
GGCGGCGGCGGCGGCCCTATAAAAAGCGAAGCGCGCGGCGGGCGG
GAGTCGCTGCGCGCTGCCTTCGCCCCGTGCCCCGCTCCGCCGCCG
CCTCGCGCCGCCCGCCCCGGCTCTGACTGACCGCGTTACTCCCAC
AGGTGAGCGGGCGGGACGGCCCTTCTCCTCCGGGCTGTAATTAGC
GCTTGGTTTAATGACGGCTTGTTTCTTTTCTGTGGCTGCGTGAAA
GCCTTGAGGGGCTCCGGGAGGGCCCTTTGTGCGGGGGGAGCGGCT
CGGGGGGTGCGTGCGTGTGTGTGTGCGTGGGGAGCGCCGCGTGCG
GCTCCGCGCTGCCCGGCGGCTGTGAGCGCTGCGGGCGCGGCGCGG
GGCTTTGTGCGCTCCGCAGTGTGCGCGAGGGGAGCGCGGCCGGGG
GCGGTGCCCCGCGGTGCGGGGGGGGCTGCGAGGGGAACAAAGGCT
GCGTGCGGGGTGTGTGCGTGGGGGGGTGAGCAGGGGGTGTGGGCG
CGTCGGTCGGGCTGCAACCCCCCCTGCACCCCCCTCCCCGAGTTG
CTGAGCACGGCCCGGCTTCGGGTGCGGGGCTCCGTACGGGGCGTG
GCGCGGGGCTCGCCGTGCCGGGCGGGGGGTGGCGGCAGGTGGGGG
TGCCGGGCGGGGCGGGGCCGCCTCGGGCCGGGGGGGGCTCGGGGG
GGGGGGGCGGCGGCCCCCGGAGCGCCGGCGGCTGTCGAGGCGCGG
CGAGCCGCAGCCATTGCCTTTTATGGTAATCGTGCGAGAGGGCGC
AGGGACTTCCTTTGTCCCAAATCTGTGCGGAGCCGAAATCTGGGA
GGCGCCGCCGCACCCCCTCTAGCGGGCGCGGGGCGAAGCGGTGCG
GCGCCGGCAGGAAGGAAATGGGCGGGGAGGGCCTTCGTGCGTCGC
CGCGCCGCCGTCCCCTTCTCCCTCTCCAGCCTCGGGGCTGTCCGC
GGGGGGACGGCTGCCTTCGGGGGGGACGGGGCAGGGCGGGGTTCG
GCTTCTGGCGTGTGACCGGCGGCTTCAGAGCCTCTGCTAACCATG
TTCATGCCTTCTTCTTTTTCCTACAGTAATACGACTCACTATAGG
GCCGGCCATGGGCGGCGCATGAGAGAAGCCCAGACCAATTACCTA
CCCAAAATGGAGAAAGTTCACGTTGACATCGAGGAAGACAGCCCA
TTCCTCAGAGCTTTGCAGCGGAGCTTCCCGCAGTTTGAGGTAGAA
GCCAAGCAGGTCACTGATAATGACCATGCTAATGCCAGAGCGTTT
TCGCATCTGGCTTCAAAACTGATCGAAACGGAGGTGGACCCATCC
GACACGATCCTTGACATTGGAAGTGCGCCCGCCCGCAGAATGTAT
TCTAAGCACAAGTATCATTGTATCTGTCCGATGAGATGTGCGGAA
GATCCGGACAGATTGTATAAGTATGCAACTAAGCTGAAGAAAAAC
TGTAAGGAAATAACTGATAAGGAATTGGACAAGAAAATGAAGGAG
CTGGCCGCCGTCATGAGCGACCCTGACCTGGAAACTGAGACTATG
TGCCTCCACGACGACGAGTCGTGTCGCTACGAAGGGCAAGTCGCT
GTTTACCAGGATGTATACGCGGTTGACGGACCGACAAGTCTCTAT
CACCAAGCCAATAAGGGAGTTAGAGTCGCCTACTGGATAGGCTTT
GACACCACCCCTTTTATGTTTAAGAACTTGGCTGGAGCATATCCA
TCATACTCTACCAACTGGGCCGACGAAACCGTGTTAACGGCTCGT
AACATAGGCCTATGCAGCTCTGACGTTATGGAGCGGTCACGTAGA
GGGATGTCCATTCTTAGAAAGAAGTATTTGAAACCATCCAACAAT
GTTCTATTCTCTGTTGGCTCGACCATCTACCACGAGAAGAGGGAC
TTACTGAGGAGCTGGCACCTGCCGTCTGTATTTCACTTACGTGGC
AAGCAA

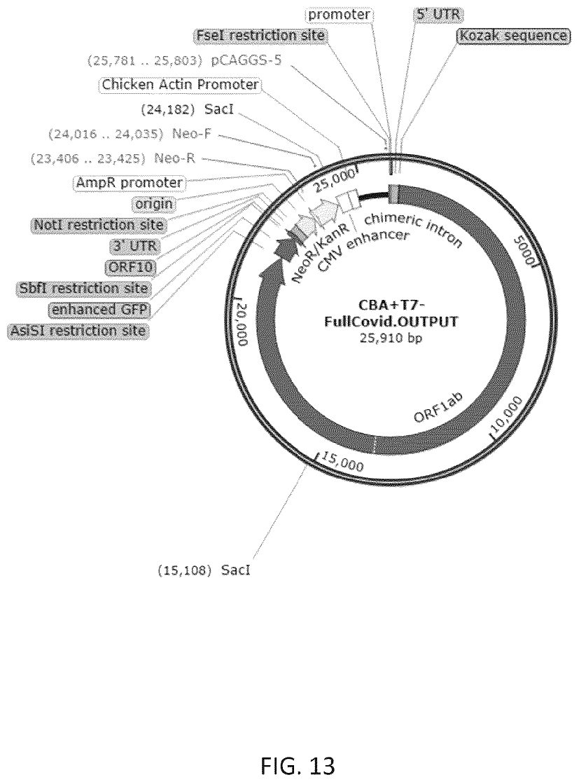

CBA + T7-FullCovid-GFP (FIG. 13)

| | |
|---|---|
| source | 1 . . . 25,910 |
| 5′ UTR | 1 . . . 265 |
| Kozak sequence | 266 . . . 271 |
| ORF1ab | 272 . . . 21,561 |
| ORF1ab | 272 . . . 21,561 |
| ORF1ab | 272 . . . 13,489 |
| mature peptide | 272 . . . 811 |
| mature peptide | 812 . . . 2725 |
| mature peptide | 2726 . . . 8560 |
| mature peptide | 8561 . . . 10,060 |
| mature peptide | 10,061 . . . 10,978 |
| mature peptide | 10,979 . . . 11,848 |
| mature peptide | 11,849 . . . 12,097 |
| mature peptide | 12,098 . . . 12,691 |
| mature peptide | 12,692 . . . 13,030 |
| mature peptide | 13,031 . . . 13,447 |
| mature peptide | 13,448 . . . 16,242 |
| mature peptide | 13,448 . . . 13,486 |
| stem loop | 13,482 . . . 13,509 |
| stem loop | 13,494 . . . 13,548 |
| mature peptide | 16,243 . . . 18,045 |
| mature peptide | 18,046 . . . 19,626 |
| mature peptide | 19,627 . . . 20,664 |
| mature peptide | 20,665 . . . 21,558 |
| AsiSI restriction site | 21,562 . . . 21,569 |
| regulatory region | 21,570 . . . 21,579 |
| enhanced GFP | 21,576 . . . 22,295 |
| EGFP, reverse primer | 21,621 . . . 21,642 |
| For distinguishing EGFP variants, reverse primer | 21,882 . . . 21,901 |
| EGFP, forward primer | 22,229 . . . 22,250 |
| SbfI restriction site | 22,296 . . . 22,303 |
| source | 22,296 . . . 22,303 |
| ORF10 | 22,304 . . . 22,420 |
| ORF10 | 22,304 . . . 22,420 |
| stem loop | 22,355 . . . 22,390 |
| stem loop | 22,375 . . . 22,403 |
| 3′ UTR | 22,421 . . . 22,649 |
| stem loop | 22,474 . . . 22,514 |
| NotI restriction site | 22,650 . . . 22,657 |
| source | 22,650 . . . 22,657 |
| origin | 22,658 . . . 23,246 |
| pBR322 origin, forward primer | 23,147 . . . 23,166 |
| source | 23,247 . . . 24,163 |
| AmpR promoter | 23,247 . . . 23,351 |
| NeoR/KanR | 23,352 . . . 24,146 |
| CMV enhancer | 24,184 . . . 24,563 |
| Chicken Beta Actin Promoter | 24,564 . . . 24,840 |
| chimeric intron | 24,841 . . . 25,857 |
| FseI restriction site | 25,858 . . . 25,865 |
| source | 25,858 . . . 25,865 |
| promoter | 25,892 . . . 25,910 |

ATTAAAGGTTTATACCTTCCCAGGTAACAAACCAACCAACTTTCGATCTCTTGTAG
ATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGGCCACCATGGAGAGC
CTTGTCCCTGGTTTCAACGAGAAAACACACGTCCAACTCAGTTTGCCTGTTTTACA
GGTTCGCGACGTGCTCGTACGTGGCTTTGGAGACTCCGTGGAGGAGGTCTTATCAG
AGGCACGTCAACATCTTAAAGATGGCACTTGTGGCTTAGTAGAAGTTGAAAAAGG
CGTTTTGCCTCAACTTGAACAGCCCTATGTGTTCATCAAACGTTCGGATGCTCGAA
CTGCACCTCATGGTCATGTTATGGTTGAGCTGGTAGCAGAACTCGAAGGCATTCAG
TACGGTCGTAGTGGTGAGACACTTGGTGTCCTTGTCCCTCATGTGGGCGAAATACC
AGTGGCTTACCGCAAGGTTCTTCTTCGTAAGAACGGTAATAAAGGAGCTGGTGGC
CATAGTTACGGCGCCGATCTAAAGTCATTTGACTTAGGCGACGAGCTTGGCACTGA
TCCTTATGAAGATTTTCAAGAAAACTGGAACACTAAACATAGCAGTGGTGTTACCC
GTGAACTCATGCGTGAGCTTAACGGAGGGGCATACACTCGCTATGTCGATAACAA
CTTCTGTGGCCCTGATGGCTACCCTCTTGAGTGCATTAAAGACCTTCTAGCACGTG
CTGGTAAAGCTTCATGCACTTTGTCCGAACAACTGGACTTTATTGACACTAAGAGG
GGTGTATACTGCTGCCGTGAACATGAGCATGAAATTGCTTGGTACACGGAACGTTC
TGAAAAGAGCTATGAATTGCAGACACCTTTTGAAATTAAATTGGCAAAGAAATTT
GACACCTTCAATGGGGAATGTCCAAATTTTGTATTTCCCTTAAATTCCATAATCAA
GACTATTCAACCAAGGGTTGAAAAGAAAAAGCTTGATGGCTTTATGGGTAGAATT
CGATCTGTCTATCCAGTTGCGTCACCAAATGAATGCAACCAAATGTGCCTTTCAAC
TCTCATGAAGTGTGATCATTGTGGTGAAACTTCATGGCAGACGGGCGATTTTGTTA
AAGCCACTTGCGAATTTTGTGGCACTGAGAATTTGACTAAAGAAGGTGCCACTACT
TGTGGTTACTTACCCCAAAATGCTGTTGTTAAAATTTATTGTCCAGCATGTCACAA
TTCAGAAGTAGGACCTGAGCATAGTCTTGCCGAATACCATAATGAATCTGGCTTGA
AAACCATTCTTCGTAAGGGTGGTCGCACTATTGCCTTTGGAGGCTGTGTGTTCTCTT
ATGTTGGTTGCCATAACAAGTGTGCCTATTGGGTTCCACGTGCTAGCGCTAACATA
GGTTGTAACCATACAGGTGTTGTTGGAGAAGGTTCCGAAGGTCTTAATGACAACCT
TCTTGAAATACTCCAAAAAGAGAAAGTCAACATCAATATTGTTGGTGACTTTAAAC
TTAATGAAGAGATCGCCATTATTTTGGCATCTTTTTCTGCTTCCACAAGTGCTTTTG
TGGAAACTGTGAAAGGTTTGGATTATAAAGCATTCAAACAAATTGTTGAATCCTGT
GGTAATTTTAAAGTTACAAAAGGAAAAGCTAAAAAAGGTGCCTGGAATATTGGTG
AACAGAAATCAATACTGAGTCCTCTTTATGCATTTGCATCAGAGGCTGCTCGTGTT
GTACGATCAATTTTCTCCCGCACTCTTGAAACTGCTCAAAATTCTGTGCGTGTTTTA
CAGAAGGCCGCTATAACAATACTAGATGGAATTTCACAGTATTCACTGAGACTCA
TTGATGCTATGATGTTCACATCTGATTTGGCTACTAACAATCTAGTTGTAATGGCCT
ACATTACAGGTGGTGTTGTTCAGTTGACTTCGCAGTGGCTAACTAACATCTTTGGC
ACTGTTTATGAAAAACTCAAACCCGTCCTTGATTGGCTTGAAGAGAAGTTTAAGGA
AGGTGTAGAGTTTCTTAGAGACGGTTGGGAAATTGTTAAATTTATCTCAACCTGTG
CTTGTGAAATTGTCGGTGGACAAATTGTCACCTGTGCAAAGGAAATTAAGGAGAG
TGTTCAGACATTCTTTAAGCTTGTAAATAAATTTTTGGCTTTGTGTGCTGACTCTAT
CATTATTGGTGGAGCTAAACTTAAAGCCTTGAATTTAGGTGAAACATTTGTCACGC
ACTCAAAGGGATTGTACAGAAAGTGTGTTAAATCCAGAGAAGAAACTGGCCTACT
CATGCCTCTAAAAGCCCCAAAAGAAATTATCTTCTTAGAGGGAGAAACACTTCCC
ACAGAAGTGTTAACAGAGGAAGTTGTCTTGAAAACTGGTGATTTACAACCATTAG
AACAACCTACTAGTGAAGCTGTTGAAGCTCCATTGGTTGGTACACCAGTTTGTATT
AACGGGCTTATGTTGCTCGAAATCAAAGACACAGAAAAGTACTGTGCCCTTGCAC
CTAATATGATGGTAACAAACAATACCTTCACACTCAAAGGCGGTGCACCAACAAA
GGTTACTTTTGGTGATGACACTGTGATAGAAGTGCAAGGTTACAAGAGTGTGAAT
ATCACTTTTGAACTTGATGAAAGGATTGATAAAGTACTTAATGAGAAGTGCTCTGC
CTATACAGTTGAACTCGGTACAGAAGTAAATGAGTTCGCCTGTGTTGTGGCAGATG
CTGTCATAAAAACTTTGCAACCAGTATCTGAATTACTTACACCACTGGGCATTGAT
TTAGATGAGTGGAGTATGGCTACATACTACTTATTTGATGAGTCTGGTGAGTTTAA
ATTGGCTTCACATATGTATTGTTCTTTCTACCCTCCAGATGAGGATGAAGAAGAAG
GTGATTGTGAAGAAGAAGAGTTTGAGCCATCAACTCAATATGAGTATGGTACTGA
AGATGATTACCAAGGTAAACCTTTGGAATTTGGTGCCACTTCTGCTGCTCTTCAAC
CTGAAGAAGAGCAAGAAGAAGATTGGTTAGATGATGATAGTCAACAAACTGTTGG
TCAACAAGACGGCAGTGAGGACAATCAGACAACTACTATTCAAACAATTGTTGAG
GTTCAACCTCAATTAGAGATGGAACTTACACCAGTTGTTCAGACTATTGAAGTGAA
TAGTTTTAGTGGTTATTTAAAACTTACTGACAATGTATACATTAAAAATGCAGACA
TTGTGGAAGAAGCTAAAAAGGTAAAACCAACAGTGGTTGTTAATGCAGCCAATGT
TTACCTTAAACATGGAGGAGGTGTTGCAGGAGCCTTAAATAAGGCTACTAACAAT
GCCATGCAAGTTGAATCTGATGATTACATAGCTACTAATGGACCACTTAAAGTGG
GTGGTAGTTGTGTTTTAAGCGGACACAATCTTGCTAAACACTGTCTTCATGTTGTC
GGCCCAAATGTTAACAAAGGTGAAGACATTCAACTTCTTAAGAGTGCTTATGAAA
ATTTTAATCAGCACGAAGTTCTACTTGCACCATTATTATCAGCTGGTATTTTTGGTG
CTGACCCTATACATTCTTTAAGAGTTTGTGTAGATACTGTTCGCACAAATGTCTACT
TAGCTGTCTTTGATAAAAATCTCTATGACAAACTTGTTTCAAGCTTTTTGGAAATG
AAGAGTGAAAAGCAAGTTGAACAAAAGATCGCTGAGATTCCTAAAGAGGAAGTT
AAGCCATTTATAACTGAAAGTAAACCTTCAGTTGAACAGAGAAAACAAGATGATA
AGAAAATCAAAGCTTGTGTTGAAGAAGTTACAACAACTCTGGAAGAAACTAAGTT
CCTCACAGAAAACTTGTTACTTTATATTGACATTAATGGCAATCTTCATCCAGATT
CTGCCACTCTTGTTAGTGACATTGACATCACTTTCTTAAAGAAAGATGCTCCATAT
ATAGTGGGTGATGTTGTTCAAGAGGGTGTTTTAACTGCTGTGGTTATACCTACTAA
AAAGGCTGGTGGCACTACTGAAATGCTAGCGAAAGCTTTGAGAAAAGTGCCAACA
GACAATTATATAACCACTTACCCGGGTCAGGGTTTAAATGGTTACACTGTAGAGG
AGGCAAAGACAGTGCTTAAAAAGTGTAAAAGTGCCTTTTACATTCTACCATCTATT
ATCTCTAATGAGAAGCAAGAAATTCTTGGAACTGTTTCTTGGAATTTGCGAGAAAT
GCTTGCACATGCAGAAGAAACACGCAAATTAATGCCTGTCTGTGTGGAAACTAAA
GCCATAGTTTCAACTATACAGCGTAAATATAAGGGTATTAAAATACAAGAGGGTG
TGGTTGATTATGGTGCTAGATTTTACTTTTACACCAGTAAAACAACTGTAGCGTCA
CTTATCAACACACTTAACGATCTAAATGAAACTCTTGTTACAATGCCACTTGGCTA
TGTAACACATGGCTTAAATTTGGAAGAAGCTGCTCGGTATATGAGATCTCTCAAAG
TGCCAGCTACAGTTTCTGTTTCTTCACCTGATGCTGTTACAGCGTATAATGGTTATC
TTACTTCTTCTTCTAAAACACCTGAAGAACATTTTATTGAAACCATCTCACTTGCTG
GTTCCTATAAAGATTGGTCCTATTCTGGACAATCTACACAACTAGGTATAGAATTT
CTTAAGAGAGGTGATAAAAGTGTATATTACACTAGTAATCCTACCACATTCCACCT
AGATGGTGAAGTTATCACCTTTGACAATCTTAAGACACTTCTTTCTTTGAGAGAAG
TGAGGACTATTAAGGTGTTTACAACAGTAGACAACATTAACCTCCACACGCAAGT
TGTGGACATGTCAATGACATATGGACAACAGTTTGGTCCAACTTATTTGGATGGAG
CTGATGTTACTAAAATAAAACCTCATAATTCACATGAAGGTAAAACATTTTATGTT
TTACCTAATGATGACACTCTACGTGTTGAGGCTTTTGAGTACTACCACACAACTGA
TCCTAGTTTTCTGGGTAGGTACATGTCAGCATTAAATCACACTAAAAAGTGGAAAT
ACCCACAAGTTAATGGTTTAACTTCTATTAAATGGGCAGATAACAACTGTTATCTT
GCCACTGCATTGTTAACACTCCAACAAATAGAGTTGAAGTTTAATCCACCTGCTCT
ACAAGATGCTTATTACAGAGCAAGGGCTGGTGAAGCTGCTAACTTTTGTGCACTTA
TCTTAGCCTACTGTAATAAGACAGTAGGTGAGTTAGGTGATGTTAGAGAAACAAT
GAGTTACTTGTTTCAACATGCCAATTTAGATTCTTGCAAAAGAGTCTTGAACGTGG
TGTGTAAAACTTGTGGACAACAGCAGACAACCCTTAAGGGTGTAGAAGCTGTTAT
GTACATGGGCACACTTTCTTATGAACAATTTAAGAAAGGTGTTCAGATACCTTGTA
CGTGTGGTAAACAAGCTACAAAATATCTAGTACAACAGGAGTCACCTTTTGTTATG
ATGTCAGCACCACCTGCTCAGTATGAACTTAAGCATGGTACATTTACTTGTGCTAG
TGAGTACACTGGTAATTACCAGTGTGGTCACTATAAACATATAACTTCTAAAGAAA
CTTTGTATTGCATAGACGGTGCTTTACTTACAAAGTCCTCAGAATACAAAGGTCCT
ATTACGGATGTTTTCTACAAAGAAAACAGTTACACAACAACCATAAAACCAGTTA
CTTATAAATTGGATGGTGTTGTTTGTACAGAAATTGACCCTAAGTTGGACAATTAT
TATAAGAAAGACAATTCTTATTTCACAGAGCAACCAATTGATCTTGTACCAAACCA
ACCATATCCAAACGCAAGCTTCGATAATTTTAAGTTTGTATGTGATAATATCAAAT
TTGCTGATGATTTAAACCAGTTAACTGGTTATAAGAAACCTGCTTCAAGAGAGCTT
AAAGTTACATTTTTCCCTGACTTAAATGGTGATGTGGTGGCTATTGATTATAAACA
CTACACACCCTCTTTTAAGAAAGGAGCTAAATTGTTACATAAACCTATTGTTTGGC
ATGTTAACAATGCAACTAATAAAGCCACGTATAAACCAAATACCTGGTGTATACG
TTGTCTTTGGAGCACAAAACCAGTTGAAACATCAAATTCGTTTGATGTACTGAAGT
CAGAGGACGCGCAGGGAATGGATAATCTTGCCTGCGAAGATCTAAAACCAGTCTC
TGAAGAAGTAGTGGAAAATCCTACCATACAGAAAGACGTTCTTGAGTGTAATGTG
AAAACTACCGAAGTTGTAGGAGACATTATACTTAAACCAGCAAATAATAGTTTAA
AAATTACAGAAGAGGTTGGCCACACAGATCTAATGGCTGCTTATGTAGACAATTC
TAGTCTTACTATTAAGAAACCTAATGAATTATCTAGAGTATTAGGTTTGAAAACCC
TTGCTACTCATGGTTTAGCTGCTGTTAATAGTGTCCCTTGGGATACTATAGCTAATT
ATGCTAAGCCTTTTCTTAACAAAGTTGTTAGTACAACTACTAACATAGTTACACGG
TGTTTAAACCGTGTTTGTACTAATTATATGCCTTATTTCTTTACTTTATTGCTACAAT
TGTGTACTTTTACTAGAAGTACAAATTCTAGAATTAAAGCATCTATGCCGACTACT
ATAGCAAAGAATACTGTTAAGAGTGTCGGTAAATTTTGTCTAGAGGCTTCATTTAA
TTATTTGAAGTCACCTAATTTTTCTAAACTGATAAATATTATAATTTGGTTTTTACT
ATTAAGTGTTTGCCTAGGTTCTTTAATCTACTCAACCGCTGCTTTAGGTGTTTTAAT
GTCTAATTTAGGCATGCCTTCTTACTGTACTGGTTACAGAGAAGGCTATTTGAACT
CTACTAATGTCACTATTGCAACCTACTGTACTGGTTCTATACCTTGTAGTGTTTGTC
TTAGTGGTTTAGATTCTTTAGACACCTATCCTTCTTTAGAAACTATACAAATTACCA
TTTCATCTTTTAAATGGGATTTAACTGCTTTTGGCTTAGTTGCAGAGTGGTTTTTGG
CATATATTCTTTTCACTAGGTTTTTCTATGTACTTGGATTGGCTGCAATCATGCAAT
TGTTTTTCAGCTATTTTGCAGTACATTTTATTAGTAATTCTTGGCTTATGTGGTTAA
TAATTAATCTTGTACAAATGGCCCCGATTTCAGCTATGGTTAGAATGTACATCTTC
TTTGCATCATTTTATTATGTATGGAAAAGTTATGTGCATGTTGTAGACGGTTGTAAT
TCATCAACTTGTATGATGTGTTACAAACGTAATAGAGCAACAAGAGTCGAATGTA
CAACTATTGTTAATGGTGTTAGAAGGTCCTTTTATGTCTATGCTAATGGAGGTAAA
GGCTTTTGCAAACTACACAATTGGAATTGTGTTAATTGTGATACATTCTGTGCTGG
TAGTACATTTATTAGTGATGAAGTTGCGAGAGACTTGTCACTACAGTTTAAAAGAC
CAATAAATCCTACTGACCAGTCTTCTTACATCGTTGATAGTGTTACAGTGAAGAAT
GGTTCCATCCATCTTTACTTTGATAAAGCTGGTCAAAAGACTTATGAAAGACATTC
TCTCTCTCATTTTGTTAACTTAGACAACCTGAGAGCTAATAACACTAAAGGTTCAT
TGCCTATTAATGTTATAGTTTTTGATGGTAAATCAAAATGTGAAGAATCATCTGCA
AAATCAGCGTCTGTTTACTACAGTCAGCTTATGTGTCAACCTATACTGTTACTAGA
TCAGGCATTAGTGTCTGATGTTGGTGATAGTGCGGAAGTTGCAGTTAAAATGTTTG
ATGCTTACGTTAATACGTTTTCATCAACTTTTAACGTACCAATGGAAAAACTCAAA
ACACTAGTTGCAACTGCAGAAGCTGAACTTGCAAAGAATGTGTCCTTAGACAATG
TCTTATCTACTTTTATTTCAGCAGCTCGGCAAGGGTTTGTTGATTCAGATGTAGAA
ACTAAAGATGTTGTTGAATGTCTTAAATTGTCACATCAATCTGACATAGAAGTTAC
TGGCGATAGTTGTAATAACTATATGCTCACCTATAACAAAGTTGAAAACATGACA
CCCCGTGACCTTGGTGCTTGTATTGACTGTAGTGCGCGTCATATTAATGCGCAGGT
AGCAAAAAGTCACAACATTGCTTTGATATGGAACGTTAAAGATTTCATGTCATTGT
CTGAACAACTACGAAAACAAATACGTAGTGCTGCTAAAAAGAATAACTTACCTTT
TAAGTTGACATGTGCAACTACTAGACAAGTTGTTAATGTTGTAACAACAAAGATA
GCACTTAAGGGTGGTAAAATTGTTAATAATTGGTTGAAGCAGTTAATTAAAGTTAC
ACTTGTGTTCCTTTTTGTTGCTGCTATTTTCTATTTAATAACACCTGTTCATGTCATG
TCTAAACATACTGACTTTTCAAGTGAAATCATAGGATACAAGGCTATTGATGGTGG
TGTCACTCGTGACATAGCATCTACAGATACTTGTTTTGCTAACAAACATGCTGATT
TTGACACATGGTTTAGCCAGCGTGGTGGTAGTTATACTAATGACAAAGCTTGCCCA
TTGATTGCTGCAGTCATAACAAGAGAAGTGGGTTTTGTCGTGCCTGGTTTGCCTGG
CACGATATTACGCACAACTAATGGTGACTTTTTGCATTTCTTACCTAGAGTTTTTAG
TGCAGTTGGTAACATCTGTTACACACCATCAAAACTTATAGAGTACACTGACTTTG
CAACATCAGCTTGTGTTTTGGCTGCTGAATGTACAATTTTTAAAGATGCTTCTGGT
AAGCCAGTACCATATTGTTATGATACCAATGTACTAGAAGGTTCTGTTGCTTATGA
AAGTTTACGCCCTGACACACGTTATGTGCTCATGGATGGCTCTATTATTCAATTTCC
TAACACCTACCTTGAAGGTTCTGTTAGAGTGGTAACAACTTTTGATTCTGAGTACT
GTAGGCACGGCACTTGTGAAAGATCAGAAGCTGGTGTTTGTGTATCTACTAGTGGT
AGATGGGTACTTAACAATGATTATTACAGATCTTTACCAGGAGTTTTCTGTGGTGT
AGATGCTGTAAATTTACTTACTAATATGTTTACACCACTAATTCAACCTATTGGTG
CTTTGGACATATCAGCATCTATAGTAGCTGGTGGTATTGTAGCTATCGTAGTAACA
TGCCTTGCCTACTATTTTATGAGGTTTAGAAGAGCTTTTGGTGAATACAGTCATGT
AGTTGCCTTTAATACTTTACTATTCCTTATGTCATTCACTGTACTCTGTTTAACACC
AGTTTACTCATTCTTACCTGGTGTTTATTCTGTTATTTACTTGTACTTGACATTTTAT
CTTACTAATGATGTTTCTTTTTTAGCACATATTCAGTGGATGGTTATGTTCACACCT
TTAGTACCTTTCTGGATAACAATTGCTTATATCATTTGTATTTCCACAAAGCATTTC
TATTGGTTCTTTAGTAATTACCTAAAGAGACGTGTAGTCTTTAATGGTGTTTCCTTT
AGTACTTTTGAAGAAGCTGCGCTGTGCACCTTTTTGTTAAATAAAGAAATGTATCT
AAAGTTGCGTAGTGATGTGCTATTACCTCTTACGCAATATAATAGATACTTAGCTC
TTTATAATAAGTACAAGTATTTTAGTGGAGCAATGGATACAACTAGCTACAGAGA
AGCTGCTTGTTGTCATCTCGCAAAGGCTCTCAATGACTTCAGTAACTCAGGTTCTG
ATGTTCTTTACCAACCACCACAAACCTCTATCACCTCAGCTGTTTTGCAGAGTGGT
TTTAGAAAAATGGCATTCCCATCTGGTAAAGTTGAGGGTTGTATGGTACAAGTAAC
TTGTGGTACAACTACACTTAACGGTCTTTGGCTTGATGACGTAGTTTACTGTCCAA
GACATGTGATCTGCACCTCTGAAGACATGCTTAACCCTAATTATGAAGATTTACTC
ATTCGTAAGTCTAATCATAATTTCTTGGTACAGGCTGGTAATGTTCAACTCAGGGT
TATTGGACATTCTATGCAAAATTGTGTACTTAAGCTTAAGGTTGATACAGCCAATC
CTAAGACACCTAAGTATAAGTTTGTTCGCATTCAACCAGGACAGACTTTTTCAGTG
TTAGCTTGTTACAATGGTTCACCATCTGGTGTTTACCAATGTGCTATGAGGCCCAA
TTTCACTATTAAGGGTTCATTCCTTAATGGTTCATGTGGTAGTGTTGGTTTTAACAT
AGATTATGACTGTGTCTCTTTTTGTTACATGCACCATATGGAATTACCAACTGGAG
TTCATGCTGGCACAGACTTAGAAGGTAACTTTTATGGACCTTTTGTTGACAGGCAA
ACAGCACAAGCAGCTGGTACGGACACAACTATTACAGTTAATGTTTTAGCTTGGTT
GTACGCTGCTGTTATAAATGGAGACAGGTGGTTTCTCAATCGATTTACCACAACTC
TTAATGACTTTAACCTTGTGGCTATGAAGTACAATTATGAACCTCTAACACAAGAC
CATGTTGACATACTAGGACCTCTTTCTGCTCAAACTGGAATTGCCGTTTTAGATAT
GTGTGCTTCATTAAAAGAATTACTGCAAAATGGTATGAATGGACGTACCATATTGG
GTAGTGCTTTATTAGAAGATGAATTTACACCTTTTGATGTTGTTAGACAATGCTCA
GGTGTTACTTTCCAAAGTGCAGTGAAAAGAACAATCAAGGGTACACACCACTGGT
TGTTACTCACAATTTTGACTTCACTTTTAGTTTTAGTCCAGAGTACTCAATGGTCTT
TGTTCTTTTTTTTGTATGAAAATGCCTTTTTACCTTTTGCTATGGGTATTATTGCTAT
GTCTGCTTTTGCAATGATGTTTGTCAAACATAAGCATGCATTTCTCTGTTTGTTTTT
GTTACCTTCTCTTGCCACTGTAGCTTATTTTAATATGGTCTATATGCCTGCTAGTTG
GGTGATGCGTATTATGACATGGTTGGATATGGTTGATACTAGTTTGTCTGGTTTTA
AGCTAAAAGACTGTGTTATGTATGCATCAGCTGTAGTGTTACTAATCCTTATGACA
GCAAGAACTGTGTATGATGATGGTGCTAGGAGAGTGTGGACACTTATGAATGTCT
TGACACTCGTTTATAAAGTTTATTATGGTAATGCTTTAGATCAAGCCATTTCCATGT
GGGCTCTTATAATCTCTGTTACTTCTAACTACTCAGGTGTAGTTACAACTGTCATGT
TTTTGGCCAGAGGTATTGTTTTTATGTGTGTTGAGTATTGCCCTATTTTCTTCATAA
CTGGTAATACACTTCAGTGTATAATGCTAGTTTATTGTTTCTTAGGCTATTTTTGTA
CTTGTTACTTTGGCCTCTTTTGTTTACTCAACCGCTACTTTAGACTGACTCTTGGTG
TTTATGATTACTTAGTTTCTACACAGGAGTTTAGATATATGAATTCACAGGGACTA
CTCCCACCCAAGAATAGCATAGATGCCTTCAAACTCAACATTAAATTGTTGGGTGT
TGGTGGCAAACCTTGTATCAAAGTAGCCACTGTACAGTCTAAAATGTCAGATGTA
AAGTGCACATCAGTAGTCTTACTCTCAGTTTTGCAACAACTCAGAGTAGAATCATC
ATCTAAATTGTGGGCTCAATGTGTCCAGTTACACAATGACATTCTCTTAGCTAAAG
ATACTACTGAAGCCTTTGAAAAAATGGTTTCACTACTTTCTGTTTTGCTTTCCATGC
AGGGTGCTGTAGACATAAACAAGCTTTGTGAAGAAATGCTGGACAACAGGGCAAC
CTTACAAGCTATAGCCTCAGAGTTTAGTTCCCTTCCATCATATGCAGCTTTTGCTAC
TGCTCAAGAAGCTTATGAGCAGGCTGTTGCTAATGGTGATTCTGAAGTTGTTCTTA
AAAAGTTGAAGAAGTCTTTGAATGTGGCTAAATCTGAATTTGACCGTGATGCAGC
CATGCAACGTAAGTTGGAAAAGATGGCTGATCAAGCTATGACCCAAATGTATAAA
CAGGCTAGATCTGAGGACAAGAGGGCAAAAGTTACTAGTGCTATGCAGACAATGC
TTTTCACTATGCTTAGAAAGTTGGATAATGATGCACTCAACAACATTATCAACAAT
GCAAGAGATGGTTGTGTTCCCTTGAACATAATACCTCTTACAACAGCAGCCAAACT
AATGGTTGTCATACCAGACTATAACACATATAAAAATACGTGTGATGGTACAACA
TTTACTTATGCATCAGCATTGTGGGAAATCCAACAGGTTGTAGATGCAGATAGTAA
AATTGTTCAACTTAGTGAAATTAGTATGGACAATTCACCTAATTTAGCATGGCCTC
TTATTGTAACAGCTTTAAGGGCCAATTCTGCTGTCAAATTACAGAATAATGAGCTT
AGTCCTGTTGCACTACGACAGATGTCTTGTGCTGCCGGTACTACACAAACTGCTTG
CACTGATGACAATGCGTTAGCTTACTACAACACAACAAAGGGAGGTAGGTTTGTA
CTTGCACTGTTATCCGATTTACAGGATTTGAAATGGGCTAGATTCCCTAAGAGTGA
TGGAACTGGTACTATCTATACAGAACTGGAACCACCTTGTAGGTTTGTTACAGACA
CACCTAAAGGTCCTAAAGTGAAGTATTTATACTTTATTAAAGGATTAAACAACCTA
AATAGAGGTATGGTACTTGGTAGTTTAGCTGCCACAGTACGTCTACAAGCTGGTAA
TGCAACAGAAGTGCCTGCCAATTCAACTGTATTATCTTTCTGTGCTTTTGCTGTAGA
TGCTGCTAAAGCTTACAAAGATTATCTAGCTAGTGGGGGACAACCAATCACTAATT
GTGTTAAGATGTTGTGTACACACACTGGTACTGGTCAGGCAATAACAGTTACACCG
GAAGCCAATATGGATCAAGAATCCTTTGGTGGTGCATCGTGTTGTCTGTACTGCCG
TTGCCACATAGATCATCCAAATCCTAAAGGATTTTGTGACTTAAAAGGTAAGTATG
TACAAATACCTACAACTTGTGCTAATGACCCTGTGGGTTTTACACTTAAAAACACA
GTCTGTACCGTCTGCGGTATGTGGAAAGGTTATGGCTGTAGTTGTGATCAACTCCG
CGAACCCATGCTTCAGTCAGCTGATGCACAATCGTTTTTAAACGGGTTTGCGGTGT
AAGTGCAGCCCGTCTTACACCGTGCGGCACAGGCACTAGTACTGATGTCGTATAC
AGGGCTTTTGACATCTACAATGATAAAGTAGCTGGTTTTGCTAAATTCCTAAAAAC
TAATTGTTGTCGCTTCCAAGAAAAGGACGAAGATGACAATTTAATTGATTCTTACT
TTGTAGTTAAGAGACACACTTTCTCTAACTACCAACATGAAGAAACAATTTATAAT
TTACTTAAGGATTGTCCAGCTGTTGCTAAACATGACTTCTTTAAGTTTAGAATAGA
CGGTGACATGGTACCACATATATCACGTCAACGTCTTACTAAATACACAATGGCA
GACCTCGTCTATGCTTTAAGGCATTTTGATGAAGGTAATTGTGACACATTAAAAGA
AATACTTGTCACATACAATTGTTGTGATGATGATTATTTCAATAAAAAGGACTGGT
ATGATTTTGTAGAAAACCCAGATATATTACGCGTATACGCCAACTTAGGTGAACGT
GTACGCCAAGCTTTGTTAAAAACAGTACAATTCTGTGATGCCATGCGAAATGCTGG
TATTGTTGGTGTACTGACATTAGATAATCAAGATCTCAATGGTAACTGGTATGATT
TCGGTGATTTCATACAAACCACGCCAGGTAGTGGAGTTCCTGTTGTAGATTCTTAT
TATTCATTGTTAATGCCTATATTAACCTTGACCAGGGCTTTAACTGCAGAGTCACA
TGTTGACACTGACTTAACAAAGCCTTACATTAAGTGGGATTTGTTAAAATATGACT
TCACGGAAGAGAGGTTAAAACTCTTTGACCGTTATTTTAAATATTGGGATCAGACA
TACCACCCAAATTGTGTTAACTGTTTGGATGACAGATGCATTCTGCATTGTGCAAA
CTTTAATGTTTTATTCTCTACAGTGTTCCCACCTACAAGTTTTGGACCACTAGTGAG
AAAAATATTTGTTGATGGTGTTCCATTTGTAGTTTCAACTGGATACCACTTCAGAG
AGCTAGGTGTTGTACATAATCAGGATGTAAACTTACATAGCTCTAGACTTAGTTTT
AAGGAATTACTTGTGTATGCTGCTGACCCTGCTATGCACGCTGCTTCTGGTAATCT
ATTACTAGATAAACGCACTACGTGCTTTTCAGTAGCTGCACTTACTAACAATGTTG
CTTTTCAAACTGTCAAACCCGGTAATTTTAACAAAGACTTCTATGACTTTGCTGTGT
CTAAGGGTTTCTTTAAGGAAGGAAGTTCTGTTGAATTAAAACACTTCTTCTTTGCT
CAGGATGGTAATGCTGCTATCAGCGATTATGACTACTATCGTTATAATCTACCAAC
AATGTGTGATATCAGACAACTACTATTTGTAGTTGAAGTTGTTGATAAGTACTTTG
ATTGTTACGATGGTGGCTGTATTAATGCTAACCAAGTCATCGTCAACAACCTAGAC
AAATCAGCTGGTTTTCCATTTAATAAATGGGGTAAGGCTAGACTTTATTATGATTC
AATGAGTTATGAGGATCAAGATGCACTTTTCGCATATACAAAACGTAATGTCATCC
CTACTATAACTCAAATGAATCTTAAGTATGCCATTAGTGCAAAGAATAGAGCTCGC
ACCGTAGCTGGTGTCTCTATCTGTAGTACTATGACCAATAGACAGTTTCATCAAAA
ATTATTGAAATCAATAGCCGCCACTAGAGGAGCTACTGTAGTAATTGGAACAAGC
AAATTCTATGGTGGTTGGCACAACATGTTAAAAACTGTTTATAGTGATGTAGAAAA
CCCTCACCTTATGGGTTGGGATTATCCTAAATGTGATAGAGCCATGCCTAACATGC
TTAGAATTATGGCCTCACTTGTTCTTGCTCGCAAACATACAACGTGTTGTAGCTTGT
CACACCGTTTCTATAGATTAGCTAATGAGTGTGCTCAAGTATTGAGTGAAATGGTC
ATGTGTGGCGGTTCACTATATGTTAAACCAGGTGGAACCTCATCAGGAGATGCCA
CAACTGCTTATGCTAATAGTGTTTTTAACATTTGTCAAGCTGTCACGGCCAATGTT
AATGCACTTTTATCTACTGATGGTAACAAAATTGCCGATAAGTATGTCCGCAATTT
ACAACACAGACTTTATGAGTGTCTCTATAGAAATAGAGATGTTGACACAGACTTTG
TGAATGAGTTTTACGCATATTTGCGTAAACATTTCTCAATGATGATACTCTCTGAC
GATGCTGTTGTGTGTTTCAATAGCACTTATGCATCTCAAGGTCTAGTGGCTAGCAT
AAAGAACTTTAAGTCAGTTCTTTATTATCAAAACAATGTTTTTATGTCTGAAGCAA
AATGTTGGACTGAGACTGACCTTACTAAAGGACCTCATGAATTTTGCTCTCAACAT
ACAATGCTAGTTAAACAGGGTGATGATTATGTGTACCTTCCTTACCCAGATCCATC
AAGAATCCTAGGGGCCGGCTGTTTTGTAGATGATATCGTAAAAACAGATGGTACA
CTTATGATTGAACGGTTCGTGTCTTTAGCTATAGATGCTTACCCACTTACTAAACAT
CCTAATCAGGAGTATGCTGATGTCTTTCATTTGTACTTACAATACATAAGAAAGCT
ACATGATGAGTTAACAGGACACATGTTAGACATGTATTCTGTTATGCTTACTAATG
ATAACACTTCAAGGTATTGGGAACCTGAGTTTTATGAGGCTATGTACACACCGCAT
ACAGTCTTACAGGCTGTTGGGGCTTGTGTTCTTTGCAATTCACAGACTTCATTAAG
ATGTGGTGCTTGCATACGTAGACCATTCTTATGTTGTAAATGCTGTTACGACCATG
TCATATCAACATCACATAAATTAGTCTTGTCTGTTAATCCGTATGTTTGCAATGCTC
CAGGTTGTGATGTCACAGATGTGACTCAACTTTACTTAGGAGGTATGAGCTATTAT
TGTAAATCACATAAACCACCCATTAGTTTTCCATTGTGTGCTAATGGACAAGTTTT
TGGTTTATATAAAAATACATGTGTTGGTAGCGATAATGTTACTGACTTTAATGCAA
TTGCAACATGTGACTGGACAAATGCTGGTGATTACATTTTAGCTAACACCTGTACT
GAAAGACTCAAGCTTTTTGCAGCAGAAACGCTCAAAGCTACTGAGGAGACATTTA
AACTGTCTTATGGTATTGCTACTGTACGTGAAGTGCTGTCTGACAGAGAATTACAT
CTTTCATGGGAAGTTGGTAAACCTAGACCACCACTTAACCGAAATTATGTCTTTAC
TGGTTATCGTGTAACTAAAAACAGTAAAGTACAAATAGGAGAGTACACCTTTGAA
AAAGGTGACTATGGTGATGCTGTTGTTTACCGAGGTACAACAACTTACAAATTAA
ATGTTGGTGATTATTTTGTGCTGACATCACATACAGTAATGCCATTAAGTGCACCT
ACACTAGTGCCACAAGAGCACTATGTTAGAATTACTGGCTTATACCCAACACTCAA
TATCTCAGATGAGTTTTCTAGCAATGTTGCAAATTATCAAAAGGTTGGTATGCAAA
AGTATTCTACACTCCAGGGACCACCTGGTACTGGTAAGAGTCATTTTGCTATTGGC
CTAGCTCTCTACTACCCTTCTGCTCGCATAGTGTATACAGCTTGCTCTCATGCCGCT
GTTGATGCACTATGTGAGAAGGCATTAAAATATTTGCCTATAGATAAATGTAGTAG
AATTATACCTGCACGTGCTCGTGTAGAGTGTTTTGATAAATTCAAAGTGAATTCAA
CATTAGAACAGTATGTCTTTTGTACTGTAAATGCATTGCCTGAGACGACAGCAGAT
ATAGTTGTCTTTGATGAAATTTCAATGGCCACAAATTATGATTTGAGTGTTGTCAA
TGCCAGATTACGTGCTAAGCACTATGTGTACATTGGCGACCCTGCTCAATTACCTG
CACCACGCACATTGCTAACTAAGGGCACACTAGAACCAGAATATTTCAATTCAGT
GTGTAGACTTATGAAAACTATAGGTCCAGACATGTTCCTCGGAACTTGTCGGCGTT
GTCCTGCTGAAATTGTTGACACTGTGAGTGCTTTGGTTTATGATAATAAGCTTAAA
GCACATAAAGACAAATCAGCTCAATGCTTTAAAATGTTTTATAAGGGTGTTATCAC
GCATGATGTTTCATCTGCAATTAACAGGCCACAAATAGGCGTGGTAAGAGAATTC
CTTACACGTAACCCTGCTTGGAGAAAAGCTGTCTTTATTTCACCTTATAATTCACA
GAATGCTGTAGCCTCAAAGATTTTGGGACTACCAACTCAAACTGTTGATTCATCAC
AGGGCTCAGAATATGACTATGTCATATTCACTCAAACCACTGAAACAGCTCACTCT
TGTAATGTAAACAGATTTAATGTTGCTATTACCAGAGCAAAAGTAGGCATACTTTG
CATAATGTCTGATAGAGACCTTTATGACAAGTTGCAATTTACAAGTCTTGAAATTC
CACGTAGGAATGTGGCAACTTTACAAGCTGAAAATGTAACAGGACTCTTTAAAGA
TTGTAGTAAGGTAATCACTGGGTTACATCCTACACAGGCACCTACACACCTCAGTG
TTGACACTAAATTCAAAACTGAAGGTTTATGTGTTGACATACCTGGCATACCTAAG
GACATGACCTATAGAAGACTCATCTCTATGATGGGTTTTAAAATGAATTATCAAGT
TAATGGTTACCCTAACATGTTTATCACCCGCGAAGAAGCTATAAGACATGTACGTG
CATGGATTGGCTTCGATGTCGAGGGGTGTCATGCTACTAGAGAAGCTGTTGGTACC
AATTTACCTTTACAGCTAGGTTTTTCTACAGGTGTTAACCTAGTTGCTGTACCTACA
GGTTATGTTGATACACCTAATAATACAGATTTTTCCAGAGTTAGTGCTAAACCACC
GCCTGGAGATCAATTTAAACACCTCATACCACTTATGTACAAAGGACTTCCTTGGA
ATGTAGTGCGTATAAAGATTGTACAAATGTTAAGTGACACACTTAAAAATCTCTCT
GACAGAGTCGTATTTGTCTTATGGGCACATGGCTTTGAGTTGACATCTATGAAGTA
TTTTGTGAAAATAGGACCTGAGCGCACCTGTTGTCTATGTGATAGACGTGCCACAT
GCTTTTCCACTGCTTCAGACACTTATGCCTGTTGGCATCATTCTATTGGATTTGATT
ACGTCTATAATCCGTTTATGATTGATGTTCAACAATGGGGTTTTACAGGTAACCTA
CAAAGCAACCATGATCTGTATTGTCAAGTCCATGGTAATGCACATGTAGCTAGTTG
TGATGCAATCATGACTAGGTGTCTAGCTGTCCACGAGTGCTTTGTTAAGCGTGTTG
ACTGGACTATTGAATATCCTATAATTGGTGATGAACTGAAGATTAATGCGGCTTGT
AGAAAGGTTCAACACATGGTTGTTAAAGCTGCATTATTAGCAGACAAATTCCCAG
TTCTTCACGACATTGGTAACCCTAAAGCTATTAAGTGTGTACCTCAAGCTGATGTA
GAATGGAAGTTCTATGATGCACAGCCTTGTAGTGACAAAGCTTATAAAATAGAAG
AATTATTCTATTCTTATGCCACACATTCTGACAAATTCACAGATGGTGTATGCCTAT
TTTGGAATTGCAATGTCGATAGATATCCTGCTAATTCCATTGTTTGTAGATTTGACA
CTAGAGTGCTATCTAACCTTAACTTGCCTGGTTGTGATGGTGGCAGTTTGTATGTA
AATAAACATGCATTCCACACACCAGCTTTTGATAAAAGTGCTTTTGTTAATTTAAA
ACAATTACCATTTTTCTATTACTCTGACAGTCCATGTGAGTCTCATGGAAAACAAG
TAGTGTCAGATATAGATTATGTACCACTAAAGTCTGCTACGTGTATAACACGTTGC
AATTTAGGTGGTGCTGTCTGTAGACATCATGCTAATGAGTACAGATTGTATCTCGA
TGCTTATAACATGATGATCTCAGCTGGCTTTAGCTTGTGGGTTTACAAACAATTTG
ATACTTATAACCTCTGGAACACTTTTACAAGACTTCAGAGTTTAGAAAATGTGGCT
TTTAATGTTGTAAATAAGGGACACTTTGATGGACAACAGGGTGAAGTACCAGTTTC
TATCATTAATAACACTGTTTACACAAAAGTTGATGGTGTTGATGTAGAATTGTTTG
AAAATAAAACAACATTACCTGTTAATGTAGCATTTGAGCTTTGGGCTAAGCGCAA
CATTAAACCAGTACCAGAGGTGAAAATACTCAATAATTTGGGTGTGGACATTGCT
GCTAATACTGTGATCTGGGACTACAAAAGAGATGCTCCAGCACATATATCTACTAT
TGGTGTTTGTTCTATGACTGACATAGCCAAGAAACCAACTGAAACGATTTGTGCAC
CACTCACTGTCTTTTTTGATGGTAGAGTTGATGGTCAAGTAGACTTATTTAGAAAT
GCCCGTAATGGTGTTCTTATTACAGAAGGTAGTGTTAAAGGTTTACAACCATCTGT
AGGTCCCAAACAAGCTAGTCTTAATGGAGTCACATTAATTGGAGAAGCCGTAAAA
ACACAGTTCAATTATTATAAGAAAGTTGATGGTGTTGTCCAACAATTACCTGAAAC
TTACTTTACTCAGAGTAGAAATTTACAAGAATTTAAACCCAGGAGTCAAATGGAA
ATTGATTTCTTAGAATTAGCTATGGATGAATTCATTGAACGGTATAAATTAGAAGG
CTATGCCTTCGAACATATCGTTTATGGAGATTTTAGTCATAGTCAGTTAGGTGGTTT
ACATCTACTGATTGGACTAGCTAAACGTTTTAAGGAATCACCTTTTGAATTAGAAG
ATTTTATTCCTATGGACAGTACAGTTAAAAACTATTTCATAACAGATGCGCAAACA
GGTTCATCTAAGTGTGTGTGTTCTGTTATTGATTTATTACTTGATGATTTTGTTGAA
ATAATAAAATCCCAAGATTTATCTGTAGTTTCTAAGGTTGTCAAAGTGACTATTGA
CTATACAGAAATTTCATTTATGCTTTGGTGTAAAGATGGCCATGTAGAAACATTTT
ACCCAAAATTACAATCTAGTCAAGCGTGGCAACCGGGTGTTGCTATGCCTAATCTT
TACAAAATGCAAAGAATGCTATTAGAAAAGTGTGACCTTCAAAATTATGGTGATA
GTGCAACATTACCTAAAGGCATAATGATGAATGTCGCAAAATATACTCAACTGTG
TCAATATTTAAACACATTAACATTAGCTGTACCCTATAATATGAGAGTTATACATT
TTGGTGCTGGTTCTGATAAAGGAGTTGCACCAGGTACAGCTGTTTTAAGACAGTGG
TTGCCTACGGGTACGCTGCTTGTCGATTCAGATCTTAATGACTTTGTCTCTGATGCA
GATTCAACTTTGATTGGTGATTGTGCAACTGTACATACAGCTAATAAATGGGATCT
CATTATTAGTGATATGTACGACCCTAAGACTAAAAATGTTACAAAAGAAAATGAC
TCTAAAGAGGGTTTTTTCACTTACATTTGTGGGTTTATACAACAAAAGCTAGCTCT
TGGAGGTTCCGTGGCTATAAAGATAACAGAACATTCTTGGAATGCTGATCTTTATA
AGCTCATGGGACACTTCGCATGGTGGACAGCCTTTGTTACTAATGTGAATGCGTCA
TCATCTGAAGCATTTTTAATTGGATGTAATTATCTTGGCAAACCACGCGAACAAAT
AGATGGTTATGTCATGCATGCAAATTACATATTTTGGAGGAATACAAATCCAATTC
AGTTGTCTTCCTATTCTTTATTTGACATGAGTAAATTTCCCCTTAAATTAAGGGGTA
CTGCTGTTATGTCTTTAAAAGAAGGTCAAATCAATGATATGATTTTATCTCTTCTTA
GTAAAGGTAGACTTATAATTAGAGAAAACAACAGAGTTGTTATTTCTAGTGATGTT
CTTGTTAACAACTAAGCGATCGCGCCACCATGGTGAGCAAGGGCGAGGAGCTGTT
CACCGGGGTGGTGCCCATCCTGGTCGAGCTGGACGGCGACGTAAACGGCCACAAG
TTCAGCGTGTCCGGCGAGGGCGAGGGCGATGCCACCTACGGCAAGCTGACCCTGA
AGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACG
ACTTCTTCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGACACC
CTGGTGAACCGCATCGAGCTGAAGGGCATCGACTTCAAGGAGGACGGCAACATCC
TGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGA
CGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAG
ACCCCAACGAGAAGCGCGATCACATGGTCCTGCTGGAGTTCGTGACCGCCGCCGG
GATCACTCTCGGCATGGACGAGCTGTACAAGTAGCCTGCAGGATGGGCTATATAA
ACGTTTTCGCTTTTCCGTTTACGATATATAGTCTACTCTTGTGCAGAATGAATTCTC
GTAACTACATAGCACAAGTAGATGTAGTTAACTTTAATCTCACATAGCAATCTTTA
ATCAGTGTGTAACATTAGGGAGGACTTGAAAGAGCCACCACATTTTCACCGAGGC
CACGCGGAGTACGATCGAGTGTACAGTGAACAATGCTAGGGAGAGCTGCCTATAT
GGAAGAGCCCTAATGTGTAAAATTAATTTTAGTAGTGCTATCCCCATGTGATTTTA
ATAGCTTCTTAGGAGAATGACAAAAAAAAAAAAAAAAAAAAAAAAAAAAAAAAA
GCGGCCGCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTCTTTTTC
CGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCTC
TGCTAATCCTGTTACCAGTGGCTGCTGCCAGTGGCGATAAGTCGTGTCTTACCGGG
TTGGACTCAAGACGATAGTTACCGGATAAGGCGCAGCGGTCGGGCTGAACGGGGG
GTTCGTGCACACAGCCCAGCTTGGAGCGAACGACCTACACCGAACTGAGATACCT
ACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAACGCGGAACCCC
TATTTGTTTATTTTTCTAAATACATTCAAATATGTATCCGCTCATGAGACAATAACC
CTGATAAATGCTTCAATAATATTGAAAAAGGAAGAGTATGATTGAACAAGATGGA
TTGCACGCAGGTTCTCCGGCCGCTTGGGTGGAGAGGCTATTCGGCTATGACTGGGC
ACAACAGACAATCGGCTGCTCTGATGCCGCCGTGTTCCGGCTGTCAGCGCAGGGG
CGCCCGGTTCTTTTTGTCAAGACCGACCTGTCCGGTGCCCTGAATGAACTGCAGGA
CGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGG
GGCAGGATCTCCTGTCATCTCACCTTGCTCCTGCCGAGAAAGTATCCATCATGGCT
GATGCAATGCGGCGGCTGCATACGCTTGATCCGGCTACCTGCCCATTCGACCACCA
AGCGAAACATCGCATCGAGCGAGCACGTACTCGGATGGAAGCCGGTCTTGTCGAT
CAGGATGATCTGGACGAAGAGCATCAGGGGCTCGCGCCAGCCGAACTGTTCGCCA
GGCTCAAGGCGCGCATGCCCGACGGCGAGGATCTCGTCGTGACCCATGGCGATGC
CTGCTTGCCGAATATCATGGTGGAAAATGGCCGCTTTTCTGGATTCATCGACTGTG
GCCGGCTGGGTGTGGCGGACCGCTATCAGGACATAGCGTTGGCTACCCGTGATAT
TGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGAA
TTGAAAAAGGAAGAGTAACGCCAGCAACGCGAGCTCGACATTGATTATTGACTAG
TTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAA
GTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCT
ACGTATTAGTCATCGCTATTACCATGGTCGAGGTGAGCCCCACGTTCTGCTTCACT
CTCCCCATCTCCCCCCCCTCCCCACCCCCAATTTTGTATTTATTTATTTTTTAATTAT
TTTGTGCAGCGATGGGGGCGGGGGGGGGGGGGGCGCGCGCCAGGGGGGGGGGGG
GGGGGGGGGGGGGGGGGGGGGGGGGGGGGGGGGGGGGCGGCGGCAGCCAATCA
GAGCGGCGCGCTCCGAAAGTTTCCTTTTATGGCGAGGCGGCGGCGGCGGCGGCCC
TATAAAAAGCGAAGCGCGCGGCGGGCGGGAGTCGCTGCGCGCTGCCTTCGCCCCG
TGCCCCGCTCCGCCGCCGCCTCGCGCCGCCCGCCCCGGCTCTGACTGACCGCGTTA
CTCCCACAGGTGAGCGGGCGGGACGGCCCTTCTCCTCCGGGCTGTAATTAGCGCTT
GGTTTAATGACGGCTTGTTTCTTTTCTGTGGCTGCGTGAAAGCCTTGAGGGGCTCC
GGGAGGGCCCTTTGTGCGGGGGGAGCGGCTCGGGGGGTGCGTGCGTGTGTGTGTG
CGTGGGGAGCGCCGCGTGCGGCTCCGCGCTGCCCGGCGGCTGTGAGCGCTGCGGG
CGCGGCGCGGGGCTTTGTGCGCTCCGCAGTGTGCGCGAGGGGAGCGCGGCCGGGG
GCGGTGCCCCGCGGTGCGGGGGGGGCTGCGAGGGGAACAAAGGCTGCGTGCGGG
GTGTGTGCGTGGGGGGGTGAGCAGGGGGTGTGGGCGCGTCGGTCGGGCTGCAACC
CCCCCTGCACCCCCCTCCCCGAGTTGCTGAGCACGGCCCGGCTTCGGGTGCGGGGC
TCCGTACGGGGCGTGGCGCGGGGCTCGCCGTGCCGGGCGGGGGGTGGCGGCAGGT
GGGGGTGCCGGGCGGGGCGGGGCCGCCTCGGGCCGGGGGGGGCTCGGGGGGGGG
GCGCGGCGGCCCCCGGAGCGCCGGCGGCTGTCGAGGCGCGGCGAGCCGCAGCCA
TTGCCTTTTATGGTAATCGTGCGAGAGGGCGCAGGGACTTCCTTTGTCCCAAATCT
GTGCGGAGCCGAAATCTGGGAGGCGCCGCCGCACCCCCTCTAGCGGGCGCGGGGC
GAAGCGGTGCGGCGCCGGCAGGAAGGAAATGGGCGGGGAGGGCCTTCGTGCGTC
GCCGCGCCGCCGTCCCCTTCTCCCTCTCCAGCCTCGGGGCTGTCCGCGGGGGGACG
GCTGCCTTCGGGGGGGACGGGGCAGGGCGGGGTTCGGCTTCTGGCGTGTGACCGG
CGGCTTCAGAGCCTCTGCTAACCATGTTCATGCCTTCTTCTTTTTCCTACAGGGCCG
GCCCGTTTAGTGAACCGTCAGAATTTTGTAATACGACTCACTATAGGG

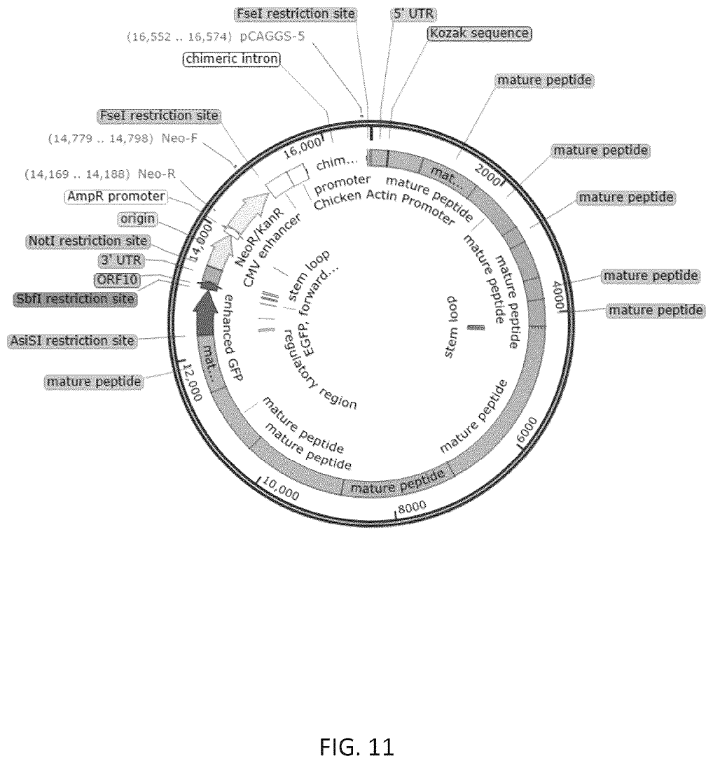

CBA + T7-RedCovid-GFP (FIG. 11)

| | |
|---|---|
| source | 1 . . . 16,681 |
| 5′ UTR | 1 . . . 265 |
| Kozak sequence | 266 . . . 271 |
| mature peptide | 272 . . . 811 |
| mature peptide | 824 . . . 1741 |
| mature peptide | 1742 . . . 2611 |
| mature peptide | 2612 . . . 2860 |
| mature peptide | 2861 . . . 3454 |
| mature peptide | 3455 . . . 3793 |
| mature peptide | 3794 . . . 4210 |
| mature peptide | 4211 . . . 7005 |
| mature peptide | 4211 . . . 4249 |
| stem loop | 4245 . . . 4272 |
| stem loop | 4257 . . . 4311 |
| mature peptide | 7006 . . . 8808 |
| mature peptide | 8809 . . . 10,389 |
| mature peptide | 10,390 . . . 11,427 |
| mature peptide | 11,428 . . . 12,321 |
| AsiSI restriction site | 12,325 . . . 12,332 |
| regulatory region | 12,333 . . . 12,342 |
| enhanced GFP | 12,339 . . . 13,058 |
| EGFP, reverse primer | 12,384 . . . 12,405 |
| For distinguishing EGFP variants, reverse primer | 12,645 . . . 12,664 |
| EGFP, forward primer | 12,992 . . . 13,013 |
| SbfI restriction site | 13,059 . . . 13,066 |
| source | 13,059 . . . 13,066 |
| source | 13,059 . . . 13,066 |
| ORF10 | 13,067 . . . 13,183 |
| ORF10 | 13,067 . . . 13,183 |
| stem loop | 13,118 . . . 13,153 |
| stem loop | 13,138 . . . 13,166 |
| 3′ UTR | 13,184 . . . 13,412 |
| stem loop | 13,237 . . . 13,277 |
| NotI restriction site | 13,413 . . . 13,420 |
| source | 13,413 . . . 13,420 |
| origin | 13,421 . . . 14,009 |
| pBR322 origin, forward primer | 13,910 . . . 13,929 |
| AmpR promoter | 14,010 . . . 14,114 |
| NeoR/KanR | 14,115 . . . 14,909 |
| Fsel restriction site | 14,947 . . . 14,954 |
| source | 14,947 . . . 14,954 |
| source | 14,947 . . . 14,954 |
| CMV enhancer | 14,955 . . . 15,334 |
| Chicken Beta Actin Promoter | 15,335 . . . 15,611 |
| chimeric intron | 15,612 . . . 16,628 |
| FseI restriction site | 16,629 . . . 16,636 |
| source | 16,629 . . . 16,636 |
| source | 16,629 . . . 16,636 |
| promoter | 16,663 . . . 16,681 |

ATTAAAGGTTTATACCTTCCCAGGTAACAAACCAACCAACTTTCGATCTCTTGTAG
ATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGT
AACTCGTCTATCTTCTGCAGGCTGCTTACGGTTTCGTCCGTGTTGCAGCCGATCATC
AGCACATCTAGGTTTCGTCCGGGTGTGACCGAAAGGTAAGGCCACCATGGAGAGC
CTTGTCCCTGGTTTCAACGAGAAAACACACGTCCAACTCAGTTTGCCTGTTTTACA
GGTTCGCGACGTGCTCGTACGTGGCTTTGGAGACTCCGTGGAGGAGGTCTTATCAG
AGGCACGTCAACATCTTAAAGATGGCACTTGTGGCTTAGTAGAAGTTGAAAAAGG
CGTTTTGCCTCAACTTGAACAGCCCTATGTGTTCATCAAACGTTCGGATGCTCGAA
CTGCACCTCATGGTCATGTTATGGTTGAGCTGGTAGCAGAACTCGAAGGCATTCAG
TACGGTCGTAGTGGTGAGACACTTGGTGTCCTTGTCCCTCATGTGGGCGAAATACC
AGTGGCTTACCGCAAGGTTCTTCTTCGTAAGAACGGTAATAAAGGAGCTGGTGGC
CATAGTTACGGCGCCGATCTAAAGTCATTTGACTTAGGCGACGAGCTTGGCACTGA
TCCTTATGAAGATTTTCAAGAAAACTGGAACACTAAACATAGCAGTGGTGTTACCC
GTGAACTCATGCGTGAGCTTAACGGAGGGGCAACTTTACAAAGTGGTTTTAGAAA
AATGGCATTCCCATCTGGTAAAGTTGAGGGTTGTATGGTACAAGTAACTTGTGGTA
CAACTACACTTAACGGTCTTTGGCTTGATGACGTAGTTTACTGTCCAAGACATGTG
ATCTGCACCTCTGAAGACATGCTTAACCCTAATTATGAAGATTTACTCATTCGTAA
GTCTAATCATAATTTCTTGGTACAGGCTGGTAATGTTCAACTCAGGGTTATTGGAC
ATTCTATGCAAAATTGTGTACTTAAGCTTAAGGTTGATACAGCCAATCCTAAGACA
CCTAAGTATAAGTTTGTTCGCATTCAACCAGGACAGACTTTTTCAGTGTTAGCTTG
TTACAATGGTTCACCATCTGGTGTTTACCAATGTGCTATGAGGCCCAATTTCACTA
TTAAGGGTTCATTCCTTAATGGTTCATGTGGTAGTGTTGGTTTTAACATAGATTATG
ACTGTGTCTCTTTTTGTTACATGCACCATATGGAATTACCAACTGGAGTTCATGCTG
GCACAGACTTAGAAGGTAACTTTTATGGACCTTTTGTTGACAGGCAAACAGCACA
AGCAGCTGGTACGGACACAACTATTACAGTTAATGTTTTAGCTTGGTTGTACGCTG
CTGTTATAAATGGAGACAGGTGGTTTCTCAATCGATTTACCACAACTCTTAATGAC
TTTAACCTTGTGGCTATGAAGTACAATTATGAACCTCTAACACAAGACCATGTTGA
CATACTAGGACCTCTTTCTGCTCAAACTGGAATTGCCGTTTTAGATATGTGTGCTTC
ATTAAAAGAATTACTGCAAAATGGTATGAATGGACGTACCATATTGGGTAGTGCT
TTATTAGAAGATGAATTTACACCTTTTGATGTTGTTAGACAATGCTCAGGTGTTAC
TTTCCAAAGTGCAGTGAAAAGAACAATCAAGGGTACACACCACTGGTTGTTACTC
ACAATTTTGACTTCACTTTTAGTTTTAGTCCAGAGTACTCAATGGTCTTTGTTCTTT
TTTTTGTATGAAAATGCCTTTTTACCTTTTGCTATGGGTATTATTGCTATGTCTGCTT
TTGCAATGATGTTTGTCAAACATAAGCATGCATTTCTCTGTTTGTTTTTGTTACCTT
CTCTTGCCACTGTAGCTTATTTTAATATGGTCTATATGCCTGCTAGTTGGGTGATGC
GTATTATGACATGGTTGGATATGGTTGATACTAGTTTGTCTGGTTTTAAGCTAAAA
GACTGTGTTATGTATGCATCAGCTGTAGTGTTACTAATCCTTATGACAGCAAGAAC
TGTGTATGATGATGGTGCTAGGAGAGTGTGGACACTTATGAATGTCTTGACACTCG
TTTATAAAGTTTATTATGGTAATGCTTTAGATCAAGCCATTTCCATGTGGGCTCTTA
TAATCTCTGTTACTTCTAACTACTCAGGTGTAGTTACAACTGTCATGTTTTTGGCCA
GAGGTATTGTTTTTATGTGTGTTGAGTATTGCCCTATTTTCTTCATAACTGGTAATA
CACTTCAGTGTATAATGCTAGTTTATTGTTTCTTAGGCTATTTTTGTACTTGTTACTT
TGGCCTCTTTTGTTTACTCAACCGCTACTTTAGACTGACTCTTGGTGTTTATGATTA
CTTAGTTTCTACACAGGAGTTTAGATATATGAATTCACAGGGACTACTCCCACCCA
AGAATAGCATAGATGCCTTCAAACTCAACATTAAATTGTTGGGTGTTGGTGGCAA
ACCTTGTATCAAAGTAGCCACTGTACAGTCTAAAATGTCAGATGTAAAGTGCACAT
CAGTAGTCTTACTCTCAGTTTTGCAACAACTCAGAGTAGAATCATCATCTAAATTG
TGGGCTCAATGTGTCCAGTTACACAATGACATTCTCTTAGCTAAAGATACTACTGA
AGCCTTTGAAAAAATGGTTTCACTACTTTCTGTTTTGCTTTCCATGCAGGGTGCTGT
AGACATAAACAAGCTTTGTGAAGAAATGCTGGACAACAGGGCAACCTTACAAGCT
ATAGCCTCAGAGTTTAGTTCCCTTCCATCATATGCAGCTTTTGCTACTGCTCAAGA
AGCTTATGAGCAGGCTGTTGCTAATGGTGATTCTGAAGTTGTTCTTAAAAAGTTGA
AGAAGTCTTTGAATGTGGCTAAATCTGAATTTGACCGTGATGCAGCCATGCAACGT
AAGTTGGAAAAGATGGCTGATCAAGCTATGACCCAAATGTATAAACAGGCTAGAT
CTGAGGACAAGAGGGCAAAAGTTACTAGTGCTATGCAGACAATGCTTTTCACTAT
GCTTAGAAAGTTGGATAATGATGCACTCAACAACATTATCAACAATGCAAGAGAT
GGTTGTGTTCCCTTGAACATAATACCTCTTACAACAGCAGCCAAACTAATGGTTGT
CATACCAGACTATAACACATATAAAAATACGTGTGATGGTACAACATTTACTTATG
CATCAGCATTGTGGGAAATCCAACAGGTTGTAGATGCAGATAGTAAAATTGTTCA
ACTTAGTGAAATTAGTATGGACAATTCACCTAATTTAGCATGGCCTCTTATTGTAA
CAGCTTTAAGGGCCAATTCTGCTGTCAAATTACAGAATAATGAGCTTAGTCCTGTT
GCACTACGACAGATGTCTTGTGCTGCCGGTACTACACAAACTGCTTGCACTGATGA
CAATGCGTTAGCTTACTACAACACAACAAAGGGAGGTAGGTTTGTACTTGCACTGT
TATCCGATTTACAGGATTTGAAATGGGCTAGATTCCCTAAGAGTGATGGAACTGGT
ACTATCTATACAGAACTGGAACCACCTTGTAGGTTTGTTACAGACACACCTAAAGG
TCCTAAAGTGAAGTATTTATACTTTATTAAAGGATTAAACAACCTAAATAGAGGTA
TGGTACTTGGTAGTTTAGCTGCCACAGTACGTCTACAAGCTGGTAATGCAACAGAA
GTGCCTGCCAATTCAACTGTATTATCTTTCTGTGCTTTTGCTGTAGATGCTGCTAAA
GCTTACAAAGATTATCTAGCTAGTGGGGGACAACCAATCACTAATTGTGTTAAGAT
GTTGTGTACACACACTGGTACTGGTCAGGCAATAACAGTTACACCGGAAGCCAAT
ATGGATCAAGAATCCTTTGGTGGTGCATCGTGTTGTCTGTACTGCCGTTGCCACAT
AGATCATCCAAATCCTAAAGGATTTTGTGACTTAAAAGGTAAGTATGTACAAATA
CCTACAACTTGTGCTAATGACCCTGTGGGTTTTACACTTAAAAACACAGTCTGTAC
CGTCTGCGGTATGTGGAAAGGTTATGGCTGTAGTTGTGATCAACTCCGCGAACCCA
TGCTTCAGTCAGCTGATGCACAATCGTTTTTAAACGGGTTTGCGGTGTAAGTGCAG
CCCGTCTTACACCGTGCGGCACAGGCACTAGTACTGATGTCGTATACAGGGCTTTT
GACATCTACAATGATAAAGTAGCTGGTTTTGCTAAATTCCTAAAAACTAATTGTTG
TCGCTTCCAAGAAAAGGACGAAGATGACAATTTAATTGATTCTTACTTTGTAGTTA
AGAGACACACTTTCTCTAACTACCAACATGAAGAAACAATTTATAATTTACTTAAG
GATTGTCCAGCTGTTGCTAAACATGACTTCTTTAAGTTTAGAATAGACGGTGACAT
GGTACCACATATATCACGTCAACGTCTTACTAAATACACAATGGCAGACCTCGTCT
ATGCTTTAAGGCATTTTGATGAAGGTAATTGTGACACATTAAAAGAAATACTTGTC
ACATACAATTGTTGTGATGATGATTATTTCAATAAAAAGGACTGGTATGATTTTGT
AGAAAACCCAGATATATTACGCGTATACGCCAACTTAGGTGAACGTGTACGCCAA
GCTTTGTTAAAAACAGTACAATTCTGTGATGCCATGCGAAATGCTGGTATTGTTGG
TGTACTGACATTAGATAATCAAGATCTCAATGGTAACTGGTATGATTTCGGTGATT
TCATACAAACCACGCCAGGTAGTGGAGTTCCTGTTGTAGATTCTTATTATTCATTG
TTAATGCCTATATTAACCTTGACCAGGGCTTTAACTGCAGAGTCACATGTTGACAC
TGACTTAACAAAGCCTTACATTAAGTGGGATTTGTTAAAATATGACTTCACGGAAG
AGAGGTTAAAACTCTTTGACCGTTATTTTAAATATTGGGATCAGACATACCACCCA
AATTGTGTTAACTGTTTGGATGACAGATGCATTCTGCATTGTGCAAACTTTAATGT
TTTATTCTCTACAGTGTTCCCACCTACAAGTTTTGGACCACTAGTGAGAAAAATAT
TTGTTGATGGTGTTCCATTTGTAGTTTCAACTGGATACCACTTCAGAGAGCTAGGT
GTTGTACATAATCAGGATGTAAACTTACATAGCTCTAGACTTAGTTTTAAGGAATT
ACTTGTGTATGCTGCTGACCCTGCTATGCACGCTGCTTCTGGTAATCTATTACTAGA
TAAACGCACTACGTGCTTTTCAGTAGCTGCACTTACTAACAATGTTGCTTTTCAAA
CTGTCAAACCCGGTAATTTTAACAAAGACTTCTATGACTTTGCTGTGTCTAAGGGT
TTCTTTAAGGAAGGAAGTTCTGTTGAATTAAAACACTTCTTCTTTGCTCAGGATGG
TAATGCTGCTATCAGCGATTATGACTACTATCGTTATAATCTACCAACAATGTGTG
ATATCAGACAACTACTATTTGTAGTTGAAGTTGTTGATAAGTACTTTGATTGTTAC
GATGGTGGCTGTATTAATGCTAACCAAGTCATCGTCAACAACCTAGACAAATCAG
CTGGTTTTCCATTTAATAAATGGGGTAAGGCTAGACTTTATTATGATTCAATGAGT
TATGAGGATCAAGATGCACTTTTCGCATATACAAAACGTAATGTCATCCCTACTAT
AACTCAAATGAATCTTAAGTATGCCATTAGTGCAAAGAATAGAGCTCGCACCGTA
GCTGGTGTCTCTATCTGTAGTACTATGACCAATAGACAGTTTCATCAAAAATTATT
GAAATCAATAGCCGCCACTAGAGGAGCTACTGTAGTAATTGGAACAAGCAAATTC
TATGGTGGTTGGCACAACATGTTAAAAACTGTTTATAGTGATGTAGAAAACCCTCA
CCTTATGGGTTGGGATTATCCTAAATGTGATAGAGCCATGCCTAACATGCTTAGAA
TTATGGCCTCACTTGTTCTTGCTCGCAAACATACAACGTGTTGTAGCTTGTCACACC
GTTTCTATAGATTAGCTAATGAGTGTGCTCAAGTATTGAGTGAAATGGTCATGTGT
GGCGGTTCACTATATGTTAAACCAGGTGGAACCTCATCAGGAGATGCCACAACTG
CTTATGCTAATAGTGTTTTTAACATTTGTCAAGCTGTCACGGCCAATGTTAATGCA
CTTTTATCTACTGATGGTAACAAAATTGCCGATAAGTATGTCCGCAATTTACAACA
CAGACTTTATGAGTGTCTCTATAGAAATAGAGATGTTGACACAGACTTTGTGAATG
AGTTTTACGCATATTTGCGTAAACATTTCTCAATGATGATACTCTCTGACGATGCT
GTTGTGTGTTTCAATAGCACTTATGCATCTCAAGGTCTAGTGGCTAGCATAAAGAA
CTTTAAGTCAGTTCTTTATTATCAAAACAATGTTTTTATGTCTGAAGCAAAATGTTG
GACTGAGACTGACCTTACTAAAGGACCTCATGAATTTTGCTCTCAACATACAATGC
TAGTTAAACAGGGTGATGATTATGTGTACCTTCCTTACCCAGATCCATCAAGAATC
CTAGGGGCCGGCTGTTTTGTAGATGATATCGTAAAAACAGATGGTACACTTATGAT
TGAACGGTTCGTGTCTTTAGCTATAGATGCTTACCCACTTACTAAACATCCTAATC
AGGAGTATGCTGATGTCTTTCATTTGTACTTACAATACATAAGAAAGCTACATGAT
GAGTTAACAGGACACATGTTAGACATGTATTCTGTTATGCTTACTAATGATAACAC
TTCAAGGTATTGGGAACCTGAGTTTTATGAGGCTATGTACACACCGCATACAGTCT
TACAGGCTGTTGGGGCTTGTGTTCTTTGCAATTCACAGACTTCATTAAGATGTGGT
GCTTGCATACGTAGACCATTCTTATGTTGTAAATGCTGTTACGACCATGTCATATC
AACATCACATAAATTAGTCTTGTCTGTTAATCCGTATGTTTGCAATGCTCCAGGTT
GTGATGTCACAGATGTGACTCAACTTTACTTAGGAGGTATGAGCTATTATTGTAAA
TCACATAAACCACCCATTAGTTTTCCATTGTGTGCTAATGGACAAGTTTTTGGTTTA
TATAAAAATACATGTGTTGGTAGCGATAATGTTACTGACTTTAATGCAATTGCAAC
ATGTGACTGGACAAATGCTGGTGATTACATTTTAGCTAACACCTGTACTGAAAGAC
TCAAGCTTTTTGCAGCAGAAACGCTCAAAGCTACTGAGGAGACATTTAAACTGTCT
TATGGTATTGCTACTGTACGTGAAGTGCTGTCTGACAGAGAATTACATCTTTCATG
GGAAGTTGGTAAACCTAGACCACCACTTAACCGAAATTATGTCTTTACTGGTTATC
GTGTAACTAAAAACAGTAAAGTACAAATAGGAGAGTACACCTTTGAAAAAGGTGA
CTATGGTGATGCTGTTGTTTACCGAGGTACAACAACTTACAAATTAAATGTTGGTG
ATTATTTTGTGCTGACATCACATACAGTAATGCCATTAAGTGCACCTACACTAGTG
CCACAAGAGCACTATGTTAGAATTACTGGCTTATACCCAACACTCAATATCTCAGA
TGAGTTTTCTAGCAATGTTGCAAATTATCAAAAGGTTGGTATGCAAAAGTATTCTA
CACTCCAGGGACCACCTGGTACTGGTAAGAGTCATTTTGCTATTGGCCTAGCTCTC
TACTACCCTTCTGCTCGCATAGTGTATACAGCTTGCTCTCATGCCGCTGTTGATGCA
CTATGTGAGAAGGCATTAAAATATTTGCCTATAGATAAATGTAGTAGAATTATACC
TGCACGTGCTCGTGTAGAGTGTTTTGATAAATTCAAAGTGAATTCAACATTAGAAC
AGTATGTCTTTTGTACTGTAAATGCATTGCCTGAGACGACAGCAGATATAGTTGTC
TTTGATGAAATTTCAATGGCCACAAATTATGATTTGAGTGTTGTCAATGCCAGATT
ACGTGCTAAGCACTATGTGTACATTGGCGACCCTGCTCAATTACCTGCACCACGCA
CATTGCTAACTAAGGGCACACTAGAACCAGAATATTTCAATTCAGTGTGTAGACTT
ATGAAAACTATAGGTCCAGACATGTTCCTCGGAACTTGTCGGCGTTGTCCTGCTGA
AATTGTTGACACTGTGAGTGCTTTGGTTTATGATAATAAGCTTAAAGCACATAAAG
ACAAATCAGCTCAATGCTTTAAAATGTTTTATAAGGGTGTTATCACGCATGATGTT
TCATCTGCAATTAACAGGCCACAAATAGGCGTGGTAAGAGAATTCCTTACACGTA
ACCCTGCTTGGAGAAAAGCTGTCTTTATTTCACCTTATAATTCACAGAATGCTGTA
GCCTCAAAGATTTTGGGACTACCAACTCAAACTGTTGATTCATCACAGGGCTCAGA
ATATGACTATGTCATATTCACTCAAACCACTGAAACAGCTCACTCTTGTAATGTAA
ACAGATTTAATGTTGCTATTACCAGAGCAAAAGTAGGCATACTTTGCATAATGTCT
GATAGAGACCTTTATGACAAGTTGCAATTTACAAGTCTTGAAATTCCACGTAGGAA
TGTGGCAACTTTACAAGCTGAAAATGTAACAGGACTCTTTAAAGATTGTAGTAAG
GTAATCACTGGGTTACATCCTACACAGGCACCTACACACCTCAGTGTTGACACTAA
ATTCAAAACTGAAGGTTTATGTGTTGACATACCTGGCATACCTAAGGACATGACCT
ATAGAAGACTCATCTCTATGATGGGTTTTAAAATGAATTATCAAGTTAATGGTTAC
CCTAACATGTTTATCACCCGCGAAGAAGCTATAAGACATGTACGTGCATGGATTG
GCTTCGATGTCGAGGGGTGTCATGCTACTAGAGAAGCTGTTGGTACCAATTTACCT
TTACAGCTAGGTTTTTCTACAGGTGTTAACCTAGTTGCTGTACCTACAGGTTATGTT
GATACACCTAATAATACAGATTTTTCCAGAGTTAGTGCTAAACCACCGCCTGGAGA
TCAATTTAAACACCTCATACCACTTATGTACAAAGGACTTCCTTGGAATGTAGTGC
GTATAAAGATTGTACAAATGTTAAGTGACACACTTAAAAATCTCTCTGACAGAGTC
GTATTTGTCTTATGGGCACATGGCTTTGAGTTGACATCTATGAAGTATTTTGTGAA
AATAGGACCTGAGCGCACCTGTTGTCTATGTGATAGACGTGCCACATGCTTTTCCA
CTGCTTCAGACACTTATGCCTGTTGGCATCATTCTATTGGATTTGATTACGTCTATA
ATCCGTTTATGATTGATGTTCAACAATGGGGTTTTACAGGTAACCTACAAAGCAAC
CATGATCTGTATTGTCAAGTCCATGGTAATGCACATGTAGCTAGTTGTGATGCAAT
CATGACTAGGTGTCTAGCTGTCCACGAGTGCTTTGTTAAGCGTGTTGACTGGACTA
TTGAATATCCTATAATTGGTGATGAACTGAAGATTAATGCGGCTTGTAGAAAGGTT
CAACACATGGTTGTTAAAGCTGCATTATTAGCAGACAAATTCCCAGTTCTTCACGA
CATTGGTAACCCTAAAGCTATTAAGTGTGTACCTCAAGCTGATGTAGAATGGAAGT
TCTATGATGCACAGCCTTGTAGTGACAAAGCTTATAAAATAGAAGAATTATTCTAT
TCTTATGCCACACATTCTGACAAATTCACAGATGGTGTATGCCTATTTTGGAATTG
CAATGTCGATAGATATCCTGCTAATTCCATTGTTTGTAGATTTGACACTAGAGTGC
TATCTAACCTTAACTTGCCTGGTTGTGATGGTGGCAGTTTGTATGTAAATAAACAT
GCATTCCACACACCAGCTTTTGATAAAAGTGCTTTTGTTAATTTAAAACAATTACC
ATTTTTCTATTACTCTGACAGTCCATGTGAGTCTCATGGAAAACAAGTAGTGTCAG
ATATAGATTATGTACCACTAAAGTCTGCTACGTGTATAACACGTTGCAATTTAGGT
GGTGCTGTCTGTAGACATCATGCTAATGAGTACAGATTGTATCTCGATGCTTATAA
CATGATGATCTCAGCTGGCTTTAGCTTGTGGGTTTACAAACAATTTGATACTTATA
ACCTCTGGAACACTTTTACAAGACTTCAGAGTTTAGAAAATGTGGCTTTTAATGTT
GTAAATAAGGGACACTTTGATGGACAACAGGGTGAAGTACCAGTTTCTATCATTA
ATAACACTGTTTACACAAAAGTTGATGGTGTTGATGTAGAATTGTTTGAAAATAAA
ACAACATTACCTGTTAATGTAGCATTTGAGCTTTGGGCTAAGCGCAACATTAAACC
AGTACCAGAGGTGAAAATACTCAATAATTTGGGTGTGGACATTGCTGCTAATACT
GTGATCTGGGACTACAAAAGAGATGCTCCAGCACATATATCTACTATTGGTGTTTG
TTCTATGACTGACATAGCCAAGAAACCAACTGAAACGATTTGTGCACCACTCACTG
TCTTTTTTGATGGTAGAGTTGATGGTCAAGTAGACTTATTTAGAAATGCCCGTAAT
GGTGTTCTTATTACAGAAGGTAGTGTTAAAGGTTTACAACCATCTGTAGGTCCCAA
ACAAGCTAGTCTTAATGGAGTCACATTAATTGGAGAAGCCGTAAAAACACAGTTC
AATTATTATAAGAAAGTTGATGGTGTTGTCCAACAATTACCTGAAACTTACTTTAC
TCAGAGTAGAAATTTACAAGAATTTAAACCCAGGAGTCAAATGGAAATTGATTTC
TTAGAATTAGCTATGGATGAATTCATTGAACGGTATAAATTAGAAGGCTATGCCTT
CGAACATATCGTTTATGGAGATTTTAGTCATAGTCAGTTAGGTGGTTTACATCTAC
TGATTGGACTAGCTAAACGTTTTAAGGAATCACCTTTTGAATTAGAAGATTTTATT
CCTATGGACAGTACAGTTAAAAACTATTTCATAACAGATGCGCAAACAGGTTCAT
CTAAGTGTGTGTGTTCTGTTATTGATTTATTACTTGATGATTTTGTTGAAATAATAA
AATCCCAAGATTTATCTGTAGTTTCTAAGGTTGTCAAAGTGACTATTGACTATACA
GAAATTTCATTTATGCTTTGGTGTAAAGATGGCCATGTAGAAACATTTTACCCAAA
ATTACAATCTAGTCAAGCGTGGCAACCGGGTGTTGCTATGCCTAATCTTTACAAAA
TGCAAAGAATGCTATTAGAAAAGTGTGACCTTCAAAATTATGGTGATAGTGCAAC
ATTACCTAAAGGCATAATGATGAATGTCGCAAAATATACTCAACTGTGTCAATATT
TAAACACATTAACATTAGCTGTACCCTATAATATGAGAGTTATACATTTTGGTGCT
GGTTCTGATAAAGGAGTTGCACCAGGTACAGCTGTTTTAAGACAGTGGTTGCCTAC
GGGTACGCTGCTTGTCGATTCAGATCTTAATGACTTTGTCTCTGATGCAGATTCAA
CTTTGATTGGTGATTGTGCAACTGTACATACAGCTAATAAATGGGATCTCATTATT
AGTGATATGTACGACCCTAAGACTAAAAATGTTACAAAAGAAAATGACTCTAAAG
AGGGTTTTTTCACTTACATTTGTGGGTTTATACAACAAAAGCTAGCTCTTGGAGGT
TCCGTGGCTATAAAGATAACAGAACATTCTTGGAATGCTGATCTTTATAAGCTCAT
GGGACACTTCGCATGGTGGACAGCCTTTGTTACTAATGTGAATGCGTCATCATCTG
AAGCATTTTTAATTGGATGTAATTATCTTGGCAAACCACGCGAACAAATAGATGGT
TATGTCATGCATGCAAATTACATATTTTGGAGGAATACAAATCCAATTCAGTTGTC
TTCCTATTCTTTATTTGACATGAGTAAATTTCCCCTTAAATTAAGGGGTACTGCTGT
TATGTCTTTAAAAGAAGGTCAAATCAATGATATGATTTTATCTCTTCTTAGTAAAG
GTAGACTTATAATTAGAGAAAACAACAGAGTTGTTATTTCTAGTGATGTTCTTGTT
AACAACTAAGCGATCGCGCCACCATGGTGAGCAAGGGCGAGGAGCTGTTCACCGG
GGTGGTGCCCATCCTGGTCGAGCTGGACGGCGACGTAAACGGCCACAAGTTCAGC
GTGTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTT
CAAGTCCGCCATGCCCGAAGGCTACGTCCAGGAGCGCACCATCTTCTTCAAGGAC
GACGGCAACTACAAGACCCGCGCCGAGGTGAAGTTCGAGGGCGACACCCTGGTG
AACCGCATCGAGCTGAAGGGCATCGACTTCAAGGAGGACGGCAACATCCTGGGGC
ACAAGCTGGAGTACAACTACAACAGCCACAACGTCTATATCATGGCCGACAAGCA
GAAGAACGGCATCAAGGTGAACTTCAAGATCCGCCACAACATCGAGGACGGCAG
CGTGCAGCTCGCCGACCACTACCAGCAGAACACCCCCATCGGCGACGGCCCCGTG
CTGCTGCCCGACAACCACTACCTGAGCACCCAGTCCGCCCTGAGCAAAGACCCCA
ACGAGAAGCGCGATCACATGGTCCTGCTGGAGTTCGTGACCGCCGCCGGGATCAC
TCTCGGCATGGACGAGCTGTACAAGTAGCCTGCAGGATGGGCTATATAAACGTTTT
CGCTTTTCCGTTTACGATATATAGTCTACTCTTGTGCAGAATGAATTCTCGTAACTA
CATAGCACAAGTAGATGTAGTTAACTTTAATCTCACATAGCAATCTTTAATCAGTG
TGTAACATTAGGGAGGACTTGAAAGAGCCACCACATTTTCACCGAGGCCACGCGG
AGTACGATCGAGTGTACAGTGAACAATGCTAGGGAGAGCTGCCTATATGGAAGAG
CCCTAATGTGTAAAATTAATTTTAGTAGTGCTATCCCCATGTGATTTTAATAGCTTC
TTAGGAGAATGACAAAAAAAAAAAAAAAAAAAAAAAAAAAAAAAAAGCGGCCG
CTTGAGATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCCGAAGGT
AACTGGCTTCAGCAGAGCGCAGATACCAAATACTGTCCTTCTAGTGTAGCCGTAGT
TAGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTG
AGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTCGATTTT
TGTGATGCTCGTCAGGGGGGCGGAGCCTATGGAAACGCGGAACCCCTATTTGTTT
ATTTTTCTAAATACATTCAAATATGTATCCGCTCATGAGACAATAACCCTGATAAA
TGCTTCAATAATATTGAAAAAGGAAGAGTATGATTGAACAAGATGGATTGCACGC
AGGTTCTCCGGCCGCTTGGGTGGAGAGGCTATTCGGCTATGACTGGGCACAACAG
ACAATCGGCTGCTCTGATGCCGCCGTGTTCCGGCTGTCAGCGCAGGGGCGCCCGG
TTCTTTTTGTCAAGACCGACCTGTCCGGTGCCCTGAATGAACTGCAGGACGAGGCA
GCGCGGCTATCGTGGCTGGCCACGACGGGCGTTCCTTGCGCAGCTGTGCTCGACGT
TGTCACTGAAGCGGGAAGGGACTGGCTGCTATTGGGCGAAGTGCCGGGGCAGGAT
CTCCTGTCATCTCACCTTGCTCCTGCCGAGAAAGTATCCATCATGGCTGATGCAAT
GCGGCGGCTGCATACGCTTGATCCGGCTACCTGCCCATTCGACCACCAAGCGAAA
CATCGCATCGAGCGAGCACGTACTCGGATGGAAGCCGGTCTTGTCGATCAGGATG
ATCTGGACGAAGAGCATCAGGGGCTCGCGCCAGCCGAACTGTTCGCCAGGCTCAA
GGCGCGCATGCCCGACGGCGAGGATCTCGTCGTGACCCATGGCGATGCCTGCTTG
CCGAATATCATGGTGGAAAATGGCCGCTTTTCTGGATTCATCGACTGTGGCCGGCT
GGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTCC
CGATTCGCAGCGCATCGCCTTCTATCGCCTTCTTGACGAGTTCTTCTGAATTGAAA
AAGGAAGAGTAACGCCAGCAACGCGAGCTCGGCCGGCCGACATTGATTATTGACT
AGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCA
CTCTCCCCATCTCCCCCCCCTCCCCACCCCCAATTTTGTATTTATTTATTTTTTAATT
ATTTTGTGCAGCGATGGGGGCGGGGGGGGGGGGGGCGCGCGCCAGGGGGGGGGG
GGGGGGGGGGGGGGGGGGGGGGGGGGGGGGGGGGGGGGGCGGCGGCAGCCAAT
CAGAGCGGCGCGCTCCGAAAGTTTCCTTTTATGGCGAGGCGGCGGCGGCGGCGGC
CCTATAAAAAGCGAAGCGCGCGGCGGGCGGGAGTCGCTGCGCGCTGCCTTCGCCC
CGTGCCCCGCTCCGCCGCCGCCTCGCGCCGCCCGCCCCGGCTCTGACTGACCGCGT
TACTCCCACAGGTGAGCGGGCGGGACGGCCCTTCTCCTCCGGGCTGTAATTAGCG
CTTGGTTTAATGACGGCTTGTTTCTTTTCTGTGGCTGCGTGAAAGCCTTGAGGGGCT
CCGGGAGGGCCCTTTGTGCGGGGGGAGCGGCTCGGGGGGTGCGTGCGTGTGTGTG
TGCGTGGGGAGCGCCGCGTGCGGCTCCGCGCTGCCCGGCGGCTGTGAGCGCTGCG
GGCGCGGCGCGGGGCTTTGTGCGCTCCGCAGTGTGCGCGAGGGGAGCGCGGCCGG
GGGCGGTGCCCCGCGGTGCGGGGGGGGCTGCGAGGGGAACAAAGGCTGCGTGCG
GGGTGTGTGCGTGGGGGGGTGAGCAGGGGGTGTGGGCGCGTCGGTCGGGCTGCAA
CCCCCCCTGCACCCCCCTCCCCGAGTTGCTGAGCACGGCCCGGCTTCGGGTGCGGG
GCTCCGTACGGGGCGTGGCGCGGGGCTCGCCGTGCCGGGCGGGGGGTGGCGGCA
GGTGGGGGTGCCGGGCGGGGCGGGGCCGCCTCGGGCCGGGGGGGGCTCGGGGGG
GGGGCGCGGCGGCCCCCGGAGCGCCGGCGGCTGTCGAGGCGCGGCGAGCCGCAG
CCATTGCCTTTTATGGTAATCGTGCGAGAGGGCGCAGGGACTTCCTTTGTCCCAAA
TCTGTGCGGAGCCGAAATCTGGGAGGCGCCGCCGCACCCCCTCTAGCGGGCGCGG
GGCGAAGCGGTGCGGCGCCGGCAGGAAGGAAATGGGCGGGGAGGGCCTTCGTGC
GTCGCCGCGCCGCCGTCCCCTTCTCCCTCTCCAGCCTCGGGGCTGTCCGCGGGGGG
ACGGCTGCCTTCGGGGGGGACGGGGCAGGGCGGGGTTCGGCTTCTGGCGTGTGAC
CGGCGGCTTCAGAGCCTCTGCTAACCATGTTCATGCCTTCTTCTTTTTCCTACAGGG
CCGGCCCGTTTAGTGAACCGTCAGAATTTTGTAATACGACTCACTATAGGG

One basic payload: Omicron spike protein without Furin cleavage site
(FCS) and two amino acid substitutions with prolines

| | |
|---|---|
| Kozak sequence | 9 . . . 14 |
| Signal Sequence | 15 . . . 50 |
| MFVFLVLLPLVS | |
| Omicron Spike_no FCS and two Prolines | 51 . . . 3815 |

SQCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCEF
QFCNDPFLDHKNNKSWMESEFRVYSSANNCTFEYVSQPFLMDLEGKQGNFKNLREFV
FKNIDGYFKIYSKHTPIIVREPEDLPQGFSALEPLVDLPIGINITRFQTLLALHRSYLTPGD
SSSGWTAGAAAYYVGYLQPRTFLLKYNENGTITDAVDCALDPLSETKCTLKSFTVEK
GIYQTSNFRVQPTESIVRFPNITNLCPFDEVFNATRFASVYAWNRKRISNCVADYSVLY
NLAPFFTFKCYGVSPTKLNDLCFTNVYADSFVIRGDEVRQIAPGQTGNIADYNYKLPD
DFTGCVIAWNSNKLDSKVSGNYNYLYRLFRKSNLKPFERDISTEIYQAGNKPCNGVAG
FNCYFPLRSYSFRPTYGVGHQPYRVVVLSFELLHAPATVCGPKKSTNLVKNKCVNFNF
NGLKGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSY
ECDIPIGAGICASYQTQTKSHASVASQSIIAYTMSLGAENSVAYSNNSIAIPTNFTISVTT
EILPVSMTKTSVDCTMYICGDSTECSNLLLQYGSFCTQLKRALTGIAVEQDKNTQEVF
AQVKQIYKTPPIKYFGGFNFSQILPDPSKPSKRSFIEDLLFNKVTLADAGFIKQYGDCLG
DIAARDLICAQKFKGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNHNAQA
LNTLVKQLSSKFGAISSVLNDIFSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNF
TTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLCCMTSCCSCLKGCCSCGS
CCKFDEDDSEPVLKGVKLHYT

| | |
|---|---|
| End of EC domain | 3627 . . . 3635 |
| Alfa Tag | 3816 . . . 3854 |
| SRLEEELRRRLTE | |

GCCACCATGTTTGTTTTTCTTGTTTTATTGCCACTAGTCTCTAGTCAGTGTGTTAAT
CTTACAACCAGAACTCAATTACCCCCTGCATACACTAATTCTTTCACACGTGGTGT
TTATTACCCTGACAAAGTTTTCAGATCCTCAGTTTTACATTCAACTCAGGACTTGTT
CTTACCTTTCTTTTCCAATGTTACTTGGTTCCATGTTATCTCTGGGACCAATGGTAC
TAAGAGGTTTGATAACCCTGTCCTACCATTTAATGATGGTGTTTATTTTGCTTCCAT
TGAGAAGTCTAACATAATAAGAGGCTGGATTTTTGGTACTACTTTAGATTCGAAGA
CCCAGTCCCTACTTATTGTTAATAACGCTACTAATGTTGTTATTAAAGTCTGTGAAT
TTCAATTTTGTAATGATCCATTTTTGGACCACAAAAACAACAAAAGTTGGATGGAA
AGTGAGTTCAGAGTTTATTCTAGTGCGAATAATTGCACTTTTGAATATGTCTCTCA
GCCTTTTCTTATGGACCTTGAAGGAAAACAGGGTAATTTCAAAAATCTTAGGGAAT
TTGTGTTTAAGAATATTGATGGTTATTTTAAAATATATTCTAAGCACACGCCTATTA
TAGTGCGTGAGCCAGAAGATCTCCCTCAGGGTTTTTCGGCTTTAGAACCATTGGTA
GATTTGCCAATAGGTATTAACATCACTAGGTTTCAAACTTTACTTGCTTTACATAG
AAGTTATTTGACTCCTGGTGATTCTTCTTCAGGTTGGACAGCTGGTGCTGCAGCTT
ATTATGTGGGTTATCTTCAACCTAGGACTTTTCTATTAAAATATAATGAAAATGGA
ACCATTACAGATGCTGTAGACTGTGCACTTGACCCTCTCTCAGAAACAAAGTGTAC
GTTGAAATCCTTCACTGTAGAAAAAGGAATCTATCAAACTTCTAACTTTAGAGTCC
AACCAACAGAATCTATTGTTAGATTTCCTAATATTACAAACTTGTGCCCTTTTGAT
GAAGTTTTTAACGCCACCAGATTTGCATCTGTTTATGCTTGGAACAGGAAGAGAAT
CAGCAACTGTGTTGCTGATTATTCTGTCCTATATAATCTCGCACCATTTTTCACTTT
TAAGTGTTATGGAGTGTCTCCTACTAAATTAAATGATCTCTGCTTTACTAATGTCTA
TGCAGATTCATTTGTAATTAGAGGTGATGAAGTCAGACAAATCGCTCCAGGGCAA
ACTGGAAATATTGCTGATTATAATTATAAATTACCAGATGATTTTACAGGCTGCGT
TATAGCTTGGAATTCTAACAAGCTTGATTCTAAGGTTAGTGGTAATTATAATTACC
TGTATAGATTGTTTAGGAAGTCTAATCTCAAACCTTTTGAGAGAGATATTTCAACT
GAAATCTATCAGGCCGGTAACAAACCTTGTAATGGTGTTGCAGGTTTTAATTGTTA
CTTTCCTTTACGATCATATAGTTTCCGACCCACTTATGGTGTTGGTCACCAACCATA
CAGAGTAGTAGTACTTTCTTTTGAACTTCTACATGCACCAGCAACTGTTTGTGGAC
CTAAAAAGTCTACTAATTTGGTTAAAAACAAATGTGTCAATTTCAACTTCAATGGT
TTAAAAGGCACAGGTGTTCTTACTGAGTCTAACAAAAAGTTTCTGCCTTTCCAACA
ATTTGGCAGAGACATTGCTGACACTACTGATGCTGTCCGTGATCCACAGACACTTG
AGATTCTTGACATTACACCATGTTCTTTTGGTGGTGTCAGTGTTATAACACCAGGA
ACAAATACTTCTAACCAGGTTGCTGTTCTTTATCAGGGTGTTAACTGCACAGAAGT
CCCTGTTGCTATTCATGCAGATCAACTTACTCCTACTTGGCGTGTTTATTCTACAGG
TTCTAATGTTTTTCAAACACGTGCAGGCTGTTTAATAGGGGCTGAATATGTCAACA
ACTCATATGAGTGTGACATACCCATTGGTGCAGGTATATGCGCTAGTTATCAGACT
CAGACTAAGTCTCATGCTAGTGTAGCTAGTCAATCCATCATTGCCTACACTATGTC
ACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCA
GTAGATTGTACAATGTACATTTGTGGTGATTCAACTGAATGCAGCAATCTTTTGTT
GCAATATGGCAGTTTTTGTACACAATTAAAACGTGCTTTAACTGGAATAGCTGTTG
AACAAGACAAAAACACCCAAGAAGTTTTTGCACAAGTCAAACAAATTTACAAAAC
ACCACCAATTAAATATTTTGGTGGTTTTAATTTTTCACAAATATTACCAGATCCATC
AAAACCAAGCAAGAGGTCATTTATTGAAGATCTACTTTTCAACAAAGTGACACTT
GCAGATGCTGGCTTCATCAAACAATATGGTGATTGCCTTGGTGATATTGCTGCTAG
AGACCTCATTTGTGCACAAAAGTTTAAAGGCCTTACTGTTTTGCCACCTTTGCTCA
CAGATGAAATGATTGCTCAATACACTTCTGCACTGTTAGCGGGTACAATCACTTCT
GGTTGGACCTTTGGTGCAGGTGCTGCATTACAAATACCATTTGCTATGCAAATGGC
TTATAGGTTTAATGGTATTGGAGTTACACAGAATGTTCTCTATGAGAACCAAAAAT
TGATTGCCAACCAATTTAATAGTGCTATTGGCAAAATTCAAGACTCACTTTCTTCC
ACAGCAAGTGCACTTGGAAAACTTCAAGATGTGGTCAACCATAATGCACAAGCTT
TAAACACGCTTGTTAAACAACTTAGCTCCAAATTTGGTGCAATTTCAAGTGTTTTA
AATGATATCTTTTCACGTCTTGACCCTCCTGAGGCTGAAGTGCAAATTGATAGGTT
GATCACAGGCAGACTTCAAAGTTTGCAGACATATGTGACTCAACAATTAATTAGA
GCTGCAGAAATCAGAGCTTCTGCTAATCTTGCTGCTACTAAAATGTCAGAGTGTGT
ACTTGGACAATCAAAAAGAGTTGATTTTTGTGGAAAGGGCTATCATCTTATGTCCT
TCCCTCAGTCAGCACCTCATGGTGTAGTCTTCTTGCATGTGACTTATGTCCCTGCAC
AAGAAAAGAACTTCACAACTGCTCCTGCCATTTGTCATGATGGAAAAGCACACTTT
CCTCGTGAAGGTGTCTTTGTTTCAAATGGCACACACTGGTTTGTAACACAAAGGAA
TTTTTATGAACCACAAATCATTACTACAGACAACACATTTGTGTCTGGTAACTGTG
ATGTTGTAATAGGAATTGTCAACAACACAGTTTATGATCCTTTGCAACCTGAATTA
GATTCATTCAAGGAGGAGTTAGATAAATATTTTAAGAATCATACATCACCAGATGT
TGATTTAGGTGACATCTCTGGCATTAATGCTTCAGTTGTAAACATTCAAAAAGAAA
TTGACCGCCTCAATGAGGTTGCCAAGAATTTAAATGAATCTCTCATCGATCTCCAA
GAACTTGGAAAGTATGAGCAGTATATAAAATGGCCATGGTACATTTGGCTAGGTT
TTATAGCTGGCTTGATTGCCATAGTAATGGTGACAATTATGCTTTGCTGTATGACC
AGTTGCTGTAGTTGTCTCAAGGGCTGTTGTTCTTGTGGATCCTGCTGCAAATTTGAT
GAAGACGACTCTGAGCCAGTGCTCAAAGGAGTCAAATTACATTACACATCACGAC
TGGAGGAAGAACTGCGCCGACGCCTGACTGAATAA

### Example 4: EGFP Expression Using a Self-Amplifying Vector of an Embodiment of the Invention

Time Course after Transfection with Self-Amplifying VEE Vector:

Rationale: The first transcription driven by CMV promoter results in the SAM for EGFP and so the number of EGFP positive cells continuously increases over time while the typical transgene disappears soon without antibiotics selection after several cell divisions because they can't self replicate

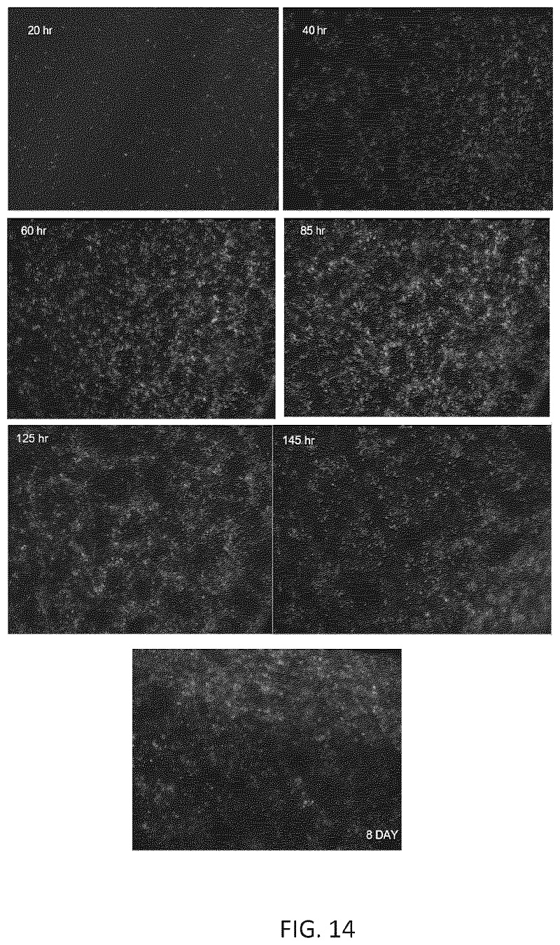

FIG. 14 provides time course images after transfection using Lipofectamine 3000 of HEK293 cells with CMV+T7_VEE_EGFP. EGFP positive cells increases in number even until 85 hr—Demonstrates self amplification for EGFP and eliminates the need of in vitro transcription by T7 Pol.

RT-PCR to Show the mRNA from the Self-Amplifying VEE Vector:

The HEK 293 cells are seeded at the cell density of 5×105 per well to achieve 70 to 90% confluency in a 6-well plate a day prior to the transfection. Transfection was performed with DNA or IVT mRNA from the vector according to the protocol for Lipofectamine 300 of Thermofisher scientific. The cells were harvested 48 hrs after the transfection for RNA extraction. Total mRNA was checked on the 0.8% agarose gel for its integrity. 1 ug of total mRNA was treated with amplification grade DNase I to remove any residual DNA. mRNA was subject to CDNA synthesis by the superscript III enzyme. The gene specific primer annealed to the (−) negative strand was used to synthesize cDNA from the mRNA of transfected cells and IVT mRNA as a negative control. PCR to amplifying GFP was done to show mRNA produced from the DNA and mRNA amplifies continuously.

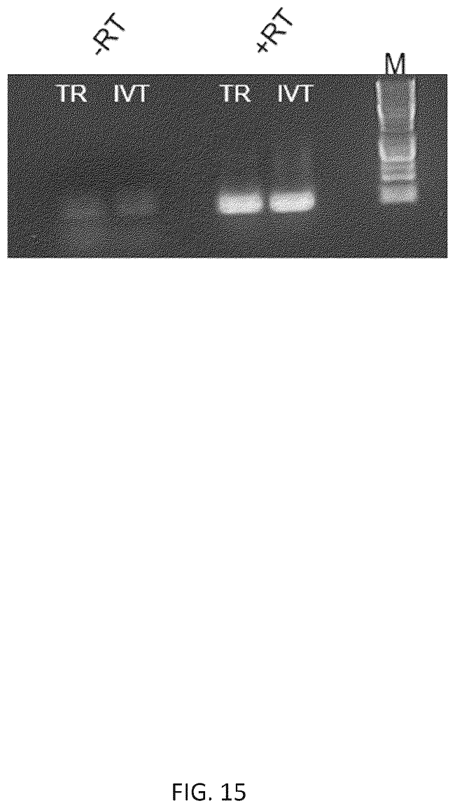

FIG. 15 provides molecular biological evidence for SAM by RT-PCR on the mRNA from transfected HEK293 to identify negative strand mRNA for EGFP. TR: mRNA from transfected HEK293 with CMV+T7-Vee_EGFP. IVT: In Vitro transcribed mRNA from CMV+T7-Vee-EGFP; −RT: Without Reverse Transcription; +RT: Reverse transcribed with EGFP FWD primer (5′-CATGAAGCAGCACGACTTCT-3′) and REV primers (5′-CTGCTTGTCGGCCATGATATAG-3′) for TR and IVT samples respectively. PCR: 94° C. for 30 sec, 56° C. for 30 sec, 72° C. for 30 sec, total 28 cycles. +RT samples showed good intensity of PCR bands.

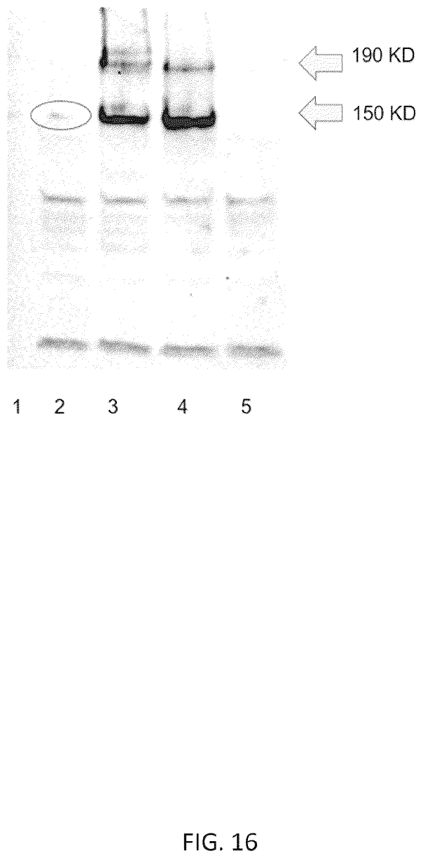

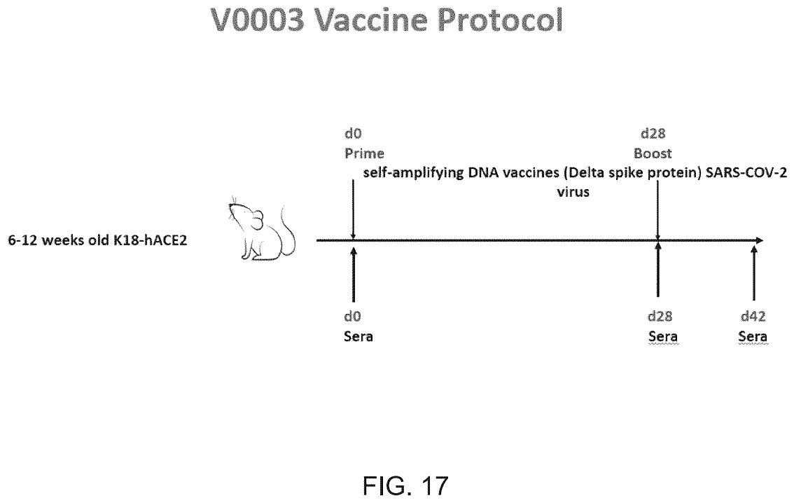

FIG. 16 provides a western blot on HEK293 Cells transfected with Delta variant spike vaccines to validate the protein expression. 1. Cell lysate of HEK 293 cells transfected with the vector having Spike (S1+S2 ECD); 2. Cell lysate of HEK 293 Cells with the vector having Spike (S1+S2 ECD) fused with HLA signal sequence, transmembrane domain and cytoplasmic domain. 3. Cell lysate from HEK 293 cells with the vector having Spike (S1+S2 ECD) fused with Cd74 cytoplasmic domain and HLA transmembrane domain; 4 Protein size marker; 5. Cell lysate from HEK 293 cells transfected with the vector having EGFP gene in the same vector backbone (Negative Control).

### Example 5: Immune Response Following Administration with SAM Vectors

### Methods

Vaccine protocol detailed in FIG. 17.

### ELISA Materials

1. 96 well assay plate #3369 (Corning Costar)
2. SARS-COV-2 protein, His Tag, Super stable trimer #SPN-C52H9 (Acrobiosystems)
3. Serum samples from the vaccine injected mice (used at the indicated dilutions)
4. Coating buffer—0.1 Molar Carbonate buffer, pH-9.5—Sodium bicarbonate (6.232 g), Sodium Carbonate anhydrous (2.737 g)
5. Blocking buffer-1× Phosphate Buffered Saline (PBS)-pH 7.4, 0.1% Tween-20, 1% Bovine Serum Albumin (BSA)
6. Washing buffer-1×PBS-pH 7.4, 0.1% Tween-20
7. Stopping solution-0.16 N Sulfuric acid
8. Secondary antibody—a) Goat Anti-Mouse IgM-HRP #1021-05 (Southern Biotech) b) Goat Anti-Mouse IgA-HRP #1040-05 (Southern Biotech) c) Goat Anti-Mouse IgG-HRP #1030-05 (Southern Biotech)
9. TMB substrate—1 Step™ Ultra TMB-ELISA #34028 (Thermofisher)
10. Antibody standards-Anti-SARS-COV-2 Spike S1 Antibody, Mouse IgG1 #S1N-58A1-100 ug (Acrobiosystems)

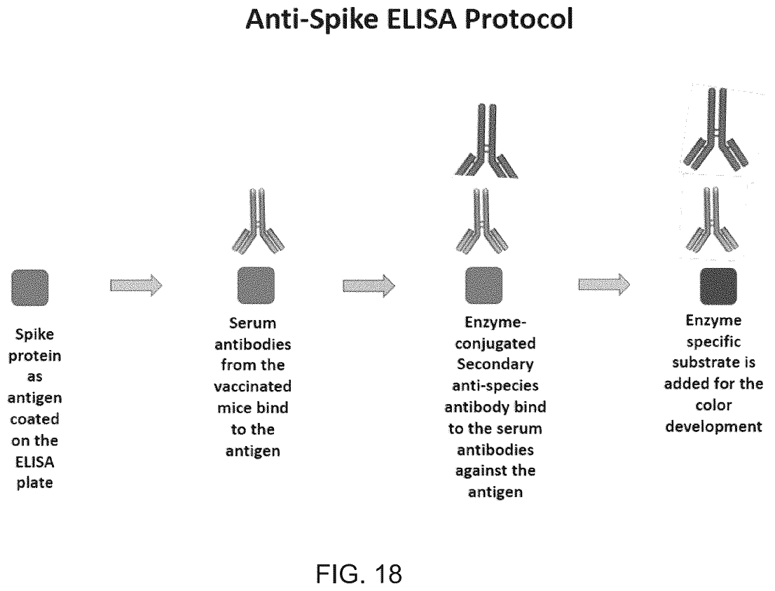

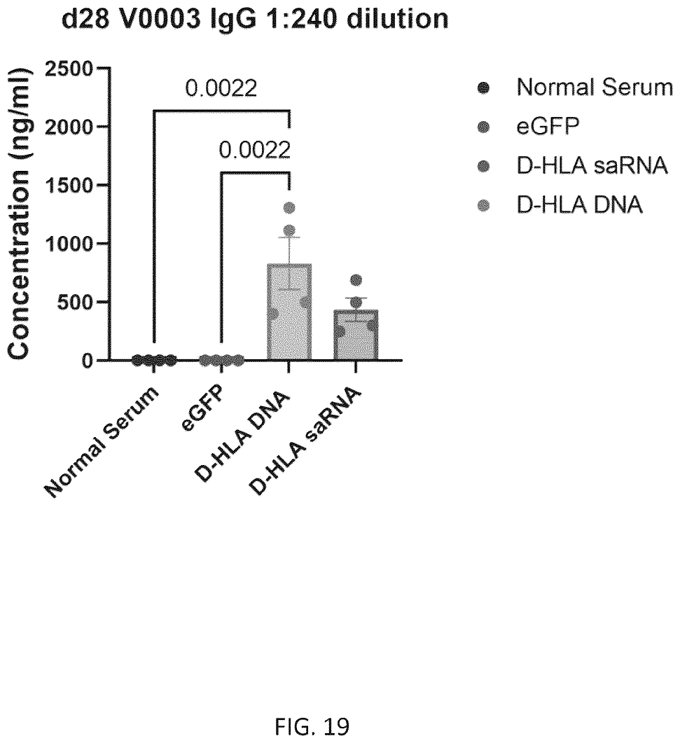

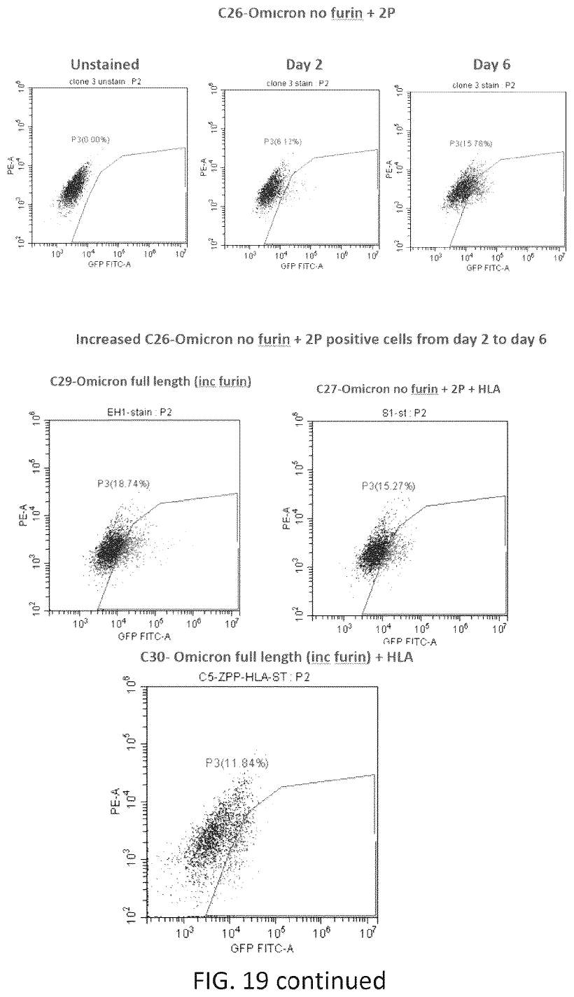

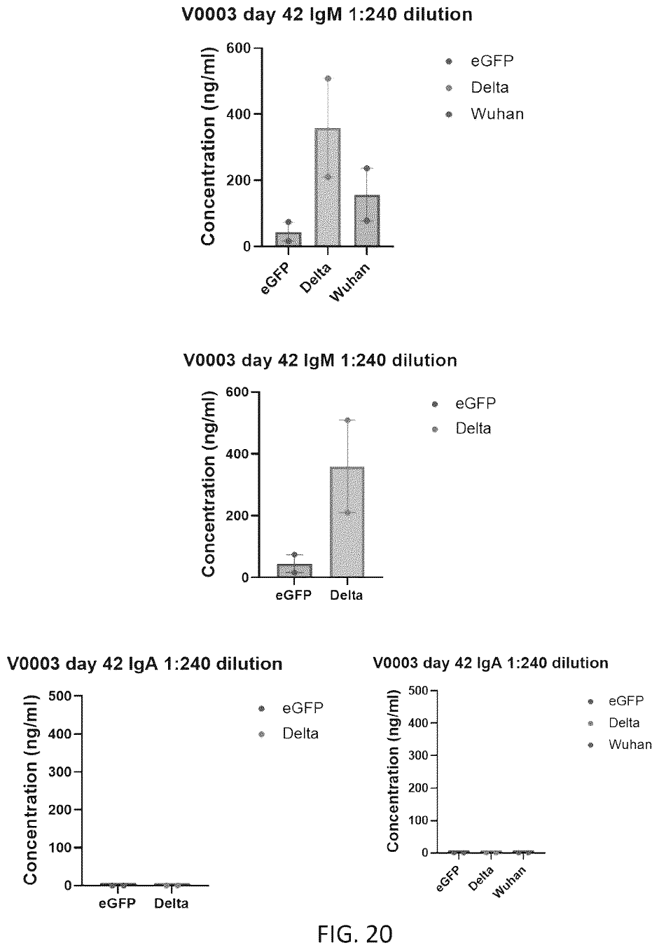

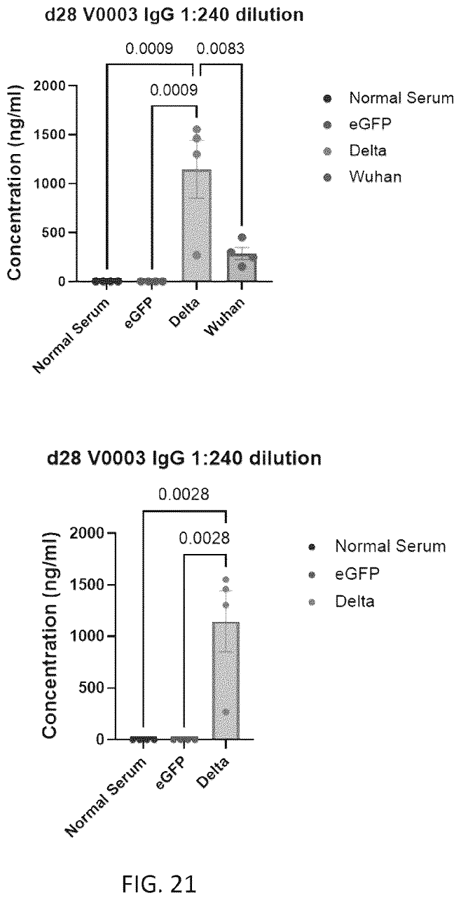

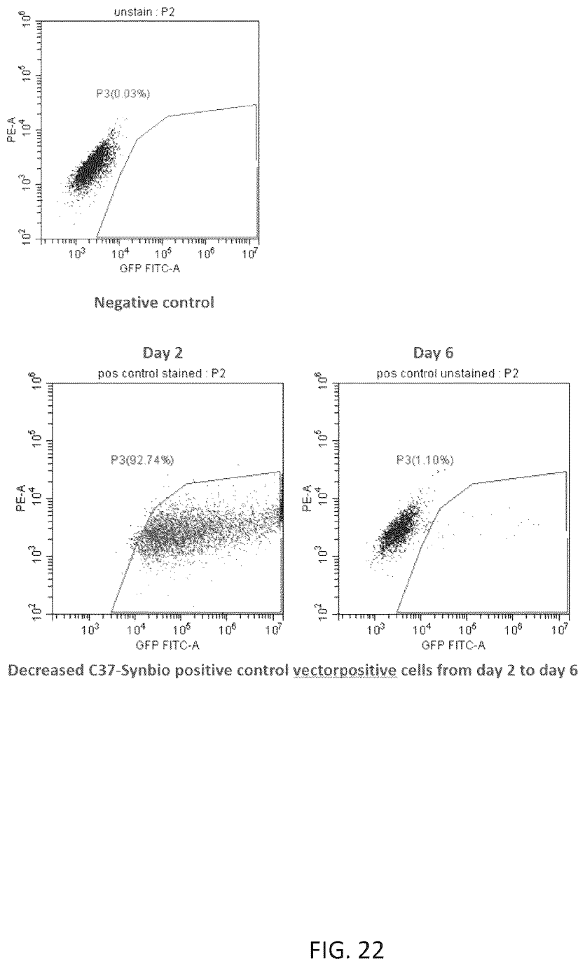

For the ELISA protocol (see FIG. 18), 100 ng/ml of the SARS-COV-2 spike protein was coated onto the 96 well plates using the coating buffer. After overnight incubation at 4° C., the plates were washed 4 times with the washing buffer. Subsequently, the plates were blocked with the blocking buffer overnight at 4° C. The next day, serum samples were diluted in blocking buffer at 1:80 to 1:2160 dilution. The plates were washed 4 times with the washing buffer and the serum samples were added and the plates were incubated in dark at 37ºC for 1 hour. After 1 hour incubation, the plates were washed again with the washing buffer 4 times following which Goat-anti mouse secondary antibody (at 1:4000 to 1:8000 dilution-dilution made in blocking buffer) was added. The plates were incubated again at 37ºC for 1 hour. The plates were finally washed with the washing buffer 4 times. 100 μl/well of TMB substrate was added to each well and the plates were incubated in dark at room temperature for 20 minutes for colour development. After 20 minutes, the reaction was stopped by adding 100 μl/well of stopping solution. The plates were then read using ELISA plate reader at 450 nm (nanometers). The values were quantified using the standard antibody coated on the plates and results expressed in nanograms/milliliter (ng/ml).

### Results

The plates were read at 450 nm and the resulting data was exported to the excel file. The data was further analyzed using Graphpad prism software. In Brief, a standard curve was set up with known antibody concentrations binding to the spike protein. This standard curve was then used to interpolate and quantify the serum sample values for IgG, IgM and IgA. Analysis of Variance (ANOVA) statistical test along with Tukey's and Dunnett's posthoc tests were used to test significant differences between the groups and p values greater than 0.05 were considered significant. Our results show a significant increase in IgG and IgM antibodies in response to our vaccinations against SARS-COV-2 spike protein. The IgG results show that the IgG response was greater with self-amplifying DNA vaccines compared to the self-amplifying RNA vaccines. The results also suggest a robust IgM antibody response against SARS-COV-2 spike protein in response to our vaccine. In comparison, our vaccines did not induce any good IgA antibodies.

### Example: Dose Response and Immunogenicity Testing for DNA COVID-19 Vaccines

### Brief Description of Project

In a small-scale preclinical study, groups of 15 week old K18-hACE2 transgenic mice will be immunized with different vaccines targeting SARS-COV-2. Including a group identical to one in a previous trial at UofT, to enable comparison between the two different facilities. Mice will be immunized by intramuscular injection and boosted with the same vaccine after 28 days (4 weeks). Mice will be monitored for any behavioural changes and weight loss. Blood samples will be taken by saphenous vein bleed before vaccination at day −1 also at day 7, day 14, and day 28 post-prime vaccination. After 42 days (6 weeks), mice will be euthanized and tissues and blood harvested for immune assay studies.

### Experimental Plan

Summary: Intramuscular immunization of mice with 4 different vaccines, total 11 groups of 4 mice per group (44 mice). Mice will be monitored throughout study, blood samples are collected at day −1, 7, 14 and 28, boost IM injection on day 28. End experiment at day 42, collect blood, leg muscle for injection site and various organs as detailed below.

Monitoring Throughout Study: Monitor daily for three days post injection and two days post blood sample collection otherwise monitor weekly. Record body weight and any body condition/behavioural changes, with an end-point at 20% overall weight loss or 10% weight loss from previous weight.

Day −3: Blood sample collection, saphenous bleed from left leg using serum/EDTA capillary tubes, approx. 50 μl.

### Day 0

Intramuscular vaccinations as detailed below, volume max. 50 μl into right hind leg (caudal thigh muscle, mark injection site). All groups, dose was 2.5 μg RNA or DNA per mouse, in liposome/LNP. C #relate to our Construct Numbers each vaccine is derived from.
D1 (Ctrl): Control (negative) group—5 μg DNA eGFP. (C1)
D2: DNA Delta full length spike. (C7)
D3: DNA for Delta spike ectodomain+HLA. (C8)
D4: DNA for Delta spike ectodomain+CD74+HLA. (C9)
D5: Wuhan (C2)
M3: saRNA for Delta full length spike, overlap group with UofT. 5 μg saRNA. (C18)

Day 13/14: Blood sample collection, saphenous bleed from left hind leg using serum/EDTA capillary tubes, approx. 50 μl.

### Day 27/28:

Blood sample collection, saphenous bleed from left hind leg using serum/EDTA capillary tubes, approx. 50 μl.

### Day 29:

Boost (IM) as for initial vaccination.

### Day 42:

Euthanize all mice, collect blood for serum, right hind leg (for injection site) in 15 ml 4% PFA and the following organs:
Spleen—in PBS on ice
Cervical lymph nodes, liver, kidney, lung, intestine, pancreas, heart and brains.
   For 2 mice per group, fix tissues in 5-10 mL 4% PFA.
   For 2 mice per group, homogenize tissues in Trizol

### Summary of Immune Assays to be Run on Samples

Serum—ELISAs, pseudovirus neutralization, secreted cytokine expression
Splenocytes—ELISPOT (intracellular cytokine expression), possibly flow cytometry (intracellular cytokines from identifiable T cell populations)
Leg muscles—IHC (spike protein expression)
PFA fixed organs—IHC (spike protein expression)
Tissue homogenates in Trizol—qRT-PCR (spike RNA expression)

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Titles, headings, or the like are provided to enhance the reader's comprehension of this document, and should not be read as limiting the scope of the present invention.

1. An expression vector that encodes all or a portion of replicon proteins from a positive stranded RNA virus.

2. The vector of claim 1, wherein the vector is a self-amplifying plasmid DNA vector or a sell-amplifying plasmid BNA vector.

3. (canceled)

4. The vector of claim 2, wherein expression of the replicon proteins is under the control of CMV and T7 promoters, and wherein expression of one or more payloads is under the independent control of sub-genomic promoters.

5. The vector of claim 1, wherein said positive stranded RNA virus is SARS-COV-2, Venezuelan Equine Encephalitis virus (VEEV) or Rubella virus (RUBV).

6-8. (canceled)

9. The vector of claim 1, wherein said vector encodes one or more payload.

10-11. (canceled)

12. The vector of claim 1 having the sequence as set forth in any one of SEQ ID NOs: 1 to 12.

13. A pharmaceutical composition comprising the vector of claim 1 and a pharmaceutically acceptable carrier and optionally an adjuvant.

14. The pharmaceutical composition of claim 13, wherein said vector is formulated in charge-altering releasable transporters (CARTs) or said vector is formulated in a lipid nanoparticle (LNP), optionally wherein said LNP comprises phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA).

15-16. (canceled)

17. A method of delivering a payload of interest to a cell, the method comprising contacting the cell with the vector of claim 9 which expresses the payload.

18-19. (canceled)

20. The method of claim 17, wherein said vector expresses a therapeutic polypeptide or RNA effective against an infectious agent and wherein said method treats, protects against and/or prevents disease associated with the infectious agent in a subject.

21. The method of claim 17, wherein said vector expresses one or more immunogens or epitopes from an infectious agent and wherein said method stimulates an antigen-specific immune response.

22. The method of claim 21, wherein said infectious agent is a positive stranded RNA virus and said vector expresses replicon proteins from the same positive stranded RNA virus.

23-26. (canceled)

27. The vector of claim 1 comprising a dual mammalian prokaryotic promoter.

28. The vector of claim 9, wherein at least one payload is a recombinant protein, siRNA, IncRNA, microRNA or an aptamer

29. The vector of claim 28, wherein said recombinant protein is an antibody, Bispecific T Cells Engager (BiTE), nanobody, chemokine, cytokine, growth factor, suicide protein such as thymidine kinase or angiogenesis inhibitors.

30. The method of claim 17, wherein said vector expresses an imaging agent.

31. The method of claim 30, wherein said imaging agent is a fluorescent protein.


SEQUENCE LISTING (22 sequences)

SEQ ID NO: 1 - DNA, 24818 bp, Artificial Sequence
  CMV+T7_FullCOVID vector
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaaccctgcagggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcctgcaggttaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagcgatcgcaacgccagcaacgcgagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattttgtaatacgactcactataggg

SEQ ID NO: 2 - DNA, 15581 bp, Artificial Sequence
  CMV+T7 RedCOVID
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaaccctgcagggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcctgcaggttaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagtttaaacttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagcgatcgcaacgccagcaacgcgagctccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagaattttgtaatacgactcactataggg

SEQ ID NO: 3 - DNA, 16319 bp, Artificial Sequence
  CBA_CoV2_SA
gcgatcgcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtaatacgactcactatagggggccggccattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcaactttacaaagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctccagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagactacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacttatatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacggcgcgccagaatttatacgtctcaatgctctagactcctgcaggatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcgagctcattaaggggtactgctgttatgtctttaaaagaaggtcaaatcaatgatatgattttatctcttcttagtaaaggtagacttataattagagaaaacaacagagttgttatttctagtgatgttcttgttaacaactaact

SEQ ID NO: 4 - DNA, 15153 bp, Artificial Sequence
  CMV +T7_Cov2_SA
gcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaatacgactcactatagggggccggccattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcaactttacaaagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctccagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagactacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacttatatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacggcgcgccagaatttatacgtctcaatgctctagactcctgcaggatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcgagctcattaaggggtactgctgttatgtctttaaaagaaggtcaaatcaatgatatgattttatctcttcttagtaaaggtagacttataattagagaaaacaacagagttgttatttctagtgatgttcttgttaacaactaact

SEQ ID NO: 5 - DNA, 17021 bp, Artificial Sequence
  CMV+T7_Cov2_SA_GFP
gcgatcgcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtaatacgactcactatagggggccggccattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcaactttacaaagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctccagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagactacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacttatatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacggcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagactcctgcaggatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgcaacgccagcaacgcgagctcattaaggggtactgctgttatgtctttaaaagaaggtcaaatcaatgatatgattttatctcttcttagtaaaggtagacttataattagagaaaacaacagagttgttatttctagtgatgttcttgttaacaactaact

SEQ ID NO: 6 - DNA, 11131 bp, Artificial Sequence
  CMV+T7-IRES_VEEV
atgggcggcgcaagagagaagcccaaaccaattacctacccaaacctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgccaccatggagaaagttcacgttgacatcgaggaagacagcccattcctcagagctttacaacggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcataagtatcattgcatctgtccgatgagatgtgcggaagatccggacagattgtacaagtatgcaactaagctgaagaaaaattgcaaggaaataactgacaaggaattggacaagaaaatgaaggagctcgccgccgtcatgagcgaccctgacctggaaactgagactatgtgcctccacgacgatgagtcatgtcgctacgaggggcaagtcgctgtttaccaggatgtatacgcagttgacggaccgacaagtctctatcaccaagccaacaagggagttagagtcgcctactggataggctttgacaccaccccttttatgtttaagaacttggctggagcatatccatcatactctaccaactgggccgacgaaaccgtgttaacggctcgtaacataggcctatgcagctccgacgtcatggagcggtcacgtagagggatgtccattcttaggaagaagtatttgaaaccatccaataatgtcctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgcatagtcgtcaacggtcgcacccaaagaaacaccaataccatgaagaattatcttttgcccgtagtggcccaggcatttgctaggtgggcaaaggaatataaggaagatcaagaagatgagaggccactaggactacgagatagacagttagtcatggggtgctgctgggcttttagaaggcacaagataacatctatttataagcgcccagatacccaaaccatcatcaaagtgaacagcgatttccactcattcgtgctgcccaggataggcagtaacacactggagatcgggctgagaacgagaatcaggaaaatgctagaagagcacaaggagccgtcacctctcattactgccgaggacatacaagaggctaagtgcgcagccgatgaggctaaggaagtgcgtgaagccgaggagctgcgcgctgctctaccacctttggcagctgattttgaggagcccactctggaagccgatgtcgacttgatgttacaagaggctggggccggctcagtggagacacctcgtggcttgataaaggttaccagctatgccggcgaggacaagatcggctcttacgcagtgctttctccacaggctgtactcaagagtgagaaactatcttgcattcaccctctcgctgaacaagtcatagtgataacacactctggccgaaaagggcgttatgccgtggaaccctaccatggaaaagtagtggtgccagagggacatgcaatacccgtccaggactttcaagctctgagtgaaagtgccaccatcgtgtacaacgaacgagagttcgtaaacaggtacctgcaccatattgccacacatggaggagcgctgaacacagatgaagaatattacaaaactgtcaagcccagcgagcacgacggcgaatacctgtacgacatcgacaggaaacaatgcgtcaagaaagaattagtcactgggctagggcttacaggcgagctggtggatcctcccttccatgaatttgcctacgagagtctgagaacacgtccggccgctccttaccaagtaccaaccataggggtgtatggcgtgccggggtcaggcaagtctggcatcattaaaagcgcagtcaccaaaaaagatctggtggtgagcgccaagaaagaaaactgcgcagaaataataagggacgtcaagaaaatgaaagggctggacgtcaatgccagaactgtggactcagtgctcttgaatggatgcaaacaccccgtagagaccctgtatattgacgaagcttttgcttgtcatgcaggcactctcagagcgctcatagccatcataagacctaaaaaggcagtgctctgcggggatccaaaacagtgtggctttttcaatatgatgtgcctgaaagtgcattttaaccacgagatttgcacgcaggtcttccacaaaagcatctctcgccgttgcactaaatccgtgacttcggtcgtctcaaccttgttttacgacaaaaggatgagaacgacgaacccgaaagagactaagattgtgattgacactactggcagtaccaaaccgaagcaggacgatctcattctcacttgtttcagagggtgggtgaagcagttgcaaatagattacaaaggcaacgaaataatgacggcagctgcctctcaagggctgacccgtaaaggcgtgtatgccgttcggtacaaggtgaatgaaaatcccctgtacgcacccacctcagaacatgtgaacgtcctactgacccgcacggaggaccgtatcgtgtggaaaacactagccggtgatccatggataaaaatactgacggccaagtatcctgggaacttcactgccacgatagaggaatggcaagcagagcatgatgccatcatgaggcacatcttggagagaccggaccctaccgacgttttccaaaataaggcgaacgtgtgttgggccaaggctttggtgccggtactgaagactgcaggcatagacatgaccactgaacaatggaacactgtggattacttcgaaacggacaaagctcactcagcagagatagtattgaaccaactatgcgtgaggttctttggactcgacctggactccggtctattttctgcacccactgttccgttatccattaggaataatcactgggataattccccgtcgcctaacatgtacgggttgaataaagaagtggtccgccagctctcccgcaggtacccacaactgcctcgagcagttgccaccggaagagtctatgacatgaacactggcacgctgcgcaattatgatccgcgcataaatctagtacctgtgaacagaagactgcctcatgctttagtcctccaccataatgaacacccacagagtgacttttcttcattcgtcagcaaactgaagggcagaactgtcttggtggtcggggagaagttgtccgtcccaggcaaaaaggtcgactggttgtcagaccagcctgaggctacctttagagctcggctggatttaggtatcccaggtgacgtgcccaaatacgacattgtatttattaacgtgaggactccatataaataccatcattatcagcagtgtgaagaccacgccattaagcttagtatgttgaccaagaaagcttgtctgcatttgaatcccggcggaacctgcgtcagcataggttatggttacgctgacagggccagcgagagcatcattggtgctatagcgcggcagttcaagttctcccgggtatgcaaaccgaaatcctcacatgaagagacagaagtactgtttgtattcattgggtacgatcgcaaggcccgtacgcacaatccttacaagctttcatctaccttgaccaacatctatacaggttccagactccacgaagccggatgcgcaccctcatatcatgtggtgcgaggggatattgccacggccaccgaaggagtgatcataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaagttgaaggggacaaacagttggcagaggcttatgagtccatcgctaaaattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatcagacgactcttcggtgacagaaccggatgcagagctggtgagggtacatccgaagagttctttggctggaaggaagggctacagcacaagtgatggcaagactttctcatatttggaagggaccaaatttcaccaggcggccaaggatatagcagaaattaatgccatgtggccagttgcaacggaggccaatgagcaagtatgcatgtatatcctcggtgaaagcatgagcagcattaggtcgaaatgccccgtcgaggagtcggaagcctccacaccacctagcacgctgccttgcttgtgcatccatgctatgactccagaaagagtacaacgcctaaaagcctcacgtccagaacaaattactgtgtgctcatcctttccattgccgaagtatagaatcactggtgtgcagaagatccagtgctcccagcctatactgttctcaccgaaggtgcctgcgtacattcatccacggaagtacctcgtggaaacaccaccggtagaagagactccggagtcgccggcagagaaccaatccacagaggggacacctgaacaaccagcacttgtaaacgtggatgcaaccaggactagaatgcctgaaccgatcatcattgaagaggaagaagaggatagtataagtttgctgtcagacggcccgacccaccaggtgctgcaagtcgaggcagacattcacgggtcgccttctgtatccagctcatcctggtccattcctcatgcatccgactttgatgtggacagcttatccatccttgacaccctggatggagctagcgtgaccagcggggcagtgtcagccgagactaactcctacttcgcaaggagcatggagtttcgggcgcgaccggtgcctgcgcctcgaaccgtattcaggaaccctccacatcccgcaccgcgcacaagaacaccgccacttgcacacagcagggccagctcgagaactagcctagtttccaccccgccaggcgtgaatagggtgattactagagaggagctcgaggcgcttaccccgtcccgcgctcctagcaggtcggcctcaagaactagcctggtctctaacccgccaggcgtaaatagggtgattacaagagaggagtttgaggcgttcgtagcacaacaacaatgacggtttgacgcgggtgcatacatcttttcctccgataccggtcaagggcatttacaacaaaaatcagtaaggcaaacggtgttatccgaagtggtgttggagaggaccgaattggagatttcgtatgccccgcgcctcgaccaggaaaaagaagaactactacgcaagaaattacagctgaatcccacacctgctaacagaagcagataccagtccaggagggtggagaatatgaaagccataacagctagacgtattctgcaaggcctagggcattatttgaaggcagaaggaaaagtggagtgctatcgaaccctgcatcctgttcctttgtattcatctagtgtgaatcgtgctttttcaagccccaaggtcgcagtggaagcctgcaatgccatgctgaaagaaaattttccgactgtagcttcctactgtattattccagagtacgatgcctatctggacatggttgacggcgcttcttgttgcttagacactgccagtttttgccctgcgaagctgcgcagctttccaaagaaacactcctatttggaacccacaatacggtcggcagtgccatcagcgattcagaacacgctccagaacgtcctggcagctgccacaaaaagaaattgcaacgtcacgcaaatgagagaattgcccgtattggattcggctgcctttaatgtggaatgcttcaagaaatatgcgtgcaataatgaatattgggaaacgtttaaagaaaaccccatcaggcttactgaagaaaatgtggtaaattacattactaaattaaaaggaccaaaagctgctgctctttttgcgaagacacataatttgaatatgttacaggacataccaatggacaggtttgtaatggacttaaagagggacgtgaaagtgactccaggaacaaaacatactgaagaacggcccaaggtacaggtgattcaggctgccgatccactagcgacagcggatctgtgcggaatccaccgggagttggttaggagattaaatgctgtcctgcttccgaacatccatacactgtttgacatgtcggctgaagactttgacgctattattgccgagcatttccagcctggggactgtgtactggaaactgacattgcgtcgtttgataaaagtgaggacgacgccatggctctgaccgcgttaatgattctggaagacctaggagtggacgcagagctgttgacgctgattgaggcggctttcggcgaaatatcatcaatacatttgcccaccaaaactaaatttaaattcggagccatgatgaaatccggaatgttcctcacactgtttgtgaacacagtcatcaacatcgtaatcgcaagcagagtgttaagagagcggctaaccggatcaccatgtgcagcattcattggagatgacaatatcgtgaaaggagtcaaatctgacaaattaatggcagacaggtgcgccacttggttgaacatggaagtcaagatcatagacgccgtggtgggcgagaaagcgccctatttttgtggagggtttatcttgtgtgactccgtgaccggcacagcgtgccgtgtggcagaccccctaaaaaggctgtttaagcttggcaaacccctggcagtagacgatgaacatgacgatgacaggagaagggcattacacgaagagtcaacacgctggaatcgagtgggaattcttccagagctgtgtaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaaggcgatcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcctgcaggtgaacatagcagcaattggcaagctgcttatatagaacttgcggcgattggcatgccgctttaaaattttattttattttcttttcttttccgaatcggattttgtttttaatatttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaagcggccgccgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtaacgccagcaacgcgagctcggccggcccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggccggcccgtttagtgaaccgtcagaattttgtaatacgactcactataggg

SEQ ID NO: 7 - DNA, 12543 bp, Artificial Sequence
  CMV+T7_VEE_SA_GFP
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccacctctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagggtttagtgaaccgtcagatccgctagtaatacgactcactatagggccggccataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 8 - DNA, 13263 bp, Artificial Sequence
  CMV+T7_VEE_SA_GFP
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagggtttagtgaaccgtcagatccgctagtaatacgactcactatagggccggccataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 9 - DNA, 11377 bp, Artificial Sequence
  CMV+T7_VEE_SA_GFP
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccacctctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagtaatacgactcactatagggccggccataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 10 - DNA, 13236 bp, Artificial Sequence
  CBA+T7_VEE_GFP
aattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggcttcagagcctctgctaaccatgttcatgccttcttctttttcctacagtaatacgactcactatagggccggccatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaa

SEQ ID NO: 11 - DNA, 25910 bp, Artificial Sequence
  CBA+T7-FullCovid-GFP
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaggccaccatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaagcgatcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcctgcaggatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtaacgccagcaacgcgagctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggcttcagagcctctgctaaccatgttcatgccttcttctttttcctacagggccggcccgtttagtgaaccgtcagaattttgtaatacgactcactataggg

SEQ ID NO: 12 - DNA, 16681 bp, Artificial Sequence
  CBA+T7-RedCovid-GFP
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaggccaccatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcaactttacaaagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaagcgatcgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcctgcaggatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagcggccgcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtaacgccagcaacgcgagctcggccggccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccagggggggggggggggggggggggggggggggggggggggggggggggggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggggggctcggggggggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggcttcagagcctctgctaaccatgttcatgccttcttctttttcctacagggccggcccgtttagtgaaccgtcagaattttgtaatacgactcactataggg

SEQ ID NO: 13 - DNA, 3849 bp, Artificial Sequence
  Sequence encoding payload Omicron spike protein without Furin cleavage site (FCS) and two amino acid substitutions with prolines
gccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatgctagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacatcacgactggaggaagaactgcgccgacgcctgactgaataa

SEQ ID NO: 14 - DNA, 20 bp, Artificial Sequence
  EGFP FWD primer
catgaagcagcacgacttct

SEQ ID NO: 15 - DNA, 22 bp, Artificial Sequence
  EGFP REV primer
ctgcttgtcggccatgatatag

SEQ ID NO: 16 - DNA, 12 bp, Artificial Sequence
  encodes the first 4 AAs of the MRS
gctgttttgcag

SEQ ID NO: 17 - DNA, 9 bp, Artificial Sequence
  TRS from SARS-CoV-2
taaacgaac

SEQ ID NO: 18 - DNA, 15 bp, Artificial Sequence
  Backtranslation of the PRS, ATLQA
gcaaccttacaagct

SEQ ID NO: 19 - DNA, 24 bp, Artificial Sequence
  TRS of VEEV
ctctctacggctaacctgaatgga

SEQ ID NO: 20 - DNA, 18 bp, Artificial Sequence
  Backtranslation of the PRS, QEAGAG
caagaggctggggccggc

SEQ ID NO: 21 - DNA, 39 bp, Artificial Sequence
  TRS of RUBV
gcctttaatcttacctactctaaccaggtcatcacccac

SEQ ID NO: 22 - DNA, 15 bp, Artificial Sequence
  Backtranslation of the PRS, LALAA
ctggccctggccgcc